(19) United States
(12) Patent Application Publication
(10) Pub. No.: US 2026/0243772 A1
(43) Pub. Date: Aug. 20, 2026

(54) BIOMARKERS AND USES THEREOF FOR TREATMENT OF CANCER WITH CD19-TARGETED ADOPTIVE CELL THERAPIES

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TAKEDA PHARMACEUTICAL COMPANY LIMITED, Osaka (JP)

(72) Inventors: Michel SADELAIN, New York, NY (US); Jae H. PARK, New York, NY (US); Varsha SUNDARESAN, Lexington, MA (US); Reshma SINGH, Lexington, MA (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TAKEDA PHARMACEUTICAL COMPANY LIMITED, Osaka (JP)

(21) Appl. No.: 19/665,177

(22) Filed: May 1, 2026

Related U.S. Application Data
(63) Continuation of application No. PCT/US2024/053498, filed on Oct. 30, 2024.
(60) Provisional application No. 63/595,170, filed on Nov. 1, 2023.

(51) Int. Cl. G01N 33/575; A61K 40/15; A61K 40/31; A61K 40/42; G01N 33/57505

(52) U.S. Cl. CPC G01N 33/57585; A61K 40/15; A61K 40/31; A61K 40/4211; G01N 33/57505; A61K 2239/48

(57) ABSTRACT

The presently disclosed subject matter provides methods for identifying subjects that are responsive or non-responsive to certain adoptive cell therapies or compositions comprising the same. In addition, methods for identifying subjects that have an increased likelihood or a reduced likelihood to response to certain adoptive cell therapies or compositions comprising the same are also provided. Moreover, the presently disclosed subject matter provides cells and compositions comprising a CD19-targeted chimeric receptor.

### CROSS-REFERENCE TO RELATED APPLICATIONS

This application is a Continuation of International Patent Application No. PCT/US2024/053498 filed Oct. 30, 2024, which claims priority to U.S. Provisional Application No. 63/595,170, filed Nov. 1, 2023, the contents of each of which are incorporated by reference in their entireties, and to which priority is claimed.

### SEQUENCE LISTING

The instant application contains a Sequence Listing which has been submitted herewith and is hereby incorporated by reference in its entirety. Said .xml copy, created on Apr. 21, 2026 is named 089339.0370.xml, and is 49,047 bytes in size. The Sequence Listing electronically filed herewith, does not extend beyond the scope of the specification and thus does not contain new matter.

### TECHNICAL FIELD

The presently disclosed subject matter provides methods for identifying subjects that are responsive or non-responsive to certain adoptive cell therapies or compositions comprising the same. In addition, methods for identifying subjects that have an increased likelihood or a reduced likelihood to respond to certain adoptive cell therapies or compositions comprising the same are also provided. Moreover, the presently disclosed subject matter provides cells and compositions comprising a CD19-targeted chimeric receptor.

### BACKGROUND

Cell-based immunotherapy is a therapy with curative potential for the treatment of cancer. T-cells and other immune cells may be modified to target tumor antigens through the introduction of genetic material coding for artificial or synthetic receptors for antigens, termed Chimeric Antigen Receptors (CARs), specific to selected antigens. Targeted T-cell therapy using CARs has shown recent clinical success in treating hematologic malignancies.

CD19-targeted chimeric antigen receptor (CAR) T cells have transformed the treatment of patients with relapsed or refractory CD19-positive hematologic malignancies (Park et al., N Engl J Med 378, 449-459 (2018); Lee et al., Lancet 385, 517-528 (2015); Davila et al., Science translational medicine 6, 224ra225 (2014); and Maude et al., N Engl J Med 378, 439-448 (2018)). Four CAR T cell therapies have been approved by the Food and Drug Administration for the treatment of CD19-positive hematologic malignancies (Maude et al., N Engl J Med 378, 439-448 (2018); Neelapu et al., N Engl J Med 377, 2531-2544 (2017); Schuster et al., N Engl J Med 380, 45-56 (2018); Wang et al., N Engl J Med 382, 1331-1342 (2020); Chong et al., N Engl J Med 384, 673-674 (2021); and Abramson et al., Lancet 396, 839-852 (2020)). Despite the promising results, most patients experience a lack of response or disease relapse, which can occur due to a lack of CAR T cell persistence, T cell exhaustion, or loss of the target antigen due to deletions spanning the CD19 locus, CAR masking or alternative splicing (Chong et al., N Engl J Med 384, 673-674 (2021); Sotillo et al., Cancer Discov 5, 1282-1295 (2015); Orlando et al., Nature medicine 24, 1504-1506 (2018); Ruella et al., Nature medicine 24, 1499-1503 (2018); Spiegel et al., Blood 137, 1832-1835 (2021)). Accordingly, there remains a need for identifying biomarkers to enable more targeted use of CD19-targeted chimeric receptors and cells comprising the same in cancer patients.

### SUMMARY OF THE INVENTION

In certain non-limiting embodiments, the presently disclosed subject matter provides a method comprising a) measuring an expression level of at least one of CD57, TIM3, KLRG1, CTLA4, LAG3 and PD1 in an immunoresponsive cell of a subject; b) comparing the expression level to a reference sample; and c) identifying the subject as responsive or as having an increased likelihood to respond to an adoptive cell therapy. In certain embodiments, the subject is responsive or has an increased likelihood to respond to the adoptive cell therapy if a) the expression level of CD57 is increased compared to the reference sample; b) the expression level of TIM3 is increased compared to the reference sample; c) the expression level of KLRG1 is reduced compared to the reference sample; d) the expression level of CTLA4 is reduced compared to the reference sample; e) the expression level of LAG3 is reduced compared to the reference sample; and/or f) the expression level of PD1 is increased compared to the reference sample.

In certain non-limiting embodiments, the presently disclosed subject matter provides a method comprising a) measuring the expression level of at least one of CD57, TIM3, KLRG1, CTLA4, LAG3 and PD1 in an immunoresponsive cell of a subject; b) comparing the expression level to a reference sample; and c) identifying the subject as non-responsive or as having a reduced likelihood to respond to an adoptive cell therapy. In certain embodiments, the subject is non-responsive or has a reduced likelihood to respond the adoptive cell therapy if a) the expression level of CD57 is reduced compared to the reference sample; b) the expression level of TIM3 is reduced compared to the reference sample; c) the expression level of KLRG1 is increased compared to the reference sample; d) the expression level of CTLA4 is increased compared to the reference sample; e) the expression level of LAG3 is increased compared to the reference sample; and/or f) the expression level of PD1 is reduced compared to the reference sample.

In certain embodiments, the adoptive cell therapy comprises a cell comprising a CD19-targeted chimeric receptor.

In certain embodiments, the methods disclosed herein further comprise administering to the subject a cell comprising a CD19-targeted chimeric receptor or a composition comprising thereof. In certain embodiments, the CD19-targeted chimeric receptor comprises an extracellular antigen-binding domain comprising a heavy chain variable region (V_{H}) comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12, and a light chain variable region (V_{L}) comprising a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15.

In certain embodiments, the extracellular antigen-binding domain comprises a scFv. In certain embodiments, the heavy chain variable region comprises an amino acid sequence that is at least about 80% identical to the amino acid sequence set forth in SEQ ID NO: 16; and the heavy chain variable region comprises an amino acid sequence that is at least about 80% identical to the amino acid sequence set forth in SEQ ID NO: 17. In certain embodiments, the heavy chain variable region comprises the amino acid sequence set forth in SEQ ID NO: 16; and the heavy chain variable region comprises the amino acid sequence set forth in SEQ ID NO: 17.

In certain embodiments, the chimeric receptor comprises a transmembrane domain comprising a CD28 polypeptide. In certain embodiments, the chimeric receptor comprises intracellular signaling domain comprises a CD3ζ polypeptide comprising a native ITAM1, an ITAM2 variant consisting of two loss-of-function mutations, and an ITAM3 consisting of two loss-of-function mutations. In certain embodiments, the native ITAM1 consists of the amino acid sequence set forth in SEQ ID NO: 28, the ITAM2 variant consists of the amino acid sequence set forth in SEQ ID NO: 34, and the ITAM3 variant consists of the amino acid sequence set forth in SEQ ID NO: 38. In certain embodiments, the chimeric receptor comprises an intracellular signaling domain comprising a CD3ζ polypeptide comprising the amino acid sequence set forth in SEQ ID NO: 40. In certain embodiments, the intracellular signaling domain further comprises a CD28 polypeptide. In certain embodiments, the chimeric receptor comprises the amino acid sequence set forth in SEQ ID NO: 45.

In certain embodiments, the chimeric receptor is expressed from a vector. In certain embodiments, the vector is a retroviral vector. In certain embodiments, the chimeric receptor is constitutively expressed on the surface of the cell.

In certain embodiments, the cell comprising the CD19-targeted chimeric receptor is an immunoresponsive cell. In certain embodiments, the cell comprising the CD19-targeted chimeric receptor is a cell of the lymphoid lineage or a cell of the myeloid lineage. In certain embodiments, the cell comprising the CD19-targeted chimeric receptor is selected from the group consisting of a T cell, a Natural Killer (NK) cell, a stem cell from which lymphoid cells may be differentiated, and a stem cell from which myeloid cells may be differentiated. In certain embodiments, the cell comprising the CD19-targeted chimeric receptor is a T cell. In certain embodiments, the T cell is selected from the group consisting of helper T cells, cytotoxic T cells, memory T cells, regulatory T cells, tumor-infiltrating lymphocyte (TIL), Natural Killer T cells, mucosal associated invariant T cells, and γδ T cells. In certain embodiments, the cell comprising the CD19-targeted chimeric receptor is a NK cell. In certain embodiments, the cell comprising the CD19-targeted chimeric receptor is derived from a stem cell. In certain embodiments, the stem cell is a pluripotent stem cell. In certain embodiments, the pluripotent stem cell is an embryoid stem cell or an induced pluripotent stem cell.

In certain embodiments, the cell comprising the CD19-targeted chimeric receptor is selected from a CD57⁺ T cell, a TIM3⁺ T cell, a KLRG1⁻ T cell, a CTLA4⁻ T cell, a LAG3⁻ T cell, a PD1⁺ T cell, a CD57⁺ TIM3⁺ T cell, a CD57⁺ KLRG1⁻ T cell, a CD57⁺ CTLA4⁻ T cell, a CD57⁺ LAG3⁻ T cell, a CD57⁺ PD1⁺ T cell, a TIM3⁺ KLRG1⁻ T cell, a TIM3⁺ CTLA4⁻ T cell, a TIM3⁺ LAG3⁻ T cell, a TIM3⁺ PD1⁺ T cell, a KLRG1⁻ CTLA4⁻ T cell, a KLRG1⁻ LAG3⁻ T cell, a KLRG1⁻ PD1⁺ T cell, a CTLA4⁻ LAG3⁻ T cell, a CTLA4⁻ PD1⁺ T cell, a LAG3⁻ PD1⁺ T cell, a CD57⁺ TIM3⁺ KLRG1⁻ T cell, a CD57⁺ TIM3⁺ CTLA4⁻ T cell, a CD57⁺ TIM3⁺ LAG3⁻ T cell, a CD57⁺ TIM3⁺ PD1⁺ T cell, a CD57⁺ KLRG1⁻ CTLA4⁻ T cell, a CD57⁺ KLRG1⁻ LAG3⁻ T cell, a CD57⁺ KLRG1⁻ PD1⁺ T cell, a CD57⁺ CTLA4⁻ LAG3⁻ T cell, a CD57⁺ CTLA4⁻ PD1⁺ T cell, a CD57⁺ LAG3⁻ PD1⁺ T cell, a TIM3⁺ KLRG1⁻ CTLA4⁻ T cell, a TIM3⁺ KLRG1⁻ LAG3⁻ T cell, a TIM3⁺ CTLA4⁻ LAG3⁻ T cell, a TIM3⁺ KLRG1⁻ PD1⁺ T cell, a TIM3⁺ CTLA4⁻ LAG3⁻ T cell, a TIM3⁺ CTLA4⁻ PD1⁺ T cell, a TIM3⁺ LAG3⁻ PD1⁺ T cell, a KLRG1⁻ CTLA4⁻ LAG3⁻ T cell, a KLRG1⁻ CTLA4⁻ PD1⁺ T cell, a KLRG1⁻ LAG3⁻ PD1⁺ T cell, a CTLA4⁻ LAG3⁻ PD1⁺ T cell, a CD57⁺ TIM3⁺ KLRG1⁻ CTLA4⁻ T cell, a CD57⁺ TIM3⁺ KLRG1⁻ LAG3⁻ T cell, a CD57⁺ TIM3⁺ KLRG1⁻ PD1⁺ T cell, a CD57⁺ TIM3⁺ CTLA4⁻ LAG3⁻ T cell, a CD57⁺ TIM3⁺ CTLA4⁻ PD1⁺ T cell, a CD57⁺ TIM3⁺ LAG3⁻ PD1⁺ T cell, a CD57⁺ KLRG1⁻ CTLA4⁻ LAG3⁻ T cell, a CD57⁺ KLRG1⁻ CTLA4⁻ PD1⁺ T cell, a CD57⁺ KLRG1⁻ LAG3⁻ PD1⁺ T cell, a CD57⁺ CTLA4⁻ LAG3⁻ PD1⁺ T cell, a TIM3⁺ KLRG1⁻ CTLA4⁻ LAG3⁻ T cell, a TIM3⁺ KLRG1⁻ CTLA4⁻ LAG3⁻ T cell, a TIM3⁺ KLRG1⁻ CTLA4⁻ PD1⁺ T cell, a TIM3⁺ KLRG1⁻ LAG3⁻ PD1⁺ T cell, a TIM3⁺ CTLA4⁻ LAG3⁻ PD1⁺ T cell, a KLRG1⁻ CTLA4⁻ LAG3⁻ PD1⁺ T cell, a CD57⁺ TIM3⁺ KLRG1⁻ CTLA4⁻ LAG3⁻ T cell, a CD57⁺ TIM3⁺ KLRG1⁻ CTLA4⁻ PD1⁺ T cell, a CD57⁺ TIM3⁺ KLRG1⁻ LAG3⁻ PD1⁺ T cell, a CD57⁺ TIM3⁺ CTLA4⁻ LAG3⁻ PD1⁺ T cell, a CD57⁺ KLRG1⁻ CTLA4⁻ LAG3⁻ PD1⁺ T cell, a TIM3⁺ KLRG1⁻ CTLA4⁻ LAG3⁻ PD1⁺ T cell, a CD57⁺ TIM3⁺ KLRG1⁻ CTLA4⁻ LAG3⁻ PD1⁺ T cell, or a combination thereof.

In certain embodiments, the composition comprises at least about 5% of a CD57⁺ T cell comprising the CD19-targeted chimeric receptor, at least about 5% of a TIM3⁺ T cell comprising the CD19-targeted chimeric receptor, at least about 5% of a KLRG1⁻ T cell comprising the CD19-targeted chimeric receptor, at least about 5% of a CTLA4⁻ T cell comprising the CD19-targeted chimeric receptor, at least about 5% of a LAG3⁻ T cell comprising the CD19-targeted chimeric receptor, at least about 5% of a PD1⁺ T cell comprising the CD19-targeted chimeric receptor, at least about 5% of a CD57⁺ TIM3⁺ T cell comprising the CD19-targeted chimeric receptor, at least about 5% of a CD57⁺ KLRG1⁻ T cell comprising the CD19-targeted chimeric receptor, at least about 5% of a CD57⁺ CTLA4⁻ T cell comprising the CD19-targeted chimeric receptor, at least about 5% of a CD57⁺ LAG3⁻ T cell comprising the CD19-targeted chimeric receptor, at least about 5% of a CD57⁺ PD1⁺ T cell comprising the CD19-targeted chimeric receptor, at least about 5% of a TIM3⁺ KLRG1⁻ T cell comprising the CD19-targeted chimeric receptor, at least about 5% of a TIM3⁺ CTLA4⁻ T cell comprising the CD19-targeted chimeric receptor, at least about 5% of a TIM3⁺ LAG3⁻ T cell comprising the CD19-targeted chimeric receptor, at least about 5% of a TIM3⁺ PD1⁺ T cell comprising the CD19-targeted chimeric receptor, at least about 5% of a KLRG1⁻ CTLA4⁻ T cell comprising the CD19-targeted chimeric receptor, at least about 5% of a KLRG1⁻ LAG3⁻ T cell comprising the CD19-targeted chimeric receptor, at least about 5% of a KLRG1⁻ PD1⁺ T cell comprising the CD19-targeted chimeric receptor, at least about 5% of a CTLA4⁻ LAG3⁻ T cell comprising the CD19-targeted chimeric receptor, at least about 5% of a CTLA4⁻ PD1⁺ T cell comprising the CD19-targeted chimeric receptor, at least about 5% of a LAG3⁻ PD1⁺ T cell comprising the CD19-targeted chimeric receptor, at least about 5% of a CD57⁺ TIM3⁺ KLRG1⁻ T cell comprising the CD19-targeted chimeric receptor, at least about 5% of a CD57⁺ TIM3⁺ CTLA4⁻ T cell comprising the CD19-targeted chimeric receptor, at least about 5% of a CD57⁺ TIM3⁺ LAG3⁻ T cell comprising the CD19-targeted chimeric receptor, at least about 5% of a CD57⁺ TIM3⁺ PD1⁺ T cell comprising the CD19-targeted chimeric receptor, at least about 5% of a CD57⁺ KLRG1⁻ CTLA4⁻ T cell comprising the CD19-targeted chimeric receptor, at least about 5% of a CD57⁺ KLRG1⁻ LAG3⁻ T cell comprising the CD19-targeted chimeric receptor, at least about 5% of a CD57⁺ KLRG1⁻ PD1⁺ T cell comprising the CD19-targeted chimeric receptor, at least about 5% of a CD57⁺ CTLA4⁻ LAG3⁻ T cell comprising the CD19-targeted chimeric receptor, at least about 5% of a CD57⁺ CTLA4⁻ PD1⁺ T cell comprising the CD19-targeted chimeric receptor, at least about 5% of a CD57⁺ LAG3⁻ PD1⁺ T cell comprising the CD19-targeted chimeric receptor, at least about 5% of a TIM3⁺ KLRG1⁻ CTLA4⁻ T cell comprising the CD19-targeted chimeric receptor, at least about 5% of a TIM3⁺ KLRG1⁻ LAG3⁻ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ KLRG1⁻ PD1⁺ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ CTLA4⁻ PD1⁺ T cell comprising the CD19-targeted chimeric receptor, at least about 5% of a TIM3⁺ LAG3⁻ PD1⁺ T cell comprising the CD19-targeted chimeric receptor, at least about 5% of a KLRG1⁻ CTLA4⁻ LAG3⁻ T cell comprising the CD19-targeted chimeric receptor, at least about 5% of a KLRG1⁻ CTLA4⁻ PD1⁺ T cell comprising the CD19-targeted chimeric receptor, at least about 5% of a KLRG1⁻ LAG3⁻ PD1⁺ T cell comprising the CD19-targeted chimeric receptor, at least about 5% of a CTLA4⁻ LAG3⁻ PD1⁺ T cell comprising the CD19-targeted chimeric receptor, at least about 5% of a CD57⁺ TIM3⁺ KLRG1⁻ CTLA4⁻ T cell comprising the CD19-targeted chimeric receptor, at least about 5% of a CD57⁺ TIM3⁺ KLRG1⁻ LAG3⁻ T cell comprising the CD19-targeted chimeric receptor, at least about 5% of a CD57⁺ TIM3⁺ KLRG1⁻ PD1⁺ T cell comprising the CD19-targeted chimeric receptor, at least about 5% of a CD57⁺ TIM3⁺ CTLA4⁻ LAG3⁻ T cell comprising the CD19-targeted chimeric receptor, at least about 5% of a CD57⁺ TIM3⁺ CTLA4⁻ PD1⁺ T cell comprising the CD19-targeted chimeric receptor, at least about 5% of a CD57⁺ TIM3⁺ LAG3⁻ PD1⁺ T cell comprising the CD19-targeted chimeric receptor, at least about 5% of a CD57⁺ KLRG1⁻ CTLA4⁻ LAG3⁻ T cell comprising the CD19-targeted chimeric receptor, at least about 5% of a CD57⁺ KLRG1⁻ CTLA4⁻ PD1⁺ T cell comprising the CD19-targeted chimeric receptor, at least about 5% of a CD57⁺ KLRG1⁻ LAG3⁻ PD1⁺ T cell comprising the CD19-targeted chimeric receptor, at least about 5% of a CD57⁺ CTLA4⁻ LAG3⁻ PD1⁺ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ PD1⁺ T cell comprising the CD19-targeted chimeric receptor, at least about 5% of a TIM3⁺ KLRG1⁻ LAG3⁻ PD1⁺ T cell comprising the CD19-targeted chimeric receptor, at least about 5% of a TIM3⁺ CTLA4⁻ LAG3⁻ PD1⁺ T cell comprising the CD19-targeted chimeric receptor, at least about 5% of a KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ T cell comprising the CD19-targeted chimeric receptor, at least about 5% of a CD57⁺ TIM3⁺ KLRG1⁻ CTLA4⁻ PD1⁺ T cell comprising the CD19-targeted chimeric receptor, at least about 5% of a CD57⁺ TIM3⁺ KLRG1⁻ LAG3⁻ PD1⁺ T cell comprising the CD19-targeted chimeric receptor, at least about 5% of a CD57⁺ TIM3⁺ CTLA4⁻ LAG3⁻ PD1⁺ T cell comprising the CD19-targeted chimeric receptor, at least about 5% of a CD57⁺ KLRG1⁻ CTLA4⁻ LAG3⁻ PD1⁺ T cell comprising the CD19-targeted chimeric receptor, at least about 5% of a TIM3⁺ KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ PD1⁺ T cell comprising the CD19-targeted chimeric receptor, or a combination thereof.

In certain embodiments, the subject has a tumor and/or a neoplasm associated with CD19. In certain embodiments, the tumor and/or neoplasm is a blood cancer. In certain embodiments, the blood cancer is selected from the group consisting of multiple myeloma, leukemia, and lymphomas. In certain embodiments, the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. In certain embodiments, the lymphoma is Hodgkin's lymphoma or non-Hodgkin's lymphoma. In certain embodiments, the tumor and/or neoplasm is a B cell malignancy. In certain embodiments, the B cell malignancy is selected from the group consisting of B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma. In certain embodiments, the tumor and/or neoplasm is B cell lymphoma. In certain embodiments, the B cell lymphoma is relapsed or refractory (R/R) B cell lymphoma.

In certain embodiments, the expression level of at least one of CD57, TIM3, KLRG1, CTLA4, LAG3 and PD1 is measured on the surface of the immunoresponsive cell. In certain embodiments, the expression level of at least one of CD57, TIM3, KLRG1, CTLA4, LAG3 and PD1 is measured by cytometry or by flow-cytometry. In certain embodiments, the immunoresponsive cell is a T cell. In certain embodiments, the T cell is selected from the group consisting of a helper T cell, a cytotoxic T cell, a memory T cell, a central memory T cell, a stem-like memory T cell, an effector memory T cell, a regulatory T cell, a tumor-infiltrating lymphocyte (TIL), a Natural killer T cells, a γδ T cell, a CD4⁺ T cells, and a CD8⁺ T cell. In certain embodiments, the T cell is a CD4⁺ T cell or a CD8⁺ T cell. In certain embodiments, the immunoresponsive cell is obtained from a sample of the subject. In certain embodiments, the sample is selected from bone marrow, thymus, tumor biopsy, tumor fragments, enzymatically digested tumor tissue, dissociated/suspended cells, a lymph node sample, or a bodily fluid sample. In certain embodiments, the bodily fluid sample is a blood sample, an ascites sample, or a lymph sample. In certain embodiments, the subject is a human subject.

In certain non-limiting embodiments, the presently disclosed subject matter provides a composition comprising a cell comprising a CD19-targeted chimeric receptor, wherein the CD19-targeted chimeric receptor comprises an extracellular antigen-binding domain, a transmembrane domain, and an intracellular domain, wherein the extracellular antigen-binding domain comprises a heavy chain variable region (V_{H}) comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12, and a light chain variable region (V_{L}) comprising a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15.

In certain embodiments, the extracellular antigen-binding domain comprises a scFv. In certain embodiments, the heavy chain variable region comprises an amino acid sequence that is at least about 80% identical to the amino acid sequence set forth in SEQ ID NO: 16; and the heavy chain variable region comprises an amino acid sequence that is at least about 80% identical to the amino acid sequence set forth in SEQ ID NO: 17. In certain embodiments, the heavy chain variable region comprises the amino acid sequence set forth in SEQ ID NO: 16; and the heavy chain variable region comprises the amino acid sequence set forth in SEQ ID NO: 17.

In certain embodiments, the transmembrane domain comprises a CD28 polypeptide. In certain embodiments, the intracellular signaling domain comprises a CD3ζ polypeptide comprising a native ITAM1, an ITAM2 variant consisting of two loss-of-function mutations, and an ITAM3 consisting of two loss-of-function mutations. In certain embodiments, the native ITAM1 consists of the amino acid sequence set forth in SEQ ID NO: 28, the ITAM2 variant consists of the amino acid sequence set forth in SEQ ID NO: 34, and the ITAM3 variant consists of the amino acid sequence set forth in SEQ ID NO: 38. In certain embodiments, the intracellular signaling domain comprises a CD3ζ polypeptide comprising the amino acid sequence set forth in SEQ ID NO: 40. In certain embodiments, the intracellular signaling domain further comprises a CD28 polypeptide. In certain embodiments, the chimeric receptor comprises the amino acid sequence set forth in SEQ ID NO: 45.

In certain embodiments, the chimeric receptor is expressed from a vector. In certain embodiments, the vector is a retroviral vector. In certain embodiments, the chimeric receptor is constitutively expressed on the surface of the cell. In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, a stem cell from which lymphoid cells may be differentiated, and a stem cell from which myeloid cells may be differentiated. In certain embodiments, the cell is a T cell. In certain embodiments, the T cell is selected from the group consisting of helper T cells, cytotoxic T cells, memory T cells, regulatory T cells, tumor-infiltrating lymphocyte (TIL), Natural Killer T cells, mucosal associated invariant T cells, and γδ T cells. In certain embodiments, the cell is an NK cell. In certain embodiments, the cell is derived from a stem cell. In certain embodiments, the stem cell is a pluripotent stem cell. In certain embodiments, the pluripotent stem cell is an embryoid stem cell or an induced pluripotent stem cell.

In certain embodiments, the the cell is selected from a CD57⁺ T cell, a TIM3⁺ T cell, a KLRG1⁻ T cell, a CTLA4⁻ T cell, a LAG3⁻ T cell, a PD1⁺ T cell, a CD57⁺ TIM3⁺ T cell, a CD57⁺ KLRG1⁻ T cell, a CD57⁺ CTLA4⁻ T cell, a CD57⁺ LAG3⁻ T cell, a CD57⁺ PD1⁺ T cell, a TIM3⁺ KLRG1⁻ T cell, a TIM3⁺ CTLA4⁻ T cell, a TIM3⁺ LAG3⁻ T cell, a TIM3⁺ PD1⁺ T cell, a KLRG1⁻ CTLA4⁻ T cell, a KLRG1⁻ LAG3⁻ T cell, a KLRG1⁻ PD1⁺ T cell, a CTLA4⁻ LAG3⁻ T cell, a CTLA4⁻ PD1⁺ T cell, a LAG3⁻ PD1⁺ T cell, a CD57⁺ TIM3⁺ KLRG1⁻ T cell, a CD57⁺ TIM3⁺ CTLA4⁻ T cell, a CD57⁺ TIM3⁺ LAG3⁻ T cell, a CD57⁺ TIM3⁺ PD1⁺ T cell, a CD57⁺ KLRG1⁻ CTLA4⁻ T cell, a CD57⁺ KLRG1⁻ LAG3⁻ T cell, a CD57⁺ KLRG1⁻ PD1⁺ T cell, a CD57⁺ CTLA4⁻ LAG3⁻ T cell, a CD57⁺ CTLA4⁻ PD1⁺ T cell, a CD57⁺ LAG3⁻ PD1⁺ T cell, a TIM3⁺ KLRG1⁻ CTLA4⁻ T cell, a TIM3⁺ KLRG1⁻ LAG3⁻ T cell, a TIM3⁺ CTLA4⁻ LAG3⁻ T cell, a TIM3⁺ KLRG1⁻ PD1⁺ T cell, a TIM3⁺ CTLA4⁻ LAG3⁻ T cell, a TIM3⁺ CTLA4⁻ PD1⁺ T cell, a TIM3⁺ LAG3⁻ PD1⁺ T cell, a KLRG1⁻ CTLA4⁻ LAG3⁻ T cell, a KLRG1⁻ CTLA4⁻ PD1⁺ T cell, a KLRG1⁻ LAG3⁻ PD1⁺ T cell, a CTLA4⁻ LAG3⁻ PD1⁺ T cell, a CD57⁺ TIM3⁺ KLRG1⁻ CTLA4⁻ T cell, a CD57⁺ TIM3⁺ KLRG1⁻ LAG3⁻ T cell, a CD57⁺ TIM3⁺ KLRG1⁻ PD1⁺ T cell, a CD57⁺ TIM3⁺ CTLA4⁻ LAG3⁻ T cell, a CD57⁺ TIM3⁺ CTLA4⁻ PD1⁺ T cell, a CD57⁺ TIM3⁺ LAG3⁻ PD1⁺ T cell, a CD57⁺ KLRG1⁻ CTLA4⁻ LAG3⁻ T cell, a CD57⁺ KLRG1⁻ CTLA4⁻ PD1⁺ T cell, a CD57⁺ KLRG1⁻ LAG3⁻ PD1⁺ T cell, a CD57⁺ CTLA4⁻ LAG3⁻ PD1⁺ T cell, a TIM3⁺ KLRG1⁻ CTLA4⁻ LAG3⁻ T cell, a TIM3⁺ KLRG1⁻ CTLA4⁻ LAG3⁻ T cell, a TIM3⁺ KLRG1⁻ CTLA4⁻ PD1⁺ T cell, a TIM3⁺ KLRG1⁻ LAG3⁻ PD1⁺ T cell, a TIM3⁺ CTLA4⁻ LAG3⁻ PD1⁺ T cell, a KLRG1⁻ CTLA4⁻ LAG3⁻ PD1⁺ T cell, a CD57⁺ TIM3⁺ KLRG1⁻ CTLA4⁻ LAG3⁻ T cell, a CD57⁺ TIM3⁺ KLRG1⁻ CTLA4⁻ PD1⁺ T cell, a CD57⁺ TIM3⁺ KLRG1⁻ LAG3⁻ PD1⁺ T cell, a CD57⁺ TIM3⁺ CTLA4⁻ LAG3⁻ PD1⁺ T cell, a CD57⁺ KLRG1⁻ CTLA4⁻ LAG3⁻ PD1⁺ T cell, a TIM3⁺ KLRG1⁻ CTLA4⁻ LAG3⁻ PD1⁺ T cell, a CD57⁺ TIM3⁺ KLRG1⁻ CTLA4⁻ LAG3⁻ PD1⁺ T cell, or a combination thereof.

In certain embodiments, the composition comprises at least about 5% of a CD57⁺ T cell comprising the CD19-targeted chimeric receptor, at least about 5% of a TIM3⁺ T cell comprising the CD19-targeted chimeric receptor, at least about 5% of a KLRG1⁻ T cell comprising the CD19-targeted chimeric receptor, at least about 5% of a CTLA4⁻ T cell comprising the CD19-targeted chimeric receptor, at least about 5% of a LAG3⁻ T cell comprising the CD19-targeted chimeric receptor, at least about 5% of a PD1⁺ T cell comprising the CD19-targeted chimeric receptor, at least about 5% of a CD57⁺ TIM3⁺ T cell comprising the CD19-targeted chimeric receptor, at least about 5% of a CD57⁺ KLRG1⁻ T cell comprising the CD19-targeted chimeric receptor, at least about 5% of a CD57⁺ CTLA4⁻ T cell comprising the CD19-targeted chimeric receptor, at least about 5% of a CD57⁺ LAG3⁻ T cell comprising the CD19-targeted chimeric receptor, at least about 5% of a CD57⁺ PD1⁺ T cell comprising the CD19-targeted chimeric receptor, at least about 5% of a TIM3⁺ KLRG1⁻ T cell comprising the CD19-targeted chimeric receptor, at least about 5% of a TIM3⁺ CTLA4⁻ T cell comprising the CD19-targeted chimeric receptor, at least about 5% of a TIM3⁺ LAG3⁻ T cell comprising the CD19-targeted chimeric receptor, at least about 5% of a TIM3⁺ PD1⁺ T cell comprising the CD19-targeted chimeric receptor, at least about 5% of a KLRG1⁻ CTLA4⁻ T cell comprising the CD19-targeted chimeric receptor, at least about 5% of a KLRG1⁻ LAG3⁻ T cell comprising the CD19-targeted chimeric receptor, at least about 5% of a KLRG1⁻ PD1⁺ T cell comprising the CD19-targeted chimeric receptor, at least about 5% of a CTLA4⁻ LAG3⁻ T cell comprising the CD19-targeted chimeric receptor, at least about 5% of a CTLA4⁻ PD1⁺ T cell comprising the CD19-targeted chimeric receptor, at least about 5% of a LAG3⁻ PD1⁺ T cell comprising the CD19-targeted chimeric receptor, at least about 5% of a CD57⁺ TIM3⁺ KLRG1⁻ T cell comprising the CD19-targeted chimeric receptor, at least about 5% of a CD57⁺ TIM3⁺ CTLA4⁻ T cell comprising the CD19-targeted chimeric receptor, at least about 5% of a CD57⁺ TIM3⁺ LAG3⁻ T cell comprising the CD19-targeted chimeric receptor, at least about 5% of a CD57⁺ TIM3⁺ PD1⁺ T cell comprising the CD19-targeted chimeric receptor, at least about 5% of a CD57⁺ KLRG1⁻ CTLA4⁻ T cell comprising the CD19-targeted chimeric receptor, at least about 5% of a CD57⁺ KLRG1⁻ LAG3⁻ T cell comprising the CD19-targeted chimeric receptor, at least about 5% of a CD57⁺ KLRG1⁻ PD1⁺ T cell comprising the CD19-targeted chimeric receptor, at least about 5% of a CD57⁺ CTLA4⁻ LAG3⁻ T cell comprising the CD19-targeted chimeric receptor, at least about 5% of a CD57⁺ CTLA4⁻ PD1⁺ T cell comprising the CD19-targeted chimeric receptor, at least about 5% of a CD57⁺ LAG3⁻ PD1⁺ T cell comprising the CD19-targeted chimeric receptor, at least about 5% of a TIM3⁺ KLRG1⁻ CTLA4⁻ T cell comprising the CD19-targeted chimeric receptor, at least about 5% of a TIM3⁺ KLRG1⁻ LAG3⁻ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ KLRG1⁻ PD1⁺ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ CTLA4⁻ PD1⁺ T cell comprising the CD19-targeted chimeric receptor, at least about 5% of a TIM3⁺ LAG3⁻ PD1⁺ T cell comprising the CD19-targeted chimeric receptor, at least about 5% of a KLRG1⁻ CTLA4⁻ LAG3⁻ T cell comprising the CD19-targeted chimeric receptor, at least about 5% of a KLRG1⁻ CTLA4⁻ PD1⁺ T cell comprising the CD19-targeted chimeric receptor, at least about 5% of a KLRG1⁻ LAG3⁻ PD1⁺ T cell comprising the CD19-targeted chimeric receptor, at least about 5% of a CTLA4⁻ LAG3⁻ PD1⁺ T cell comprising the CD19-targeted chimeric receptor, at least about 5% of a CD57⁺ TIM3⁺ KLRG1⁻ CTLA4⁻ T cell comprising the CD19-targeted chimeric receptor, at least about 5% of a CD57⁺ TIM3⁺ KLRG1⁻ LAG3⁻ T cell comprising the CD19-targeted chimeric receptor, at least about 5% of a CD57⁺ TIM3⁺ KLRG1⁻ PD1⁺ T cell comprising the CD19-targeted chimeric receptor, at least about 5% of a CD57⁺ TIM3⁺ CTLA4⁻ LAG3⁻ T cell comprising the CD19-targeted chimeric receptor, at least about 5% of a CD57⁺ TIM3⁺ CTLA4⁻ PD1⁺ T cell comprising the CD19-targeted chimeric receptor, at least about 5% of a CD57⁺ TIM3⁺ LAG3⁻ PD1⁺ T cell comprising the CD19-targeted chimeric receptor, at least about 5% of a CD57⁺ KLRG1⁻ CTLA4⁻ LAG3⁻ T cell comprising the CD19-targeted chimeric receptor, at least about 5% of a CD57⁺ KLRG1⁻ CTLA4⁻ PD1⁺ T cell comprising the CD19-targeted chimeric receptor, at least about 5% of a CD57⁺ KLRG1⁻ LAG3⁻ PD1⁺ T cell comprising the CD19-targeted chimeric receptor, at least about 5% of a CD57⁺ CTLA4⁻ LAG3⁻ PD1⁺ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ PD1⁺ T cell comprising the CD19-targeted chimeric receptor, at least about 5% of a TIM3⁺ KLRG1⁻ LAG3⁻ PD1⁺ T cell comprising the CD19-targeted chimeric receptor, at least about 5% of a TIM3⁺ CTLA4⁻ LAG3⁻ PD1⁺ T cell comprising the CD19-targeted chimeric receptor, at least about 5% of a KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ T cell comprising the CD19-targeted chimeric receptor, at least about 5% of a CD57⁺ TIM3⁺ KLRG1⁻ CTLA4⁻ PD1⁺ T cell comprising the CD19-targeted chimeric receptor, at least about 5% of a CD57⁺ TIM3⁺ KLRG1⁻ LAG3⁻ PD1⁺ T cell comprising the CD19-targeted chimeric receptor, at least about 5% of a CD57⁺ TIM3⁺ CTLA4⁻ LAG3⁻ PD1⁺ T cell comprising the CD19-targeted chimeric receptor, at least about 5% of a CD57⁺ KLRG1⁻ CTLA4⁻ LAG3⁻ PD1⁺ T cell comprising the CD19-targeted chimeric receptor, at least about 5% of a TIM3⁺ KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ PD1⁺ T cell comprising the CD19-targeted chimeric receptor, or a combination thereof.

In certain embodiments, the composition is a pharmaceutical composition further comprising a pharmaceutically acceptable carrier. In certain embodiments, the composition comprises between about 1×10⁶ and about 5×10⁸ cells. In certain embodiments, the composition comprises between about 1×10⁶ and about 1×10⁸ cells. In certain embodiments, the composition comprises about 1×10⁶ and about 5×10⁷ cells. In certain embodiments, the composition comprises about 2.5×10⁷ cells.

In certain non-limiting embodiments, the presently disclosed subject matter provides a method of reducing tumor burden in a subject, comprising administering to the subject a composition disclosed herein. In certain embodiments, the method reduces the number of tumor cells, reduces tumor size, and/or eradicates the tumor in the subject.

In certain non-limiting embodiments, the presently disclosed subject matter provides a method of increasing or lengthening survival of a subject having a neoplasm, comprising administering to the subject a composition disclosed herein.

In certain non-limiting embodiments, the presently disclosed subject matter provides a method of treating and/or preventing a neoplasm in a subject, comprising administering to the subject a composition of disclosed herein.

In certain embodiments, the tumor and/or neoplasm is associated with CD19. In certain embodiments, the tumor and/or neoplasm is a blood cancer. In certain embodiments, the blood cancer is selected from the group consisting of multiple myeloma, leukemia, and lymphomas. In certain embodiments, the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. In certain embodiments, the lymphoma is Hodgkin's lymphoma or non-Hodgkin's lymphoma. In certain embodiments, the tumor and/or neoplasm is a B cell malignancy. In certain embodiments, the B cell malignancy is selected from the group consisting of B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma. In certain embodiments, the tumor and/or neoplasm is B cell lymphoma. In certain embodiments, the B cell lymphoma is relapsed or refractory (R/R) B cell lymphoma. In certain embodiments, the subject is a human subject.

In certain non-limiting embodiments, the presently disclosed subject matter provides a method of reducing tumor burden in a subject that is non-responsive or has a reduced likelihood to respond to an adoptive cell therapy, comprising:
a) measuring the expression level of at least one of CD57, TIM3, KLRG1, CTLA4, LAG3 and PD1 in an immunoresponsive cell of a subject;
b) comparing the expression level to a reference sample;
c) identifying the subject as non-responsive or as having a reduced likelihood to respond to an adoptive cell therapy if i) the expression level of CD57 is reduced compared to the reference sample, ii) the expression level of TIM3 is reduced compared to the reference sample, iii) the expression level of KLRG1 is increased compared to the reference sample, iv) the expression level of CTLA4 is increased compared to the reference sample, v) the expression level of LAG3 is increased compared to the reference sample, nd/or vi) the expression level of PD1 is reduced compared to the reference sample; and
d) administering to the subject a composition disclosed herein.

In certain non-limiting embodiments, the presently disclosed subject matter provides a method of increasing or lengthening survival of a subject that is non-responsive or has a reduced likelihood to respond to an adoptive cell therapy, comprising:
a) measuring the expression level of at least one of CD57, TIM3, KLRG1, CTLA4, LAG3 and PD1 in an immunoresponsive cell of a subject;
b) comparing the expression level to a reference sample;
c) identifying the subject as non-responsive or as having a reduced likelihood to respond to an adoptive cell therapy if i) the expression level of CD57 is reduced compared to the reference sample, ii) the expression level of TIM3 is reduced compared to the reference sample, iii) the expression level of KLRG1 is increased compared to the reference sample, iv) the expression level of CTLA4 is increased compared to the reference sample, v) the expression level of LAG3 is increased compared to the reference sample, and/or vi) the expression level of PD1 is reduced compared to the reference sample; and
d) administering to the subject a composition disclosed herein.

In certain non-limiting embodiments, the presently disclosed subject matter provides a method of treating and/or preventing a neoplasm in a subject that is non-responsive or has a reduced likelihood to respond to an adoptive cell therapy, comprising:
a) measuring the expression level of at least one of CD57, TIM3, KLRG1, CTLA4, LAG3 and PD1 in an immunoresponsive cell of a subject;
b) comparing the expression level to a reference sample;
c) identifying the subject as non-responsive or as having a reduced likelihood to respond to an adoptive cell therapy if i) the expression level of CD57 is reduced compared to the reference sample, ii) the expression level of TIM3 is reduced compared to the reference sample, iii) the expression level of KLRG1 is increased compared to the reference sample, iv) the expression level of CTLA4 is increased compared to the reference sample, v) the expression level of LAG3 is increased compared to the reference sample, and/or vi) the expression level of PD1 is reduced compared to the reference sample; and
d) administering to the subject a composition disclosed herein.

In certain embodiments, the tumor and/or neoplasm is associated with CD19. In certain embodiments, the tumor and/or neoplasm is a blood cancer. In certain embodiments, the blood cancer is selected from the group consisting of multiple myeloma, leukemia, and lymphomas. In certain embodiments, the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. In certain embodiments, the lymphoma is Hodgkin's lymphoma or non-Hodgkin's lymphoma. In certain embodiments, the tumor and/or neoplasm is a B cell malignancy. In certain embodiments, the B cell malignancy is selected from the group consisting of B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma. In certain embodiments, the tumor and/or neoplasm is B cell lymphoma. In certain embodiments, the B cell lymphoma is relapsed or refractory (R/R) B cell lymphoma. In certain embodiments, the subject is a human subject.

Finally, in certain non-limiting embodiments, the presently disclosed subject matter provides a kit for performing any of the methods disclosed herein.

### BRIEF DESCRIPTION OF THE DRAWINGS

FIGS. 1A and 1B illustrate positive association between response and CD57 and TIM3 expression independently on CAR⁺ Cells and CAR⁻ Cells as well as negative association between response and CTLA4, KLRG1, LAG3 expression independently in the administered compositions/final infusion product. CR: complete response; PR: partial response; NR: no-response.

FIG. 2 illustrates that untransduced cells (UTD) show a similar trend as the final infusion product for these markers. CR: complete response; PR: partial response; NR: no-response.

FIG. 3 illustrates associations between response and CD57, CTLA4, and PD1 are not seen in starting/apheresis material (e.g., a sample from a subject), potential association with KLRG1, LAG3 and TIM3.

### DETAILED DESCRIPTION

The presently disclosed subject matter is based, at least in part, on the discovery that expression levels of certain cell surface markers (e.g., CD57, KLRG1, CTLA4, LAG3, TIM3 and PD1) in CD8⁺ and/or CD4⁺ T cells can predict the responsiveness of patients treated with adoptive cell therapies including an anti-CD19 chimeric antigen receptor (e.g., CD19(T2)28z1xx).

In certain non-limiting embodiments, the presently disclosed subject matter provides a method of identifying a subject having cancer that is responsive to the adoptive cell therapy. In certain embodiments, the method comprises measuring the expression level of CD57, KLRG1, CTLA4, LAG3, TIM3, and/or PD1 in CD8⁺ and/or CD4⁺ T cells from the subject. In certain embodiments, the subject is responsive to the adoptive cell therapy if the expression level of CTLA4, LAG3, and/or KLRG1 in the CD8⁺ and/or CD4⁺ T cells from the subject is reduced as compared to a reference sample. In certain embodiments, the subject is responsive to the adoptive cell therapy if the expression level of CD57, TIM3, and/or PD1 in the CD8⁺ and/or CD4⁺ T cells from the subject is increased as compared to a reference sample.

Additionally, in certain non-limiting embodiments, the presently disclosed subject matter provides an immunoresponsive cell (e.g., a T cell) that has increased therapeutic efficacy. In certain embodiments, the immunoresponsive cell comprises a reduced expression level of CTLA4, LAG3, and/or KLRG1 as compared to a reference sample. In certain embodiments, the immunoresponsive cell comprises an increased expression level of CD57, TIM3, and/or PD1 as compared to a reference sample. Without being bound by any theory, it is believed that CD8⁺ and/or CD4⁺ T cells that express reduced levels of CTLA4, LAG3, and/or KLRG1 and/or increased levels of CD57, TIM3, and/or PD1 have increased functional activity (e.g., cytotoxicity, proliferation, cytokine secretion) in the context of adoptive cell therapies.

Non-limiting embodiments of the present disclosure are described by the present specification and Examples.

For purposes of clarity of disclosure and not by way of limitation, the detailed description is divided into the following subsections:
1. Definitions;
2. Cells;
3. Nucleic Acid Molecules and Vectors;
4. Formulations and Administration;
5. Biomarkers;
6. Methods of Treatment;
7. Kits; and
8. Exemplary Embodiments.

### 1. Definitions

Unless defined otherwise, all technical and scientific terms used herein have the meaning commonly understood by a person skilled in the art to which the presently disclosed subject matter belongs. The following references provide one of skill with a general definition of many of the terms used in the presently disclosed subject matter: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3g/d/e/z, etc.). This clustering of membrane bound signaling molecules allows for ITAM motifs contained within the CD3 chains to become phosphorylated. This phosphorylation in turn initiates a T cell activation pathway ultimately activating transcription factors, such as NF-kB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., Nucl Med (1983); 24:316-325). As used herein, include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the IMGT numbering system. In certain embodiments, the CDR regions are delineated using the IMGT numbering system accessible at http://www.imgt.org/IMGT_vquest/input.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., 10, 15, 20, 25 amino acids), which connects the N-terminus of the V_{H} with the Cterminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker.

In certain embodiments, the linker comprises or consists of the amino acid sequence set forth in SEQ ID NO: 1, which is provided below:

[SEQ ID NO: 1]
GGGGSGGGGSGGGSGGGGS

In certain embodiments, the linker comprise or consists of the amino acid sequence set forth in SEQ ID NO: 2, which is provided below:

[SEQ ID NO: 2]
GGGGSGGGGSGGGGS

In certain embodiments, the linker comprises or consists of the amino acid sequence set forth in SEQ ID NO: 3, which is provided below:

[SEQ ID NO: 3]
GGGGSGGGGSGGGGSGGGSGGGGS

In certain embodiments, the linker comprises or consists of the amino acid sequence set forth in SEQ ID NO: 4, which is provided below:

[SEQ ID NO: 4]
GGGGSGGGGSGGGGSGGGGSGGGSGGGGS

In certain embodiments, the linker comprises or consists of the amino acid sequence set forth in SEQ ID NO: 5, which is provided below:

[SEQ ID NO: 5]
GGGGS

In certain embodiments, the linker comprises or consists of the amino acid sequence set forth in SEQ ID NO: 6, which is provided below:

[SEQ ID NO: 6]
GGGGSGGGGS

In certain embodiments, the linker comprises the first three amino acids of the heavy chain constant region. In certain embodiments, the linker comprises or consists of the amino acid sequence set forth in SEQ ID NO: 7, which is provided below:

[SEQ ID NO: 7]
ASTGGGGSGGGGSGGGGS

Additionally or alternately, the linker can be a Whitlow/218 linker disclosed in Whitlow, M. et al. (1993) Protein Eng 6, 989-95, the contents of which are hereby incorporated by reference in their entireties.

Despite removal of the constant regions and the introduction of a linker, scFv proteins retain the specificity of the original immunoglobulin. Single chain Fv polypeptide antibodies can be expressed from a nucleic acid comprising V_{H}- and V_{L} encoding sequences as described by Huston, et al. Proc. Nat. Acad. Sci. USA, (1988); 85:5879-5883; U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) (2008); 27(6):445-51; Peter et al., J Cachexia Sarcopenia Muscle (2012); August 12; Shieh et al., J Imunol (2009); 183(4):2277-85; Giomarelli et al., Thromb Haemost (2007); 97(6):955-63; Fife eta., J Clin Invst (2006); 116(8):2252-61; Brocks et al., Immunotechnology 1997 3(3):173-84; Moosmayer et al., Ther Immunol 1995 2(10:31-40). Agonistic scFvs having stimulatory activity have been described (Peter et al., J Biol Chem (2003); 25278(38):36740-7; Xie et al., Nat Biotech 1997 15(8):768-71; Ledbetter et al., Crit Rev Immunol (1997); 17(5-6):427-55; Ho et al., BioChim Biophys Acta (2003); 1638(3):257-66).

The term “chimeric antigen receptor” or “CAR” as used herein refers to a molecule comprising an extracellular antigen-binding domain that is fused to an intracellular signaling domain that is capable of activating or stimulating an immunoresponsive cell. In certain embodiments, the CAR also comprises a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain.

The term “chimeric co-stimulating receptor” or “CCR” refers to a chimeric receptor that binds to an antigen and provides co-stimulatory signals, but does not alone provide an activation signal. CCR is described in Krause, et al., J Exp. Med. (1998); 188(4):619-626, and US20020018783, the contents of which are incorporated by reference in their entirety. CCRs mimic co-stimulatory signals, but unlike, CARs, do not alone provide an activation signal, e.g., CCRs lack a CD3ζ polypeptide.

By “substantially identical” or “substantially homologous” is meant a polypeptide or nucleic acid molecule exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions ×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “receptor” is meant a polypeptide, or portion thereof, present on a cell membrane that selectively binds one or more ligand.

By “signal sequence” or “leader sequence” is meant a peptide sequence (e.g., 5, 10, 15, 20, 25 or 30 amino acids) present at the N-terminus of newly synthesized proteins that directs their entry to the secretory pathway

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “a conservative sequence modification” refers to an amino acid modification that does not significantly affect or alter the binding characteristics of the presently disclosed chimeric receptors comprising the amino acid sequence. Conservative modifications can include amino acid substitutions, additions and deletions. Modifications can be introduced into the extracellular antigen-binding domain of the presently disclosed chimeric receptors by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “reference sample” refers to a representative sample of normal measurement of a biomarker (e.g., CD57, TIM3, KLRG1, CTLA4, LAG3, PD1) obtained from a cohort of healthy subject or a cohort of heathy and diseased subjects. In certain embodiments, the reference sample refers to a baseline amount of a biomarker. In certain embodiments, the reference sample refers to isotype controls. In certain embodiments, the reference sample refers to untransduced (UTD) cells or patient apheresis/starting material (SM). In certain embodiments, the reference sample refers to a measurement of a biomarker (e.g., CD57, TIM3, PD1, KLRG1, CTLA4, LAG3) obtained from NR (no response) patients.

Other aspects of the presently disclosed subject matter are described in the following disclosure and are within the ambit of the presently disclosed subject matter.

### 2. Cells

The presently disclosed subject matter provides cells comprising a chimeric receptor comprising an extracellular antigen-binding domain that binds to CD19.

In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide the production of antibodies, regulation of the cellular immune system, detection of foreign agents in the blood, detection of cells foreign to the host, and the like. Non-limiting examples of cells of the lymphoid lineage include T cells, Natural Killer (NK) cells, B cells, dendritic cells, and stem cells from which lymphoid cells may be differentiated. In certain embodiments, the stem cell is a pluripotent stem cell. In certain embodiments, the pluripotent stem cell is an embryonic stem cell (ESC) or an induced pluripotent stem cell (iPSC).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of a chimeric receptor, e.g., a CAR or a HIT. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is an NK cell. Natural killer (NK) cells can be lymphocytes that are part of cell-mediated immunity and act during the innate immune response. NK cells do not require prior activation in order to perform their cytotoxic effect on target cells. In certain embodiments, the cell is a genetically modified NK cell. In certain embodiments, the cell is an edited NK cell. In certain embodiments, the cell is an NK cell derived from a stem cell. In certain embodiments, the cell is an NK cell derived from a pluripotent stem cell. In certain embodiments, the cell is an induced pluripotent stem cell (iPSC)-derived NK cell.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells or NK cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cells, erythrocytes, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid-derived suppressor cells (MDSCs), tumor-associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however, within the tumor microenvironment, these mechanisms prevent an effective anti-tumor immune response. Collectively these immune suppressive factors can induce either marked anergy or apoptosis of adoptively transferred modified T cells upon encounter with targeted tumor cells.

In certain embodiments, the cells can be transduced with a CD19-targeted chimeric receptor disclosed herein such that the cells express the chimeric receptor(s).

### 2.1. CD19

CD19 is a cell-surface 95 kDa glycoprotein present on normal B cells from early in their development until differentiation into plasma cells. CD19 is not present in other normal tissues, including pluripotent blood stem cells. It is expressed in B cell lymphomas, acute lymphoblastic leukemia (ALL), chronic lymphocytic leukemia (CLL), hairy cell leukemias, and a subset of acute myelogenous leukemias making it an attractive target for immunotherapy with minimal risk of autoimmune disease (other than B cell aplasia) or irreversible bone marrow toxicity.

CD19 is a member of the immunoglobulin superfamily and a component of a cell surface signal transduction complex that includes Leu13, CD81, and CD21, which positively regulates signal transduction through the B cell receptor.

In certain embodiments, the presently disclosed chimeric receptor (e.g., CAR) binds to human CD19. In certain embodiments, the human CD19 comprises or consists of the amino acid sequence with an NCBI Reference No: NP_001171569.1 (SEQ ID NO: 8), or a fragment thereof.

SEQ ID NO: 8 is provided below:

[SEQ ID NO: 8]
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLAGSQS
YEDMRGILYAAPQLRSIRGQPGPNHEEDADSYENMDNPDGPDPAWGGGGR
MGTWSTR

In certain embodiments, the human CD19 comprises or consists of the amino acid sequence with an NCBI Reference No: NP_001761.3 (SEQ ID NO: 9), or a fragment thereof. SEQ ID NO: 9 is provided below:

[SEQ ID NO: 9]
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR

In certain embodiments, the chimeric receptor binds to the extracellular domain of CD19. In certain embodiments, the chimeric receptor binds to the extracellular domain of human CD19. In certain embodiments, the extracellular domain of human CD19 comprises or consists of amino acids 20 to 291 of SEQ ID NO: 8. In certain embodiments, the extracellular domain of human CD19 comprises or consists of amino acids 20 to 291 of SEQ ID NO: 9.

In certain embodiments, the CD19 comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 8 or a fragment thereof.

In certain embodiments, the CD19 comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 9 or a fragment thereof.

### 2.2. Chimeric Receptors

In certain embodiments, the presently disclosed chimeric receptor comprises an extracellular antigen-binding domain that binds to CD19. The extracellular antigen-binding domain can be an antigen-binding fragment of an antibody, an antigen-binding fragment of a heavy chain variable region (V_{H}) of an antibody, an antigen-binding fragment of a light chain variable region (V_{L}) of an antibody, a single chain variable fragment (scFv), a Fab, or F(ab)₂. In certain embodiments, the extracellular antigen-binding fragment is a single chain variable fragment (scFv). In certain embodiments, the scFv is a human scFv. In certain embodiments, the scFv is a humanized scFv. In certain embodiments, the scFv is a murine scFv. In certain embodiments, the Fab is crosslinked.

In certain embodiments, the presently disclosed chimeric receptor is a chimeric antigen receptor (CAR). In certain embodiments, the chimeric receptor is a TCR like fusion molecule.

### 2.2.1. Chimeric Antigen Receptor (CAR)

In certain embodiments, the chimeric receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to the cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the chimeric receptor is a second generation CAR. In certain embodiments, the chimeric receptor is a CAR that comprises an intracellular domain of a co-stimulatory molecule or a fragment thereof.

### 2.2.1.1. Extracellular Antigen-Binding Domain of a CAR

In certain embodiments, the extracellular antigen-binding domain is a single chain variable fragment (scFv). In certain embodiments, the scFv is a human scFv. In certain embodiments, the scFv is a humanized scFv. In certain embodiments, the scFv is a murine scFv. In certain embodiments, the scFv is identified by screening scFv phage library with an antigen-Fc fusion protein.

In certain embodiments, the extracellular antigen-binding domain is a Fab. In certain embodiments, the Fab is crosslinked. In certain embodiments, the extracellular antigen-binding domain is a F(ab)₂. Any of the foregoing molecules may be comprised in a fusion protein with a heterologous sequence to form the extracellular antigen-binding domain.

Binding of the extracellular antigen-binding domain of a chimeric receptor, e.g., a CAR, can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassay, Seventh Training Course on Radioligand Assay Techniques, The Endocrine Society, March, 1986, which is incorporated by reference herein). The radioactive isotope can be detected by such means as the use of a γ counter, a scintillation counter, or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

In certain embodiments, the extracellular antigen-binding domain of the CAR binds to CD19 (e.g., human CD19) with a dissociation constant (K_{d}) of at least about 1×10⁻⁶ M, at least about 1×10⁻⁷ M, at least about 1×10⁻⁸M, at least about 1×10⁻⁹M, or at least about 1×10⁻¹⁰ M. In certain embodiments, the extracellular antigen-binding domain of the CAR binds to CD19 (e.g., human CD19) with a dissociation constant (K_{d}) of at least about 2×10⁻⁸ M. In certain embodiments, the extracellular antigen-binding domain of the CAR binds to CD19 (e.g., human CD19) with a dissociation constant (K_{d}) of between about 2×10⁻⁸ M and about 8×10⁻⁹ M.

In certain embodiments, the extracellular antigen-binding domain of the CAR binds to CD19 (e.g., human CD19) with a dissociation constant (K_{d}) of between about 1 nM and 50 nM, between about 5 nM and 30 nM, between about 5 nM and 25 nM, or between about 8 nM and 20 nM. In certain embodiments, the extracellular antigen-binding domain of the CAR binds to CD19 (e.g., human CD19) with a dissociation constant (K_{d}) of at least about 50 nM, at least about 40 nM, at least about 35 nM, at least about 30 nM, at least about 25 nM, at least about 20 nM, at least about 19 nM, at least about 18 nM, at least about 17 nM, at least about 16 nM, at least about 15 nM, at least about 14 nM, at least about 13 nM, at least about 12 nM, at least about 11 nM, at least about 10 nM, at least about 9 nM, at least about 8 nM, at least about 7 nM, at least about 6 nM, or at least about 5 nM.

In certain embodiments, the extracellular antigen-binding domain of the CD19-targeted chimeric receptor (e.g., a CD19-targeted scFv) comprises a V_{H} comprising a CDR1 comprising the amino acid sequence set forth in SEQ ID NO: 10 or a conservative modification thereof, a CDR2 comprising the amino acid sequence set forth in SEQ ID NO: 11 or a conservative modification thereof, and a CDR3 comprising the amino acid sequence set forth in SEQ ID NO: 12 or a conservative modification thereof. SEQ ID NOs: 10-12 are provided in Table 1.

In certain embodiments, the extracellular antigen-binding domain of the CD19-targeted chimeric receptor (e.g., a CD19-targeted scFv) comprises a V_{L} comprising a CDR1 comprising the amino acid sequence set forth in SEQ ID NO: 13 or a conservative modification thereof, a CDR2 comprising the amino acid sequence set forth in SEQ ID NO: 14 or a conservative modification thereof, and a CDR3 comprising the amino acid sequence set forth in SEQ ID NO: 15 or a conservative modification thereof. SEQ ID NOs: 13-15 are provided in Table 1.

In certain embodiments, the extracellular antigen-binding domain of the CD19-targeted chimeric receptor (e.g., a CD19-targeted scFv) comprises a V_{H} comprising a CDR1 comprising the amino acid sequence set forth in SEQ ID NO: 10 or a conservative modification thereof, a CDR2 comprising the amino acid sequence set forth in SEQ ID NO: 11 or a conservative modification thereof, and a CDR3 comprising the amino acid sequence set forth in SEQ ID NO: 12 or a conservative modification thereof, and a V_{L} comprising a CDR1 comprising the amino acid sequence set forth in SEQ ID NO: 13 or a conservative modification thereof, a CDR2 comprising the amino acid sequence set forth in SEQ ID NO: 14 or a conservative modification, and a CDR3 comprising the amino acid sequence set forth in SEQ ID NO: 15 or a conservative modification thereof.

In certain embodiments, the extracellular antigen-binding domain of the CD19-targeted chimeric receptor (e.g., a CD19-targeted scFv) comprises a V_{H} comprising a CDR1 comprising the amino acid sequence set forth in SEQ ID NO: 10, a CDR2 comprising the amino acid sequence set forth in SEQ ID NO: 11, a CDR3 comprising the amino acid sequence set forth in SEQ ID NO: 12; and a V_{L} comprising a CDR1 comprising the amino acid sequence set forth in SEQ ID NO: 13, a V_{L} CDR2 comprising the amino acid sequence set forth in SEQ ID NO: 14, and a V_{L} CDR3 comprising the amino acid sequence set forth in SEQ ID NO: 15.

In certain embodiments, the extracellular antigen-binding domain of the CD19-targeted chimeric receptor (e.g., a CD19-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 16. For example, the extracellular antigen-binding domain of the CD19-targeted chimeric receptor (e.g., a CD19-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 16. In certain embodiments, the extracellular antigen-binding domain of the CD19-targeted chimeric receptor (e.g., a CD19-targeted scFv) comprises a V_{H} comprising the amino acid sequence set forth in SEQ ID NO: 16. SEQ ID NO: 16 is provided in Table 1 below.

In certain embodiments, the extracellular antigen-binding domain of the CD19-targeted chimeric receptor (e.g., a CD19-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 17. For example, the extracellular antigen-binding domain of the CD19-targeted chimeric receptor (e.g., a CD19-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 17. In certain embodiments, the extracellular antigen-binding domain of the CD19-targeted chimeric receptor (e.g., a CD19-targeted scFv) comprises a V_{L} comprising the amino acid sequence set forth in SEQ ID NO: 17. SEQ ID NO: 17 is provided in Table 1 below.

In certain embodiments, the extracellular antigen-binding domain of the CD19-targeted chimeric receptor (e.g., a CD19-targeted scFv) comprises a V_{H} comprising the amino acid sequence set forth in SEQ ID NO: 16, and a V_{L} comprising the amino acid sequence set forth in SEQ ID NO: 17. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

In certain embodiments, the variable regions within the extracellular antigen-binding domain of the CD19-targeted chimeric receptor have to be linked one after another such that at the N-terminus of the extracellular antigen-binding domain, a heavy chain variable region (V_{H}) is positioned. In certain embodiments, if the extracellular antigen-binding domain of the CD19-targeted chimeric receptor is an scFv, the variable regions are positioned from the N- to the C-terminus: V_{H}-V_{L}. In certain embodiments, the CD19-targeted scFv comprises or consists of the amino acid sequence set forth in SEQ ID NO: 18. An exemplary nucleotide sequence encoding the amino acid sequence of SEQ ID NO: 18 is set forth in SEQ ID NO: 19. SEQ ID NOs: 18 and 19 are provided in Table 1. The CDRs provided in Table 1 are identified according to the Kabat numbering system.

TABLE 1

| CDRs | 1 | 2 | 3 |
|---|---|---|---|
| V_{H} | SYGMH | LIWYDGSNKYYADSVKG | PVEGLLRGFDY |
| | [SEQ ID NO: 10] | [SEQ ID NO: 11] | [SEQ ID NO: 12] |
| V_{L} | RASQSVSSSYLA | GASSRAT | QQAGAVPIT |
| | [SEQ ID NO: 13] | [SEQ ID NO: 14] | [SEQ ID NO: 15] |

| | |
|---|---|
| Full V_{H} | QVQLVESGGGVVQPGRSLRLSCAASGFTFSSYGMHWVRQAPGKGLEWVA |
| | LIWYDGSNKYYADSVKGRFTISRDNSKNTLYLQMNSLRAEDTAVYYCAKP |
| | VEGLLRGFDYWGQGTLVTVSS [SEQ ID NO: 16] |
| Full V_{L} | EIVLTQSPGTLSLSPGERATLSCRASQSVSSSYLAWYQQKPGQAPRLLIYGA |
| | SSRATGIPDRFSGSGSGTDFTLTISRLEPEDFAVYYCQQAGAVPITFGGGTK |
| | VEIK [SEQ ID NO: 17] |
| V_{L}-V_{H} | EIVLTQSPGTLSLSPGERATLSCRASQSVSSSYLAWYQQKPGQAPRLLIYGA |
| scFv | SSRATGIPDRFSGSGSGTDFTLTISRLEPEDFAVYYCQQAGAVPITFGGGTK |
| | VEIKGGGGSGGGGSGGGGSQVQLVESGGGVVQPGRSLRLSCAASGFTFSS |
| | YGMHWVRQAPGKGLEWVALIWYDGSNKYYADSVKGRFTISRDNSKNTL |
| | YLQMNSLRAEDTAVYYCAKPVEGLLRGFDYWGQGTLVTVSS |
| | [SEQ ID NO: 181] |

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 16 or SEQ ID NO: 17) may contain substitutions (e.g., conservative substitutions), insertions, or deletions relative to the specified sequence(s), but retain the ability to bind to a target antigen (e.g., CD19). In certain embodiments, a total of 1 to 10 amino acids are substituted, inserted, and/or deleted in a specific sequence (e.g., SEQ ID NO: 16 or SEQ ID NO: 17). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NO: 16 or SEQ ID NO: 17, including post-translational modifications of that sequence (SEQ ID NO: 16 or SEQ ID NO: 17).

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain. In certain embodiments, the signal peptide comprises a CD8 polypeptide, e.g., the CAR comprises a truncated CD8 signal peptide. In certain embodiments, the signal peptide is generated from the antibody from it is derived. In certain embodiments, the signal peptide is set forth in SEQ ID NO: 19, which is provided below:

[SEQ ID NO: 19]
MDMRVPAQLLGLLLLWLPDTRC

Additionally or alternatively, the extracellular antigen-binding domain of the CD19-targeted chimeric receptor (e.g., a CD19-targeted scFv) comprises an extracellular antigen-binding domain disclosed in International Patent Application No. PCT/US2021/029138, the content of which is incorporated by reference in its entirety.

### 2.2.1.2. Transmembrane Domain of a CAR

In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain of the CAR comprises a CD8 polypeptide (e.g., a transmembrane domain of CD8 or a fragment thereof).

In certain embodiments, the transmembrane domain of the CAR comprises a CD8 polypeptide (e.g., a transmembrane domain of CD8 or a fragment thereof). In certain embodiments, the transmembrane domain of the CAR comprises a CD8 polypeptide (e.g., a transmembrane domain of human CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 20) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 20, which is at least about 20, or at least about 30, or at least about 40, or at least about 50, at least about 60, at least about 70, and up to about 235 amino acids in length. In certain embodiments, the CD8 polypeptide comprises or consists of amino acids 1 to 235, 1 to 50, 50 to 100, 100 to 150, 150 to 200, 137 to 207, or 200 to 235 of SEQ ID NO: 20. In certain embodiments, the transmembrane domain of the CAR comprises a CD8 polypeptide comprising or consisting of amino acids 137 to 207 of SEQ ID NO: 20. SEQ ID NO: 20 is provided below.

[SEQ ID NO: 20]
MALPVTALLLPLALLLHAARPSQFRVSPLDRTWNLGETVELKCQVLLSNPTSGCSWLFQ
PRGAAASPTFLLYLSQNKPKAAEGLDTQRFSGKRLGDTFVLTLSDFRRENEGYYFCSAL
SNSIMYFSHFVPVFLPAKPTTTPAPRPPTPAPTIASQPLSLRPEACRPAAGGAVHTRGLDF
ACDIYIWAPLAGTCGVLLLSLVITLYCNHRNRRRVCKCPRPVVKSGDKPSLSARYV

An exemplary nucleotide sequence encoding amino acids 137 to 207 of SEQ ID NO: 20 is set forth in SEQ ID NO: 21, which is provided below.

[SEQ ID NO: 21]
CCCACCACGACGCCAGCGCCGCGACCACCAACCCCGGCGCCCACGATCGCGTCGCA
GCCCCTGTCCCTGCGCCCAGAGGCGTGCCGGCCAGCGGCGGGGGGCGCAGTGCACA
CGAGGGGGCTGGACTTCGCCTGTGATATCTACATCTGGGCGCCCCTGGCCGGGACTT
GTGGGGTCCTTCTCCTGTCACTGGTTATCACCCTTTACTGCAAC

In certain embodiments, the transmembrane domain of the CAR comprises a CD8 polypeptide (e.g., a transmembrane domain of mouse CD8 or a fragment thereof). In certain embodiments, the CD8 polypeptide comprises or consists of an amino acid sequence that is at least about 85%, about 90%, about 95%, about 96%, about 97%, about 98%, about 99%, or about 100% homologous or identical to the amino acid sequence having a NCBI Reference No: AAA92533.1 (SEQ ID NO: 22) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 22, which is at least about 20, or at least about 30, or at least about 40, or at least about 50, or at least about 60, or at least about 70, or at least about 100, or at least about 200, and up to about 247 amino acids in length. In certain embodiments, the CD8 polypeptide comprises or consists of amino acids 1 to 247, 1 to 50, 50 to 100, 100 to 150, 150 to 200, 151 to 219, or 200 to 247 of SEQ ID NO: 22. In certain embodiments, the transmembrane domain of the CAR comprises a CD8 polypeptide comprising or consisting of amino acids 151 to 219 of SEQ ID NO: 22. SEQ ID NO: 22 is provided below.

[SEQ ID NO: 22]
MASPLTRFLSLNLLLMGESIILGSGEAKPQAPELRIFPKKMDAELGQKVDLVCEVLGSVS
QGCSWLFQNSSSKLPQPTFVVYMASSHNKITWDEKLNSSKLFSAVRDTNNKYVLTLNK
FSKENEGYYFCSVISNSVMYFSSVVPVLQKVNSTTTKPVLRTPSPVHPTGTSQPQRPEDC
RPRGSVKGTGLDFACDIYIWAPLAGICVAPLLSLIITLICYHRSRKRVCKCPRPLVRQEGK
PRPSEKIV

In certain embodiments, the transmembrane domain of a presently disclosed CAR comprises a CD28 polypeptide (e.g., a transmembrane domain of CD28 or a fragment thereof).

In certain embodiments, the transmembrane domain of the CAR comprises a CD28 polypeptide (e.g., a transmembrane domain of human CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having an NCBI Reference No: NP_006130 (SEQ ID NO: 23) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 10, which is at least about 20, or at least about 30, or at least about 40, or at least about 50, and up to about 220 amino acids in length. In certain embodiments, the CD28 polypeptide comprises or consists of amino acids 1 to 220, 1 to 50, 50 to 100, 100 to 150, 150 to 200, 153 to 179, or 200 to 220 of SEQ ID NO: 23. In certain embodiments, the transmembrane domain of the CAR comprises a CD28 polypeptide comprising or consisting of amino acids 153 to 179 of SEQ ID NO: 23. SEQ ID NO: 23 is provided below:

[SEQ ID NO: 23]
MLRLLLALNLFPSIQVTGNKILVKQSPMLVAYDNAVNLSCKYSYNLFSREFRASLHKGL
DSAVEVCVVYGNYSQQLQVYSKTGFNCDGKLGNESVTFYLQNLYVNQTDIYFCKIEV
MYPPPYLDNEKSNGTIIHVKGKHLCPSPLFPGPSKPFWVLVVVGGVLACYSLLVTVAFII
FWVRSKRSRLLHSDYMNMTPRRPGPTRKHYQPYAPPRDFAAYRS

An exemplary nucleotide sequence encoding amino acids 153 to 179 of SEQ ID NO: 23 is set forth in SEQ ID NO: 24, which is provided below.

[SEQ ID NO: 24]
TTTTGGGTGCTGGTGGTGGTTGGTGGAGTCCTGGCTTGCTATAGCTTGC
TAGTAACAGTGGCCTTTATTATTTTCTGGGTG

In certain embodiments, the transmembrane domain of the CAR comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99%, or 100% homologous or identical to the amino acid sequence having an NCBI Reference No: NP_031668.3 (SEQ ID NO: 25) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 25, which is at least about 20, or at least about 30, or at least about 40, or at least about 50, and up to about 218 amino acids in length. In certain embodiments, the CD28 polypeptide comprises or consists of amino acids 1 to 220, 1 to 50, 50 to 100, 100 to 150, 150 to 200, 151 to 177, or 200 to 218 of SEQ ID NO: 25. In certain embodiments, the transmembrane domain of the CAR comprises a CD28 polypeptide comprising or consisting of amino acids 151 to 177 of SEQ ID NO: 25. SEQ ID NO: 25 is provided below:

[SEQ ID NO: 25]
MTLRLLFLALNFFSVQVTENKILVKQSPLLVVDSNEVSLSCRYSYNLLAKEFRASLYKG
VNSDVEVCVGNGNFTYQPQFRSNAEFNCDGDFDNETVTFRLWNLHVNHTDIYFCKIEF
MYPPPYLDNERSNGTIIHIKEKHLCHTQSSPKLFWALVVVAGVLFCYGLLVTVALCVIW
TNSRRNRLLQSDYMNMTPRRPGLTRKPYQPYAPARDFAAYRP

In certain embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, the CH₂CH₃ region of immunoglobulin and portions of CD3, a portion of a CD28 polypeptide (e.g., a portion of SEQ ID NO: 23 or SEQ ID NO: 25), a portion of a CD8 polypeptide (e.g., a portion of SEQ ID NO: 20 or SEQ ID NO: 22), a variation of any of the foregoing which is at least about 80%, at least about 85%, at least about 90%, at least about 95%, or at least about 100% homologous or identical thereto, or a synthetic spacer sequence.

In certain embodiments, the hinge/spacer region of the CAR comprises a CD28 polypeptide. In certain embodiments, the hinge/spacer region of the CAR comprises a CD28 polypeptide comprising or consisting of amino acids 114 to 152 of SEQ ID NO: 23.

An exemplary nucleotide sequence encoding amino acids 114 to 152 of SEQ ID NO: 23 is set forth in SEQ ID NO: 26, which is provided below.

[SEQ ID NO: 26]
ATTGAAGTTATGTATCCTCCTCCTTACCTAGACAATGAGAAGAGCAATG
GAACCATTATCCATGTGAAAGGGAAACACCTTTGTCCAAGTCCCCTATT
TCCCGGACCTTCTAAGCCC

### 2.2.1.3. Intracellular Signaling Domain of a CAR

In certain embodiments, the CAR comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T cell). Wild type (“native”) CD3ζ comprises three functional immunoreceptor tyrosine-based activation motifs (ITAMs), and three functional basic-rich stretch (BRS) regions (BRS1, BRS2, and BRS3). CD3ζ transmits an activation signal to the cell (e.g., a cell of the lymphoid lineage, e.g., a T cell) after the antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the native CD3ζ polypeptide comprises or consists of an amino acid sequence that is at least about 85%, about 90%, about 95%, about 96%, about 97%, about 98%, about 99% or about 100% homologous or identical to the amino acid sequence having an NCBI Reference No: NP_932170 (SEQ ID NO: 27) or a fragment thereof, and/or may optionally comprise up to one or up to two or up to three conservative amino acid substitutions. In certain embodiments, the native CD3ζ polypeptide comprises or consists of an amino acid sequence that is a consecutive portion of SEQ ID NO: 27, which is at least about 20, or at least about 30, or at least about 40, or at least about 50, at least about 100, at least about 110, and up to about 164 amino acids in length. In certain embodiments, the native CD3ζ polypeptide comprises or consists of amino acids 1 to 164, 1 to 50, 50 to 100, 52 to 164, 100 to 150, or 150 to 164 of SEQ ID NO: 27. In certain embodiments, the intracellular signaling domain of the CAR comprises a CD3ζ polypeptide comprising or consisting of amino acids 52 to 164 of SEQ ID NO: 27. SEQ ID NO: 27 is provided below:

[SEQ ID NO: 27]
MKWKALFTAAILQAQLPITEAQSFGLLDPKLCYLLDGILFIYGVILTALFLRVKFSRSAD
APAYQQGQNQLYNELNLGRREEYDVLDKRRGRDPEMGGKPQRRKNPQEGLYNELQK
DKMAEAYSEIGMKGERRRGKGHDGLYQGLSTATKDTYDALHMQALPPR

In certain embodiments, the intracellular signaling domain of the CAR comprises a modified CD3ζ polypeptide. In certain embodiments, the modified CD3ζ polypeptide comprises one, two, or three ITAMs. In certain embodiments, the modified CD3ζ polypeptide comprises a native ITAM1. In certain embodiments, the native ITAM1 comprises or consists of the amino acid sequence set forth in SEQ ID NO: 28.

[SEQ ID NO: 28]
QNQLYNELNLGRREEYDVLDKR

An exemplary nucleotide sequence encoding the amino acid sequence of SEQ ID NO: 28 is set forth in SEQ ID NO: 29, which is provided below.

[SEQ ID NO: 29]
CAGAACCAGCTCTATAACGAGCTCAATCTAGGACGAAGAGAGGAGTACG
ATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 30, which is provided below.

[SEQ ID NO: 30]
QNQLFNELNLGRREEFDVLDKR

An exemplary nucleotide sequence encoding the amino acid sequence of SEQ ID NO: 30 is set forth in SEQ ID NO: 31, which is provided below.

[SEQ ID NO: 31]
CAGAACCAGCTCTTTAACGAGCTCAATCTAGGACGAAGAGAGGAGTTCG
ATGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 32, which is provided below.

[SEQ ID NO: 32]
QEGLYNELQKDKMAEAYSEIGMK

An exemplary nucleotide sequence encoding the amino acid sequence of SEQ ID NO: 32 is set forth in SEQ ID NO: 33, which is provided below.

[SEQ ID NO: 33]
CAGGAAGGCCTGTACAATGAACTGCAGAAAGATAAGATGGCGGAGGCCT
ACAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) loss of function mutations comprises a mutation of a tyrosine residue in ITAM2. In certain embodiments, the ITAM2 variant consists of two loss-of-function mutations. In certain embodiments, the ITAM2 variant comprises or consists of the amino acid sequence set forth in SEQ ID NO: 21, which is provided below.

[SEQ ID NO: 34]
QEGLFNELQKDKMAEAFSEIGMK

An exemplary nucleotide sequence encoding the amino acid sequence of SEQ ID NO: 34 is set forth in SEQ ID NO: 35, which is provided below.

[SEQ ID NO: 35]
CAGGAAGGCCTGTTCAATGAACTGCAGAAAGATAAGATGGCGGAGGCCT
TCAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 36, which is provided below.

[SEQ ID NO: 36]
HDGLYQGLSTATKDTYDALHMQ

An exemplary nucleotide sequence encoding the amino acid sequence of SEQ ID NO: 36 is set forth in SEQ ID NO: 37, which is provided below.

[SEQ ID NO: 37]
CACGATGGCCTTTACCAGGGTCTCAGTACAGCCACCAAGGACACCTACG
ACGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 38, which is provided below.

[SEQ ID NO: 38]
HDGLFQGLSTATKDTFDALHMQ

An exemplary nucleotide sequence encoding the amino acid sequence of SEQ ID NO: 38 is set forth in SEQ ID NO: 39, which is provided below.

[SEQ ID NO: 39]
CACGATGGCCTTTTCCAGGGTCTCAGTACAGCCACCAAGGACACCTTCG
ACGCCCTTCACATGCAG

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 28, an ITAM2 variant consisting of the amino acid sequence set forth in SEQ ID NO: 34, and an ITAM3 variant consisting of the amino acid sequence set forth in SEQ ID NO: 38. In certain embodiments, the modified CD3ζ polypeptide is designated as “1XX”. In certain embodiments, the modified CD3ζ polypeptide comprises or consists of the amino acid sequence set forth in SEQ ID NO: 40. SEQ ID NO: 40 is provided below.

[SEQ ID NO: 40]
RVKFSRSADAPAYQQGQNQLYNELNLGRREEYDVLDKRRGRDPEMGGKP
RRKNPQEGLFNELQKDKMAEAFSEIGMKGERRRGKGHDGLFQGLSTATK
DTFDALHMQALPPR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 40 or a fragment thereof, and/or may optionally comprise up to one or up to two or up to three conservative amino acid substitutions.

An exemplary nucleotide sequence encoding the amino acid sequence of SEQ ID NO: 40 is set forth in SEQ ID NO: 41, which is provided below.

[SEQ ID NO: 41]
AGAGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACAAG
AGACGTGGCCGGGACCCTGAGATGGGGGGAAAGCCGAGAAGGAAGAACCCTCAGG
AAGGCCTGTTCAATGAACTGCAGAAAGATAAGATGGCGGAGGCCTTCAGTGAGATT
GGGATGAAAGGCGAGCGCCGGAGGGGCAAGGGGCACGATGGCCTTTTCCAGGGTC
TCAGTACAGCCACCAAGGACACCTTCGACGCCCTTCACATGCAGGCCCTGCCCCCTC
GC

In certain embodiments, the CAR is a second-generation CAR. In certain embodiments, the intracellular signaling domain of the CAR further comprises at least a co-stimulatory signaling region. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, and NKGD2. The co-stimulatory molecule can bind to a co-stimulatory ligand, which is a protein expressed on cell surface that upon binding to its receptor produces a co-stimulatory response, i.e., an intracellular response that effects the stimulation provided when an chimeric receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 23 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 23, which is at least about 20, or at least about 30, or at least about 40, or at least about 50, and up to about 220 amino acids in length. In certain embodiments, the CD28 polypeptide comprises or consists of amino acids 1 to 220, 1 to 50, 50 to 100, 100 to 150, 114 to 220, 150 to 200, 180 to 220, or 200 to 220 of SEQ ID NO: 25. In certain embodiments, the intracellular signaling domain of the CAR comprises a co-stimulatory signaling region that comprises a CD28 polypeptide comprising or consisting of amino acids 180 to 220 of SEQ ID NO: 23.

An exemplary nucleic acid sequence encoding amino acids 180 to 220 of SEQ ID NO: 23 is set forth in SEQ ID NO: 42, which is provided below.

[SEQ ID NO: 42]
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC

In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 25 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 25, which is at least about 20, or at least about 30, or at least about 40, or at least about 50, and up to 218 amino acids in length. In certain embodiments, the CD28 polypeptide comprises or consists of amino acids 1 to 218, 1 to 50, 50 to 100, 100 to 150, 150 to 218, 178 to 218, or 200 to 218 of SEQ ID NO: 5. In certain embodiments, the co-stimulatory signaling region of a presently disclosed CAR comprises a CD28 polypeptide that comprises or consists of the amino acids 178 to 218 of SEQ ID NO: 25.

In certain embodiments, the intracellular signaling domain of the CAR comprises a co-stimulatory signaling region that comprises a 4-1BB polypeptide, e.g., an intracellular domain of 4-1BB or a fragment thereof (e.g., an intracellular domain of human 4-1BB or a fragment thereof). The 4-1BB polypeptide can comprise or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref No.: NP_001552 (SEQ ID NO: 43) or a fragment thereof, and/or may optionally comprise up to one or up to two or up to three conservative amino acid substitutions. In certain embodiments, the 4-1BB polypeptide comprises or consists of an amino acid sequence that is a consecutive portion of SEQ ID NO: 43, which is at least about 20, or at least about 30, or at least about 40, or at least about 50, or at least about 100, or at least about 150, or at least about 150, and up to about 255 amino acids in length. In certain embodiments, the 4-1BB polypeptide comprises or consists of amino acids 1 to 255, 1 to 50, 50 to 100, 100 to 150, 150 to 200, 200 to 255, or 214 to 255 of SEQ ID NO: 43. In certain embodiments, the intracellular signaling domain of the CAR comprises a co-stimulatory signaling region that comprises a 4-1BB polypeptide comprising or consisting of amino acids 214 to 255 of SEQ ID NO: 43. SEQ ID NO: 43 is provided below.

[SEQ ID NO: 43]
MGNSCYNIVATLLLVLNFERTRSLQDPCSNCPAGTFCDNNRNQICSPCPPNSFSSAGGQR
TCDICRQCKGVFRTRKECSSTSNAECDCTPGFHCLGAGCSMCEQDCKQGQELTKKGCK
DCCFGTFNDQKRGICRPWTNCSLDGKSVLVNGTKERDVVCGPSPADLSPGASSVTPPAP
AREPGHSPQIISFFLALTSTALLFLLFFLTLRFSVVKRGRKKLLYIFKQPFMRPVQTTQEE
DGCSCRFPEEEEGGCEL

An exemplary nucleic acid sequence encoding amino acids 214 to 255 of SEQ ID NO: 43 is set forth in SEQ ID NO: 44, which is provided below.

[SEQ ID NO: 44]
AAACGGGGCAGAAAGAAGCTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG

In certain embodiments, the intracellular signaling domain of the CAR comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

### 2.2.1.4. Exemplified CAR

In certain embodiments, the CD19-targeted chimeric receptor is a CD19-targeted CAR. In certain embodiments, the CD19-targeted CAR comprises (a) an extracellular antigen-binding domain comprising (i) a V_{H} comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12, and (ii) a V_{L} comprising a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15, (b) a hinge domain comprising a CD28 polypeptide (e.g., a hinge domain of human CD28 or a portion thereof, e.g., a CD28 polypeptide consisting of the amino acid sequence of amino acids 114 to 153 of SEQ ID NO: 23); (c) a transmembrane domain comprising a CD28 polypeptide (e.g., a transmembrane domain of human CD28 or a portion thereof, e.g., a CD28 polypeptide consisting of the amino acid sequence of amino acids 154 to 179 of SEQ ID NO: 23), and (d) an intracellular signaling domain comprising (i) a CD3ζ polypeptide comprising the amino acid sequence set forth in SEQ ID NO: 40, and (ii) a co-stimulatory signaling region comprising a CD28 polypeptide (e.g., an intracellular domain of human CD28 or a portion thereof, e.g., a CD28 polypeptide consisting of the amino acid sequence of amino acids 180 to 220 of SEQ ID NO: 23). In certain embodiments, the V_{H} and V_{L} are linked via a linker consisting of the amino acid sequence set forth in SEQ ID NO: 1. In certain embodiments, the V_{H} and V_{L} are positioned from the N- to the C-terminus: V_{L}-V_{H}. In certain embodiments, the CAR is designed as “19(T2)28z1XX”. In certain embodiments, the CAR comprises the amino acid sequence set forth in SEQ ID NO: 45, which is provided below.

[SEQ ID NO: 45]
MDMRVPAQLLGLLLLWLPDTRCEIVLTQSPGTLSLSPGERATLSCRASQSVSSSYLAWY
QQKPGQAPRLLIYGASSRATGIPDRFSGSGSGTDFTLTISRLEPEDFAVYYCQQAGAVPIT
FGGGTKVEIKGGGGSGGGGSGGGGSQVQLVESGGGVVQPGRSLRLSCAASGFTFSSYG
MHWVRQAPGKGLEWVALIWYDGSNKYYADSVKGRFTISRDNSKNTLYLQMNSLRAE
DTAVYYCAKPVEGLLRGFDYWGQGTLVTVSSRAAAIEVMYPPPYLDNEKSNGTIIHVK
GKHLCPSPLFPGPSKPFWVLVVVGGVLACYSLLVTVAFIIFWVRSKRSRLLHSDYMNMT
PRRPGPTRKHYQPYAPPRDFAAYRSRVKFSRSADAPAYQQGQNQLYNELNLGRREEYD
VLDKRRGRDPEMGGKPRRKNPQEGLFNELQKDKMAEAFSEIGMKGERRRGKGHDGLF
QGLSTATKDTFDALHMQALPPR

An exemplary nucleic acid sequence encoding the amino acid sequence of SEQ ID NO: 45 is set forth in SEQ ID NO: 46, which is provided below.

[SEQ ID NO: 46]
ATGGATATGAGAGTACCAGCTCAGCTGCTGGGCCTGCTGCTTTTGTGGTTGCCGGAC
ACACGCTGTGAGATTGTCCTGACTCAGTCTCCCGGGACTCTGTCCCTCAGCCCCGGT
GAACGCGCTACCCTTTCATGCAGAGCCTCTCAGTCTGTGTCCAGCAGCTACCTCGCA
TGGTATCAGCAGAAGCCCGGACAGGCTCCCAGGCTGTTGATCTATGGAGCTAGTAG
TCGAGCAACAGGCATCCCAGATCGCTTCTCAGGGAGCGGTTCAGGTACAGACTTCA
CGCTGACGATTTCAAGGCTGGAACCCGAAGATTTTGCCGTCTATTATTGTCAACAGG
CAGGGGCTGTGCCAATCACTTTCGGGGGCGGGACCAAGGTGGAAATCAAAGGAGG
CGGAGGAAGTGGAGGAGGAGGGAGCGGTGGAGGAGGGTCACAGGTGCAGCTGGTA
GAATCTGGCGGAGGGGTCGTTCAACCAGGGAGGTCATTGCGGTTGAGCTGCGCAGC
GAGTGGTTTTACCTTCAGCAGTTATGGAATGCATTGGGTGAGACAAGCACCAGGAA
AAGGTCTGGAGTGGGTGGCTTTGATTTGGTACGACGGCAGTAATAAATACTACGCC
GATTCTGTTAAGGGCAGATTTACTATTTCTCGCGACAACAGCAAGAACACGCTGTAC
CTGCAGATGAACTCTCTGAGAGCCGAAGATACAGCAGTGTACTATTGTGCTAAGCC
CGTAGAAGGGCTCCTGAGGGGATTCGATTATTGGGGCCAGGGTACGCTTGTGACAG
TGTCTAGTCGGGCGGCCGCAATTGAAGTTATGTATCCTCCTCCTTACCTAGACAATG
AGAAGAGCAATGGAACCATTATCCATGTGAAAGGGAAACACCTTTGTCCAAGTCCC
CTATTTCCCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAGGAGTAAG
AGGAGCAGGCTCCTGCACAGTGACTACATGAACATGACTCCCCGCCGCCCCGGGCC
CACCCGCAAGCATTACCAGCCCTATGCCCCACCACGCGACTTCGCAGCCTATCGCTC
CAGAGTGAAGTTCAGCAGGAGCGCAGACGCCCCCGCGTACCAGCAGGGCCAGAAC
CAGCTCTATAACGAGCTCAATCTAGGACGAAGAGAGGAGTACGATGTTTTGGACAA
GAGACGTGGCCGGGACCCTGAGATGGGGGGAAAGCCGAGAAGGAAGAACCCTCAG
GAAGGCCTGTTCAATGAACTGCAGAAAGATAAGATGGCGGAGGCCTTCAGTGAGAT
TGGGATGAAAGGCGAGCGCCGGAGGGGCAAGGGGCACGATGGCCTTTTCCAGGGT
CTCAGTACAGCCACCAAGGACACCTTCGACGCCCTTCACATGCAGGCCCTGCCCCCT
CGC

2.3. TCR like Fusion Molecules

In certain embodiments, the chimeric receptor is a TCR like fusion molecule. Non-limiting examples of TCR fusion molecules include HLA-Independent TCR-based Chimeric Antigen Receptor (also known as “HIT”, e.g., those disclosed in International Patent Application No. PCT/US19/017525, which is incorporated by reference in its entirety), T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety), synthetic T cell receptor and antigen receptor (STAR) (e.g., those disclosed in Liu et al. Science Translational Medicine (2021); 13(586):eabb5191, which is incorporated by reference in its entirety), antibody-T-cell receptor (AbTCR) (e.g., those disclosed in Xu et al. Cell Discovery (2018) 4:62, which is incorporated by reference in its entirety), and T cell antigen coupler (TAC) (e.g., those disclosed in Helsen et al. Nature Communications (2018); 9:3049, which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen (e.g., CD19), is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

### 2.4. Exemplified Cells

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is KLRG1⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ KLRG1⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ KLRG1⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is KLRG1⁻ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is KLRG1⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is KLRG1⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ KLRG1⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ KLRG1⁻ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ KLRG1⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ KLRG1⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ KLRG1⁻ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ KLRG1⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ KLRG1⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is KLRG1⁻ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is KLRG1⁻ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is KLRG1⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ KLRG1⁻ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ KLRG1⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ KLRG1⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ KLRG1⁻ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ KLRG1⁻ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ KLRG1⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ KLRG1⁻ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ KLRG1⁻ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ KLRG1⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is KLRG1⁻ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ KLRG1⁻ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ KLRG1⁻ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ KLRG1⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ KLRG1⁻ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is TIM3⁺ KLRG1⁻ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD8⁺ T cell. In certain embodiments, the T cell is CD57⁺ TIM3⁺ KLRG1⁻ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺. In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is KLRG1⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ KLRG1⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ KLRG1⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is KLRG1⁻ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is KLRG1⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is KLRG1⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ KLRG1⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ KLRG1⁻ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ KLRG1⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ KLRG1⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ KLRG1⁻ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ KLRG1⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ KLRG1⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is KLRG1⁻ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is KLRG1⁻ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is KLRG1⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ KLRG1⁻ CTLA4⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ KLRG1⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ KLRG1⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ KLRG1⁻ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ KLRG1⁻ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ KLRG1⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ KLRG1⁻ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ KLRG1⁻ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ KLRG1⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is KLRG1⁻ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ KLRG1⁻ CTLA4⁻ LAG3⁻.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ KLRG1⁻ CTLA4⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ KLRG1⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ KLRG1⁻ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is TIM3⁺ KLRG1⁻ CTLA4⁻ LAG3⁻ PD1⁺.

In certain embodiments, the cell comprises a CD19-targeted chimeric receptor. In certain embodiments, the CD19-targeted chimeric receptor is a CD19-targeted CAR. In certain embodiments, the CAR is 19(T2)28z1XX. In certain embodiments, the cell is a T cell. In certain embodiments, the T cell is a CD4⁺ T cell. In certain embodiments, the T cell is CD57⁺ TIM3⁺ KLRG1⁻ CTLA4⁻ LAG3⁻ PD1⁺.

### 3. Nucleic Acid Molecules, Vector and Genetic Modifications

The presently disclosed subject matter provides nucleic acid molecules encoding the presently disclosed CD19-targeted chimeric receptors (e.g., those disclosed in Section 2.2). In certain embodiments, the nucleic acid molecule further comprises a promoter that is operably linked to the presently disclosed CD19-targeted chimeric receptor. Also provided are cells comprising such nucleic acid molecules.

In certain embodiments, the promoter is endogenous or exogenous. In certain embodiments, the exogenous promoter is selected from the group consisting of an elongation factor (EF)-1 promoter, a cytomegalovirus immediate-early promoter (CMV) promoter, a simian virus 40 early promoter (SV40) promoter, a phosphoglycerate kinase (PGK) promoter, a metallothionein promoter, and Ubiquitin C promoter. In certain embodiments, the endogenous promoter is selected from a TCR alpha promoter, a TCR beta promoter, and a beta 2-microglobulin promoter. In certain embodiments, the promoter is an inducible promoter. In certain embodiments, the inducible promoter is selected from the group consisting of a NFAT transcriptional response element (TRE) promoter, a CD69 promoter, a CD25 promoter, an IL-2 promoter, a 4-1BB promoter, a PD1 promoter, and a LAG3 promoter.

The presently disclosed subject matter also provides vectors comprising the presently disclosed nucleic acid molecules. In certain embodiments, the vector is a viral vector. In certain embodiments, the viral vector is a retroviral vector. In certain embodiments, the retroviral vector is a gamma retroviral vector or lentiviral vector.

In certain embodiments, the vector comprises a nucleic acid molecule encoding a presently disclosed CD19-targeted chimeric receptor. In certain embodiments, the CD19-targeted chimeric receptor is a CD19-targeted CAR. In certain embodiments, the nucleic acid molecule encodes a polypeptide comprising the amino acid sequence set forth in SEQ ID NO: 45. In certain embodiments, the nucleic acid molecule comprises the nucleotide sequence set forth in SEQ ID NO: 46.

The nucleic acid molecules can be delivered into cells by art-known methods or as described herein. Genetic modification of a cell can be accomplished by transducing a substantially homogeneous cell composition with a recombinant DNA construct. In certain embodiments, a retroviral vector (e.g., gammaretroviral vector or lentiviral vector) is employed for the introduction of the DNA construct into the cell. For example, a polynucleotide encoding a presently disclosed chimeric receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include the presently disclosed chimeric receptor, a retroviral vector can be employed for transduction, however, any other suitable viral vector or non-viral delivery system can be used. The chimeric receptor can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). An exemplary P2A peptide comprises or consists of the amino acid sequence set forth in SEQ ID NO: 47, which is provided below:

[SEQ ID NO: 47]
GSGATNFSLLKQAGDVEENPGP

An exemplary nucleotide sequence encoding the amino acid sequence of SEQ ID NO: 47 is set forth in 48, which is provided below:

[SEQ ID NO: 48]
GGAAGCGGAGCTACTAACTTCAGCCTGCTGAAGCAGGCTGGAGACGTGG
AGGAGAACCCTGGACCC

Combinations of retroviral vectors and an appropriate packaging lines are also suitable, where the capsid proteins will be functional for infecting human cells. Various amphotropic virus-producing cell lines are known, including, but not limited to, PA12 (Miller et al., (1985)Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD 114, or GALV envelope, and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., Bio Techniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995)107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALEN, CRISPR). Transient expression may be obtained by RNA electroporation.

Any targeted genome editing methods can also be used to deliver the presently disclosed chimeric receptor (e.g., a presently disclosed CD19-targeted chimeric receptor) to a cell. In certain embodiments, a CRISPR system is used to deliver the presently disclosed chimeric receptor (e.g., a presently disclosed CD19-targeted chimeric receptor). In certain embodiments, zinc-finger nucleases are used to deliver the presently disclosed chimeric receptor (e.g., a presently disclosed CD19-targeted chimeric receptor). In certain embodiments, a TALEN system is used to deliver the presently disclosed chimeric receptor (e.g., a presently disclosed CD19-targeted chimeric receptor).

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying a chimeric receptor expression cassette (e.g., a CAR expression cassette) needs also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of base pairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying a chimeric receptor expression cassette (e.g., a CAR expression cassette), ZFNs can be used to insert the CAR expression cassette into the genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), metallothionein promoters, or Ubiquitin C promoter), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10⁻⁹ to about 10⁻⁶ m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC). As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3″′-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12′″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS and PD-1).

In certain embodiments, the delivery methods are in vivo delivery methods. In certain embodiments, the delivery methods are ex vivo delivery methods.

### 4. Formulations and Administration

The presently disclosed subject matter also provides compositions comprising the presently disclosed cells (e.g., those disclosed in Section 2). For example, but without any limitation, the cells of the presently disclosed compositions comprise a chimeric receptor (e.g., those disclosed in Section 2.2). In certain embodiments, the composition is a pharmaceutical composition that further comprises a pharmaceutically acceptable carrier.

In certain embodiments, the presently disclosed compositions comprise cells (e.g., those disclosed in Section 2) that have been selected for the presence or absence of certain biomarkers. In certain embodiments, the biomarkers are selected from CD57, TIM3, KLRG1, CTLA4, LAG3, PD1, or any combination thereof.

In certain embodiments, the presently disclosed compositions comprise cells (e.g., those disclosed in Section 2) that have been selected for the expression level of certain biomarkers compared to the expression level of a reference sample. In certain embodiments, the compositions comprise cells having an expression level of CD57, TIM3, and/or PD1 that is increased compared to the expression level of a reference sample. In certain embodiments, the compositions comprise cells having an expression level of KLRG1, CTLA4, and/or LAG3 that is reduced compared to the expression level of a reference sample.

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ cells (e.g., T cells). In certain embodiments, the composition comprises at least about 5% CD57⁺ cells (e.g., T cells). In certain embodiments, the composition comprises at least about 10% CD57⁺ cells (e.g., T cells). In certain embodiments, the composition comprises at least about 15% CD57⁺ cells (e.g., T cells). In certain embodiments, the composition comprises at least about 20% CD57⁺ cells (e.g., T cells). In certain embodiments, the composition comprises at least about 25% CD57⁺ cells (e.g., T cells). In certain embodiments, the composition comprises at least about 30% CD57⁺ cells (e.g., T cells). In certain embodiments, the composition comprises at least about 35% CD57⁺ cells (e.g., T cells). In certain embodiments, the composition comprises at least about 40% CD57⁺ cells (e.g., T cells). In certain embodiments, the composition comprises at least about 45% CD57⁺ cells (e.g., T cells). In certain embodiments, the composition comprises at least about 50% CD57⁺ cells (e.g., T cells). In certain embodiments, the composition comprises at least about 55% CD57⁺ cells (e.g., T cells). In certain embodiments, the composition comprises at least about 60% CD57⁺ cells (e.g., T cells). In certain embodiments, the composition comprises at least about 65% CD57⁺ cells (e.g., T cells). In certain embodiments, the composition comprises at least about 70% CD57⁺ cells (e.g., T cells). In certain embodiments, the composition comprises at least about 75% CD57⁺ cells (e.g., T cells). In certain embodiments, the composition comprises at least about 80% CD57⁺ cells (e.g., T cells). In certain embodiments, the composition comprises at least about 85% CD57⁺ cells (e.g., T cells). In certain embodiments, the composition comprises at least about 90% CD57⁺ cells (e.g., T cells). In certain embodiments, the composition comprises at least about 95% CD57⁺ cells (e.g., T cells). In certain embodiments, the composition comprises about 100% CD57⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ cells (e.g., T cells). In certain embodiments, the composition comprises at least about 5% TIM3⁺ cells (e.g., T cells). In certain embodiments, the composition comprises at least about 10% TIM3⁺ cells (e.g., T cells). In certain embodiments, the composition comprises at least about 15% TIM3⁺ cells (e.g., T cells). In certain embodiments, the composition comprises at least about 20% TIM3⁺ cells (e.g., T cells). In certain embodiments, the composition comprises at least about 25% TIM3⁺ cells (e.g., T cells). In certain embodiments, the composition comprises at least about 30% TIM3⁺ cells (e.g., T cells). In certain embodiments, the composition comprises at least about 35% TIM3⁺ cells (e.g., T cells). In certain embodiments, the composition comprises at least about 40% TIM3⁺ cells (e.g., T cells). In certain embodiments, the composition comprises at least about 45% TIM3⁺ cells (e.g., T cells). In certain embodiments, the composition comprises at least about 50% TIM3⁺ cells (e.g., T cells). In certain embodiments, the composition comprises at least about 55% TIM3⁺ cells (e.g., T cells). In certain embodiments, the composition comprises at least about 60% TIM3⁺ cells (e.g., T cells). In certain embodiments, the composition comprises at least about 65% TIM3⁺ cells (e.g., T cells). In certain embodiments, the composition comprises at least about 70% TIM3⁺ cells (e.g., T cells). In certain embodiments, the composition comprises at least about 75% TIM3⁺ cells (e.g., T cells). In certain embodiments, the composition comprises at least about 80% TIM3⁺ cells (e.g., T cells). In certain embodiments, the composition comprises at least about 85% TIM3⁺ cells (e.g., T cells). In certain embodiments, the composition comprises at least about 90% TIM3⁺ cells (e.g., T cells). In certain embodiments, the composition comprises at least about 95% TIM3⁺ cells (e.g., T cells). In certain embodiments, the composition comprises about 100% TIM3⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% KLRG1⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% KLRG1 cells (e.g., T cells). In certain embodiments, the composition comprises at least about 5% KLRG1⁻ cells (e.g., T cells). In certain embodiments, the composition comprises at least about 10% KLRG1⁻ cells (e.g., T cells). In certain embodiments, the composition comprises at least about 15% KLRG1⁻ cells (e.g., T cells). In certain embodiments, the composition comprises at least about 20% KLRG1⁻ cells (e.g., T cells). In certain embodiments, the composition comprises at least about 25% KLRG1⁻ cells (e.g., T cells). In certain embodiments, the composition comprises at least about 30% KLRG1⁻ cells (e.g., T cells). In certain embodiments, the composition comprises at least about 35% KLRG1⁻ cells (e.g., T cells). In certain embodiments, the composition comprises at least about 40% KLRG1⁻ cells (e.g., T cells). In certain embodiments, the composition comprises at least about 45% KLRG1⁻ cells (e.g., T cells). In certain embodiments, the composition comprises at least about 50% KLRG1⁻ cells (e.g., T cells). In certain embodiments, the composition comprises at least about 55% KLRG1⁻ cells (e.g., T cells). In certain embodiments, the composition comprises at least about 60% KLRG1⁻ cells (e.g., T cells). In certain embodiments, the composition comprises at least about 65% KLRG1⁻ cells (e.g., T cells). In certain embodiments, the composition comprises at least about 70% KLRG1⁻ cells (e.g., T cells). In certain embodiments, the composition comprises at least about 75% KLRG1⁻ cells (e.g., T cells). In certain embodiments, the composition comprises at least about 80% KLRG1⁻ cells (e.g., T cells). In certain embodiments, the composition comprises at least about 85% KLRG1⁻ cells (e.g., T cells). In certain embodiments, the composition comprises at least about 90% KLRG1⁻ cells (e.g., T cells). In certain embodiments, the composition comprises at least about 95% KLRG1⁻ cells (e.g., T cells). In certain embodiments, the composition comprises about 100% KLRG1⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CTLA4⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CTLA4⁻ cells (e.g., T cells). In certain embodiments, the composition comprises at least about 5% CTLA4⁻ cells (e.g., T cells). In certain embodiments, the composition comprises at least about 10% CTLA4⁻ cells (e.g., T cells). In certain embodiments, the composition comprises at least about 15% CTLA4⁻ cells (e.g., T cells). In certain embodiments, the composition comprises at least about 20% CTLA4⁻ cells (e.g., T cells). In certain embodiments, the composition comprises at least about 25% CTLA4⁻ cells (e.g., T cells). In certain embodiments, the composition comprises at least about 30% CTLA4⁻ cells (e.g., T cells). In certain embodiments, the composition comprises at least about 35% CTLA4⁻ cells (e.g., T cells). In certain embodiments, the composition comprises at least about 40% CTLA4⁻ cells (e.g., T cells). In certain embodiments, the composition comprises at least about 45% CTLA4⁻ cells (e.g., T cells). In certain embodiments, the composition comprises at least about 50% CTLA4⁻ cells (e.g., T cells). In certain embodiments, the composition comprises at least about 55% CTLA4⁻ cells (e.g., T cells). In certain embodiments, the composition comprises at least about 60% CTLA4⁻ cells (e.g., T cells). In certain embodiments, the composition comprises at least about 65% CTLA4⁻ cells (e.g., T cells). In certain embodiments, the composition comprises at least about 70% CTLA4⁻ cells (e.g., T cells). In certain embodiments, the composition comprises at least about 75% CTLA4⁻ cells (e.g., T cells). In certain embodiments, the composition comprises at least about 80% CTLA4⁻ cells (e.g., T cells). In certain embodiments, the composition comprises at least about 85% CTLA4⁻ cells (e.g., T cells). In certain embodiments, the composition comprises at least about 90% CTLA4⁻ cells (e.g., T cells). In certain embodiments, the composition comprises at least about 95% CTLA4⁻ cells (e.g., T cells). In certain embodiments, the composition comprises about 100% CTLA4⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% LAG3⁻ cells (e.g., T cells). In certain embodiments, the composition comprises at least about 5% LAG3⁻ cells (e.g., T cells). In certain embodiments, the composition comprises at least about 10% LAG3⁻ cells (e.g., T cells). In certain embodiments, the composition comprises at least about 15% LAG3⁻ cells (e.g., T cells). In certain embodiments, the composition comprises at least about 20% LAG3⁻ cells (e.g., T cells). In certain embodiments, the composition comprises at least about 25% LAG3⁻ cells (e.g., T cells). In certain embodiments, the composition comprises at least about 30% LAG3⁻ cells (e.g., T cells). In certain embodiments, the composition comprises at least about 35% LAG3⁻ cells (e.g., T cells). In certain embodiments, the composition comprises at least about 40% LAG3⁻ cells (e.g., T cells). In certain embodiments, the composition comprises at least about 45% LAG3⁻ cells (e.g., T cells). In certain embodiments, the composition comprises at least about 50% LAG3⁻ cells (e.g., T cells). In certain embodiments, the composition comprises at least about 55% LAG3⁻ cells (e.g., T cells). In certain embodiments, the composition comprises at least about 60% LAG3⁻ cells (e.g., T cells). In certain embodiments, the composition comprises at least about 65% LAG3⁻ cells (e.g., T cells). In certain embodiments, the composition comprises at least about 70% LAG3⁻ cells (e.g., T cells). In certain embodiments, the composition comprises at least about 75% LAG3⁻ cells (e.g., T cells). In certain embodiments, the composition comprises at least about 80% LAG3⁻ cells (e.g., T cells). In certain embodiments, the composition comprises at least about 85% LAG3⁻ cells (e.g., T cells). In certain embodiments, the composition comprises at least about 90% LAG3⁻ cells (e.g., T cells). In certain embodiments, the composition comprises at least about 95% LAG3⁻ cells (e.g., T cells). In certain embodiments, the composition comprises about 100% LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% PD1⁺ cells (e.g., T cells). In certain embodiments, the composition comprises at least about 5% PD1⁺ cells (e.g., T cells). In certain embodiments, the composition comprises at least about 10% PD1⁺ cells (e.g., T cells). In certain embodiments, the composition comprises at least about 15% PD1⁺ cells (e.g., T cells). In certain embodiments, the composition comprises at least about 20% PD1⁺ cells (e.g., T cells). In certain embodiments, the composition comprises at least about 25% PD1⁺ cells (e.g., T cells). In certain embodiments, the composition comprises at least about 30% PD1⁺ cells (e.g., T cells). In certain embodiments, the composition comprises at least about 35% PD1⁺ cells (e.g., T cells). In certain embodiments, the composition comprises at least about 40% PD1⁺ cells (e.g., T cells). In certain embodiments, the composition comprises at least about 45% PD1⁺ cells (e.g., T cells). In certain embodiments, the composition comprises at least about 50% PD1⁺ cells (e.g., T cells). In certain embodiments, the composition comprises at least about 55% PD1⁺ cells (e.g., T cells). In certain embodiments, the composition comprises at least about 60% PD1⁺ cells (e.g., T cells). In certain embodiments, the composition comprises at least about 65% PD1⁺ cells (e.g., T cells). In certain embodiments, the composition comprises at least about 70% PD1⁺ cells (e.g., T cells). In certain embodiments, the composition comprises at least about 75% PD1⁺ cells (e.g., T cells). In certain embodiments, the composition comprises at least about 80% PD1⁺ cells (e.g., T cells). In certain embodiments, the composition comprises at least about 85% PD1⁺ cells (e.g., T cells). In certain embodiments, the composition comprises at least about 90% PD1⁺ cells (e.g., T cells). In certain embodiments, the composition comprises at least about 95% PD1⁺ cells (e.g., T cells). In certain embodiments, the composition comprises about 100% PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ cells (e.g., T cells). In certain embodiments, the composition comprises at least about 5% CD57⁺ TIM3⁺ cells (e.g., T cells). In certain embodiments, the composition comprises at least about 10% CD57⁺ TIM3⁺ cells (e.g., T cells). In certain embodiments, the composition comprises at least about 15% CD57⁺ TIM3⁺ cells (e.g., T cells). In certain embodiments, the composition comprises at least about 20% CD57⁺ TIM3⁺ cells (e.g., T cells). In certain embodiments, the composition comprises at least about 25% CD57⁺ TIM3⁺ cells (e.g., T cells). In certain embodiments, the composition comprises at least about 30% CD57⁺ TIM3⁺ cells (e.g., T cells). In certain embodiments, the composition comprises at least about 35% CD57⁺ TIM3⁺ cells (e.g., T cells). In certain embodiments, the composition comprises at least about 40% CD57⁺ TIM3⁺ cells (e.g., T cells). In certain embodiments, the composition comprises at least about 45% CD57⁺ TIM3⁺ cells (e.g., T cells). In certain embodiments, the composition comprises at least about 50% CD57⁺ TIM3⁺ cells (e.g., T cells). In certain embodiments, the composition comprises at least about 55% CD57⁺ TIM3⁺ cells (e.g., T cells). In certain embodiments, the composition comprises at least about 60% CD57⁺ TIM3⁺ cells (e.g., T cells). In certain embodiments, the composition comprises at least about 65% CD57⁺ TIM3⁺ cells (e.g., T cells). In certain embodiments, the composition comprises at least about 70% CD57⁺ TIM3⁺ cells (e.g., T cells). In certain embodiments, the composition comprises at least about 75% CD57⁺ TIM3⁺ cells (e.g., T cells). In certain embodiments, the composition comprises at least about 80% CD57⁺ TIM3⁺ cells (e.g., T cells). In certain embodiments, the composition comprises at least about 85% CD57⁺ TIM3⁺ cells (e.g., T cells). In certain embodiments, the composition comprises at least about 90% CD57⁺ TIM3⁺ cells (e.g., T cells). In certain embodiments, the composition comprises at least about 95% CD57⁺ TIM3⁺ cells (e.g., T cells). In certain embodiments, the composition comprises about 100% CD57⁺ TIM3⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ KLRG1⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ KLRG1⁻ cells (e.g., T cells). In certain embodiments, the composition comprises at least about 5% CD57⁺ KLRG1⁻ cells (e.g., T cells). In certain embodiments, the composition comprises at least about 10% CD57⁺ KLRG1⁻ cells (e.g., T cells). In certain embodiments, the composition comprises at least about 15% CD57⁺ KLRG1⁻ KLRG1⁻ cells (e.g., T cells). In certain embodiments, the composition comprises at least about 20% CD57⁺ KLRG1⁻ cells (e.g., T cells). In certain embodiments, the composition comprises at least about 25% CD57⁺ KLRG1⁻ cells (e.g., T cells). In certain embodiments, the composition comprises at least about 30% CD57⁺ KLRG1⁻ cells (e.g., T cells). In certain embodiments, the composition comprises at least about 35% CD57⁺ KLRG1⁻ cells (e.g., T cells). In certain embodiments, the composition comprises at least about 40% CD57⁺ KLRG1⁻ cells (e.g., T cells). In certain embodiments, the composition comprises at least about 45% CD57⁺ KLRG1⁻ cells (e.g., T cells). In certain embodiments, the composition comprises at least about 50% CD57⁺ KLRG1⁻ cells (e.g., T cells). In certain embodiments, the composition comprises at least about 55% CD57⁺ KLRG1⁻ cells (e.g., T cells). In certain embodiments, the composition comprises at least about 60% CD57⁺ KLRG1⁻ cells (e.g., T cells). In certain embodiments, the composition comprises at least about 65% CD57⁺ KLRG1⁻ cells (e.g., T cells). In certain embodiments, the composition comprises at least about 70% CD57⁺ KLRG1⁻ cells (e.g., T cells). In certain embodiments, the composition comprises at least about 75% CD57⁺ KLRG1⁻ cells (e.g., T cells). In certain embodiments, the composition comprises at least about 80% CD57⁺ KLRG1⁻ cells (e.g., T cells). In certain embodiments, the composition comprises at least about 85% CD57⁺ KLRG1⁻ cells (e.g., T cells). In certain embodiments, the composition comprises at least about 90% CD57⁺ KLRG1⁻ cells (e.g., T cells). In certain embodiments, the composition comprises at least about 95% CD57⁺ KLRG1⁻ cells (e.g., T cells). In certain embodiments, the composition comprises about 100% CD57⁺ KLRG1⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ CTLA4⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ CTLA4⁻ cells (e.g., T cells). In certain embodiments, the composition comprises at least about 5% CD57⁺ CTLA4⁻ cells (e.g., T cells). In certain embodiments, the composition comprises at least about 10% CD57⁺ CTLA4⁻ cells (e.g., T cells). In certain embodiments, the composition comprises at least about 15% CD57⁺ CTLA4⁻ CTLA4⁻ cells (e.g., T cells). In certain embodiments, the composition comprises at least about 20% CD57⁺ CTLA4⁻ cells (e.g., T cells). In certain embodiments, the composition comprises at least about 25% CD57⁺ CTLA4⁻ cells (e.g., T cells). In certain embodiments, the composition comprises at least about 30% CD57⁺ CTLA4⁻ cells (e.g., T cells). In certain embodiments, the composition comprises at least about 35% CD57⁺ CTLA4⁻ cells (e.g., T cells). In certain embodiments, the composition comprises at least about 40% CD57⁺ CTLA4⁻ cells (e.g., T cells). In certain embodiments, the composition comprises at least about 45% CD57⁺ CTLA4⁻ cells (e.g., T cells). In certain embodiments, the composition comprises at least about 50% CD57⁺ CTLA4⁻ cells (e.g., T cells). In certain embodiments, the composition comprises at least about 55% CD57⁺ CTLA4⁻ cells (e.g., T cells). In certain embodiments, the composition comprises at least about 60% CD57⁺ CTLA4⁻ cells (e.g., T cells). In certain embodiments, the composition comprises at least about 65% CD57⁺ CTLA4⁻ cells (e.g., T cells). In certain embodiments, the composition comprises at least about 70% CD57⁺ CTLA4⁻ cells (e.g., T cells). In certain embodiments, the composition comprises at least about 75% CD57⁺ CTLA4⁻ cells (e.g., T cells). In certain embodiments, the composition comprises at least about 80% CD57⁺ CTLA4⁻ cells (e.g., T cells). In certain embodiments, the composition comprises at least about 85% CD57⁺ CTLA4⁻ cells (e.g., T cells). In certain embodiments, the composition comprises at least about 90% CD57⁺ CTLA4⁻ cells (e.g., T cells). In certain embodiments, the composition comprises at least about 95% CD57⁺ CTLA4⁻ cells (e.g., T cells). In certain embodiments, the composition comprises about 100% CD57⁺ CTLA4⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ LAG3⁻ cells (e.g., T cells). In certain embodiments, the composition comprises at least about 5% CD57⁺ LAG3⁻ cells (e.g., T cells). In certain embodiments, the composition comprises at least about 10% CD57⁺ LAG3⁻ cells (e.g., T cells). In certain embodiments, the composition comprises at least about 15% CD57⁺ LAG3⁻ LAG3⁻ cells (e.g., T cells). In certain embodiments, the composition comprises at least about 20% CD57⁺ LAG3⁻ cells (e.g., T cells). In certain embodiments, the composition comprises at least about 25% CD57⁺ LAG3⁻ cells (e.g., T cells). In certain embodiments, the composition comprises at least about 30% CD57⁺ LAG3⁻ cells (e.g., T cells). In certain embodiments, the composition comprises at least about 35% CD57⁺ LAG3⁻ cells (e.g., T cells). In certain embodiments, the composition comprises at least about 40% CD57⁺ LAG3⁻ cells (e.g., T cells). In certain embodiments, the composition comprises at least about 45% CD57⁺ LAG3⁻ cells (e.g., T cells). In certain embodiments, the composition comprises at least about 50% CD57⁺ LAG3⁻ cells (e.g., T cells). In certain embodiments, the composition comprises at least about 55% CD57⁺ LAG3⁻ cells (e.g., T cells). In certain embodiments, the composition comprises at least about 60% CD57⁺ LAG3⁻ cells (e.g., T cells). In certain embodiments, the composition comprises at least about 65% CD57⁺ LAG3⁻ cells (e.g., T cells). In certain embodiments, the composition comprises at least about 70% CD57⁺ LAG3⁻ cells (e.g., T cells). In certain embodiments, the composition comprises at least about 75% CD57⁺ LAG3⁻ cells (e.g., T cells). In certain embodiments, the composition comprises at least about 80% CD57⁺ LAG3⁻ cells (e.g., T cells). In certain embodiments, the composition comprises at least about 85% CD57⁺ LAG3⁻ cells (e.g., T cells). In certain embodiments, the composition comprises at least about 90% CD57⁺ LAG3⁻ cells (e.g., T cells). In certain embodiments, the composition comprises at least about 95% CD57⁺ LAG3⁻ cells (e.g., T cells). In certain embodiments, the composition comprises about 100% CD57⁺ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ PD1⁺ cells (e.g., T cells). In certain embodiments, the composition comprises at least about 5% CD57⁺ PD1⁺ cells (e.g., T cells). In certain embodiments, the composition comprises at least about 10% CD57⁺ PD1⁺ cells (e.g., T cells). In certain embodiments, the composition comprises at least about 15% CD57⁺ PD1⁺ cells (e.g., T cells). In certain embodiments, the composition comprises at least about 20% CD57⁺ PD1⁺ cells (e.g., T cells). In certain embodiments, the composition comprises at least about 25% CD57⁺ PD1⁺ cells (e.g., T cells). In certain embodiments, the composition comprises at least about 30% CD57⁺ PD1⁺ cells (e.g., T cells). In certain embodiments, the composition comprises at least about 35% CD57⁺ PD1⁺ cells (e.g., T cells). In certain embodiments, the composition comprises at least about 40% CD57⁺ PD1⁺ cells (e.g., T cells). In certain embodiments, the composition comprises at least about 45% CD57⁺ PD1⁺ cells (e.g., T cells). In certain embodiments, the composition comprises at least about 50% CD57⁺ PD1⁺ cells (e.g., T cells). In certain embodiments, the composition comprises at least about 55% CD57⁺ PD1⁺ cells (e.g., T cells). In certain embodiments, the composition comprises at least about 60% CD57⁺ PD1⁺ cells (e.g., T cells). In certain embodiments, the composition comprises at least about 65% CD57⁺ PD1⁺ cells (e.g., T cells). In certain embodiments, the composition comprises at least about 70% CD57⁺ PD1⁺ cells (e.g., T cells). In certain embodiments, the composition comprises at least about 75% CD57⁺ PD1⁺ cells (e.g., T cells). In certain embodiments, the composition comprises at least about 80% CD57⁺ PD1⁺ cells (e.g., T cells). In certain embodiments, the composition comprises at least about 85% CD57⁺ PD1⁺ cells (e.g., T cells). In certain embodiments, the composition comprises at least about 90% CD57⁺ PD1⁺ cells (e.g., T cells). In certain embodiments, the composition comprises at least about 95% CD57⁺ PD1⁺ cells (e.g., T cells). In certain embodiments, the composition comprises about 100% CD57⁺ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ KLRG1⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ KLRG1⁻ cells (e.g., T cells). In certain embodiments, the composition comprises at least about 5% TIM3⁺ KLRG1⁻ cells (e.g., T cells). In certain embodiments, the composition comprises at least about 10% TIM3⁺ KLRG1⁻ cells (e.g., T cells). In certain embodiments, the composition comprises at least about 15% TIM3⁺ KLRG1⁻ KLRG1⁻ cells (e.g., T cells). In certain embodiments, the composition comprises at least about 20% TIM3⁺ KLRG1⁻ cells (e.g., T cells). In certain embodiments, the composition comprises at least about 25% TIM3⁺ KLRG1⁻ cells (e.g., T cells). In certain embodiments, the composition comprises at least about 30% TIM3⁺ KLRG1⁻ cells (e.g., T cells). In certain embodiments, the composition comprises at least about 35% TIM3⁺ KLRG1⁻ cells (e.g., T cells). In certain embodiments, the composition comprises at least about 40% TIM3⁺ KLRG1⁻ cells (e.g., T cells). In certain embodiments, the composition comprises at least about 45% TIM3⁺ KLRG1⁻ cells (e.g., T cells). In certain embodiments, the composition comprises at least about 50% TIM3⁺ KLRG1⁻ cells (e.g., T cells). In certain embodiments, the composition comprises at least about 55% TIM3⁺ KLRG1⁻ cells (e.g., T cells). In certain embodiments, the composition comprises at least about 60% TIM3⁺ KLRG1⁻ cells (e.g., T cells). In certain embodiments, the composition comprises at least about 65% TIM3⁺ KLRG1⁻ cells (e.g., T cells). In certain embodiments, the composition comprises at least about 70% TIM3⁺ KLRG1⁻ cells (e.g., T cells). In certain embodiments, the composition comprises at least about 75% TIM3⁺ KLRG1⁻ cells (e.g., T cells). In certain embodiments, the composition comprises at least about 80% TIM3⁺ KLRG1⁻ cells (e.g., T cells). In certain embodiments, the composition comprises at least about 85% TIM3⁺ KLRG1⁻ cells (e.g., T cells). In certain embodiments, the composition comprises at least about 90% TIM3⁺ KLRG1⁻ cells (e.g., T cells). In certain embodiments, the composition comprises at least about 95% TIM3⁺ KLRG1⁻ cells (e.g., T cells). In certain embodiments, the composition comprises about 100% TIM3⁺ KLRG1⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ CTLA4⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ CTLA4⁻ cells (e.g., T cells). In certain embodiments, the composition comprises at least about 5% TIM3⁺ CTLA4⁻ cells (e.g., T cells). In certain embodiments, the composition comprises at least about 10% TIM3⁺ CTLA4⁻ cells (e.g., T cells). In certain embodiments, the composition comprises at least about 15% TIM3⁺ CTLA4⁻ CTLA4⁻ cells (e.g., T cells). In certain embodiments, the composition comprises at least about 20% TIM3⁺ CTLA4⁻ cells (e.g., T cells). In certain embodiments, the composition comprises at least about 25% TIM3⁺ CTLA4⁻ cells (e.g., T cells). In certain embodiments, the composition comprises at least about 30% TIM3⁺ CTLA4⁻ cells (e.g., T cells). In certain embodiments, the composition comprises at least about 35% TIM3⁺ CTLA4⁻ cells (e.g., T cells). In certain embodiments, the composition comprises at least about 40% TIM3⁺ CTLA4⁻ cells (e.g., T cells). In certain embodiments, the composition comprises at least about 45% TIM3⁺ CTLA4⁻ cells (e.g., T cells). In certain embodiments, the composition comprises at least about 50% TIM3⁺ CTLA4⁻ cells (e.g., T cells). In certain embodiments, the composition comprises at least about 55% TIM3⁺ CTLA4⁻ cells (e.g., T cells). In certain embodiments, the composition comprises at least about 60% TIM3⁺ CTLA4⁻ cells (e.g., T cells). In certain embodiments, the composition comprises at least about 65% TIM3⁺ CTLA4⁻ cells (e.g., T cells). In certain embodiments, the composition comprises at least about 70% TIM3⁺ CTLA4⁻ cells (e.g., T cells). In certain embodiments, the composition comprises at least about 75% TIM3⁺ CTLA4⁻ cells (e.g., T cells). In certain embodiments, the composition comprises at least about 80% TIM3⁺ CTLA4⁻ cells (e.g., T cells). In certain embodiments, the composition comprises at least about 85% TIM3⁺ CTLA4⁻ cells (e.g., T cells). In certain embodiments, the composition comprises at least about 90% TIM3⁺ CTLA4⁻ cells (e.g., T cells). In certain embodiments, the composition comprises at least about 95% TIM3⁺ CTLA4⁻ cells (e.g., T cells). In certain embodiments, the composition comprises about 100% TIM3⁺ CTLA4⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ LAG3⁻ cells (e.g., T cells). In certain embodiments, the composition comprises at least about 5% TIM3⁺ LAG3⁻ cells (e.g., T cells). In certain embodiments, the composition comprises at least about 10% TIM3⁺ LAG3⁻ cells (e.g., T cells). In certain embodiments, the composition comprises at least about 15% TIM3⁺ LAG3⁻ LAG3⁻ cells (e.g., T cells). In certain embodiments, the composition comprises at least about 20% TIM3⁺ LAG3⁻ cells (e.g., T cells). In certain embodiments, the composition comprises at least about 25% TIM3⁺ LAG3⁻ cells (e.g., T cells). In certain embodiments, the composition comprises at least about 30% TIM3⁺ LAG3⁻ cells (e.g., T cells). In certain embodiments, the composition comprises at least about 35% TIM3⁺ LAG3⁻ cells (e.g., T cells). In certain embodiments, the composition comprises at least about 40% TIM3⁺ LAG3⁻ cells (e.g., T cells). In certain embodiments, the composition comprises at least about 45% TIM3⁺ LAG3⁻ cells (e.g., T cells). In certain embodiments, the composition comprises at least about 50% TIM3⁺ LAG3⁻ cells (e.g., T cells). In certain embodiments, the composition comprises at least about 55% TIM3⁺ LAG3⁻ cells (e.g., T cells). In certain embodiments, the composition comprises at least about 60% TIM3⁺ LAG3⁻ cells (e.g., T cells). In certain embodiments, the composition comprises at least about 65% TIM3⁺ LAG3⁻ cells (e.g., T cells). In certain embodiments, the composition comprises at least about 70% TIM3⁺ LAG3⁻ cells (e.g., T cells). In certain embodiments, the composition comprises at least about 75% TIM3⁺ LAG3⁻ cells (e.g., T cells). In certain embodiments, the composition comprises at least about 80% TIM3⁺ LAG3⁻ cells (e.g., T cells). In certain embodiments, the composition comprises at least about 85% TIM3⁺ LAG3⁻ cells (e.g., T cells). In certain embodiments, the composition comprises at least about 90% TIM3⁺ LAG3⁻ cells (e.g., T cells). In certain embodiments, the composition comprises at least about 95% TIM3⁺ LAG3⁻ cells (e.g., T cells). In certain embodiments, the composition comprises about 100% TIM3⁺ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ PD1⁺ cells (e.g., T cells). In certain embodiments, the composition comprises at least about 5% TIM3⁺ PD1⁺ cells (e.g., T cells). In certain embodiments, the composition comprises at least about 10% TIM3⁺ PD1⁺ cells (e.g., T cells). In certain embodiments, the composition comprises at least about 15% TIM3⁺ PD1⁺ cells (e.g., T cells). In certain embodiments, the composition comprises at least about 20% TIM3⁺ PD1⁺ cells (e.g., T cells). In certain embodiments, the composition comprises at least about 25% TIM3⁺ PD1⁺ cells (e.g., T cells). In certain embodiments, the composition comprises at least about 30% TIM3⁺ PD1⁺ cells (e.g., T cells). In certain embodiments, the composition comprises at least about 35% TIM3⁺ PD1⁺ cells (e.g., T cells). In certain embodiments, the composition comprises at least about 40% TIM3⁺ PD1⁺ cells (e.g., T cells). In certain embodiments, the composition comprises at least about 45% TIM3⁺ PD1⁺ cells (e.g., T cells). In certain embodiments, the composition comprises at least about 50% TIM3⁺ PD1⁺ cells (e.g., T cells). In certain embodiments, the composition comprises at least about 55% TIM3⁺ PD1⁺ cells (e.g., T cells). In certain embodiments, the composition comprises at least about 60% TIM3⁺ PD1⁺ cells (e.g., T cells). In certain embodiments, the composition comprises at least about 65% TIM3⁺ PD1⁺ cells (e.g., T cells). In certain embodiments, the composition comprises at least about 70% TIM3⁺ PD1⁺ cells (e.g., T cells). In certain embodiments, the composition comprises at least about 75% TIM3⁺ PD1⁺ cells (e.g., T cells). In certain embodiments, the composition comprises at least about 80% TIM3⁺ PD1⁺ cells (e.g., T cells). In certain embodiments, the composition comprises at least about 85% TIM3⁺ PD1⁺ cells (e.g., T cells). In certain embodiments, the composition comprises at least about 90% TIM3⁺ PD1⁺ cells (e.g., T cells). In certain embodiments, the composition comprises at least about 95% TIM3⁺ PD1⁺ cells (e.g., T cells). In certain embodiments, the composition comprises about 100% TIM3⁺ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% KLRG1⁻ CTLA4⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% KLRG1⁻ CTLA4⁻ cells (e.g., T cells). In certain embodiments, the composition comprises at least about 5% KLRG1⁻ CTLA4⁻ cells (e.g., T cells). In certain embodiments, the composition comprises at least about 10% KLRG1⁻ CTLA4⁻ cells (e.g., T cells). In certain embodiments, the composition comprises at least about 15% KLRG1⁻ CTLA4⁻ CTLA4⁻ cells (e.g., T cells). In certain embodiments, the composition comprises at least about 20% KLRG1⁻ CTLA4⁻ cells (e.g., T cells). In certain embodiments, the composition comprises at least about 25% KLRG1⁻ CTLA4⁻ cells (e.g., T cells). In certain embodiments, the composition comprises at least about 30% KLRG1⁻ CTLA4⁻ cells (e.g., T cells). In certain embodiments, the composition comprises at least about 35% KLRG1⁻ CTLA4⁻ cells (e.g., T cells). In certain embodiments, the composition comprises at least about 40% KLRG1⁻ CTLA4⁻ cells (e.g., T cells). In certain embodiments, the composition comprises at least about 45% KLRG1⁻ CTLA4⁻ cells (e.g., T cells). In certain embodiments, the composition comprises at least about 50% KLRG1⁻ CTLA4⁻ cells (e.g., T cells). In certain embodiments, the composition comprises at least about 55% KLRG1⁻ CTLA4⁻ cells (e.g., T cells). In certain embodiments, the composition comprises at least about 60% KLRG1⁻ CTLA4⁻ cells (e.g., T cells). In certain embodiments, the composition comprises at least about 65% KLRG1⁻ CTLA4⁻ cells (e.g., T cells). In certain embodiments, the composition comprises at least about 70% KLRG1⁻ CTLA4⁻ cells (e.g., T cells). In certain embodiments, the composition comprises at least about 75% KLRG1⁻ CTLA4⁻ cells (e.g., T cells). In certain embodiments, the composition comprises at least about 80% KLRG1⁻ CTLA4⁻ cells (e.g., T cells). In certain embodiments, the composition comprises at least about 85% KLRG1⁻ CTLA4⁻ cells (e.g., T cells). In certain embodiments, the composition comprises at least about 90% KLRG1⁻ CTLA4⁻ cells (e.g., T cells). In certain embodiments, the composition comprises at least about 95% KLRG1⁻ CTLA4⁻ cells (e.g., T cells). In certain embodiments, the composition comprises about 100% KLRG1⁻ CTLA4⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% KLRG1⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% KLRG1⁻ LAG3⁻ cells (e.g., T cells). In certain embodiments, the composition comprises at least about 5% KLRG1⁻ LAG3⁻ cells (e.g., T cells). In certain embodiments, the composition comprises at least about 10% KLRG1⁻ LAG3⁻ cells (e.g., T cells). In certain embodiments, the composition comprises at least about 15% KLRG1⁻ LAG3⁻ cells (e.g., T cells). In certain embodiments, the composition comprises at least about 20% KLRG1⁻ LAG3⁻ cells (e.g., T cells). In certain embodiments, the composition comprises at least about 25% KLRG1⁻ LAG3⁻ cells (e.g., T cells). In certain embodiments, the composition comprises at least about 30% KLRG1⁻ LAG3⁻ cells (e.g., T cells). In certain embodiments, the composition comprises at least about 35% KLRG1⁻ LAG3⁻ cells (e.g., T cells). In certain embodiments, the composition comprises at least about 40% KLRG1⁻ LAG3⁻ cells (e.g., T cells). In certain embodiments, the composition comprises at least about 45% KLRG1⁻ LAG3⁻ cells (e.g., T cells). In certain embodiments, the composition comprises at least about 50% KLRG1⁻ LAG3⁻ cells (e.g., T cells). In certain embodiments, the composition comprises at least about 55% KLRG1⁻ LAG3⁻ cells (e.g., T cells). In certain embodiments, the composition comprises at least about 60% KLRG1⁻ LAG3⁻ cells (e.g., T cells). In certain embodiments, the composition comprises at least about 65% KLRG1⁻ LAG3⁻ cells (e.g., T cells). In certain embodiments, the composition comprises at least about 70% KLRG1⁻ LAG3⁻ cells (e.g., T cells). In certain embodiments, the composition comprises at least about 75% KLRG1⁻ LAG3⁻ cells (e.g., T cells). In certain embodiments, the composition comprises at least about 80% KLRG1⁻ LAG3⁻ cells (e.g., T cells). In certain embodiments, the composition comprises at least about 85% KLRG1⁻ LAG3⁻ cells (e.g., T cells). In certain embodiments, the composition comprises at least about 90% KLRG1⁻ LAG3⁻ cells (e.g., T cells). In certain embodiments, the composition comprises at least about 95% KLRG1⁻ LAG3⁻ cells (e.g., T cells). In certain embodiments, the composition comprises about 100% KLRG1⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% KLRG1⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% KLRG1⁻ PD1⁺ cells (e.g., T cells). In certain embodiments, the composition comprises at least about 5% KLRG1⁻ PD1⁺ cells (e.g., T cells). In certain embodiments, the composition comprises at least about 10% KLRG1⁻ PD1⁺ cells (e.g., T cells). In certain embodiments, the composition comprises at least about 15% KLRG1⁻ PD1⁺ cells (e.g., T cells). In certain embodiments, the composition comprises at least about 20% KLRG1⁻ PD1⁺ cells (e.g., T cells). In certain embodiments, the composition comprises at least about 25% KLRG1⁻ PD1 cells (e.g., T cells). In certain embodiments, the composition comprises at least about 30% KLRG1⁻ PD1⁺ cells (e.g., T cells). In certain embodiments, the composition comprises at least about 35% KLRG1⁻ PD1⁺ cells (e.g., T cells). In certain embodiments, the composition comprises at least about 40% KLRG1⁻ PD1⁺ cells (e.g., T cells). In certain embodiments, the composition comprises at least about 45% KLRG1⁻ PD1⁺ cells (e.g., T cells). In certain embodiments, the composition comprises at least about 50% KLRG1⁻ PD1⁺ cells (e.g., T cells). In certain embodiments, the composition comprises at least about 55% KLRG1⁻ PD1⁺ cells (e.g., T cells). In certain embodiments, the composition comprises at least about 60% KLRG1⁻ PD1⁺ cells (e.g., T cells). In certain embodiments, the composition comprises at least about 65% KLRG1⁻ PD1⁺ cells (e.g., T cells). In certain embodiments, the composition comprises at least about 70% KLRG1⁻ PD1⁺ cells (e.g., T cells). In certain embodiments, the composition comprises at least about 75% KLRG1⁻ PD1⁺ cells (e.g., T cells). In certain embodiments, the composition comprises at least about 80% KLRG1⁻ PD1⁺ cells (e.g., T cells). In certain embodiments, the composition comprises at least about 85% KLRG1⁻ PD1⁺ cells (e.g., T cells). In certain embodiments, the composition comprises at least about 90% KLRG1⁻ PD1⁺ cells (e.g., T cells). In certain embodiments, the composition comprises at least about 95% KLRG1⁻ PD1⁺ cells (e.g., T cells). In certain embodiments, the composition comprises about 100% KLRG1⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CTLA4⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CTLA4⁻ LAG3⁻ cells (e.g., T cells). In certain embodiments, the composition comprises at least about 5% CTLA4⁻ LAG3⁻ cells (e.g., T cells). In certain embodiments, the composition comprises at least about 10% CTLA4⁻ LAG3⁻ cells (e.g., T cells). In certain embodiments, the composition comprises at least about 15% CTLA4⁻ LAG3⁻ LAG3⁻ cells (e.g., T cells). In certain embodiments, the composition comprises at least about 20% CTLA4⁻ LAG3⁻ cells (e.g., T cells). In certain embodiments, the composition comprises at least about 25% CTLA4⁻ LAG3⁻ cells (e.g., T cells). In certain embodiments, the composition comprises at least about 30% CTLA4⁻ LAG3⁻ cells (e.g., T cells). In certain embodiments, the composition comprises at least about 35% CTLA4⁻ LAG3⁻ cells (e.g., T cells). In certain embodiments, the composition comprises at least about 40% CTLA4⁻ LAG3⁻ cells (e.g., T cells). In certain embodiments, the composition comprises at least about 45% CTLA4⁻ LAG3⁻ cells (e.g., T cells). In certain embodiments, the composition comprises at least about 50% CTLA4⁻ LAG3⁻ cells (e.g., T cells). In certain embodiments, the composition comprises at least about 55% CTLA4⁻ LAG3⁻ cells (e.g., T cells). In certain embodiments, the composition comprises at least about 60% CTLA4⁻ LAG3⁻ cells (e.g., T cells). In certain embodiments, the composition comprises at least about 65% CTLA4⁻ LAG3⁻ cells (e.g., T cells). In certain embodiments, the composition comprises at least about 70% CTLA4⁻ LAG3⁻ cells (e.g., T cells). In certain embodiments, the composition comprises at least about 75% CTLA4⁻ LAG3⁻ cells (e.g., T cells). In certain embodiments, the composition comprises at least about 80% CTLA4⁻ LAG3⁻ cells (e.g., T cells). In certain embodiments, the composition comprises at least about 85% CTLA4⁻ LAG3⁻ cells (e.g., T cells). In certain embodiments, the composition comprises at least about 90% CTLA4⁻ LAG3⁻ cells (e.g., T cells). In certain embodiments, the composition comprises at least about 95% CTLA4⁻ LAG3⁻ cells (e.g., T cells). In certain embodiments, the composition comprises about 100% CTLA4⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CTLA4⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CTLA4⁻ PD1⁺ cells (e.g., T cells). In certain embodiments, the composition comprises at least about 5% CTLA4⁻ PD1⁺ cells (e.g., T cells). In certain embodiments, the composition comprises at least about 10% CTLA4⁻ PD1⁺ cells (e.g., T cells). In certain embodiments, the composition comprises at least about 15% CTLA4⁻ PD1⁺ cells (e.g., T cells). In certain embodiments, the composition comprises at least about 20% CTLA4⁻ PD1⁺ cells (e.g., T cells). In certain embodiments, the composition comprises at least about 25% CTLA4⁻ PD1⁺ cells (e.g., T cells). In certain embodiments, the composition comprises at least about 30% CTLA4⁻ PD1⁺ cells (e.g., T cells). In certain embodiments, the composition comprises at least about 35% CTLA4⁻ PD1⁺ cells (e.g., T cells). In certain embodiments, the composition comprises at least about 40% CTLA4⁻ PD1⁺ cells (e.g., T cells). In certain embodiments, the composition comprises at least about 45% CTLA4⁻ PD1⁺ cells (e.g., T cells). In certain embodiments, the composition comprises at least about 50% CTLA4⁻ PD1⁺ cells (e.g., T cells). In certain embodiments, the composition comprises at least about 55% CTLA4⁻ PD1⁺ cells (e.g., T cells). In certain embodiments, the composition comprises at least about 60% CTLA4⁻ PD1⁺ cells (e.g., T cells). In certain embodiments, the composition comprises at least about 65% CTLA4⁻ PD1⁺ cells (e.g., T cells). In certain embodiments, the composition comprises at least about 70% CTLA4⁻ PD1⁺ cells (e.g., T cells). In certain embodiments, the composition comprises at least about 75% CTLA4⁻ PD1⁺ cells (e.g., T cells). In certain embodiments, the composition comprises at least about 80% CTLA4⁻ PD1⁺ cells (e.g., T cells). In certain embodiments, the composition comprises at least about 85% CTLA4⁻ PD1⁺ cells (e.g., T cells). In certain embodiments, the composition comprises at least about 90% CTLA4⁻ PD1⁺ cells (e.g., T cells). In certain embodiments, the composition comprises at least about 95% CTLA4⁻ PD1⁺ cells (e.g., T cells). In certain embodiments, the composition comprises about 100% CTLA4⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% LAG3⁻ PD1⁺ cells (e.g., T cells). In certain embodiments, the composition comprises at least about 5% LAG3⁻ PD1⁺ cells (e.g., T cells). In certain embodiments, the composition comprises at least about 10% LAG3⁻ PD1⁺ cells (e.g., T cells). In certain embodiments, the composition comprises at least about 15% LAG3⁻ PD1⁺ cells (e.g., T cells). In certain embodiments, the composition comprises at least about 20% LAG3⁻ PD1⁺ cells (e.g., T cells). In certain embodiments, the composition comprises at least about 25% LAG3⁻ PD1⁺ cells (e.g., T cells). In certain embodiments, the composition comprises at least about 30% LAG3⁻ PD1⁺ cells (e.g., T cells). In certain embodiments, the composition comprises at least about 35% LAG3⁻ PD1⁺ cells (e.g., T cells). In certain embodiments, the composition comprises at least about 40% LAG3⁻ PD1⁺ cells (e.g., T cells). In certain embodiments, the composition comprises at least about 45% LAG3⁻ PD1⁺ cells (e.g., T cells). In certain embodiments, the composition comprises at least about 50% LAG3⁻ PD1⁺ cells (e.g., T cells). In certain embodiments, the composition comprises at least about 55% LAG3⁻ PD1⁺ cells (e.g., T cells). In certain embodiments, the composition comprises at least about 60% LAG3⁻ PD1⁺ cells (e.g., T cells). In certain embodiments, the composition comprises at least about 65% LAG3⁻ PD1⁺ cells (e.g., T cells). In certain embodiments, the composition comprises at least about 70% LAG3⁻ PD1⁺ cells (e.g., T cells). In certain embodiments, the composition comprises at least about 75% LAG3⁻ PD1⁺ cells (e.g., T cells). In certain embodiments, the composition comprises at least about 80% LAG3⁻ PD1⁺ cells (e.g., T cells). In certain embodiments, the composition comprises at least about 85% LAG3⁻ PD1⁺ cells (e.g., T cells). In certain embodiments, the composition comprises at least about 90% LAG3⁻ PD1⁺ cells (e.g., T cells). In certain embodiments, the composition comprises at least about 95% LAG3⁻ PD1⁺ cells (e.g., T cells). In certain embodiments, the composition comprises about 100% LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ KLRG1⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ KLRG1⁻ cells (e.g., T cells). In certain embodiments, the composition comprises at least about 5% CD57⁺ TIM3⁺ KLRG1⁻ cells (e.g., T cells). In certain embodiments, the composition comprises at least about 10% CD57⁺ TIM3⁺ KLRG1⁻ cells (e.g., T cells). In certain embodiments, the composition comprises at least about 15% CD57⁺ TIM3⁺ KLRG1⁻ cells (e.g., T cells). In certain embodiments, the composition comprises at least about 20% CD57⁺ TIM3⁺ KLRG1⁻ cells (e.g., T cells). In certain embodiments, the composition comprises at least about 25% CD57⁺ TIM3⁺ KLRG1⁻ cells (e.g., T cells). In certain embodiments, the composition comprises at least about 30% CD57⁺ TIM3⁺ KLRG1⁻ cells (e.g., T cells). In certain embodiments, the composition comprises at least about 35% CD57⁺ TIM3⁺ KLRG1⁻ cells (e.g., T cells). In certain embodiments, the composition comprises at least about 40% CD57⁺ TIM3⁺ KLRG1⁻ cells (e.g., T cells). In certain embodiments, the composition comprises at least about 45% CD57⁺ TIM3⁺ KLRG1⁻ cells (e.g., T cells). In certain embodiments, the composition comprises at least about 50% CD57⁺ TIM3⁺ KLRG1⁻ cells (e.g., T cells). In certain embodiments, the composition comprises at least about 55% CD57⁺ TIM3⁺ KLRG1⁻ cells (e.g., T cells). In certain embodiments, the composition comprises at least about 60% CD57⁺ TIM3⁺ KLRG1⁻ cells (e.g., T cells). In certain embodiments, the composition comprises at least about 65% CD57⁺ TIM3⁺ KLRG1⁻ cells (e.g., T cells). In certain embodiments, the composition comprises at least about 70% CD57⁺ TIM3⁺ KLRG1⁻ cells (e.g., T cells). In certain embodiments, the composition comprises at least about 75% CD57⁺ TIM3⁺ KLRG1⁻ cells (e.g., T cells). In certain embodiments, the composition comprises at least about 80% CD57⁺ TIM3⁺ KLRG1⁻ cells (e.g., T cells). In certain embodiments, the composition comprises at least about 85% CD57⁺ TIM3⁺ KLRG1⁻ cells (e.g., T cells). In certain embodiments, the composition comprises at least about 90% CD57⁺ TIM3⁺ KLRG1⁻ cells (e.g., T cells). In certain embodiments, the composition comprises at least about 95% CD57⁺ TIM3⁺ KLRG1⁻ cells (e.g., T cells). In certain embodiments, the composition comprises about 100% CD57⁺ TIM3⁺ KLRG1⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ CTLA4⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ CTLA4⁻ cells (e.g., T cells). In certain embodiments, the composition comprises at least about 5% CD57⁺ TIM3⁺ CTLA4⁻ cells (e.g., T cells). In certain embodiments, the composition comprises at least about 10% CD57⁺ TIM3⁺ CTLA4⁻ cells (e.g., T cells). In certain embodiments, the composition comprises at least about 15% CD57⁺ TIM3⁺ CTLA4⁻ cells (e.g., T cells). In certain embodiments, the composition comprises at least about 20% CD57⁺ TIM3⁺ CTLA4⁻ cells (e.g., T cells). In certain embodiments, the composition comprises at least about 25% CD57⁺ TIM3⁺ CTLA4⁻ cells (e.g., T cells). In certain embodiments, the composition comprises at least about 30% CD57⁺ TIM3⁺ CTLA4⁻ cells (e.g., T cells). In certain embodiments, the composition comprises at least about 35% CD57⁺ TIM3⁺ CTLA4⁻ cells (e.g., T cells). In certain embodiments, the composition comprises at least about 40% CD57⁺ TIM3⁺ CTLA4⁻ cells (e.g., T cells). In certain embodiments, the composition comprises at least about 45% CD57⁺ TIM3⁺ CTLA4⁻ cells (e.g., T cells). In certain embodiments, the composition comprises at least about 50% CD57⁺ TIM3⁺ CTLA4⁻ cells (e.g., T cells). In certain embodiments, the composition comprises at least about 55% CD57⁺ TIM3⁺ CTLA4⁻ cells (e.g., T cells). In certain embodiments, the composition comprises at least about 60% CD57⁺ TIM3⁺ CTLA4⁻ cells (e.g., T cells). In certain embodiments, the composition comprises at least about 65% CD57⁺ TIM3⁺ CTLA4⁻ cells (e.g., T cells). In certain embodiments, the composition comprises at least about 70% CD57⁺ TIM3⁺ CTLA4⁻ cells (e.g., T cells). In certain embodiments, the composition comprises at least about 75% CD57⁺ TIM3⁺ CTLA4⁻ cells (e.g., T cells). In certain embodiments, the composition comprises at least about 80% CD57⁺ TIM3⁺ CTLA4⁻ cells (e.g., T cells). In certain embodiments, the composition comprises at least about 85% CD57⁺ TIM3⁺ CTLA4⁻ cells (e.g., T cells). In certain embodiments, the composition comprises at least about 90% CD57⁺ TIM3⁺ CTLA4⁻ cells (e.g., T cells). In certain embodiments, the composition comprises at least about 95% CD57⁺ TIM3⁺ CTLA4⁻ cells (e.g., T cells). In certain embodiments, the composition comprises about 100% CD57⁺ TIM3⁺ CTLA4⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ LAG3⁻ cells (e.g., T cells). In certain embodiments, the composition comprises at least about 5% CD57⁺ TIM3⁺ LAG3⁻ cells (e.g., T cells). In certain embodiments, the composition comprises at least about 10% CD57⁺ TIM3⁺ LAG3⁻ cells (e.g., T cells). In certain embodiments, the composition comprises at least about 15% CD57⁺ TIM3⁺ LAG3⁻ cells (e.g., T cells). In certain embodiments, the composition comprises at least about 20% CD57⁺ TIM3⁺ LAG3⁻ cells (e.g., T cells). In certain embodiments, the composition comprises at least about 25% CD57⁺ TIM3⁺ LAG3⁻ cells (e.g., T cells). In certain embodiments, the composition comprises at least about 30% CD57⁺ TIM3⁺ LAG3⁻ cells (e.g., T cells). In certain embodiments, the composition comprises at least about 35% CD57⁺ TIM3⁺ LAG3⁻ cells (e.g., T cells). In certain embodiments, the composition comprises at least about 40% CD57⁺ TIM3⁺ LAG3⁻ cells (e.g., T cells). In certain embodiments, the composition comprises at least about 45% CD57⁺ TIM3⁺ LAG3⁻ cells (e.g., T cells). In certain embodiments, the composition comprises at least about 50% CD57⁺ TIM3⁺ LAG3⁻ cells (e.g., T cells). In certain embodiments, the composition comprises at least about 55% CD57⁺ TIM3⁺ LAG3⁻ cells (e.g., T cells). In certain embodiments, the composition comprises at least about 60% CD57⁺ TIM3⁺ LAG3⁻ cells (e.g., T cells). In certain embodiments, the composition comprises at least about 65% CD57⁺ TIM3⁺ LAG3⁻ cells (e.g., T cells). In certain embodiments, the composition comprises at least about 70% CD57⁺ TIM3⁺ LAG3⁻ cells (e.g., T cells). In certain embodiments, the composition comprises at least about 75% CD57⁺ TIM3⁺ LAG3⁻ cells (e.g., T cells). In certain embodiments, the composition comprises at least about 80% CD57⁺ TIM3⁺ LAG3⁻ cells (e.g., T cells). In certain embodiments, the composition comprises at least about 85% CD57⁺ TIM3⁺ LAG3⁻ cells (e.g., T cells). In certain embodiments, the composition comprises at least about 90% CD57⁺ TIM3⁺ LAG3⁻ cells (e.g., T cells). In certain embodiments, the composition comprises at least about 95% CD57⁺ TIM3⁺ LAG3⁻ cells (e.g., T cells). In certain embodiments, the composition comprises about 100% CD57⁺ TIM3⁺ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ PD1⁺ cells (e.g., T cells). In certain embodiments, the composition comprises at least about 5% CD57⁺ TIM3⁺ PD1⁺ cells (e.g., T cells). In certain embodiments, the composition comprises at least about 10% CD57⁺ TIM3⁺ PD1⁺ cells (e.g., T cells). In certain embodiments, the composition comprises at least about 15% CD57⁺ TIM3⁺ PD1⁺ cells (e.g., T cells). In certain embodiments, the composition comprises at least about 20% CD57⁺ TIM3⁺ PD1⁺ cells (e.g., T cells). In certain embodiments, the composition comprises at least about 25% CD57⁺ TIM3⁺ PD1⁺ cells (e.g., T cells). In certain embodiments, the composition comprises at least about 30% CD57⁺ TIM3⁺ PD1⁺ cells (e.g., T cells). In certain embodiments, the composition comprises at least about 35% CD57⁺ TIM3⁺ PD1⁺ cells (e.g., T cells). In certain embodiments, the composition comprises at least about 40% CD57⁺ TIM3⁺ PD1⁺ cells (e.g., T cells). In certain embodiments, the composition comprises at least about 45% CD57⁺ TIM3⁺ PD1⁺ cells (e.g., T cells). In certain embodiments, the composition comprises at least about 50% CD57⁺ TIM3⁺ PD1⁺ cells (e.g., T cells). In certain embodiments, the composition comprises at least about 55% CD57⁺ TIM3⁺ PD1⁺ cells (e.g., T cells). In certain embodiments, the composition comprises at least about 60% CD57⁺ TIM3⁺ PD1⁺ cells (e.g., T cells). In certain embodiments, the composition comprises at least about 65% CD57⁺ TIM3⁺ PD1⁺ cells (e.g., T cells). In certain embodiments, the composition comprises at least about 70% CD57⁺ TIM3⁺ PD1⁺⁻ cells (e.g., T cells). In certain embodiments, the composition comprises at least about 75% CD57⁺ TIM3⁺ PD1⁺ cells (e.g., T cells). In certain embodiments, the composition comprises at least about 80% CD57⁺ TIM3⁺ PD1⁺ cells (e.g., T cells). In certain embodiments, the composition comprises at least about 85% CD57⁺ TIM3⁺ PD1⁺ cells (e.g., T cells). In certain embodiments, the composition comprises at least about 90% CD57⁺ TIM3⁺ PD1⁺ cells (e.g., T cells). In certain embodiments, the composition comprises at least about 95% CD57⁺ TIM3⁺ PD1⁺ cells (e.g., T cells). In certain embodiments, the composition comprises about 100% CD57⁺ TIM3⁺ PD1⁺⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ KLRG1⁻ CTLA4⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ KLRG1⁻ CTLA4⁻ cells (e.g., T cells). In certain embodiments, the composition comprises at least about 5% CD57⁺ KLRG1⁻ CTLA4⁻ cells (e.g., T cells). In certain embodiments, the composition comprises at least about 10% CD57⁺ KLRG1⁻ CTLA4⁻ cells (e.g., T cells). In certain embodiments, the composition comprises at least about 15% CD57⁺ KLRG1⁻ CTLA4⁻ cells (e.g., T cells). In certain embodiments, the composition comprises at least about 20% CD57⁺ KLRG1⁻ CTLA4⁻ cells (e.g., T cells). In certain embodiments, the composition comprises at least about 25% CD57⁺ KLRG1⁻ CTLA4⁻ cells (e.g., T cells). In certain embodiments, the composition comprises at least about 30% CD57⁺ KLRG1⁻ CTLA4⁻ cells (e.g., T cells). In certain embodiments, the composition comprises at least about 35% CD57⁺ KLRG1⁻ CTLA4⁻ cells (e.g., T cells). In certain embodiments, the composition comprises at least about 40% CD57⁺ KLRG1⁻ CTLA4⁻ cells (e.g., T cells). In certain embodiments, the composition comprises at least about 45% CD57⁺ KLRG1⁻ CTLA4⁻ cells (e.g., T cells). In certain embodiments, the composition comprises at least about 50% CD57⁺ KLRG1⁻ CTLA4⁻ cells (e.g., T cells). In certain embodiments, the composition comprises at least about 55% CD57⁺ KLRG1⁻ CTLA4⁻ cells (e.g., T cells). In certain embodiments, the composition comprises at least about 60% CD57⁺ KLRG1⁻ CTLA4⁻ cells (e.g., T cells). In certain embodiments, the composition comprises at least about 65% CD57⁺ KLRG1⁻ CTLA4⁻ cells (e.g., T cells). In certain embodiments, the composition comprises at least about 70% CD57⁺ KLRG1⁻ CTLA4⁻ cells (e.g., T cells). In certain embodiments, the composition comprises at least about 75% CD57⁺ KLRG1⁻ CTLA4⁻ cells (e.g., T cells). In certain embodiments, the composition comprises at least about 80% CD57⁺ KLRG1⁻ CTLA4⁻ cells (e.g., T cells). In certain embodiments, the composition comprises at least about 85% CD57⁺ KLRG1⁻ CTLA4⁻ cells (e.g., T cells). In certain embodiments, the composition comprises at least about 90% CD57⁺ KLRG1⁻ CTLA4⁻ cells (e.g., T cells). In certain embodiments, the composition comprises at least about 95% CD57⁺ KLRG1⁻ CTLA4⁻ cells (e.g., T cells). In certain embodiments, the composition comprises about 100% CD57⁺ KLRG1⁻ CTLA4⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ KLRG1⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ KLRG1⁻ LAG3⁻ cells (e.g., T cells). In certain embodiments, the composition comprises at least about 5% CD57⁺ KLRG1⁻ LAG3⁻ cells (e.g., T cells). In certain embodiments, the composition comprises at least about 10% CD57⁺ KLRG1⁻ LAG3⁻ cells (e.g., T cells). In certain embodiments, the composition comprises at least about 15% CD57⁺ KLRG1⁻ LAG3⁻ cells (e.g., T cells). In certain embodiments, the composition comprises at least about 20% CD57⁺ KLRG1⁻ LAG3⁻ cells (e.g., T cells). In certain embodiments, the composition comprises at least about 25% CD57⁺ KLRG1⁻ LAG3⁻ cells (e.g., T cells). In certain embodiments, the composition comprises at least about 30% CD57⁺ KLRG1⁻ LAG3⁻ cells (e.g., T cells). In certain embodiments, the composition comprises at least about 35% CD57⁺ KLRG1⁻ LAG3⁻ cells (e.g., T cells). In certain embodiments, the composition comprises at least about 40% CD57⁺ KLRG1⁻ LAG3⁻ cells (e.g., T cells). In certain embodiments, the composition comprises at least about 45% CD57⁺ KLRG1⁻ LAG3⁻ cells (e.g., T cells). In certain embodiments, the composition comprises at least about 50% CD57⁺ KLRG1⁻ LAG3⁻ cells (e.g., T cells). In certain embodiments, the composition comprises at least about 55% CD57⁺ KLRG1⁻ LAG3⁻ cells (e.g., T cells). In certain embodiments, the composition comprises at least about 60% CD57⁺ KLRG1⁻ LAG3⁻ cells (e.g., T cells). In certain embodiments, the composition comprises at least about 65% CD57⁺ KLRG1⁻ LAG3⁻ cells (e.g., T cells). In certain embodiments, the composition comprises at least about 70% CD57⁺ KLRG1⁻ LAG3⁻ cells (e.g., T cells). In certain embodiments, the composition comprises at least about 75% CD57⁺ KLRG1⁻ LAG3⁻ cells (e.g., T cells). In certain embodiments, the composition comprises at least about 80% CD57⁺ KLRG1⁻ LAG3⁻ cells (e.g., T cells). In certain embodiments, the composition comprises at least about 85% CD57⁺ KLRG1⁻ LAG3⁻ cells (e.g., T cells). In certain embodiments, the composition comprises at least about 90% CD57⁺ KLRG1⁻ LAG3⁻ cells (e.g., T cells). In certain embodiments, the composition comprises at least about 95% CD57⁺ KLRG1⁻ LAG3⁻ cells (e.g., T cells). In certain embodiments, the composition comprises about 100% CD57⁺ KLRG1⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ KLRG1⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ KLRG1⁻ PD1⁺ cells (e.g., T cells). In certain embodiments, the composition comprises at least about 5% CD57⁺ KLRG1⁻ PD1⁺ cells (e.g., T cells). In certain embodiments, the composition comprises at least about 10% CD57⁺ KLRG1⁻ PD1⁺ cells (e.g., T cells). In certain embodiments, the composition comprises at least about 15% CD57⁺ KLRG1⁻ PD1⁺ cells (e.g., T cells). In certain embodiments, the composition comprises at least about 20% CD57⁺ KLRG1⁻ PD1⁺ cells (e.g., T cells). In certain embodiments, the composition comprises at least about 25% CD57⁺ KLRG1⁻ PD1⁺ cells (e.g., T cells). In certain embodiments, the composition comprises at least about 30% CD57⁺ KLRG1⁻ PD1⁺ cells (e.g., T cells). In certain embodiments, the composition comprises at least about 35% CD57⁺ KLRG1⁻ PD1⁺ cells (e.g., T cells). In certain embodiments, the composition comprises at least about 40% CD57⁺ KLRG1⁻ PD1⁺ cells (e.g., T cells). In certain embodiments, the composition comprises at least about 45% CD57⁺ KLRG1⁻ PD1⁺ cells (e.g., T cells). In certain embodiments, the composition comprises at least about 50% CD57⁺ KLRG1⁻ PD1⁺ cells (e.g., T cells). In certain embodiments, the composition comprises at least about 55% CD57⁺ KLRG1⁻ PD1⁺ cells (e.g., T cells). In certain embodiments, the composition comprises at least about 60% CD57⁺ KLRG1⁻ PD1⁺ cells (e.g., T cells). In certain embodiments, the composition comprises at least about 65% CD57⁺ KLRG1⁻ PD1⁺ cells (e.g., T cells). In certain embodiments, the composition comprises at least about 70% CD57⁺ KLRG1⁻ PD1⁺ cells (e.g., T cells). In certain embodiments, the composition comprises at least about 75% CD57⁺ KLRG1⁻ PD1⁺ cells (e.g., T cells). In certain embodiments, the composition comprises at least about 80% CD57⁺ KLRG1⁻ PD1⁺ cells (e.g., T cells). In certain embodiments, the composition comprises at least about 85% CD57⁺ KLRG1⁻ PD1⁺ cells (e.g., T cells). In certain embodiments, the composition comprises at least about 90% CD57⁺ KLRG1⁻ PD1⁺ cells (e.g., T cells). In certain embodiments, the composition comprises at least about 95% CD57⁺ KLRG1⁻ PD1⁺ cells (e.g., T cells). In certain embodiments, the composition comprises about 100% CD57⁺ KLRG1⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ CTLA4⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ CTLA4⁻ LAG3⁻ cells (e.g., T cells). In certain embodiments, the composition comprises at least about 5% CD57⁺ CTLA4⁻ LAG3⁻ cells (e.g., T cells). In certain embodiments, the composition comprises at least about 10% CD57⁺ CTLA4⁻ LAG3⁻ cells (e.g., T cells). In certain embodiments, the composition comprises at least about 15% CD57⁺ CTLA4⁻ LAG3⁻ cells (e.g., T cells). In certain embodiments, the composition comprises at least about 20% CD57⁺ CTLA4⁻ LAG3⁻ cells (e.g., T cells). In certain embodiments, the composition comprises at least about 25% CD57⁺ CTLA4⁻ LAG3⁻ cells (e.g., T cells). In certain embodiments, the composition comprises at least about 30% CD57⁺ CTLA4⁻ LAG3⁻ cells (e.g., T cells). In certain embodiments, the composition comprises at least about 35% CD57⁺ CTLA4⁻ LAG3⁻ cells (e.g., T cells). In certain embodiments, the composition comprises at least about 40% CD57⁺ CTLA4⁻ LAG3⁻ cells (e.g., T cells). In certain embodiments, the composition comprises at least about 45% CD57⁺ CTLA4⁻ LAG3⁻ cells (e.g., T cells). In certain embodiments, the composition comprises at least about 50% CD57⁺ CTLA4⁻ LAG3⁻ cells (e.g., T cells). In certain embodiments, the composition comprises at least about 55% CD57⁺ CTLA4⁻ LAG3⁻ cells (e.g., T cells). In certain embodiments, the composition comprises at least about 60% CD57⁺ CTLA4⁻ LAG3⁻ cells (e.g., T cells). In certain embodiments, the composition comprises at least about 65% CD57⁺ CTLA4⁻ LAG3⁻ cells (e.g., T cells). In certain embodiments, the composition comprises at least about 70% CD57⁺ CTLA4⁻ LAG3⁻ cells (e.g., T cells). In certain embodiments, the composition comprises at least about 75% CD57⁺ CTLA4⁻ LAG3⁻ cells (e.g., T cells). In certain embodiments, the composition comprises at least about 80% CD57⁺ CTLA4⁻ LAG3⁻ cells (e.g., T cells). In certain embodiments, the composition comprises at least about 85% CD57⁺ CTLA4⁻ LAG3⁻ cells (e.g., T cells). In certain embodiments, the composition comprises at least about 90% CD57⁺ CTLA4⁻ LAG3⁻ cells (e.g., T cells). In certain embodiments, the composition comprises at least about 95% CD57⁺ CTLA4⁻ LAG3⁻ cells (e.g., T cells). In certain embodiments, the composition comprises about 100% CD57⁺ CTLA4⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ CTLA4⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ CTLA4⁻ PD1⁺ cells (e.g., T cells). In certain embodiments, the composition comprises at least about 5% CD57⁺ CTLA4⁻ PD1⁺ cells (e.g., T cells). In certain embodiments, the composition comprises at least about 10% CD57⁺ CTLA4⁻ PD1⁺ cells (e.g., T cells). In certain embodiments, the composition comprises at least about 15% CD57⁺ CTLA4⁻ PD1⁺ cells (e.g., T cells). In certain embodiments, the composition comprises at least about 20% CD57⁺ CTLA4⁻ PD1⁺ cells (e.g., T cells). In certain embodiments, the composition comprises at least about 25% CD57⁺ CTLA4⁻ PD1⁺ cells (e.g., T cells). In certain embodiments, the composition comprises at least about 30% CD57⁺ CTLA4⁻ PD1⁺ cells (e.g., T cells). In certain embodiments, the composition comprises at least about 35% CD57⁺ CTLA4⁻ PD1⁺ cells (e.g., T cells). In certain embodiments, the composition comprises at least about 40% CD57⁺ CTLA4⁻ PD1⁺ cells (e.g., T cells). In certain embodiments, the composition comprises at least about 45% CD57⁺ CTLA4⁻ PD1⁺ cells (e.g., T cells). In certain embodiments, the composition comprises at least about 50% CD57⁺ CTLA4⁻ PD1⁺ cells (e.g., T cells). In certain embodiments, the composition comprises at least about 55% CD57⁺ CTLA4⁻ PD1⁺ cells (e.g., T cells). In certain embodiments, the composition comprises at least about 60% CD57⁺ CTLA4⁻ PD1⁺ cells (e.g., T cells). In certain embodiments, the composition comprises at least about 65% CD57⁺ CTLA4⁻ PD1⁺ cells (e.g., T cells). In certain embodiments, the composition comprises at least about 70% CD57⁺ CTLA4⁻ PD1⁺ cells (e.g., T cells). In certain embodiments, the composition comprises at least about 75% CD57⁺ CTLA4⁻ PD1⁺ cells (e.g., T cells). In certain embodiments, the composition comprises at least about 80% CD57⁺ CTLA4⁻ PD1⁺ cells (e.g., T cells). In certain embodiments, the composition comprises at least about 85% CD57⁺ CTLA4⁻ PD1⁺ cells (e.g., T cells). In certain embodiments, the composition comprises at least about 90% CD57⁺ CTLA4⁻ PD1⁺ cells (e.g., T cells). In certain embodiments, the composition comprises at least about 95% CD57⁺ CTLA4⁻ PD1⁺ cells (e.g., T cells). In certain embodiments, the composition comprises about 100% CD57⁺ CTLA4⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ LAG3⁻ PD1⁺ cells (e.g., T cells). In certain embodiments, the composition comprises at least about 5% CD57⁺ LAG3⁻ PD1⁺ cells (e.g., T cells). In certain embodiments, the composition comprises at least about 10% CD57⁺ LAG3⁻ PD1⁺ cells (e.g., T cells). In certain embodiments, the composition comprises at least about 15% CD57⁺ LAG3⁻ PD1⁺ cells (e.g., T cells). In certain embodiments, the composition comprises at least about 20% CD57⁺ LAG3⁻ PD1⁺ cells (e.g., T cells). In certain embodiments, the composition comprises at least about 25% CD57⁺ LAG3⁻ PD1⁺ cells (e.g., T cells). In certain embodiments, the composition comprises at least about 30% CD57⁺ LAG3⁻ PD1⁺ cells (e.g., T cells). In certain embodiments, the composition comprises at least about 35% CD57⁺ LAG3⁻ PD1⁺ cells (e.g., T cells). In certain embodiments, the composition comprises at least about 40% CD57⁺ LAG3⁻ PD1⁺ cells (e.g., T cells). In certain embodiments, the composition comprises at least about 45% CD57⁺ LAG3⁻ PD1⁺ cells (e.g., T cells). In certain embodiments, the composition comprises at least about 50% CD57⁺ LAG3⁻ PD1⁺ cells (e.g., T cells). In certain embodiments, the composition comprises at least about 55% CD57⁺ LAG3⁻ PD1⁺ cells (e.g., T cells). In certain embodiments, the composition comprises at least about 60% CD57⁺ LAG3⁻ PD1⁺ cells (e.g., T cells). In certain embodiments, the composition comprises at least about 65% CD57⁺ LAG3⁻ PD1⁺ cells (e.g., T cells). In certain embodiments, the composition comprises at least about 70% CD57⁺ LAG3⁻ PD1⁺ cells (e.g., T cells). In certain embodiments, the composition comprises at least about 75% CD57⁺ LAG3⁻ PD1⁺ cells (e.g., T cells). In certain embodiments, the composition comprises at least about 80% CD57⁺ LAG3⁻ PD1⁺ cells (e.g., T cells). In certain embodiments, the composition comprises at least about 85% CD57⁺ LAG3⁻ PD1⁺ cells (e.g., T cells). In certain embodiments, the composition comprises at least about 90% CD57⁺ LAG3⁻ PD1⁺ cells (e.g., T cells). In certain embodiments, the composition comprises at least about 95% CD57⁺ LAG3⁻ PD1⁺ cells (e.g., T cells). In certain embodiments, the composition comprises about 100% CD57⁺ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ KLRG1⁻ CTLA4⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ KLRG1⁻ CTLA4⁻ cells (e.g., T cells). In certain embodiments, the composition comprises at least about 5% TIM3⁺ KLRG1⁻ CTLA4⁻ cells (e.g., T cells). In certain embodiments, the composition comprises at least about 10% TIM3⁺ KLRG1⁻ CTLA4⁻ cells (e.g., T cells). In certain embodiments, the composition comprises at least about 15% TIM3⁺ KLRG1⁻ CTLA4⁻ cells (e.g., T cells). In certain embodiments, the composition comprises at least about 20% TIM3⁺ KLRG1⁻ CTLA4⁻ cells (e.g., T cells). In certain embodiments, the composition comprises at least about 25% TIM3⁺ KLRG1⁻ CTLA4⁻ cells (e.g., T cells). In certain embodiments, the composition comprises at least about 30% TIM3⁺ KLRG1⁻ CTLA4⁻ cells (e.g., T cells). In certain embodiments, the composition comprises at least about 35% TIM3⁺ KLRG1⁻ CTLA4⁻ cells (e.g., T cells). In certain embodiments, the composition comprises at least about 40% TIM3⁺ KLRG1⁻ CTLA4⁻ cells (e.g., T cells). In certain embodiments, the composition comprises at least about 45% TIM3⁺ KLRG1⁻ CTLA4⁻ cells (e.g., T cells). In certain embodiments, the composition comprises at least about 50% TIM3⁺ KLRG1⁻ CTLA4⁻ cells (e.g., T cells). In certain embodiments, the composition comprises at least about 55% TIM3⁺ KLRG1⁻ CTLA4⁻ cells (e.g., T cells). In certain embodiments, the composition comprises at least about 60% TIM3⁺ KLRG1⁻ CTLA4⁻ cells (e.g., T cells). In certain embodiments, the composition comprises at least about 65% TIM3⁺ KLRG1⁻ CTLA4⁻ cells (e.g., T cells). In certain embodiments, the composition comprises at least about 70% TIM3⁺ KLRG1⁻ CTLA4⁻ cells (e.g., T cells). In certain embodiments, the composition comprises at least about 75% TIM3⁺ KLRG1⁻ CTLA4⁻ cells (e.g., T cells). In certain embodiments, the composition comprises at least about 80% TIM3⁺ KLRG1⁻ CTLA4⁻ cells (e.g., T cells). In certain embodiments, the composition comprises at least about 85% TIM3⁺ KLRG1⁻ CTLA4⁻ cells (e.g., T cells). In certain embodiments, the composition comprises at least about 90% TIM3⁺ KLRG1⁻ CTLA4⁻ cells (e.g., T cells). In certain embodiments, the composition comprises at least about 95% TIM3⁺ KLRG1⁻ CTLA4⁻ cells (e.g., T cells). In certain embodiments, the composition comprises about 100% TIM3⁺ KLRG1⁻ CTLA4⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ KLRG1⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ KLRG1⁻ LAG3⁻ cells (e.g., T cells). In certain embodiments, the composition comprises at least about 5% TIM3⁺ KLRG1⁻ LAG3⁻ cells (e.g., T cells). In certain embodiments, the composition comprises at least about 10% TIM3⁺ KLRG1⁻ LAG3⁻ cells (e.g., T cells). In certain embodiments, the composition comprises at least about 15% TIM3⁺ KLRG1⁻ LAG3⁻ cells (e.g., T cells). In certain embodiments, the composition comprises at least about 20% TIM3⁺ KLRG1⁻ LAG3⁻ cells (e.g., T cells). In certain embodiments, the composition comprises at least about 25% TIM3⁺ KLRG1⁻ LAG3⁻ cells (e.g., T cells). In certain embodiments, the composition comprises at least about 30% TIM3⁺ KLRG1⁻ LAG3⁻ cells (e.g., T cells). In certain embodiments, the composition comprises at least about 35% TIM3⁺ KLRG1⁻ LAG3⁻ cells (e.g., T cells). In certain embodiments, the composition comprises at least about 40% TIM3⁺ KLRG1⁻ LAG3⁻ cells (e.g., T cells). In certain embodiments, the composition comprises at least about 45% TIM3⁺ KLRG1⁻ LAG3⁻ cells (e.g., T cells). In certain embodiments, the composition comprises at least about 50% TIM3⁺ KLRG1⁻ LAG3⁻ cells (e.g., T cells). In certain embodiments, the composition comprises at least about 55% TIM3⁺ KLRG1⁻ LAG3⁻ cells (e.g., T cells). In certain embodiments, the composition comprises at least about 60% TIM3⁺ KLRG1⁻ LAG3⁻ cells (e.g., T cells). In certain embodiments, the composition comprises at least about 65% TIM3⁺ KLRG1⁻ LAG3⁻ cells (e.g., T cells). In certain embodiments, the composition comprises at least about 70% TIM3⁺ KLRG1⁻ LAG3⁻ cells (e.g., T cells). In certain embodiments, the composition comprises at least about 75% TIM3⁺ KLRG1⁻ LAG3⁻ cells (e.g., T cells). In certain embodiments, the composition comprises at least about 80% TIM3⁺ KLRG1⁻ LAG3⁻ cells (e.g., T cells). In certain embodiments, the composition comprises at least about 85% TIM3⁺ KLRG1⁻ LAG3⁻ cells (e.g., T cells). In certain embodiments, the composition comprises at least about 90% TIM3⁺ KLRG1⁻ LAG3⁻ cells (e.g., T cells). In certain embodiments, the composition comprises at least about 95% TIM3⁺ KLRG1⁻ LAG3⁻ cells (e.g., T cells). In certain embodiments, the composition comprises about 100% TIM3⁺ KLRG1⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ KLRG1⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ KLRG1⁻ PD1⁺ cells (e.g., T cells). In certain embodiments, the composition comprises at least about 5% TIM3⁺ KLRG1⁻ PD1⁺ cells (e.g., T cells). In certain embodiments, the composition comprises at least about 10% TIM3⁺ KLRG1⁻ PD1⁺ cells (e.g., T cells). In certain embodiments, the composition comprises at least about 15% TIM3⁺ KLRG1⁻ PD1⁺ cells (e.g., T cells). In certain embodiments, the composition comprises at least about 20% TIM3⁺ KLRG1⁻ PD1⁺ cells (e.g., T cells). In certain embodiments, the composition comprises at least about 25% TIM3⁺ KLRG1⁻ PD1⁺ cells (e.g., T cells). In certain embodiments, the composition comprises at least about 30% TIM3⁺ KLRG1⁻ PD1⁺ cells (e.g., T cells). In certain embodiments, the composition comprises at least about 35% TIM3⁺ KLRG1⁻ PD1⁺ cells (e.g., T cells). In certain embodiments, the composition comprises at least about 40% TIM3⁺ KLRG1⁻ PD1⁺ cells (e.g., T cells). In certain embodiments, the composition comprises at least about 45% TIM3⁺ KLRG1⁻ PD1⁺ cells (e.g., T cells). In certain embodiments, the composition comprises at least about 50% TIM3⁺ KLRG1⁻ PD1⁺ cells (e.g., T cells). In certain embodiments, the composition comprises at least about 55% TIM3⁺ KLRG1⁻ PD1⁺ cells (e.g., T cells). In certain embodiments, the composition comprises at least about 60% TIM3⁺ KLRG1⁻ PD1⁺ cells (e.g., T cells). In certain embodiments, the composition comprises at least about 65% TIM3⁺ KLRG1⁻ PD1⁺ cells (e.g., T cells). In certain embodiments, the composition comprises at least about 70% TIM3⁺ KLRG1⁻ PD1⁺ cells (e.g., T cells). In certain embodiments, the composition comprises at least about 75% TIM3⁺ KLRG1⁻ PD1⁺ cells (e.g., T cells). In certain embodiments, the composition comprises at least about 80% TIM3⁺ KLRG1⁻ PD1⁺ cells (e.g., T cells). In certain embodiments, the composition comprises at least about 85% TIM3⁺ KLRG1⁻ PD1⁺ cells (e.g., T cells). In certain embodiments, the composition comprises at least about 90% TIM3⁺ KLRG1⁻ PD1⁺ cells (e.g., T cells). In certain embodiments, the composition comprises at least about 95% TIM3⁺ KLRG1⁻ PD1⁺ cells (e.g., T cells). In certain embodiments, the composition comprises about 100% TIM3⁺ KLRG1⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ CTLA4⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ CTLA4⁻ LAG3⁻ cells (e.g., T cells). In certain embodiments, the composition comprises at least about 5% TIM3⁺ CTLA4⁻ LAG3⁻ cells (e.g., T cells). In certain embodiments, the composition comprises at least about 10% TIM3⁺ CTLA4⁻ LAG3⁻ cells (e.g., T cells). In certain embodiments, the composition comprises at least about 15% TIM3⁺ CTLA4⁻ LAG3⁻ cells (e.g., T cells). In certain embodiments, the composition comprises at least about 20% TIM3⁺ CTLA4⁻ LAG3⁻ cells (e.g., T cells). In certain embodiments, the composition comprises at least about 25% TIM3⁺ CTLA4⁻ LAG3⁻ cells (e.g., T cells). In certain embodiments, the composition comprises at least about 30% TIM3⁺ CTLA4⁻ LAG3⁻ cells (e.g., T cells). In certain embodiments, the composition comprises at least about 35% TIM3⁺ CTLA4⁻ LAG3⁻ cells (e.g., T cells). In certain embodiments, the composition comprises at least about 40% TIM3⁺ CTLA4⁻ LAG3⁻ cells (e.g., T cells). In certain embodiments, the composition comprises at least about 45% TIM3⁺ CTLA4⁻ LAG3⁻ cells (e.g., T cells). In certain embodiments, the composition comprises at least about 50% TIM3⁺ CTLA4⁻ LAG3⁻ cells (e.g., T cells). In certain embodiments, the composition comprises at least about 55% TIM3⁺ CTLA4⁻ LAG3⁻ cells (e.g., T cells). In certain embodiments, the composition comprises at least about 60% TIM3⁺ CTLA4⁻ LAG3⁻ cells (e.g., T cells). In certain embodiments, the composition comprises at least about 65% TIM3⁺ CTLA4⁻ LAG3⁻ cells (e.g., T cells). In certain embodiments, the composition comprises at least about 70% TIM3⁺ CTLA4⁻ LAG3⁻ cells (e.g., T cells). In certain embodiments, the composition comprises at least about 75% TIM3⁺ CTLA4⁻ LAG3⁻ cells (e.g., T cells). In certain embodiments, the composition comprises at least about 80% TIM3⁺ CTLA4⁻ LAG3⁻ cells (e.g., T cells). In certain embodiments, the composition comprises at least about 85% TIM3⁺ CTLA4⁻ LAG3⁻ cells (e.g., T cells). In certain embodiments, the composition comprises at least about 90% TIM3⁺ CTLA4⁻ LAG3⁻ cells (e.g., T cells). In certain embodiments, the composition comprises at least about 95% TIM3⁺ CTLA4⁻ LAG3⁻ cells (e.g., T cells). In certain embodiments, the composition comprises about 100% TIM3⁺ CTLA4⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ CTLA4⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ CTLA4⁻ PD1⁺ cells (e.g., T cells). In certain embodiments, the composition comprises at least about 5% TIM3⁺ CTLA4⁻ PD1⁺ cells (e.g., T cells). In certain embodiments, the composition comprises at least about 10% TIM3⁺ CTLA4⁻ PD1⁺ cells (e.g., T cells). In certain embodiments, the composition comprises at least about 15% TIM3⁺ CTLA4⁻ PD1⁺ cells (e.g., T cells). In certain embodiments, the composition comprises at least about 20% TIM3⁺ CTLA4⁻ PD1⁺ cells (e.g., T cells). In certain embodiments, the composition comprises at least about 25% TIM3⁺ CTLA4⁻ PD1⁺ cells (e.g., T cells). In certain embodiments, the composition comprises at least about 30% TIM3⁺ CTLA4⁻ PD1⁺ cells (e.g., T cells). In certain embodiments, the composition comprises at least about 35% TIM3⁺ CTLA4⁻ PD1⁺ cells (e.g., T cells). In certain embodiments, the composition comprises at least about 40% TIM3⁺ CTLA4⁻ PD1⁺ cells (e.g., T cells). In certain embodiments, the composition comprises at least about 45% TIM3⁺ CTLA4⁻ PD1⁺ cells (e.g., T cells). In certain embodiments, the composition comprises at least about 50% TIM3⁺ CTLA4⁻ PD1⁺ cells (e.g., T cells). In certain embodiments, the composition comprises at least about 55% TIM3⁺ CTLA4⁻ PD1⁺ cells (e.g., T cells). In certain embodiments, the composition comprises at least about 60% TIM3⁺ CTLA4⁻ PD1⁺ cells (e.g., T cells). In certain embodiments, the composition comprises at least about 65% TIM3⁺ CTLA4⁻ PD1⁺ cells (e.g., T cells). In certain embodiments, the composition comprises at least about 70% TIM3⁺ CTLA4⁻ PD1⁺ cells (e.g., T cells). In certain embodiments, the composition comprises at least about 75% TIM3⁺ CTLA4⁻ PD1⁺ cells (e.g., T cells). In certain embodiments, the composition comprises at least about 80% TIM3⁺ CTLA4⁻ PD1⁺ cells (e.g., T cells). In certain embodiments, the composition comprises at least about 85% TIM3⁺ CTLA4⁻ PD1⁺ cells (e.g., T cells). In certain embodiments, the composition comprises at least about 90% TIM3⁺ CTLA4⁻ PD1⁺ cells (e.g., T cells). In certain embodiments, the composition comprises at least about 95% TIM3⁺ CTLA4⁻ PD1⁺ cells (e.g., T cells). In certain embodiments, the composition comprises about 100% TIM3⁺ CTLA4⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ LAG3⁻ PD1⁺ cells (e.g., T cells). In certain embodiments, the composition comprises at least about 5% TIM3⁺ LAG3⁻ PD1⁺ cells (e.g., T cells). In certain embodiments, the composition comprises at least about 10% TIM3⁺ LAG3⁻ PD1⁺ cells (e.g., T cells). In certain embodiments, the composition comprises at least about 15% TIM3⁺ LAG3⁻ PD1⁺ cells (e.g., T cells). In certain embodiments, the composition comprises at least about 20% TIM3⁺ LAG3⁻ PD1⁺ cells (e.g., T cells). In certain embodiments, the composition comprises at least about 25% TIM3⁺ LAG3⁻ PD1⁺ cells (e.g., T cells). In certain embodiments, the composition comprises at least about 30% TIM3⁺ LAG3⁻ PD1⁺ cells (e.g., T cells). In certain embodiments, the composition comprises at least about 35% TIM3⁺ LAG3⁻ PD1⁺ cells (e.g., T cells). In certain embodiments, the composition comprises at least about 40% TIM3⁺ LAG3⁻ PD1⁺ cells (e.g., T cells). In certain embodiments, the composition comprises at least about 45% TIM3⁺ LAG3⁻ PD1⁺ cells (e.g., T cells). In certain embodiments, the composition comprises at least about 50% TIM3⁺ LAG3⁻ PD1⁺ cells (e.g., T cells). In certain embodiments, the composition comprises at least about 55% TIM3⁺ LAG3⁻ PD1⁺ cells (e.g., T cells). In certain embodiments, the composition comprises at least about 60% TIM3⁺ LAG3⁻ PD1⁺ cells (e.g., T cells). In certain embodiments, the composition comprises at least about 65% TIM3⁺ LAG3⁻ PD1⁺ cells (e.g., T cells). In certain embodiments, the composition comprises at least about 70% TIM3⁺ LAG3⁻ PD1⁺ cells (e.g., T cells). In certain embodiments, the composition comprises at least about 75% TIM3⁺ LAG3⁻ PD1⁺ cells (e.g., T cells). In certain embodiments, the composition comprises at least about 80% TIM3⁺ LAG3⁻ PD1⁺ cells (e.g., T cells). In certain embodiments, the composition comprises at least about 85% TIM3⁺ LAG3⁻ PD1⁺ cells (e.g., T cells). In certain embodiments, the composition comprises at least about 90% TIM3⁺ LAG3⁻ PD1⁺ cells (e.g., T cells). In certain embodiments, the composition comprises at least about 95% TIM3⁺ LAG3⁻ PD1⁺ cells (e.g., T cells). In certain embodiments, the composition comprises about 100% TIM3⁺ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% KLRG1⁻ CTLA4⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% KLRG1⁻ CTLA4⁻ LAG3⁻ cells (e.g., T cells). In certain embodiments, the composition comprises at least about 5% KLRG1⁻ CTLA4⁻ LAG3⁻ cells (e.g., T cells). In certain embodiments, the composition comprises at least about 10% KLRG1⁻ CTLA4⁻ LAG3⁻ cells (e.g., T cells). In certain embodiments, the composition comprises at least about 15% KLRG1⁻ CTLA4⁻ LAG3⁻ cells (e.g., T cells). In certain embodiments, the composition comprises at least about 20% KLRG1⁻ CTLA4⁻ LAG3⁻ cells (e.g., T cells). In certain embodiments, the composition comprises at least about 25% KLRG1⁻ CTLA4⁻ LAG3⁻ cells (e.g., T cells). In certain embodiments, the composition comprises at least about 30% KLRG1⁻ CTLA4⁻ LAG3⁻ cells (e.g., T cells). In certain embodiments, the composition comprises at least about 35% KLRG1⁻ CTLA4⁻ LAG3⁻ cells (e.g., T cells). In certain embodiments, the composition comprises at least about 40% KLRG1⁻ CTLA4⁻ LAG3⁻ cells (e.g., T cells). In certain embodiments, the composition comprises at least about 45% KLRG1⁻ CTLA4⁻ LAG3⁻ cells (e.g., T cells). In certain embodiments, the composition comprises at least about 50% KLRG1⁻ CTLA4⁻ LAG3⁻ cells (e.g., T cells). In certain embodiments, the composition comprises at least about 55% KLRG1⁻ CTLA4⁻ LAG3⁻ cells (e.g., T cells). In certain embodiments, the composition comprises at least about 60% KLRG1⁻ CTLA4⁻ LAG3⁻ cells (e.g., T cells). In certain embodiments, the composition comprises at least about 65% KLRG1⁻ CTLA4⁻ LAG3⁻ cells (e.g., T cells). In certain embodiments, the composition comprises at least about 70% KLRG1⁻ CTLA4⁻ LAG3⁻ cells (e.g., T cells). In certain embodiments, the composition comprises at least about 75% KLRG1⁻ CTLA4⁻ LAG3⁻ cells (e.g., T cells). In certain embodiments, the composition comprises at least about 80% KLRG1⁻ CTLA4⁻ LAG3⁻ cells (e.g., T cells). In certain embodiments, the composition comprises at least about 85% KLRG1⁻ CTLA4⁻ LAG3⁻ cells (e.g., T cells). In certain embodiments, the composition comprises at least about 90% KLRG1⁻ CTLA4⁻ LAG3⁻ cells (e.g., T cells). In certain embodiments, the composition comprises at least about 95% KLRG1⁻ CTLA4⁻ LAG3⁻ cells (e.g., T cells). In certain embodiments, the composition comprises about 100% KLRG1⁻ CTLA4⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% KLRG1⁻ CTLA4⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% KLRG1⁻ CTLA4⁻ PD1⁺ cells (e.g., T cells). In certain embodiments, the composition comprises at least about 5% KLRG1⁻ CTLA4⁻ PD1⁺ cells (e.g., T cells). In certain embodiments, the composition comprises at least about 10% KLRG1⁻ CTLA4⁻ PD1⁺ cells (e.g., T cells). In certain embodiments, the composition comprises at least about 15% KLRG1⁻ CTLA4⁻ PD1⁺ cells (e.g., T cells). In certain embodiments, the composition comprises at least about 20% KLRG1⁻ CTLA4⁻ PD1⁺ cells (e.g., T cells). In certain embodiments, the composition comprises at least about 25% KLRG1⁻ CTLA4⁻ PD1⁺ cells (e.g., T cells). In certain embodiments, the composition comprises at least about 30% KLRG1⁻ CTLA4⁻ PD1⁺ cells (e.g., T cells). In certain embodiments, the composition comprises at least about 35% KLRG1⁻ CTLA4⁻ PD1⁺ cells (e.g., T cells). In certain embodiments, the composition comprises at least about 40% KLRG1⁻ CTLA4⁻ PD1⁺ cells (e.g., T cells). In certain embodiments, the composition comprises at least about 45% KLRG1⁻ CTLA4⁻ PD1⁺ cells (e.g., T cells). In certain embodiments, the composition comprises at least about 50% KLRG1⁻ CTLA4⁻ PD1⁺ cells (e.g., T cells). In certain embodiments, the composition comprises at least about 55% KLRG1⁻ CTLA4⁻ PD1⁺ cells (e.g., T cells). In certain embodiments, the composition comprises at least about 60% KLRG1⁻ CTLA4⁻ PD1⁺ cells (e.g., T cells). In certain embodiments, the composition comprises at least about 65% KLRG1⁻ CTLA4⁻ PD1⁺ cells (e.g., T cells). In certain embodiments, the composition comprises at least about 70% KLRG1⁻ CTLA4⁻ PD1⁺ cells (e.g., T cells). In certain embodiments, the composition comprises at least about 75% KLRG1⁻ CTLA4⁻ PD1⁺ cells (e.g., T cells). In certain embodiments, the composition comprises at least about 80% KLRG1⁻ CTLA4⁻ PD1⁺ cells (e.g., T cells). In certain embodiments, the composition comprises at least about 85% KLRG1⁻ CTLA4⁻ PD1⁺ cells (e.g., T cells). In certain embodiments, the composition comprises at least about 90% KLRG1⁻ CTLA4⁻ PD1⁺ cells (e.g., T cells). In certain embodiments, the composition comprises at least about 95% KLRG1⁻ CTLA4⁻ PD1⁺ cells (e.g., T cells). In certain embodiments, the composition comprises about 100% KLRG1⁻ CTLA4⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% KLRG1⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% KLRG1⁻ LAG3⁻ PD1⁺ cells (e.g., T cells). In certain embodiments, the composition comprises at least about 5% KLRG1⁻ LAG3⁻ PD1⁺ cells (e.g., T cells). In certain embodiments, the composition comprises at least about 10% KLRG1⁻ LAG3⁻ PD1⁺ cells (e.g., T cells). In certain embodiments, the composition comprises at least about 15% KLRG1⁻ LAG3⁻ PD1⁺ cells (e.g., T cells). In certain embodiments, the composition comprises at least about 20% KLRG1⁻ LAG3⁻ PD1⁺ cells (e.g., T cells). In certain embodiments, the composition comprises at least about 25% KLRG1⁻ LAG3⁻ PD1⁺ cells (e.g., T cells). In certain embodiments, the composition comprises at least about 30% KLRG1⁻ LAG3⁻ PD1⁺ cells (e.g., T cells). In certain embodiments, the composition comprises at least about 35% KLRG1⁻ LAG3⁻ PD1⁺ cells (e.g., T cells). In certain embodiments, the composition comprises at least about 40% KLRG1⁻ LAG3⁻ PD1⁺ cells (e.g., T cells). In certain embodiments, the composition comprises at least about 45% KLRG1⁻ LAG3⁻ PD1⁺ cells (e.g., T cells). In certain embodiments, the composition comprises at least about 50% KLRG1⁻ LAG3⁻ PD1⁺ cells (e.g., T cells). In certain embodiments, the composition comprises at least about 55% KLRG1⁻ LAG3⁻ PD1⁺ cells (e.g., T cells). In certain embodiments, the composition comprises at least about 60% KLRG1⁻ LAG3⁻ PD1⁺ cells (e.g., T cells). In certain embodiments, the composition comprises at least about 65% KLRG1⁻ LAG3⁻ PD1⁺ cells (e.g., T cells). In certain embodiments, the composition comprises at least about 70% KLRG1⁻ LAG3⁻ PD1⁺ cells (e.g., T cells). In certain embodiments, the composition comprises at least about 75% KLRG1⁻ LAG3⁻ PD1⁺ cells (e.g., T cells). In certain embodiments, the composition comprises at least about 80% KLRG1⁻ LAG3⁻ PD1⁺ cells (e.g., T cells). In certain embodiments, the composition comprises at least about 85% KLRG1⁻ LAG3⁻ PD1⁺ cells (e.g., T cells). In certain embodiments, the composition comprises at least about 90% KLRG1⁻ LAG3⁻ PD1⁺ cells (e.g., T cells). In certain embodiments, the composition comprises at least about 95% KLRG1⁻ LAG3⁻ PD1⁺ cells (e.g., T cells). In certain embodiments, the composition comprises about 100% KLRG1⁻ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CTLA4⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CTLA4⁻ LAG3⁻ PD1⁺ cells (e.g., T cells). In certain embodiments, the composition comprises at least about 5% CTLA4⁻ LAG3⁻ PD1⁺ cells (e.g., T cells). In certain embodiments, the composition comprises at least about 10% CTLA4⁻ LAG3⁻ PD1⁺ cells (e.g., T cells). In certain embodiments, the composition comprises at least about 15% CTLA4⁻ LAG3⁻ PD1⁺ cells (e.g., T cells). In certain embodiments, the composition comprises at least about 20% CTLA4⁻ LAG3⁻ PD1⁺ cells (e.g., T cells). In certain embodiments, the composition comprises at least about 25% CTLA4⁻ LAG3⁻ PD1⁺ cells (e.g., T cells). In certain embodiments, the composition comprises at least about 30% CTLA4⁻ LAG3⁻ PD1⁺ cells (e.g., T cells). In certain embodiments, the composition comprises at least about 35% CTLA4⁻ LAG3⁻ PD1⁺ cells (e.g., T cells). In certain embodiments, the composition comprises at least about 40% CTLA4⁻ LAG3⁻ PD1⁺ cells (e.g., T cells). In certain embodiments, the composition comprises at least about 45% CTLA4⁻ LAG3⁻ PD1⁺ cells (e.g., T cells). In certain embodiments, the composition comprises at least about 50% CTLA4⁻ LAG3⁻ PD1⁺ cells (e.g., T cells). In certain embodiments, the composition comprises at least about 55% CTLA4⁻ LAG3⁻ PD1⁺ cells (e.g., T cells). In certain embodiments, the composition comprises at least about 60% CTLA4⁻ LAG3⁻ PD1⁺ cells (e.g., T cells). In certain embodiments, the composition comprises at least about 65% CTLA4⁻ LAG3⁻ PD1⁺ cells (e.g., T cells). In certain embodiments, the composition comprises at least about 70% CTLA4⁻ LAG3⁻ PD1⁺ cells (e.g., T cells). In certain embodiments, the composition comprises at least about 75% CTLA4⁻ LAG3⁻ PD1⁺ cells (e.g., T cells). In certain embodiments, the composition comprises at least about 80% CTLA4⁻ LAG3⁻ PD1⁺ cells (e.g., T cells). In certain embodiments, the composition comprises at least about 85% CTLA4⁻ LAG3⁻ PD1⁺ cells (e.g., T cells). In certain embodiments, the composition comprises at least about 90% CTLA4⁻ LAG3⁻ PD1⁺ cells (e.g., T cells). In certain embodiments, the composition comprises at least about 95% CTLA4⁻ LAG3⁻ PD1⁺ cells (e.g., T cells). In certain embodiments, the composition comprises about 100% CTLA4⁻ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ KLRG1⁻ CTLA4⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ KLRG1⁻ CTLA4⁻ cells (e.g., T cells). In certain embodiments, the composition comprises at least about 5% CD57⁺ TIM3⁺ KLRG1⁻ CTLA4⁻ cells (e.g., T cells). In certain embodiments, the composition comprises at least about 10% CD57⁺ TIM3⁺ KLRG1⁻ CTLA4⁻ cells (e.g., T cells). In certain embodiments, the composition comprises at least about 15% CD57⁺ TIM3⁺ KLRG1⁻ CTLA4⁻ cells (e.g., T cells). In certain embodiments, the composition comprises at least about 20% CD57⁺ TIM3⁺ KLRG1⁻ CTLA4⁻ cells (e.g., T cells). In certain embodiments, the composition comprises at least about 25% CD57⁺ TIM3⁺ KLRG1⁻ CTLA4⁻ cells (e.g., T cells). In certain embodiments, the composition comprises at least about 30% CD57⁺ TIM3⁺ KLRG1⁻ CTLA4⁻ cells (e.g., T cells). In certain embodiments, the composition comprises at least about 35% CD57⁺ TIM3⁺ KLRG1⁻ CTLA4⁻ cells (e.g., T cells). In certain embodiments, the composition comprises at least about 40% CD57⁺ TIM3⁺ KLRG1⁻ CTLA4⁻ cells (e.g., T cells). In certain embodiments, the composition comprises at least about 45% CD57⁺ TIM3⁺ KLRG1⁻ CTLA4⁻ cells (e.g., T cells). In certain embodiments, the composition comprises at least about 50% CD57⁺ TIM3⁺ KLRG1⁻ CTLA4⁻ cells (e.g., T cells). In certain embodiments, the composition comprises at least about 55% CD57⁺ TIM3⁺ KLRG1⁻ CTLA4⁻ cells (e.g., T cells). In certain embodiments, the composition comprises at least about 60% CD57⁺ TIM3⁺ KLRG1⁻ CTLA4⁻ cells (e.g., T cells). In certain embodiments, the composition comprises at least about 65% CD57⁺ TIM3⁺ KLRG1⁻ CTLA4⁻ cells (e.g., T cells). In certain embodiments, the composition comprises at least about 70% CD57⁺ TIM3⁺ KLRG1⁻ CTLA4⁻ cells (e.g., T cells). In certain embodiments, the composition comprises at least about 75% CD57⁺ TIM3⁺ KLRG1⁻ CTLA4⁻ cells (e.g., T cells). In certain embodiments, the composition comprises at least about 80% CD57⁺ TIM3⁺ KLRG1⁻ CTLA4⁻ cells (e.g., T cells). In certain embodiments, the composition comprises at least about 85% CD57⁺ TIM3⁺ KLRG1⁻ CTLA4⁻ cells (e.g., T cells). In certain embodiments, the composition comprises at least about 90% CD57⁺ TIM3⁺ KLRG1⁻ CTLA4⁻ cells (e.g., T cells). In certain embodiments, the composition comprises at least about 95% CD57⁺ TIM3⁺ KLRG1⁻ CTLA4⁻ cells (e.g., T cells). In certain embodiments, the composition comprises about 100% CD57⁺ TIM3⁺ KLRG1⁻ CTLA4⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ KLRG1⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ KLRG1⁻ LAG3⁻ cells (e.g., T cells). In certain embodiments, the composition comprises at least about 5% CD57⁺ TIM3⁺ KLRG1⁻ LAG3⁻ cells (e.g., T cells). In certain embodiments, the composition comprises at least about 10% CD57⁺ TIM3⁺ KLRG1⁻ LAG3⁻ cells (e.g., T cells). In certain embodiments, the composition comprises at least about 15% CD57⁺ TIM3⁺ KLRG1⁻ LAG3⁻ cells (e.g., T cells). In certain embodiments, the composition comprises at least about 20% CD57⁺ TIM3⁺ KLRG1⁻ LAG3⁻ cells (e.g., T cells). In certain embodiments, the composition comprises at least about 25% CD57⁺ TIM3⁺ KLRG1⁻ LAG3⁻ cells (e.g., T cells). In certain embodiments, the composition comprises at least about 30% CD57⁺ TIM3⁺ KLRG1⁻ LAG3⁻ cells (e.g., T cells). In certain embodiments, the composition comprises at least about 35% CD57⁺ TIM3⁺ KLRG1⁻ LAG3⁻ cells (e.g., T cells). In certain embodiments, the composition comprises at least about 40% CD57⁺ TIM3⁺ KLRG1⁻ LAG3⁻ cells (e.g., T cells). In certain embodiments, the composition comprises at least about 45% CD57⁺ TIM3⁺ KLRG1⁻ LAG3⁻ cells (e.g., T cells). In certain embodiments, the composition comprises at least about 50% CD57⁺ TIM3⁺ KLRG1⁻ LAG3⁻ cells (e.g., T cells). In certain embodiments, the composition comprises at least about 55% CD57⁺ TIM3⁺ KLRG1⁻ LAG3⁻ cells (e.g., T cells). In certain embodiments, the composition comprises at least about 60% CD57⁺ TIM3⁺ KLRG1⁻ LAG3⁻ cells (e.g., T cells). In certain embodiments, the composition comprises at least about 65% CD57⁺ TIM3⁺ KLRG1⁻ LAG3⁻ cells (e.g., T cells). In certain embodiments, the composition comprises at least about 70% CD57⁺ TIM3⁺ KLRG1⁻ LAG3⁻ cells (e.g., T cells). In certain embodiments, the composition comprises at least about 75% CD57⁺ TIM3⁺ KLRG1⁻ LAG3⁻ cells (e.g., T cells). In certain embodiments, the composition comprises at least about 80% CD57⁺ TIM3⁺ KLRG1⁻ LAG3⁻ cells (e.g., T cells). In certain embodiments, the composition comprises at least about 85% CD57⁺ TIM3⁺ KLRG1⁻ LAG3⁻ cells (e.g., T cells). In certain embodiments, the composition comprises at least about 90% CD57⁺ TIM3⁺ KLRG1⁻ LAG3⁻ cells (e.g., T cells). In certain embodiments, the composition comprises at least about 95% CD57⁺ TIM3⁺ KLRG1⁻ LAG3⁻ cells (e.g., T cells). In certain embodiments, the composition comprises about 100% CD57⁺ TIM3⁺ KLRG1⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ KLRG1⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ KLRG1⁻ PD1⁺ cells (e.g., T cells). In certain embodiments, the composition comprises at least about 5% CD57⁺ TIM3⁺ KLRG1⁻ PD1⁺ cells (e.g., T cells). In certain embodiments, the composition comprises at least about 10% CD57⁺ TIM3⁺ KLRG1⁻ PD1⁺ cells (e.g., T cells). In certain embodiments, the composition comprises at least about 15% CD57⁺ TIM3⁺ KLRG1⁻ PD1⁺ cells (e.g., T cells). In certain embodiments, the composition comprises at least about 20% CD57⁺ TIM3⁺ KLRG1⁻ PD1⁺ cells (e.g., T cells). In certain embodiments, the composition comprises at least about 25% CD57⁺ TIM3⁺ KLRG1⁻ PD1⁺ cells (e.g., T cells). In certain embodiments, the composition comprises at least about 30% CD57⁺ TIM3⁺ KLRG1⁻ PD1⁺ cells (e.g., T cells). In certain embodiments, the composition comprises at least about 35% CD57⁺ TIM3⁺ KLRG1⁻ PD1⁺ cells (e.g., T cells). In certain embodiments, the composition comprises at least about 40% CD57⁺ TIM3⁺ KLRG1⁻ PD1⁺ cells (e.g., T cells). In certain embodiments, the composition comprises at least about 45% CD57⁺ TIM3⁺ KLRG1⁻ PD1⁺ cells (e.g., T cells). In certain embodiments, the composition comprises at least about 50% CD57⁺ TIM3⁺ KLRG1⁻ PD1⁺ cells (e.g., T cells). In certain embodiments, the composition comprises at least about 55% CD57⁺ TIM3⁺ KLRG1⁻ PD1⁺ cells (e.g., T cells). In certain embodiments, the composition comprises at least about 60% CD57⁺ TIM3⁺ KLRG1⁻ PD1⁺ cells (e.g., T cells). In certain embodiments, the composition comprises at least about 65% CD57⁺ TIM3⁺ KLRG1⁻ PD1⁺ cells (e.g., T cells). In certain embodiments, the composition comprises at least about 70% CD57⁺ TIM3⁺ KLRG1⁻ PD1⁺ cells (e.g., T cells). In certain embodiments, the composition comprises at least about 75% CD57⁺ TIM3⁺ KLRG1⁻ PD1⁺ cells (e.g., T cells). In certain embodiments, the composition comprises at least about 80% CD57⁺ TIM3⁺ KLRG1⁻ PD1⁺ cells (e.g., T cells). In certain embodiments, the composition comprises at least about 85% CD57⁺ TIM3⁺ KLRG1⁻ PD1⁺ cells (e.g., T cells). In certain embodiments, the composition comprises at least about 90% CD57⁺ TIM3⁺ KLRG1⁻ PD1⁺ cells (e.g., T cells). In certain embodiments, the composition comprises at least about 95% CD57⁺ TIM3⁺ KLRG1⁻ PD1⁺ cells (e.g., T cells). In certain embodiments, the composition comprises about 100% CD57⁺ TIM3⁺ KLRG1⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ CTLA4⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ CTLA4⁻ LAG3⁻ cells (e.g., T cells). In certain embodiments, the composition comprises at least about 5% CD57⁺ TIM3⁺ CTLA4⁻ LAG3⁻ cells (e.g., T cells). In certain embodiments, the composition comprises at least about 10% CD57⁺ TIM3⁺ CTLA4⁻ LAG3⁻ cells (e.g., T cells). In certain embodiments, the composition comprises at least about 15% CD57⁺ TIM3⁺ CTLA4⁻ LAG3⁻ cells (e.g., T cells). In certain embodiments, the composition comprises at least about 20% CD57⁺ TIM3⁺ CTLA4⁻ LAG3⁻ cells (e.g., T cells). In certain embodiments, the composition comprises at least about 25% CD57⁺ TIM3⁺ CTLA4⁻ LAG3⁻ cells (e.g., T cells). In certain embodiments, the composition comprises at least about 30% CD57⁺ TIM3⁺ CTLA4⁻ LAG3⁻ cells (e.g., T cells). In certain embodiments, the composition comprises at least about 35% CD57⁺ TIM3⁺ CTLA4⁻ LAG3⁻ cells (e.g., T cells). In certain embodiments, the composition comprises at least about 40% CD57⁺ TIM3⁺ CTLA4⁻ LAG3⁻ cells (e.g., T cells). In certain embodiments, the composition comprises at least about 45% CD57⁺ TIM3⁺ CTLA4⁻ LAG3⁻ cells (e.g., T cells). In certain embodiments, the composition comprises at least about 50% CD57⁺ TIM3⁺ CTLA4⁻ LAG3⁻ cells (e.g., T cells). In certain embodiments, the composition comprises at least about 55% CD57⁺ TIM3⁺ CTLA4⁻ LAG3⁻ cells (e.g., T cells). In certain embodiments, the composition comprises at least about 60% CD57⁺ TIM3⁺ CTLA4⁻ LAG3⁻ cells (e.g., T cells). In certain embodiments, the composition comprises at least about 65% CD57⁺ TIM3⁺ CTLA4⁻ LAG3⁻ cells (e.g., T cells). In certain embodiments, the composition comprises at least about 70% CD57⁺ TIM3⁺ CTLA4⁻ LAG3⁻ cells (e.g., T cells). In certain embodiments, the composition comprises at least about 75% CD57⁺ TIM3⁺ CTLA4⁻ LAG3⁻ cells (e.g., T cells). In certain embodiments, the composition comprises at least about 80% CD57⁺ TIM3⁺ CTLA4⁻ LAG3⁻ cells (e.g., T cells). In certain embodiments, the composition comprises at least about 85% CD57⁺ TIM3⁺ CTLA4⁻ LAG3⁻ cells (e.g., T cells). In certain embodiments, the composition comprises at least about 90% CD57⁺ TIM3⁺ CTLA4⁻ LAG3⁻ cells (e.g., T cells). In certain embodiments, the composition comprises at least about 95% CD57⁺ TIM3⁺ CTLA4⁻ LAG3⁻ cells (e.g., T cells). In certain embodiments, the composition comprises about 100% CD57⁺ TIM3⁺ CTLA4⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ CTLA4⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ CTLA4⁻ PD1⁺ cells (e.g., T cells). In certain embodiments, the composition comprises at least about 5% CD57⁺ TIM3⁺ CTLA4⁻ PD1⁺ cells (e.g., T cells). In certain embodiments, the composition comprises at least about 10% CD57⁺ TIM3⁺ CTLA4⁻ PD1⁺ cells (e.g., T cells). In certain embodiments, the composition comprises at least about 15% CD57⁺ TIM3⁺ CTLA4⁻ PD1⁺ cells (e.g., T cells). In certain embodiments, the composition comprises at least about 20% CD57⁺ TIM3⁺ CTLA4⁻ PD1⁺ cells (e.g., T cells). In certain embodiments, the composition comprises at least about 25% CD57⁺ TIM3⁺ CTLA4⁻ PD1⁺ cells (e.g., T cells). In certain embodiments, the composition comprises at least about 30% CD57⁺ TIM3⁺ CTLA4⁻ PD1⁺ cells (e.g., T cells). In certain embodiments, the composition comprises at least about 35% CD57⁺ TIM3⁺ CTLA4⁻ PD1⁺ cells (e.g., T cells). In certain embodiments, the composition comprises at least about 40% CD57⁺ TIM3⁺ CTLA4⁻ PD1⁺ cells (e.g., T cells). In certain embodiments, the composition comprises at least about 45% CD57⁺ TIM3⁺ CTLA4⁻ PD1⁺ cells (e.g., T cells). In certain embodiments, the composition comprises at least about 50% CD57⁺ TIM3⁺ CTLA4⁻ PD1⁺ cells (e.g., T cells). In certain embodiments, the composition comprises at least about 55% CD57⁺ TIM3⁺ CTLA4⁻ PD1⁺ cells (e.g., T cells). In certain embodiments, the composition comprises at least about 60% CD57⁺ TIM3⁺ CTLA4⁻ PD1⁺ cells (e.g., T cells). In certain embodiments, the composition comprises at least about 65% CD57⁺ TIM3⁺ CTLA4⁻ PD1⁺ cells (e.g., T cells). In certain embodiments, the composition comprises at least about 70% CD57⁺ TIM3⁺ CTLA4⁻ PD1⁺ cells (e.g., T cells). In certain embodiments, the composition comprises at least about 75% CD57⁺ TIM3⁺ CTLA4⁻ PD1⁺ cells (e.g., T cells). In certain embodiments, the composition comprises at least about 80% CD57⁺ TIM3⁺ CTLA4⁻ PD1⁺ cells (e.g., T cells). In certain embodiments, the composition comprises at least about 85% CD57⁺ TIM3⁺ CTLA4⁻ PD1⁺ cells (e.g., T cells). In certain embodiments, the composition comprises at least about 90% CD57⁺ TIM3⁺ CTLA4⁻ PD1⁺ cells (e.g., T cells). In certain embodiments, the composition comprises at least about 95% CD57⁺ TIM3⁺ CTLA4⁻ PD1⁺ cells (e.g., T cells). In certain embodiments, the composition comprises about 100% CD57⁺ TIM3⁺ CTLA4⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ LAG3⁻ PD1⁺ cells (e.g., T cells). In certain embodiments, the composition comprises at least about 5% CD57⁺ TIM3⁺ LAG3⁻ PD1⁺ cells (e.g., T cells). In certain embodiments, the composition comprises at least about 10% CD57⁺ TIM3⁺ LAG3⁻ PD1⁺ cells (e.g., T cells). In certain embodiments, the composition comprises at least about 15% CD57⁺ TIM3⁺ LAG3⁻ PD1⁺ cells (e.g., T cells). In certain embodiments, the composition comprises at least about 20% CD57⁺ TIM3⁺ LAG3⁻ PD1⁺ cells (e.g., T cells). In certain embodiments, the composition comprises at least about 25% CD57⁺ TIM3⁺ LAG3⁻ PD1⁺ cells (e.g., T cells). In certain embodiments, the composition comprises at least about 30% CD57⁺ TIM3⁺ LAG3⁻ PD1⁺ cells (e.g., T cells). In certain embodiments, the composition comprises at least about 35% CD57⁺ TIM3⁺ LAG3⁻ PD1⁺ cells (e.g., T cells). In certain embodiments, the composition comprises at least about 40% CD57⁺ TIM3⁺ LAG3⁻ PD1⁺ cells (e.g., T cells). In certain embodiments, the composition comprises at least about 45% CD57⁺ TIM3⁺ LAG3⁻ PD1⁺ cells (e.g., T cells). In certain embodiments, the composition comprises at least about 50% CD57⁺ TIM3⁺ LAG3⁻ PD1⁺ cells (e.g., T cells). In certain embodiments, the composition comprises at least about 55% CD57⁺ TIM3⁺ LAG3⁻ PD1⁺ cells (e.g., T cells). In certain embodiments, the composition comprises at least about 60% CD57⁺ TIM3⁺ LAG3⁻ PD1⁺ cells (e.g., T cells). In certain embodiments, the composition comprises at least about 65% CD57⁺ TIM3⁺ LAG3⁻ PD1⁺ cells (e.g., T cells). In certain embodiments, the composition comprises at least about 70% CD57⁺ TIM3⁺ LAG3⁻ PD1⁺ cells (e.g., T cells). In certain embodiments, the composition comprises at least about 75% CD57⁺ TIM3⁺ LAG3⁻ PD1⁺ cells (e.g., T cells). In certain embodiments, the composition comprises at least about 80% CD57⁺ TIM3⁺ LAG3⁻ PD1⁺ cells (e.g., T cells). In certain embodiments, the composition comprises at least about 85% CD57⁺ TIM3⁺ LAG3⁻ PD1⁺ cells (e.g., T cells). In certain embodiments, the composition comprises at least about 90% CD57⁺ TIM3⁺ LAG3⁻ PD1⁺ cells (e.g., T cells). In certain embodiments, the composition comprises at least about 95% CD57⁺ TIM3⁺ LAG3⁻ PD1⁺ cells (e.g., T cells). In certain embodiments, the composition comprises about 100% CD57⁺ TIM3⁺ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ KLRG1⁻ CTLA4⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ KLRG1⁻ CTLA4⁻ LAG3⁻ cells (e.g., T cells). In certain embodiments, the composition comprises at least about 5% CD57⁺ KLRG1⁻ CTLA4⁻ LAG3⁻ cells (e.g., T cells). In certain embodiments, the composition comprises at least about 10% CD57⁺ KLRG1⁻ CTLA4⁻ LAG3⁻ cells (e.g., T cells). In certain embodiments, the composition comprises at least about 15% CD57⁺ KLRG1⁻ CTLA4⁻ LAG3⁻ cells (e.g., T cells). In certain embodiments, the composition comprises at least about 20% CD57⁺ KLRG1⁻ CTLA4⁻ LAG3⁻ cells (e.g., T cells). In certain embodiments, the composition comprises at least about 25% CD57⁺ KLRG1⁻ CTLA4⁻ LAG3⁻ cells (e.g., T cells). In certain embodiments, the composition comprises at least about 30% CD57⁺ KLRG1⁻ CTLA4⁻ LAG3⁻ cells (e.g., T cells). In certain embodiments, the composition comprises at least about 35% CD57⁺ KLRG1⁻ CTLA4⁻ LAG3⁻ cells (e.g., T cells). In certain embodiments, the composition comprises at least about 40% CD57⁺ KLRG1⁻ CTLA4⁻ LAG3⁻ cells (e.g., T cells). In certain embodiments, the composition comprises at least about 45% CD57⁺ KLRG1⁻ CTLA4⁻ LAG3⁻ cells (e.g., T cells). In certain embodiments, the composition comprises at least about 50% CD57⁺ KLRG1⁻ CTLA4⁻ LAG3⁻ cells (e.g., T cells). In certain embodiments, the composition comprises at least about 55% CD57⁺ KLRG1⁻ CTLA4⁻ LAG3⁻ cells (e.g., T cells). In certain embodiments, the composition comprises at least about 60% CD57⁺ KLRG1⁻ CTLA4⁻ LAG3⁻ cells (e.g., T cells). In certain embodiments, the composition comprises at least about 65% CD57⁺ KLRG1⁻ CTLA4⁻ LAG3⁻ cells (e.g., T cells). In certain embodiments, the composition comprises at least about 70% CD57⁺ KLRG1⁻ CTLA4⁻ LAG3⁻ cells (e.g., T cells). In certain embodiments, the composition comprises at least about 75% CD57⁺ KLRG1⁻ CTLA4⁻ LAG3⁻ cells (e.g., T cells). In certain embodiments, the composition comprises at least about 80% CD57⁺ KLRG1⁻ CTLA4⁻ LAG3⁻ cells (e.g., T cells). In certain embodiments, the composition comprises at least about 85% CD57⁺ KLRG1⁻ CTLA4⁻ LAG3⁻ cells (e.g., T cells). In certain embodiments, the composition comprises at least about 90% CD57⁺ KLRG1⁻ CTLA4⁻ LAG3⁻ cells (e.g., T cells). In certain embodiments, the composition comprises at least about 95% CD57⁺ KLRG1⁻ CTLA4⁻ LAG3⁻ cells (e.g., T cells). In certain embodiments, the composition comprises about 100% CD57⁺ KLRG1⁻ CTLA4⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ KLRG1⁻ CTLA4⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ KLRG1⁻ CTLA4⁻ PD1⁺ cells (e.g., T cells). In certain embodiments, the composition comprises at least about 5% CD57⁺ KLRG1⁻ CTLA4⁻ PD1⁺ cells (e.g., T cells). In certain embodiments, the composition comprises at least about 10% CD57⁺ KLRG1⁻ CTLA4⁻ PD1⁺ cells (e.g., T cells). In certain embodiments, the composition comprises at least about 15% CD57⁺ KLRG1⁻ CTLA4⁻ PD1⁺ cells (e.g., T cells). In certain embodiments, the composition comprises at least about 20% CD57⁺ KLRG1⁻ CTLA4⁻ PD1⁺ cells (e.g., T cells). In certain embodiments, the composition comprises at least about 25% CD57⁺ KLRG1⁻ CTLA4⁻ PD1⁺ cells (e.g., T cells). In certain embodiments, the composition comprises at least about 30% CD57⁺ KLRG1⁻ CTLA4⁻ PD1⁺ cells (e.g., T cells). In certain embodiments, the composition comprises at least about 35% CD57⁺ KLRG1⁻ CTLA4⁻ PD1⁺ cells (e.g., T cells). In certain embodiments, the composition comprises at least about 40% CD57⁺ KLRG1⁻ CTLA4⁻ PD1⁺ cells (e.g., T cells). In certain embodiments, the composition comprises at least about 45% CD57⁺ KLRG1⁻ CTLA4⁻ PD1⁺ cells (e.g., T cells). In certain embodiments, the composition comprises at least about 50% CD57⁺ KLRG1⁻ CTLA4⁻ PD1⁺ cells (e.g., T cells). In certain embodiments, the composition comprises at least about 55% CD57⁺ KLRG1⁻ CTLA4⁻ PD1⁺ cells (e.g., T cells). In certain embodiments, the composition comprises at least about 60% CD57⁺ KLRG1⁻ CTLA4⁻ PD1⁺ cells (e.g., T cells). In certain embodiments, the composition comprises at least about 65% CD57⁺ KLRG1⁻ CTLA4⁻ PD1⁺ cells (e.g., T cells). In certain embodiments, the composition comprises at least about 70% CD57⁺ KLRG1⁻ CTLA4⁻ PD1⁺ cells (e.g., T cells). In certain embodiments, the composition comprises at least about 75% CD57⁺ KLRG1⁻ CTLA4⁻ PD1⁺ cells (e.g., T cells). In certain embodiments, the composition comprises at least about 80% CD57⁺ KLRG1⁻ CTLA4⁻ PD1⁺ cells (e.g., T cells). In certain embodiments, the composition comprises at least about 85% CD57⁺ KLRG1⁻ CTLA4⁻ PD1⁺ cells (e.g., T cells). In certain embodiments, the composition comprises at least about 90% CD57⁺ KLRG1⁻ CTLA4⁻ PD1⁺ cells (e.g., T cells). In certain embodiments, the composition comprises at least about 95% CD57⁺ KLRG1⁻ CTLA4⁻ PD1⁺ cells (e.g., T cells). In certain embodiments, the composition comprises about 100% CD57⁺ KLRG1⁻ CTLA4⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ KLRG1⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ KLRG1⁻ LAG3⁻ PD1⁺ cells (e.g., T cells). In certain embodiments, the composition comprises at least about 5% CD57⁺ KLRG1⁻ LAG3⁻ PD1⁺ cells (e.g., T cells). In certain embodiments, the composition comprises at least about 10% CD57⁺ KLRG1⁻ LAG3⁻ PD1⁺ cells (e.g., T cells). In certain embodiments, the composition comprises at least about 15% CD57⁺ KLRG1⁻ LAG3⁻ PD1⁺ cells (e.g., T cells). In certain embodiments, the composition comprises at least about 20% CD57⁺ KLRG1⁻ LAG3⁻ PD1⁺ cells (e.g., T cells). In certain embodiments, the composition comprises at least about 25% CD57⁺ KLRG1⁻ LAG3⁻ PD1⁺ cells (e.g., T cells). In certain embodiments, the composition comprises at least about 30% CD57⁺ KLRG1⁻ LAG3⁻ PD1⁺ cells (e.g., T cells). In certain embodiments, the composition comprises at least about 35% CD57⁺ KLRG1⁻ LAG3⁻ PD1⁺ cells (e.g., T cells). In certain embodiments, the composition comprises at least about 40% CD57⁺ KLRG1⁻ LAG3⁻ PD1⁺ cells (e.g., T cells). In certain embodiments, the composition comprises at least about 45% CD57⁺ KLRG1⁻ LAG3⁻ PD1⁺ cells (e.g., T cells). In certain embodiments, the composition comprises at least about 50% CD57⁺ KLRG1⁻ LAG3⁻ PD1⁺ cells (e.g., T cells). In certain embodiments, the composition comprises at least about 55% CD57⁺ KLRG1⁻ LAG3⁻ PD1⁺ cells (e.g., T cells). In certain embodiments, the composition comprises at least about 60% CD57⁺ KLRG1⁻ LAG3⁻ PD1⁺ cells (e.g., T cells). In certain embodiments, the composition comprises at least about 65% CD57⁺ KLRG1⁻ LAG3⁻ PD1⁺ cells (e.g., T cells). In certain embodiments, the composition comprises at least about 70% CD57⁺ KLRG1⁻ LAG3⁻ PD1⁺ cells (e.g., T cells). In certain embodiments, the composition comprises at least about 75% CD57⁺ KLRG1⁻ LAG3⁻ PD1⁺ cells (e.g., T cells). In certain embodiments, the composition comprises at least about 80% CD57⁺ KLRG1⁻ LAG3⁻ PD1⁺ cells (e.g., T cells). In certain embodiments, the composition comprises at least about 85% CD57⁺ KLRG1⁻ LAG3⁻ PD1⁺ cells (e.g., T cells). In certain embodiments, the composition comprises at least about 90% CD57⁺ KLRG1⁻ LAG3⁻ PD1⁺ cells (e.g., T cells). In certain embodiments, the composition comprises at least about 95% CD57⁺ KLRG1⁻ LAG3⁻ PD1⁺ cells (e.g., T cells). In certain embodiments, the composition comprises about 100% CD57⁺ KLRG1⁻ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ CTLA4⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ CTLA4⁻ LAG3⁻ PD1⁺ cells (e.g., T cells). In certain embodiments, the composition comprises at least about 5% CD57⁺ CTLA4⁻ LAG3⁻ PD1⁺ cells (e.g., T cells). In certain embodiments, the composition comprises at least about 10% CD57⁺ CTLA4⁻ LAG3⁻ PD1⁺ cells (e.g., T cells). In certain embodiments, the composition comprises at least about 15% CD57⁺ CTLA4⁻ LAG3⁻ PD1⁺ cells (e.g., T cells). In certain embodiments, the composition comprises at least about 20% CD57⁺ CTLA4⁻ LAG3⁻ PD1⁺ cells (e.g., T cells). In certain embodiments, the composition comprises at least about 25% CD57⁺ CTLA4⁻ LAG3⁻ PD1⁺ cells (e.g., T cells). In certain embodiments, the composition comprises at least about 30% CD57⁺ CTLA4⁻ LAG3⁻ PD1⁺ cells (e.g., T cells). In certain embodiments, the composition comprises at least about 35% CD57⁺ CTLA4⁻ LAG3⁻ PD1⁺ cells (e.g., T cells). In certain embodiments, the composition comprises at least about 40% CD57⁺ CTLA4⁻ LAG3⁻ PD1⁺ cells (e.g., T cells). In certain embodiments, the composition comprises at least about 45% CD57⁺ CTLA4⁻ LAG3⁻ PD1⁺ cells (e.g., T cells). In certain embodiments, the composition comprises at least about 50% CD57⁺ CTLA4⁻ LAG3⁻ PD1⁺ cells (e.g., T cells). In certain embodiments, the composition comprises at least about 55% CD57⁺ CTLA4⁻ LAG3⁻ PD1⁺ cells (e.g., T cells). In certain embodiments, the composition comprises at least about 60% CD57⁺ CTLA4⁻ LAG3⁻ PD1⁺ cells (e.g., T cells). In certain embodiments, the composition comprises at least about 65% CD57⁺ CTLA4⁻ LAG3⁻ PD1⁺ cells (e.g., T cells). In certain embodiments, the composition comprises at least about 70% CD57⁺ CTLA4⁻ LAG3⁻ PD1⁺ cells (e.g., T cells). In certain embodiments, the composition comprises at least about 75% CD57⁺ CTLA4⁻ LAG3⁻ PD1⁺ cells (e.g., T cells). In certain embodiments, the composition comprises at least about 80% CD57⁺ CTLA4⁻ LAG3⁻ PD1⁺ cells (e.g., T cells). In certain embodiments, the composition comprises at least about 85% CD57⁺ CTLA4⁻ LAG3⁻ PD1⁺ cells (e.g., T cells). In certain embodiments, the composition comprises at least about 90% CD57⁺ CTLA4⁻ LAG3⁻ PD1⁺ cells (e.g., T cells). In certain embodiments, the composition comprises at least about 95% CD57⁺ CTLA4⁻ LAG3⁻ PD1⁺ cells (e.g., T cells). In certain embodiments, the composition comprises about 100% CD57⁺ CTLA4⁻ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ KLRG1⁻ CTLA4⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ KLRG1⁻ CTLA4⁻ LAG3⁻ cells (e.g., T cells). In certain embodiments, the composition comprises at least about 5% TIM3⁺ KLRG1⁻ CTLA4⁻ LAG3⁻ cells (e.g., T cells). In certain embodiments, the composition comprises at least about 10% TIM3⁺ KLRG1⁻ CTLA4⁻ LAG3⁻ cells (e.g., T cells). In certain embodiments, the composition comprises at least about 15% TIM3⁺ KLRG1⁻ CTLA4⁻ LAG3⁻ cells (e.g., T cells). In certain embodiments, the composition comprises at least about 20% TIM3⁺ KLRG1⁻ CTLA4⁻ LAG3⁻ cells (e.g., T cells). In certain embodiments, the composition comprises at least about 25% TIM3⁺ KLRG1⁻ CTLA4⁻ LAG3⁻ cells (e.g., T cells). In certain embodiments, the composition comprises at least about 30% TIM3⁺ KLRG1⁻ CTLA4⁻ LAG3⁻ cells (e.g., T cells). In certain embodiments, the composition comprises at least about 35% TIM3⁺ KLRG1⁻ CTLA4⁻ LAG3⁻ cells (e.g., T cells). In certain embodiments, the composition comprises at least about 40% TIM3⁺ KLRG1⁻ CTLA4⁻ LAG3⁻ cells (e.g., T cells). In certain embodiments, the composition comprises at least about 45% TIM3⁺ KLRG1⁻ CTLA4⁻ LAG3⁻ cells (e.g., T cells). In certain embodiments, the composition comprises at least about 50% TIM3⁺ KLRG1⁻ CTLA4⁻ LAG3⁻ cells (e.g., T cells). In certain embodiments, the composition comprises at least about 55% TIM3⁺ KLRG1⁻ CTLA4⁻ LAG3⁻ cells (e.g., T cells). In certain embodiments, the composition comprises at least about 60% TIM3⁺ KLRG1⁻ CTLA4⁻ LAG3⁻ cells (e.g., T cells). In certain embodiments, the composition comprises at least about 65% TIM3⁺ KLRG1⁻ CTLA4⁻ LAG3⁻ cells (e.g., T cells). In certain embodiments, the composition comprises at least about 70% TIM3⁺ KLRG1⁻ CTLA4⁻ LAG3⁻ cells (e.g., T cells). In certain embodiments, the composition comprises at least about 75% TIM3⁺ KLRG1⁻ CTLA4⁻ LAG3⁻ cells (e.g., T cells). In certain embodiments, the composition comprises at least about 80% TIM3⁺ KLRG1⁻ CTLA4⁻ LAG3⁻ cells (e.g., T cells). In certain embodiments, the composition comprises at least about 85% TIM3⁺ KLRG1⁻ CTLA4⁻ LAG3⁻ cells (e.g., T cells). In certain embodiments, the composition comprises at least about 90% TIM3⁺ KLRG1⁻ CTLA4⁻ LAG3⁻ cells (e.g., T cells). In certain embodiments, the composition comprises at least about 95% TIM3⁺ KLRG1⁻ CTLA4⁻ LAG3⁻ cells (e.g., T cells). In certain embodiments, the composition comprises about 100% TIM3⁺ KLRG1⁻ CTLA4⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ KLRG1⁻ CTLA4⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ KLRG1⁻ CTLA4⁻ PD1⁺ cells (e.g., T cells). In certain embodiments, the composition comprises at least about 5% TIM3⁺ KLRG1⁻ CTLA4⁻ PD1⁺ cells (e.g., T cells). In certain embodiments, the composition comprises at least about 10% TIM3⁺ KLRG1⁻ CTLA4⁻ PD1⁺ cells (e.g., T cells). In certain embodiments, the composition comprises at least about 15% TIM3⁺ KLRG1⁻ CTLA4⁻ PD1⁺ cells (e.g., T cells). In certain embodiments, the composition comprises at least about 20% TIM3⁺ KLRG1⁻ CTLA4⁻ PD1⁺ cells (e.g., T cells). In certain embodiments, the composition comprises at least about 25% TIM3⁺ KLRG1⁻ CTLA4⁻ PD1⁺ cells (e.g., T cells). In certain embodiments, the composition comprises at least about 30% TIM3⁺ KLRG1⁻ CTLA4⁻ PD1⁺ cells (e.g., T cells). In certain embodiments, the composition comprises at least about 35% TIM3⁺ KLRG1⁻ CTLA4⁻ PD1⁺ cells (e.g., T cells). In certain embodiments, the composition comprises at least about 40% TIM3⁺ KLRG1⁻ CTLA4⁻ PD1⁺ cells (e.g., T cells). In certain embodiments, the composition comprises at least about 45% TIM3⁺ KLRG1⁻ CTLA4⁻ PD1⁺ cells (e.g., T cells). In certain embodiments, the composition comprises at least about 50% TIM3⁺ KLRG1⁻ CTLA4⁻ PD1⁺ cells (e.g., T cells). In certain embodiments, the composition comprises at least about 55% TIM3⁺ KLRG1⁻ CTLA4⁻ PD1⁺ cells (e.g., T cells). In certain embodiments, the composition comprises at least about 60% TIM3⁺ KLRG1⁻ CTLA4⁻ PD1⁺ cells (e.g., T cells). In certain embodiments, the composition comprises at least about 65% TIM3⁺ KLRG1⁻ CTLA4⁻ PD1⁺ cells (e.g., T cells). In certain embodiments, the composition comprises at least about 70% TIM3⁺ KLRG1⁻ CTLA4⁻ PD1⁺ cells (e.g., T cells). In certain embodiments, the composition comprises at least about 75% TIM3⁺ KLRG1⁻ CTLA4⁻ PD1⁺ cells (e.g., T cells). In certain embodiments, the composition comprises at least about 80% TIM3⁺ KLRG1⁻ CTLA4⁻ PD1⁺ cells (e.g., T cells). In certain embodiments, the composition comprises at least about 85% TIM3⁺ KLRG1⁻ CTLA4⁻ PD1⁺ cells (e.g., T cells). In certain embodiments, the composition comprises at least about 90% TIM3⁺ KLRG1⁻ CTLA4⁻ PD1⁺ cells (e.g., T cells). In certain embodiments, the composition comprises at least about 95% TIM3⁺ KLRG1⁻ CTLA4⁻ PD1⁺ cells (e.g., T cells). In certain embodiments, the composition comprises about 100% TIM3⁺ KLRG1⁻ CTLA4⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ KLRG1⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ KLRG1⁻ LAG3⁻ PD1⁺ cells (e.g., T cells). In certain embodiments, the composition comprises at least about 5% TIM3⁺ KLRG1⁻ LAG3⁻ PD1⁺ cells (e.g., T cells). In certain embodiments, the composition comprises at least about 10% TIM3⁺ KLRG1⁻ LAG3⁻ PD1⁺ cells (e.g., T cells). In certain embodiments, the composition comprises at least about 15% TIM3⁺ KLRG1⁻ LAG3⁻ PD1⁺ cells (e.g., T cells). In certain embodiments, the composition comprises at least about 20% TIM3⁺ KLRG1⁻ LAG3⁻ PD1⁺ cells (e.g., T cells). In certain embodiments, the composition comprises at least about 25% TIM3⁺ KLRG1⁻ LAG3⁻ PD1⁺ cells (e.g., T cells). In certain embodiments, the composition comprises at least about 30% TIM3⁺ KLRG1⁻ LAG3⁻ PD1⁺ cells (e.g., T cells). In certain embodiments, the composition comprises at least about 35% TIM3⁺ KLRG1⁻ LAG3⁻ PD1⁺ cells (e.g., T cells). In certain embodiments, the composition comprises at least about 40% TIM3⁺ KLRG1⁻ LAG3⁻ PD1⁺ cells (e.g., T cells). In certain embodiments, the composition comprises at least about 45% TIM3⁺ KLRG1⁻ LAG3⁻ PD1⁺ cells (e.g., T cells). In certain embodiments, the composition comprises at least about 50% TIM3⁺ KLRG1⁻ LAG3⁻ PD1⁺ cells (e.g., T cells). In certain embodiments, the composition comprises at least about 55% TIM3⁺ KLRG1⁻ LAG3⁻ PD1⁺ cells (e.g., T cells). In certain embodiments, the composition comprises at least about 60% TIM3⁺ KLRG1⁻ LAG3⁻ PD1⁺ cells (e.g., T cells). In certain embodiments, the composition comprises at least about 65% TIM3⁺ KLRG1⁻ LAG3⁻ PD1⁺ cells (e.g., T cells). In certain embodiments, the composition comprises at least about 70% TIM3⁺ KLRG1⁻ LAG3⁻ PD1⁺ cells (e.g., T cells). In certain embodiments, the composition comprises at least about 75% TIM3⁺ KLRG1⁻ LAG3⁻ PD1⁺ cells (e.g., T cells). In certain embodiments, the composition comprises at least about 80% TIM3⁺ KLRG1⁻ LAG3⁻ PD1⁺ cells (e.g., T cells). In certain embodiments, the composition comprises at least about 85% TIM3⁺ KLRG1⁻ LAG3⁻ PD1⁺ cells (e.g., T cells). In certain embodiments, the composition comprises at least about 90% TIM3⁺ KLRG1⁻ LAG3⁻ PD1⁺ cells (e.g., T cells). In certain embodiments, the composition comprises at least about 95% TIM3⁺ KLRG1⁻ LAG3⁻ PD1⁺ cells (e.g., T cells). In certain embodiments, the composition comprises about 100% TIM3⁺ KLRG1⁻ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ CTLA4⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ CTLA4⁻ LAG3⁻ PD1⁺ cells (e.g., T cells). In certain embodiments, the composition comprises at least about 5% TIM3⁺ CTLA4⁻ LAG3⁻ PD1⁺ cells (e.g., T cells). In certain embodiments, the composition comprises at least about 10% TIM3⁺ CTLA4⁻ LAG3⁻ PD1⁺ cells (e.g., T cells). In certain embodiments, the composition comprises at least about 15% TIM3⁺ CTLA4⁻ LAG3⁻ PD1⁺ cells (e.g., T cells). In certain embodiments, the composition comprises at least about 20% TIM3⁺ CTLA4⁻ LAG3⁻ PD1⁺ cells (e.g., T cells). In certain embodiments, the composition comprises at least about 25% TIM3⁺ CTLA4⁻ LAG3⁻ PD1⁺ cells (e.g., T cells). In certain embodiments, the composition comprises at least about 30% TIM3⁺ CTLA4⁻ LAG3⁻ PD1⁺ cells (e.g., T cells). In certain embodiments, the composition comprises at least about 35% TIM3⁺ CTLA4⁻ LAG3⁻ PD1⁺ cells (e.g., T cells). In certain embodiments, the composition comprises at least about 40% TIM3⁺ CTLA4⁻ LAG3⁻ PD1⁺ cells (e.g., T cells). In certain embodiments, the composition comprises at least about 45% TIM3⁺ CTLA4⁻ LAG3⁻ PD1⁺ cells (e.g., T cells). In certain embodiments, the composition comprises at least about 50% TIM3⁺ CTLA4⁻ LAG3⁻ PD1⁺ cells (e.g., T cells). In certain embodiments, the composition comprises at least about 55% TIM3⁺ CTLA4⁻ LAG3⁻ PD1⁺ cells (e.g., T cells). In certain embodiments, the composition comprises at least about 60% TIM3⁺ CTLA4⁻ LAG3⁻ PD1⁺ cells (e.g., T cells). In certain embodiments, the composition comprises at least about 65% TIM3⁺ CTLA4⁻ LAG3⁻ PD1⁺ cells (e.g., T cells). In certain embodiments, the composition comprises at least about 70% TIM3⁺ CTLA4⁻ LAG3⁻ PD1⁺ cells (e.g., T cells). In certain embodiments, the composition comprises at least about 75% TIM3⁺ CTLA4⁻ LAG3⁻ PD1⁺ cells (e.g., T cells). In certain embodiments, the composition comprises at least about 80% TIM3⁺ CTLA4⁻ LAG3⁻ PD1⁺ cells (e.g., T cells). In certain embodiments, the composition comprises at least about 85% TIM3⁺ CTLA4⁻ LAG3⁻ PD1⁺ cells (e.g., T cells). In certain embodiments, the composition comprises at least about 90% TIM3⁺ CTLA4⁻ LAG3⁻ PD1⁺ cells (e.g., T cells). In certain embodiments, the composition comprises at least about 95% TIM3⁺ CTLA4⁻ LAG3⁻ PD1⁺ cells (e.g., T cells). In certain embodiments, the composition comprises about 100% TIM3⁺ CTLA4⁻ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% PD1⁺ KLRG1⁻ CTLA4⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% PD1⁺ KLRG1⁻ CTLA4⁻ LAG3⁻ cells (e.g., T cells). In certain embodiments, the composition comprises at least about 5% PD1⁺ KLRG1⁻ CTLA4⁻ LAG3⁻ cells (e.g., T cells). In certain embodiments, the composition comprises at least about 10% PD1⁺ KLRG1⁻ CTLA4⁻ LAG3⁻ cells (e.g., T cells). In certain embodiments, the composition comprises at least about 15% PD1⁺ KLRG1⁻ CTLA4⁻ LAG3⁻ cells (e.g., T cells). In certain embodiments, the composition comprises at least about 20% PD1⁺ KLRG1⁻ CTLA4⁻ LAG3⁻ cells (e.g., T cells). In certain embodiments, the composition comprises at least about 25% PD1⁺ KLRG1⁻ CTLA4⁻ LAG3⁻ cells (e.g., T cells). In certain embodiments, the composition comprises at least about 30% PD1⁺ KLRG1⁻ CTLA4⁻ LAG3⁻ cells (e.g., T cells). In certain embodiments, the composition comprises at least about 35% PD1⁺ KLRG1⁻ CTLA4⁻ LAG3⁻ cells (e.g., T cells). In certain embodiments, the composition comprises at least about 40% PD1⁺ KLRG1⁻ CTLA4⁻ LAG3⁻ cells (e.g., T cells). In certain embodiments, the composition comprises at least about 45% PD1⁺ KLRG1⁻ CTLA4⁻ LAG3⁻ cells (e.g., T cells). In certain embodiments, the composition comprises at least about 50% PD1⁺ KLRG1⁻ CTLA4⁻ LAG3⁻ cells (e.g., T cells). In certain embodiments, the composition comprises at least about 55% PD1⁺ KLRG1⁻ CTLA4⁻ LAG3⁻ cells (e.g., T cells). In certain embodiments, the composition comprises at least about 60% PD1⁺ KLRG1⁻ CTLA4⁻ LAG3⁻ cells (e.g., T cells). In certain embodiments, the composition comprises at least about 65% PD1⁺ KLRG1⁻ CTLA4⁻ LAG3⁻ cells (e.g., T cells). In certain embodiments, the composition comprises at least about 70% PD1⁺ KLRG1⁻ CTLA4⁻ LAG3⁻ cells (e.g., T cells). In certain embodiments, the composition comprises at least about 75% PD1⁺ KLRG1⁻ CTLA4⁻ LAG3⁻ cells (e.g., T cells). In certain embodiments, the composition comprises at least about 80% PD1⁺ KLRG1⁻ CTLA4⁻ LAG3⁻ cells (e.g., T cells). In certain embodiments, the composition comprises at least about 85% PD1⁺ KLRG1⁻ CTLA4⁻ LAG3⁻ cells (e.g., T cells). In certain embodiments, the composition comprises at least about 90% PD1⁺ KLRG1⁻ CTLA4⁻ LAG3⁻ cells (e.g., T cells). In certain embodiments, the composition comprises at least about 95% PD1⁺ KLRG1⁻ CTLA4⁻ LAG3⁻ cells (e.g., T cells). In certain embodiments, the composition comprises about 100% PD1⁺ KLRG1⁻ CTLA4⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ KLRG1⁻ CTLA4⁻ LAG3⁻ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ KLRG1⁻ CTLA4⁻ LAG3⁻ cells (e.g., T cells). In certain embodiments, the composition comprises at least about 5% CD57⁺ TIM3⁺ KLRG1⁻ CTLA4⁻ LAG3⁻ cells (e.g., T cells). In certain embodiments, the composition comprises at least about 10% CD57⁺ TIM3⁺ KLRG1⁻ CTLA4⁻ LAG3⁻ cells (e.g., T cells). In certain embodiments, the composition comprises at least about 15% CD57⁺ TIM3⁺ KLRG1⁻ CTLA4⁻ LAG3⁻ cells (e.g., T cells). In certain embodiments, the composition comprises at least about 20% CD57⁺ TIM3⁺ KLRG1⁻ CTLA4⁻ LAG3⁻ cells (e.g., T cells). In certain embodiments, the composition comprises at least about 25% CD57⁺ TIM3⁺ KLRG1⁻ CTLA4⁻ LAG3⁻ cells (e.g., T cells). In certain embodiments, the composition comprises at least about 30% CD57⁺ TIM3⁺ KLRG1⁻ CTLA4⁻ LAG3⁻ cells (e.g., T cells). In certain embodiments, the composition comprises at least about 35% CD57⁺ TIM3⁺ KLRG1⁻ CTLA4⁻ LAG3⁻ cells (e.g., T cells). In certain embodiments, the composition comprises at least about 40% CD57⁺ TIM3⁺ KLRG1⁻ CTLA4⁻ LAG3⁻ cells (e.g., T cells). In certain embodiments, the composition comprises at least about 45% CD57⁺ TIM3⁺ KLRG1⁻ CTLA4⁻ LAG3⁻ cells (e.g., T cells). In certain embodiments, the composition comprises at least about 50% CD57⁺ TIM3⁺ KLRG1⁻ CTLA4⁻ LAG3⁻ cells (e.g., T cells). In certain embodiments, the composition comprises at least about 55% CD57⁺ TIM3⁺ KLRG1⁻ CTLA4⁻ LAG3⁻ cells (e.g., T cells). In certain embodiments, the composition comprises at least about 60% CD57⁺ TIM3⁺ KLRG1⁻ CTLA4⁻ LAG3⁻ cells (e.g., T cells). In certain embodiments, the composition comprises at least about 65% CD57⁺ TIM3⁺ KLRG1⁻ CTLA4⁻ LAG3⁻ cells (e.g., T cells). In certain embodiments, the composition comprises at least about 70% CD57⁺ TIM3⁺ KLRG1⁻ CTLA4⁻ LAG3⁻ cells (e.g., T cells). In certain embodiments, the composition comprises at least about 75% CD57⁺ TIM3⁺ KLRG1⁻ CTLA4⁻ LAG3⁻ cells (e.g., T cells). In certain embodiments, the composition comprises at least about 80% CD57⁺ TIM3⁺ KLRG1⁻ CTLA4⁻ LAG3⁻ cells (e.g., T cells). In certain embodiments, the composition comprises at least about 85% CD57⁺ TIM3⁺ KLRG1⁻ CTLA4⁻ LAG3⁻ cells (e.g., T cells). In certain embodiments, the composition comprises at least about 90% CD57⁺ TIM3⁺ KLRG1⁻ CTLA4⁻ LAG3⁻ cells (e.g., T cells). In certain embodiments, the composition comprises at least about 95% CD57⁺ TIM3⁺ KLRG1⁻ CTLA4⁻ LAG3⁻ cells (e.g., T cells). In certain embodiments, the composition comprises about 100% CD57⁺ TIM3⁺ KLRG1⁻ CTLA4⁻ LAG3⁻ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ KLRG1⁻ CTLA4⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ KLRG1⁻ CTLA4⁻ PD1⁺ cells (e.g., T cells). In certain embodiments, the composition comprises at least about 5% CD57⁺ TIM3⁺ KLRG1⁻ CTLA4⁻ PD1⁺ cells (e.g., T cells). In certain embodiments, the composition comprises at least about 10% CD57⁺ TIM3⁺ KLRG1⁻ CTLA4⁻ PD1⁺ cells (e.g., T cells). In certain embodiments, the composition comprises at least about 15% CD57⁺ TIM3⁺ KLRG1⁻ CTLA4⁻ PD1⁺ cells (e.g., T cells). In certain embodiments, the composition comprises at least about 20% CD57⁺ TIM3⁺ KLRG1⁻ CTLA4⁻ PD1⁺ cells (e.g., T cells). In certain embodiments, the composition comprises at least about 25% CD57⁺ TIM3⁺ KLRG1⁻ CTLA4⁻ PD1⁺ cells (e.g., T cells). In certain embodiments, the composition comprises at least about 30% CD57⁺ TIM3⁺ KLRG1⁻ CTLA4⁻ PD1⁺ cells (e.g., T cells). In certain embodiments, the composition comprises at least about 35% CD57⁺ TIM3⁺ KLRG1⁻ CTLA4⁻ PD1⁺ cells (e.g., T cells). In certain embodiments, the composition comprises at least about 40% CD57⁺ TIM3⁺ KLRG1⁻ CTLA4⁻ PD1⁺ cells (e.g., T cells). In certain embodiments, the composition comprises at least about 45% CD57⁺ TIM3⁺ KLRG1⁻ CTLA4⁻ PD1⁺ cells (e.g., T cells). In certain embodiments, the composition comprises at least about 50% CD57⁺ TIM3⁺ KLRG1⁻ CTLA4⁻ PD1⁺ cells (e.g., T cells). In certain embodiments, the composition comprises at least about 55% CD57⁺ TIM3⁺ KLRG1⁻ CTLA4⁻ PD1⁺ cells (e.g., T cells). In certain embodiments, the composition comprises at least about 60% CD57⁺ TIM3⁺ KLRG1⁻ CTLA4⁻ PD1⁺ cells (e.g., T cells). In certain embodiments, the composition comprises at least about 65% CD57⁺ TIM3⁺ KLRG1⁻ CTLA4⁻ PD1⁺ cells (e.g., T cells). In certain embodiments, the composition comprises at least about 70% CD57⁺ TIM3⁺ KLRG1⁻ CTLA4⁻ PD1⁺ cells (e.g., T cells). In certain embodiments, the composition comprises at least about 75% CD57⁺ TIM3⁺ KLRG1⁻ CTLA4⁻ PD1⁺ cells (e.g., T cells). In certain embodiments, the composition comprises at least about 80% CD57⁺ TIM3⁺ KLRG1⁻ CTLA4⁻ PD1⁺ cells (e.g., T cells). In certain embodiments, the composition comprises at least about 85% CD57⁺ TIM3⁺ KLRG1⁻ CTLA4⁻ PD1⁺ cells (e.g., T cells). In certain embodiments, the composition comprises at least about 90% CD57⁺ TIM3⁺ KLRG1⁻ CTLA4⁻ PD1⁺ cells (e.g., T cells). In certain embodiments, the composition comprises at least about 95% CD57⁺ TIM3⁺ KLRG1⁻ CTLA4⁻ PD1⁺ cells (e.g., T cells). In certain embodiments, the composition comprises about 100% CD57⁺ TIM3⁺ KLRG1⁻ CTLA4⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ KLRG1⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ KLRG1⁻ LAG3⁻ PD1⁺ cells (e.g., T cells). In certain embodiments, the composition comprises at least about 5% CD57⁺ TIM3⁺ KLRG1⁻ LAG3⁻ PD1⁺ cells (e.g., T cells). In certain embodiments, the composition comprises at least about 10% CD57⁺ TIM3⁺ KLRG1⁻ LAG3⁻ PD1⁺ cells (e.g., T cells). In certain embodiments, the composition comprises at least about 15% CD57⁺ TIM3⁺ KLRG1⁻ LAG3⁻ PD1⁺ cells (e.g., T cells). In certain embodiments, the composition comprises at least about 20% CD57⁺ TIM3⁺ KLRG1⁻ LAG3⁻ PD1⁺ cells (e.g., T cells). In certain embodiments, the composition comprises at least about 25% CD57⁺ TIM3⁺ KLRG1⁻ LAG3⁻ PD1⁺ cells (e.g., T cells). In certain embodiments, the composition comprises at least about 30% CD57⁺ TIM3⁺ KLRG1⁻ LAG3⁻ PD1⁺ cells (e.g., T cells). In certain embodiments, the composition comprises at least about 35% CD57⁺ TIM3⁺ KLRG1⁻ LAG3⁻ PD1⁺ cells (e.g., T cells). In certain embodiments, the composition comprises at least about 40% CD57⁺ TIM3⁺ KLRG1⁻ LAG3⁻ PD1⁺ cells (e.g., T cells). In certain embodiments, the composition comprises at least about 45% CD57⁺ TIM3⁺ KLRG1⁻ LAG3⁻ PD1⁺ cells (e.g., T cells). In certain embodiments, the composition comprises at least about 50% CD57⁺ TIM3⁺ KLRG1⁻ LAG3⁻ PD1⁺ cells (e.g., T cells). In certain embodiments, the composition comprises at least about 55% CD57⁺ TIM3⁺ KLRG1⁻ LAG3⁻ PD1⁺ cells (e.g., T cells). In certain embodiments, the composition comprises at least about 60% CD57⁺ TIM3⁺ KLRG1⁻ LAG3⁻ PD1⁺ cells (e.g., T cells). In certain embodiments, the composition comprises at least about 65% CD57⁺ TIM3⁺ KLRG1⁻ LAG3⁻ PD1⁺ cells (e.g., T cells). In certain embodiments, the composition comprises at least about 70% CD57⁺ TIM3⁺ KLRG1⁻ LAG3⁻ PD1⁺ cells (e.g., T cells). In certain embodiments, the composition comprises at least about 75% CD57⁺ TIM3⁺ KLRG1⁻ LAG3⁻ PD1⁺ cells (e.g., T cells). In certain embodiments, the composition comprises at least about 80% CD57⁺ TIM3⁺ KLRG1⁻ LAG3⁻ PD1⁺ cells (e.g., T cells). In certain embodiments, the composition comprises at least about 85% CD57⁺ TIM3⁺ KLRG1⁻ LAG3⁻ PD1⁺ cells (e.g., T cells). In certain embodiments, the composition comprises at least about 90% CD57⁺ TIM3⁺ KLRG1⁻ LAG3⁻ PD1⁺ cells (e.g., T cells). In certain embodiments, the composition comprises at least about 95% CD57⁺ TIM3⁺ KLRG1⁻ LAG3⁻ PD1⁺ cells (e.g., T cells). In certain embodiments, the composition comprises about 100% CD57⁺ TIM3⁺ KLRG1⁻ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ CTLA4⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ CTLA4⁻ LAG3⁻ PD1⁺ cells (e.g., T cells). In certain embodiments, the composition comprises at least about 5% CD57⁺ TIM3⁺ CTLA4⁻ LAG3⁻ PD1⁺ cells (e.g., T cells). In certain embodiments, the composition comprises at least about 10% CD57⁺ TIM3⁺ CTLA4⁻ LAG3⁻ PD1⁺ cells (e.g., T cells). In certain embodiments, the composition comprises at least about 15% CD57⁺ TIM3⁺ CTLA4⁻ LAG3⁻ PD1⁺ cells (e.g., T cells). In certain embodiments, the composition comprises at least about 20% CD57⁺ TIM3⁺ CTLA4⁻ LAG3⁻ PD1⁺ cells (e.g., T cells). In certain embodiments, the composition comprises at least about 25% CD57⁺ TIM3⁺ CTLA4⁻ LAG3⁻ PD1⁺ cells (e.g., T cells). In certain embodiments, the composition comprises at least about 30% CD57⁺ TIM3⁺ CTLA4⁻ LAG3⁻ PD1⁺ cells (e.g., T cells). In certain embodiments, the composition comprises at least about 35% CD57⁺ TIM3⁺ CTLA4⁻ LAG3⁻ PD1⁺ cells (e.g., T cells). In certain embodiments, the composition comprises at least about 40% CD57⁺ TIM3⁺ CTLA4⁻ LAG3⁻ PD1⁺ cells (e.g., T cells). In certain embodiments, the composition comprises at least about 45% CD57⁺ TIM3⁺ CTLA4⁻ LAG3⁻ PD1⁺ cells (e.g., T cells). In certain embodiments, the composition comprises at least about 50% CD57⁺ TIM3⁺ CTLA4⁻ LAG3⁻ PD1⁺ cells (e.g., T cells). In certain embodiments, the composition comprises at least about 55% CD57⁺ TIM3⁺ CTLA4⁻ LAG3⁻ PD1⁺ cells (e.g., T cells). In certain embodiments, the composition comprises at least about 60% CD57⁺ TIM3⁺ CTLA4⁻ LAG3⁻ PD1⁺ cells (e.g., T cells). In certain embodiments, the composition comprises at least about 65% CD57⁺ TIM3⁺ CTLA4⁻ LAG3⁻ PD1⁺ cells (e.g., T cells). In certain embodiments, the composition comprises at least about 70% CD57⁺ TIM3⁺ CTLA4⁻ LAG3⁻ PD1⁺ cells (e.g., T cells). In certain embodiments, the composition comprises at least about 75% CD57⁺ TIM3⁺ CTLA4⁻ LAG3⁻ PD1⁺ cells (e.g., T cells). In certain embodiments, the composition comprises at least about 80% CD57⁺ TIM3⁺ CTLA4⁻ LAG3⁻ PD1⁺ cells (e.g., T cells). In certain embodiments, the composition comprises at least about 85% CD57⁺ TIM3⁺ CTLA4⁻ LAG3⁻ PD1⁺ cells (e.g., T cells). In certain embodiments, the composition comprises at least about 90% CD57⁺ TIM3⁺ CTLA4⁻ LAG3⁻ PD1⁺ cells (e.g., T cells). In certain embodiments, the composition comprises at least about 95% CD57⁺ TIM3⁺ CTLA4⁻ LAG3⁻ PD1⁺ cells (e.g., T cells). In certain embodiments, the composition comprises about 100% CD57⁺ TIM3⁺ CTLA4⁻ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ KLRG1⁻ CTLA4⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ KLRG1⁻ CTLA4⁻ LAG3⁻ PD1⁺ cells (e.g., T cells). In certain embodiments, the composition comprises at least about 5% CD57⁺ KLRG1⁻ CTLA4⁻ LAG3⁻ PD1⁺ cells (e.g., T cells). In certain embodiments, the composition comprises at least about 10% CD57⁺ KLRG1⁻ CTLA4⁻ LAG3⁻ PD1⁺ cells (e.g., T cells). In certain embodiments, the composition comprises at least about 15% CD57⁺ KLRG1⁻ CTLA4⁻ LAG3⁻ PD1⁺ cells (e.g., T cells). In certain embodiments, the composition comprises at least about 20% CD57⁺ KLRG1⁻ CTLA4⁻ LAG3⁻ PD1⁺ cells (e.g., T cells). In certain embodiments, the composition comprises at least about 25% CD57⁺ KLRG1⁻ CTLA4⁻ LAG3⁻ PD1⁺ cells (e.g., T cells). In certain embodiments, the composition comprises at least about 30% CD57⁺ KLRG1⁻ CTLA4⁻ LAG3⁻ PD1⁺ cells (e.g., T cells). In certain embodiments, the composition comprises at least about 35% CD57⁺ KLRG1⁻ CTLA4⁻ LAG3⁻ PD1⁺ cells (e.g., T cells). In certain embodiments, the composition comprises at least about 40% CD57⁺ KLRG1⁻ CTLA4⁻ LAG3⁻ PD1⁺ cells (e.g., T cells). In certain embodiments, the composition comprises at least about 45% CD57⁺ KLRG1⁻ CTLA4⁻ LAG3⁻ PD1⁺ cells (e.g., T cells). In certain embodiments, the composition comprises at least about 50% CD57⁺ KLRG1⁻ CTLA4⁻ LAG3⁻ PD1⁺ cells (e.g., T cells). In certain embodiments, the composition comprises at least about 55% CD57⁺ KLRG1⁻ CTLA4⁻ LAG3⁻ PD1⁺ cells (e.g., T cells). In certain embodiments, the composition comprises at least about 60% CD57⁺ KLRG1⁻ CTLA4⁻ LAG3⁻ PD1⁺ cells (e.g., T cells). In certain embodiments, the composition comprises at least about 65% CD57⁺ KLRG1⁻ CTLA4⁻ LAG3⁻ PD1⁺ cells (e.g., T cells). In certain embodiments, the composition comprises at least about 70% CD57⁺ KLRG1⁻ CTLA4⁻ LAG3⁻ PD1⁺ cells (e.g., T cells). In certain embodiments, the composition comprises at least about 75% CD57⁺ KLRG1⁻ CTLA4⁻ LAG3⁻ PD1⁺ cells (e.g., T cells). In certain embodiments, the composition comprises at least about 80% CD57⁺ KLRG1⁻ CTLA4⁻ LAG3⁻ PD1⁺ cells (e.g., T cells). In certain embodiments, the composition comprises at least about 85% CD57⁺ KLRG1⁻ CTLA4⁻ LAG3⁻ PD1⁺ cells (e.g., T cells). In certain embodiments, the composition comprises at least about 90% CD57⁺ KLRG1⁻ CTLA4⁻ LAG3⁻ PD1⁺ cells (e.g., T cells). In certain embodiments, the composition comprises at least about 95% CD57⁺ KLRG1⁻ CTLA4⁻ LAG3⁻ PD1⁺ cells (e.g., T cells). In certain embodiments, the composition comprises about 100% CD57⁺ KLRG1⁻ CTLA4⁻ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% TIM3⁺ KLRG1⁻ CTLA4⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% TIM3⁺ KLRG1⁻ CTLA4⁻ LAG3⁻ PD1⁺ cells (e.g., T cells). In certain embodiments, the composition comprises at least about 5% TIM3⁺ KLRG1⁻ CTLA4⁻ LAG3⁻ PD1⁺ cells (e.g., T cells). In certain embodiments, the composition comprises at least about 10% TIM3⁺ KLRG1⁻ CTLA4⁻ LAG3⁻ PD1⁺ cells (e.g., T cells). In certain embodiments, the composition comprises at least about 15% TIM3⁺ KLRG1⁻ CTLA4⁻ LAG3⁻ PD1⁺ cells (e.g., T cells). In certain embodiments, the composition comprises at least about 20% TIM3⁺ KLRG1⁻ CTLA4⁻ LAG3⁻ PD1⁺ cells (e.g., T cells). In certain embodiments, the composition comprises at least about 25% TIM3⁺ KLRG1⁻ CTLA4⁻ LAG3⁻ PD1⁺ cells (e.g., T cells). In certain embodiments, the composition comprises at least about 30% TIM3⁺ KLRG1⁻ CTLA4⁻ LAG3⁻ PD1⁺ cells (e.g., T cells). In certain embodiments, the composition comprises at least about 35% TIM3⁺ KLRG1⁻ CTLA4⁻ LAG3⁻ PD1⁺ cells (e.g., T cells). In certain embodiments, the composition comprises at least about 40% TIM3⁺ KLRG1⁻ CTLA4⁻ LAG3⁻ PD1⁺ cells (e.g., T cells). In certain embodiments, the composition comprises at least about 45% TIM3⁺ KLRG1⁻ CTLA4⁻ LAG3⁻ PD1⁺ cells (e.g., T cells). In certain embodiments, the composition comprises at least about 50% TIM3⁺ KLRG1⁻ CTLA4⁻ LAG3⁻ PD1⁺ cells (e.g., T cells). In certain embodiments, the composition comprises at least about 55% TIM3⁺ KLRG1⁻ CTLA4⁻ LAG3⁻ PD1⁺ cells (e.g., T cells). In certain embodiments, the composition comprises at least about 60% TIM3⁺ KLRG1⁻ CTLA4⁻ LAG3⁻ PD1⁺ cells (e.g., T cells). In certain embodiments, the composition comprises at least about 65% TIM3⁺ KLRG1⁻ CTLA4⁻ LAG3⁻ PD1⁺ cells (e.g., T cells). In certain embodiments, the composition comprises at least about 70% TIM3⁺ KLRG1⁻ CTLA4⁻ LAG3⁻ PD1⁺ cells (e.g., T cells). In certain embodiments, the composition comprises at least about 75% TIM3⁺ KLRG1⁻ CTLA4⁻ LAG3⁻ PD1⁺ cells (e.g., T cells). In certain embodiments, the composition comprises at least about 80% TIM3⁺ KLRG1⁻ CTLA4⁻ LAG3⁻ PD1⁺ cells (e.g., T cells). In certain embodiments, the composition comprises at least about 85% TIM3⁺ KLRG1⁻ CTLA4⁻ LAG3⁻ PD1⁺ cells (e.g., T cells). In certain embodiments, the composition comprises at least about 90% TIM3⁺ KLRG1⁻ CTLA4⁻ LAG3⁻ PD1⁺ cells (e.g., T cells). In certain embodiments, the composition comprises at least about 95% TIM3⁺ KLRG1⁻ CTLA4⁻ LAG3⁻ PD1⁺ cells (e.g., T cells). In certain embodiments, the composition comprises about 100% TIM3⁺ KLRG1⁻ CTLA4⁻ LAG3⁻ PD1⁺ cells (e.g., T cells).

In certain embodiments, the composition comprises from about 5% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 5% to about 10%, from about 5% to about 15%, from about 5% to about 25%, from about 5% to about 35%, from about 5% to about 45%, from about 5% to about 55%, from about 5% to about 65%, from about 5% to about 75%, from about 5% to about 85%, from about 5% to about 95% CD57⁺ TIM3⁺ KLRG1⁻ CTLA4⁻ LAG3⁻ PD1⁺ cells (e.g., T cells). In certain embodiments, the composition comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% CD57⁺ TIM3⁺ KLRG1⁻ CTLA4⁻ LAG3⁻ PD1⁺ cells (e.g., T cells). In certain embodiments, the composition comprises at least about 5% CD57⁺ TIM3⁺ KLRG1⁻ CTLA4⁻ LAG3⁻ PD1⁺ cells (e.g., T cells). In certain embodiments, the composition comprises at least about 10% CD57⁺ TIM3⁺ KLRG1⁻ CTLA4⁻ LAG3⁻ PD1⁺ cells (e.g., T cells). In certain embodiments, the composition comprises at least about 15% CD57⁺ TIM3⁺ KLRG1⁻ CTLA4⁻ LAG3⁻ PD1⁺ cells (e.g., T cells). In certain embodiments, the composition comprises at least about 20% CD57⁺ TIM3⁺ KLRG1⁻ CTLA4⁻ LAG3⁻ PD1⁺ cells (e.g., T cells). In certain embodiments, the composition comprises at least about 25% CD57⁺ TIM3⁺ KLRG1⁻ CTLA4⁻ LAG3⁻ PD1⁺ cells (e.g., T cells). In certain embodiments, the composition comprises at least about 30% CD57⁺ TIM3⁺ KLRG1⁻ CTLA4⁻ LAG3⁻ PD1⁺ cells (e.g., T cells). In certain embodiments, the composition comprises at least about 35% CD57⁺ TIM3⁺ KLRG1⁻ CTLA4⁻ LAG3⁻ PD1⁺ cells (e.g., T cells). In certain embodiments, the composition comprises at least about 40% CD57⁺ TIM3⁺ KLRG1⁻ CTLA4⁻ LAG3⁻ PD1⁺ cells (e.g., T cells). In certain embodiments, the composition comprises at least about 45% CD57⁺ TIM3⁺ KLRG1⁻ CTLA4⁻ LAG3⁻ PD1⁺ cells (e.g., T cells). In certain embodiments, the composition comprises at least about 50% CD57⁺ TIM3⁺ KLRG1⁻ CTLA4⁻ LAG3⁻ PD1⁺ cells (e.g., T cells). In certain embodiments, the composition comprises at least about 55% CD57⁺ TIM3⁺ KLRG1⁻ CTLA4⁻ LAG3⁻ PD1⁺ cells (e.g., T cells). In certain embodiments, the composition comprises at least about 60% CD57⁺ TIM3⁺ KLRG1⁻ CTLA4⁻ LAG3⁻ PD1⁺ cells (e.g., T cells). In certain embodiments, the composition comprises at least about 65% CD57⁺ TIM3⁺ KLRG1⁻ CTLA4⁻ LAG3⁻ PD1⁺ cells (e.g., T cells). In certain embodiments, the composition comprises at least about 70% CD57⁺ TIM3⁺ KLRG1⁻ CTLA4⁻ LAG3⁻ PD1⁺ cells (e.g., T cells). In certain embodiments, the composition comprises at least about 75% CD57⁺ TIM3⁺ KLRG1⁻ CTLA4⁻ LAG3⁻ PD1⁺ cells (e.g., T cells). In certain embodiments, the composition comprises at least about 80% CD57⁺ TIM3⁺ KLRG1⁻ CTLA4⁻ LAG3⁻ PD1⁺ cells (e.g., T cells). In certain embodiments, the composition comprises at least about 85% CD57⁺ TIM3⁺ KLRG1⁻ CTLA4⁻ LAG3⁻ PD1⁺ cells (e.g., T cells). In certain embodiments, the composition comprises at least about 90% CD57⁺ TIM3⁺ KLRG1⁻ CTLA4⁻ LAG3⁻ PD1⁺ cells (e.g., T cells). In certain embodiments, the composition comprises at least about 95% CD57⁺ TIM3⁺ KLRG1⁻ CTLA4⁻ LAG3⁻ PD1⁺ cells (e.g., T cells). In certain embodiments, the composition comprises about 100% CD57⁺ TIM3⁺ KLRG1⁻ CTLA4⁻ LAG3⁻ PD1⁺ cells (e.g., T cells).

In certain embodiments, the composition can include multiple type of cells. For example, but without any limitation, the composition can include CD57⁺ cells, TIM3⁺ cells, KLRG1⁻ cells, CTLA4⁻ cells, LAG3⁻ cells, PD1⁺ cells, CD57⁺ TIM3⁺ cells, CD57⁺ KLRG1⁻ cells, CD57⁺ CTLA4⁻ cells, CD57⁺ LAG3⁻ cells, CD57⁺ PD1⁺ cells, TIM3⁺ KLRG1⁻ cells, TIM3⁺ CTLA4⁻ cells, TIM3⁺ LAG3⁻ cells, TIM3⁺ PD1⁺ cells, KLRG1⁻ CTLA4⁻ cells, KLRG1⁻ LAG3⁻ cells, KLRG1⁻ PD1⁺ cells, CTLA4⁻ LAG3⁻ cells, CTLA4⁻ PD1⁺ cells, LAG3⁻ PD1⁺ cells, CD57⁺ TIM3⁺ KLRG1⁻ cells, CD57⁺ TIM3⁺ CTLA4⁻ cells, CD57⁺ TIM3⁺ LAG3⁻ cells, CD57⁺ TIM3⁺ PD1⁺ cells, CD57⁺ KLRG1⁻ CTLA4⁻ cells, CD57⁺ KLRG1⁻ LAG3⁻ cells, CD57⁺ KLRG1⁻ PD1⁺ cells, CD57⁺ CTLA4⁻ LAG3⁻ cells, CD57⁺ CTLA4⁻ PD1⁺ cells, CD57⁺ LAG3⁻ PD1⁺ cells, TIM3⁺ KLRG1⁻ CTLA4⁻ cells, TIM3⁺ KLRG1⁻ LAG3⁻ cells, TIM3⁺ KLRG1⁻ PD1⁺ cells, TIM3⁺ CTLA4⁻ LAG3⁻ cells, TIM3⁺ CTLA4⁻ PD1⁺ cells, TIM3⁺ LAG3⁻ PD1⁺ cells, KLRG1⁻ CTLA4⁻ LAG3⁻ cells, KLRG1⁻ CTLA4⁻ PD1⁺ cells, KLRG1⁻ LAG3⁻ PD1⁺ cells, KLRG1⁻ CTLA4⁻ LAG3⁻ cells, KLRG1⁻ CTLA4⁻ PD1⁺ cells, KLRG1⁻ LAG3⁻ PD1⁺ cells, CTLA4⁻ LAG3⁻ PD1⁺ cells, CD57⁺ TIM3⁺ KLRG1⁻ CTLA4⁻ cells, CD57⁺ TIM3⁺ KLRG1⁻ LAG3⁻ cells, CD57⁺ TIM3⁺ KLRG1⁻ PD1⁺ cells, CD57⁺ TIM3⁺ CTLA4⁻ LAG3⁻ cells, CD57⁺ TIM3⁺ CTLA4⁻ PD1⁺ cells, CD57⁺ TIM3⁺ LAG3⁻ PD1⁺ cells, CD57⁺ KLRG1⁻ CTLA4⁻ LAG3⁻ cells, CD57⁺ KLRG1⁻ CTLA4⁻ PD1⁺ cells, CD57⁺ KLRG1⁻ LAG3⁻ PD1⁺ cells, CD57⁺ CTLA4⁻ LAG3⁻ PD1⁺ cells, TIM3⁺ KLRG1⁻ CTLA4⁻ LAG3⁻ cells, TIM3⁺ KLRG1⁻ CTLA4⁻ PD1⁺ cells, TIM3⁺ KLRG1⁻ LAG3⁻ PD1⁺ cells, TIM3⁺ CTLA4⁻ LAG3⁻ PD1⁺ cells, PD1⁺ KLRG1⁻ CTLA4⁻ LAG3⁻ cells, CD57⁺ TIM3⁺ KLRG1⁻ CTLA4⁻ LAG3⁻ cells, CD57⁺ TIM3⁺ KLRG1⁻ CTLA4⁻ PD1⁺ cells, CD57⁺ TIM3⁺ KLRG1⁻ LAG3⁻ PD1⁺ cells, CD57⁺ TIM3⁺ CTLA4⁻ LAG3⁻ PD1⁺ cells, CD57⁺ KLRG1⁻ CTLA4⁻ LAG3⁻ PD1⁺ cells, TIM3⁺ KLRG1⁻ CTLA4⁻ LAG3⁻ PD1⁺ cells, CD57⁺ TIM3⁺ KLRG1⁻ CTLA4⁻ LAG3⁻ PD1⁺ cells, or any combination thereof. The quantity of cells present in the presently disclosed composition can vary. For example, without any limitation, the composition can include about 5% CD57⁺ cells, about 5% TIM3⁺ cells, about 10% CD57⁺ TIM3⁺ cells, about 10% KLRG1⁻ CTLA4⁻ LAG3⁻ cells, about 10% CD57⁺ TIM3⁺ KLRG1⁻ CTLA4⁻ cells, about 10% CD57⁺ TIM3⁺ KLRG1⁻ LAG3⁻ cells, about 10% TIM3⁺ KLRG1⁻ CTLA4⁻ LAG3⁻ cells, and about 40% CD57⁺ TIM3⁺ KLRG1⁻ CTLA4⁻ LAG3⁻ cells. In another example, but without any limitation, the composition can include about 5% PD1⁺ cells, about 5% TIM3⁺ cells, about 10% CD57⁺ TIM3⁺ cells, about 10% KLRG1⁻ CTLA4⁻ LAG3⁻ cells, about 10% PD1⁺ TIM3⁺ KLRG1⁻ CTLA4⁻ cells, about 10% CD57⁺ PD1⁺ KLRG1⁻ LAG3⁻ cells, about 10% TIM3⁺ KLRG1⁻ CTLA4⁻ LAG3⁻ cells, and about 40% CD57⁺ TIM3⁺ KLRG1⁻ CTLA4⁻ LAG3⁻ PD1 cells.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a neoplasm. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, between about 10×10⁶ and about 150×10⁶ of the presently disclosed cells are administered to a subject. In certain embodiments, between about 25×10⁶ and about 150×10⁶ of the presently disclosed cells are administered to a subject. In certain embodiments, between about 25×10⁶ and about 50×10⁶ of the presently disclosed cells are administered to a subject. In certain embodiments, at least about 10×10⁶, about 25×10⁶, about 50×10⁶, about 100×10⁶, or about 150×10⁶ of the presently disclosed cells are administered to a subject. In certain embodiments, about 25×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus).

### 5. Biomarkers

In certain embodiments, the presently disclosed subject matter includes the analysis of the immunoresponsive cells, e.g., CD4⁺ T cells and/or CD8⁺ T cells, of a subject. For example, but not by way of limitation, the presently disclosed subject matter includes the measuring of the expression level of a biomarker in a sample of the subject compared to a reference sample. In certain embodiments, the presently disclosed subject matter includes the measuring of the expression level of a biomarker in a sample from the subject compared to a reference sample.

In certain embodiments, the methods of the presently disclosed subject matter include measuring the expression level of a biomarker selected from CD57, TIM3, KLRG1, CTLA4, LAG3, PD1, or a combination thereof in immunoresponsive cells (e.g., CD4⁺ T cells, CD8⁺ T cells).

In certain embodiments, the methods of the presently disclosed subject matter include measuring the expression level of CD57 in immunoresponsive cells (e.g., CD4⁺ T cells, CD8⁺ T cells). CD57, also called human natural killer-1 (HNK-1) or LEU-7, is present on CD4⁺ T cells, CD8⁺ T cells, and natural killer (NK) cells at the late stages of differentiation. It is associated with terminally differentiated cells with lower proliferative capacity and altered functional activities. CD57-positive lymphocytes (CD57⁺ lymphocytes) are often increased in solid tumors. However, while recent studies have associated tumor-infiltrating CD57⁺ lymphocytes and cancer prognosis, their results were controversial (Nielsen et al., Frontiers in immunology 4 (2013): 422).

In certain embodiments, the methods of the presently disclosed subject matter include measuring the expression level of TIM3 in immunoresponsive cells (e.g., CD4⁺ T cells, CD8⁺ T cells). T-cell immunoglobulin and mucin-dominant containing-3 (TIM-3), also known as hepatitis A virus cellular receptor 2 (HAVCR2), has been reported as an immune-checkpoint molecule. TIM-3 plays an important role in suppressing cytotoxic T lymphocytes (CTL) and Th1 responses and the expression of cytokines such as tumor necrosis factor and interferon-γ. In addition, TIM-3 regulates innate and adaptive immune responses, possibly exerting either positive or negative effects. Numerous studies have reported that TIM-3 is expressed in cancer and that its expression is correlated with poor prognosis in many cancers. However, the role of TIM-3 in clinical cancer studies is still conflicting (Zang et al., Frontiers in oncology 11 (2021): 579351).

In certain embodiments, the methods of the presently disclosed subject matter include measuring the expression level of KLRG1 in immunoresponsive cells (e.g., CD4⁺ T cells, CD8⁺ T cells). Killer cell lectin-like receptor G1 (KLRG1) is a homodimeric member of the lectin-like type 2 transmembrane receptor family whose members contain characteristic immunoreceptor tyrosine-based inhibitory motifs (ITIMs) in their cytoplasmic domains. These ITIMs interact with the SH2 domains of protein phosphatases such as SHP-1. KLRG1 is an inhibitory receptor (e.g., immune checkpoint) that is expressed on natural killer (NK) cells and certain T cells.

In certain embodiments, the methods of the presently disclosed subject matter include measuring the expression level of CTLA4 in immunoresponsive cells (e.g., CD4⁺ T cells, CD8⁺ T cells). Cytotoxic T lymphocyte antigen-4 (CTLA-4) is an important inhibitory receptor that regulates T cell function and plays a role in the priming phase of the immune response. Following T cell activation through CD28 binding, CTLA-4 is expressed on the surface of T cells. Among its functions, its inhibitory signal is transmitted through the binding of B7-1 and B7-2 on activated B cells and monocytes (Buchbinder and Hodi, The Journal of clinical investigation 125.9 (2015): 3377-3383).

In certain embodiments, the methods of the presently disclosed subject matter include measuring the expression level of LAG3 in immunoresponsive cells (e.g., CD4⁺ T cells, CD8⁺ T cells). Lymphocyte activation gene 3 (LAG3) is a glycoprotein capable of binding to MHC class II with higher affinity than CD4 and is thought to be involved in the negative regulation of T cell activation and homeostatic proliferation. Surface expression of LAG3 has been reported on activated T cells (including regulatory T cells) and NK cells.

In certain embodiments, the methods of the presently disclosed subject matter include measuring the expression level of PD1 in immunoresponsive cells (e.g., CD4⁺ T cells, CD8⁺ T cells). PD1 is an inhibitor of both adaptive and innate immune responses, and is expressed on activated T, natural killer (NK) and B lymphocytes, macrophages, dendritic cells (DCs) and monocytes. Notably, PD1 can be highly expressed on tumor-specific T cells. PD1 plays two opposing roles, as it can be both beneficial and harmful. As regards its beneficial effects, it plays a key role in reducing the regulation of ineffective or harmful immune responses and maintaining immune tolerance. However, PD-1 causes the dilation of malignant cells by interfering with the protective immune response (Han et al., American journal of cancer research 10.3 (2020): 727).

In certain embodiments, the biomarker is a protein present on the surface of a tissue (e.g., tumor cell) or of a cell (e.g., a T cell). Proteins can be isolated and detected using any number of methods, which are well-known in the art. Non-limiting examples of methods for the detection of the biomarkers disclosed herein include mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation, immunohistochemistry, cytometry, and flow cytometry.

In certain embodiments, the biomarker is detected and/or measured on the surface of a cell obtained from a sample. In certain embodiments, the sample is known to have or suspected to have a T cell. Non-limiting examples of samples encompassed by the presently disclosed subject matter include bone marrow, thymus, tumor biopsy, tumor fragments, enzymatically digested tumor tissue, dissociated/suspended cells, a lymph node sample, or a bodily fluid sample (e.g., blood, ascites, lymph).

In certain embodiments, the biomarker is detected and/or measured using cytometry or flow cytometry. As used herein, the term “cytometry” refers to a technique for identifying, sorting, or analyzing cells. For example, but without any limitation, cytometry can be used to analyze cell size, cell count, cell morphology (e.g., shape and structure), cell cycle phase, DNA content, and the existence or absence of specific proteins on the cell surface or in the cytoplasm.

Additionally, as used herein, the term “flow cytometry” refers to a cytometric technique in which cells present in a fluid flow can be identified, sorted, or analyzed. In certain non-limiting embodiments, flow cytometry requires labeling the cells with fluorescent markers and detecting the fluorescent markers via radiative excitation. Conventionally, a sample containing cells (e.g., T cells) is suspended in a fluid and injected into the flow cytometer instrument. The sample allows the flow of one cell at a time through a laser beam, where the light scattered is characteristic of the cells and their components. Additional information on flow cytometry and its use can be found in Selliah et al., Current Protocols 3.8 (2023): e868, Shevach, Current Protocols in Immunology 87.1 (2009): 5-0, and McKinnon, Current protocols in immunology 120.1 (2018): 5-1, the contents of each of which are incorporated by reference in their entireties.

In certain embodiments, the biomarker is detected and/or measured using immunohistochemistry (IHC).

In certain embodiments, the biomarker is measured on the surface of an immunoresponsive cell. In certain embodiments, the biomarker is measured on the surface of a T cell. For example, but without any limitation, the biomarkers can be measured on the surface of helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, γδ T cells, CD4⁺ T cells, and CD8⁺ T cells.

In certain embodiments, the biomarker is a nucleic acid (e.g., RNA) present in a tissue (e.g., tumor cell) or a cell (e.g., a T cell). In certain embodiments, the nucleic acid is RNA. RNA can be isolated and detected using any number of methods, which are well-known in the art. Non-limiting examples of methods for the detection of the biomarkers disclosed herein include gene expression profiling, polymerase chain reaction (PCR), quantitative polymerase chain reaction (qPCR), microarray analysis, serial analysis of gene expression (SAGE), microarray, gene sequencing, and the like.

In certain embodiments, the presently disclosed subject matter includes the analysis of CD4⁺ T cells and/or CD8⁺ T cells of a subject. In certain embodiments, the analysis of CD4⁺ T cells and/or CD8⁺ T cells of a subject includes measuring the expression level of a biomarker (e.g., CD57, TIM3, KLRG1, CTLA4, LAG3, PD1, or a combination thereof) in a sample (e.g., bone marrow, thymus, tumor biopsy, tumor fragments, enzymatically digested tumor tissue, dissociated/suspended cells, a lymph node sample, or a blood sample) of the subject. In certain embodiments, the analysis of CD4⁺ T cells and/or CD8⁺ T cells of a subject includes analyzing the expression level of a biomarker (e.g., CD57, TIM3, KLRG1, CTLA4, LAG3, PD1, or a combination thereof) in a sample (e.g., bone marrow, thymus, tumor biopsy, tumor fragments, enzymatically digested tumor tissue, dissociated/suspended cells, a lymph node sample, or a blood sample) of the subject compared to a reference sample.

In certain embodiments, the presently disclosed subject matter provides for methods of identifying a subject having cancer that is responsive to an adoptive cell therapy (e.g., a cell disclosed in Section 2 or Section 2.4, or a composition disclosed in Section 4). In certain embodiments, the presently disclosed subject matter provides for methods of identifying a subject having cancer that is non-responsive to an adoptive cell therapy (e.g., a cell disclosed in Section 2 or Section 2.4). In certain embodiments, the presently disclosed subject matter provides for methods of determining whether a subject having a cancer has an increased likelihood or reduced likelihood to respond to an adoptive cell therapy (e.g., a cell disclosed in Section 2 or Section 2.4, or a composition disclosed in Section 4).

In certain embodiments, the presently disclosed subject matter provides a method of identifying a subject having cancer that is responsive to an adoptive cell therapy including measuring the expression level of a biomarker (e.g., CD57, TIM3, KLRG1, CTLA4, LAG3, PD1, or a combination thereof) in immunoresponsive cells (e.g., CD4⁺ T cells, CD8⁺ T cells), comparing the expression level to a reference sample, and identifying the subject as responsive to the adoptive cell therapy.

In certain embodiments, the presently disclosed subject matter provides a method of identifying a subject having an increased likelihood to respond to an adoptive cell therapy including measuring the expression level of a biomarker (e.g., CD57, TIM3, KLRG1, CTLA4, LAG3, PD1, or a combination thereof) in immunoresponsive cells (e.g., CD4⁺ T cells, CD8⁺ T cells), comparing the expression level to a reference sample, and identifying the subject as having an increased likelihood to respond to the adoptive cell therapy.

In certain embodiments, the presently disclosed subject matter provides a method of identifying a subject having cancer that is non-responsive to an adoptive cell therapy including measuring the expression level of a biomarker (e.g., CD57, TIM3, KLRG1, CTLA4, LAG3, PD1, or a combination thereof) in immunoresponsive cells (e.g., CD4⁺ T cells, CD8⁺ T cells), comparing the expression level to a reference sample, and identifying the subject as non-responsive to the adoptive cell therapy.

In certain embodiments, the presently disclosed subject matter provides a method of identifying a subject having a reduced likelihood to respond to an adoptive cell therapy including measuring the expression level of a biomarker (e.g., CD57, TIM3, KLRG1, CTLA4, LAG3, PD1, or a combination thereof) in immunoresponsive cells (e.g., CD4⁺ T cells, CD8⁺ T cells), comparing the expression level to a reference sample, and identifying the subject as having a reduced likelihood to respond to the adoptive cell therapy.

In certain embodiments, the methods of the presently disclosed subject matter include measuring the expression level of a biomarker selected from CD57, TIM3, KLRG1, CTLA4, LAG3, TIM3, PD1, or a combination thereof in immunoresponsive cells (e.g., CD4⁺ T cells, CD8⁺ T cells). In certain embodiments, the methods of the presently disclosed subject matter include measuring the expression level of a biomarker selected from CD57, TIM3, KLRG1, CTLA4, LAG3, TIM3, PD1, or a combination thereof in T cells (e.g., CD4⁺ T cells, CD8⁺ T cells).

In certain embodiments, the methods of the presently disclosed subject matter include measuring the expression level of CD57 in T cells (e.g., CD4⁺ T cells, CD8⁺ T cells). In certain embodiments, the methods of the presently disclosed subject matter include measuring the expression level of TIM3 in T cells (e.g., CD4⁺ T cells, CD8⁺ T cells). In certain embodiments, the methods of the presently disclosed subject matter include measuring the expression level of KLRG1 in T cells (e.g., CD4⁺ T cells, CD8⁺ T cells). In certain embodiments, the methods of the presently disclosed subject matter include measuring the expression level of CTLA4 in T cells (e.g., CD4⁺ T cells, CD8⁺ T cells). In certain embodiments, the methods of the presently disclosed subject matter include measuring the expression level of LAG3 in T cells (e.g., CD4⁺ T cells, CD8⁺ T cells). In certain embodiments, the methods of the presently disclosed subject matter include measuring the expression level of PD1 in T cells (e.g., CD4⁺ T cells, CD8⁺ T cells).

In certain embodiments, the methods of the presently disclosed subject matter include measuring the expression level of CD57 and TIM3 in T cells (e.g., CD4⁺ T cells, CD8⁺ T cells). In certain embodiments, the methods of the presently disclosed subject matter include measuring the expression level of CD57 and KLRG1 in T cells (e.g., CD4⁺ T cells, CD8⁺ T cells). In certain embodiments, the methods of the presently disclosed subject matter include measuring the expression level of CD57 and CTLA4 in T cells (e.g., CD4⁺ T cells, CD8⁺ T cells). In certain embodiments, the methods of the presently disclosed subject matter include measuring the expression level of CD57 and LAG3 in T cells (e.g., CD4⁺ T cells, CD8⁺ T cells). In certain embodiments, the methods of the presently disclosed subject matter include measuring the expression level of CD57 and PD1 in T cells (e.g., CD4⁺ T cells, CD8⁺ T cells).

In certain embodiments, the methods of the presently disclosed subject matter include measuring the expression level of TIM3 and KLRG1 in T cells (e.g., CD4⁺ T cells, CD8⁺ T cells). In certain embodiments, the methods of the presently disclosed subject matter include measuring the expression level of TIM3 and CTLA4 in T cells (e.g., CD4⁺ T cells, CD8⁺ T cells). In certain embodiments, the methods of the presently disclosed subject matter include measuring the expression level of TIM3 and LAG3 in T cells (e.g., CD4⁺ T cells, CD8⁺ T cells). In certain embodiments, the methods of the presently disclosed subject matter include measuring the expression level of TIM3 and PD1 in T cells (e.g., CD4⁺ T cells, CD8⁺ T cells).

In certain embodiments, the methods of the presently disclosed subject matter include measuring the expression level of KLRG1 and CTLA4 in T cells (e.g., CD4⁺ T cells, CD8⁺ T cells). In certain embodiments, the methods of the presently disclosed subject matter include measuring the expression level of KLRG1 and LAG3 in T cells (e.g., CD4⁺ T cells, CD8⁺ T cells). In certain embodiments, the methods of the presently disclosed subject matter include measuring the expression level of KLRG1 and PD1 in T cells (e.g., CD4⁺ T cells, CD8⁺ T cells).

In certain embodiments, the methods of the presently disclosed subject matter include measuring the expression level of CTLA4 and LAG3 in T cells (e.g., CD4⁺ T cells, CD8⁺ T cells). In certain embodiments, the methods of the presently disclosed subject matter include measuring the expression level of CTLA4 and PD1 in T cells (e.g., CD4⁺ T cells, CD8⁺ T cells). In certain embodiments, the methods of the presently disclosed subject matter include measuring the expression level of LAG3 and PD1 in T cells (e.g., CD4⁺ T cells, CD8⁺ T cells). In certain embodiments, the methods of the presently disclosed subject matter include measuring the expression level of CD57, TIM3, and KLRG1 in T cells (e.g., CD4⁺ T cells, CD8⁺ T cells). In certain embodiments, the methods of the presently disclosed subject matter include measuring the expression level of CD57, TIM3, and CTLA4, in T cells (e.g., CD4⁺ T cells, CD8⁺ T cells). In certain embodiments, the methods of the presently disclosed subject matter include measuring the expression level of CD57, TIM3, and LAG3 in T cells (e.g., CD4⁺ T cells, CD8⁺ T cells). In certain embodiments, the methods of the presently disclosed subject matter include measuring the expression level of CD57, TIM3, and PD1 in T cells (e.g., CD4⁺ T cells, CD8⁺ T cells).

In certain embodiments, the methods of the presently disclosed subject matter include measuring the expression level of CD57, KLRG1, and CTLA4 in T cells (e.g., CD4⁺ T cells, CD8⁺ T cells). In certain embodiments, the methods of the presently disclosed subject matter include measuring the expression level of CD57, KLRG1, and LAG3 in T cells (e.g., CD4⁺ T cells, CD8⁺ T cells). In certain embodiments, the methods of the presently disclosed subject matter include measuring the expression level of CD57, KLRG1, and PD1 in T cells (e.g., CD4⁺ T cells, CD8⁺ T cells). In certain embodiments, the methods of the presently disclosed subject matter include measuring the expression level of CD57, CTLA4, and LAG3 in T cells (e.g., CD4⁺ T cells, CD8⁺ T cells). In certain embodiments, the methods of the presently disclosed subject matter include measuring the expression level of CD57, CTLA4, and PD1 in T cells (e.g., CD4⁺ T cells, CD8⁺ T cells). In certain embodiments, the methods of the presently disclosed subject matter include measuring the expression level of CD57, LAG3, and PD1 in T cells (e.g., CD4⁺ T cells, CD8⁺ T cells).

In certain embodiments, the methods of the presently disclosed subject matter include measuring the expression level of TIM3, KLRG1, and CTLA4 in T cells (e.g., CD4⁺ T cells, CD8⁺ T cells). In certain embodiments, the methods of the presently disclosed subject matter include measuring the expression level of TIM3, KLRG1, and LAG3 in T cells (e.g., CD4⁺ T cells, CD8⁺ T cells). In certain embodiments, the methods of the presently disclosed subject matter include measuring the expression level of TIM3, KLRG1, and PD1 in T cells (e.g., CD4⁺ T cells, CD8⁺ T cells). In certain embodiments, the methods of the presently disclosed subject matter include measuring the expression level of TIM3, CTLA4, and LAG3 in T cells (e.g., CD4⁺ T cells, CD8⁺ T cells). In certain embodiments, the methods of the presently disclosed subject matter include measuring the expression level of TIM3, CTLA4, and PD1 in T cells (e.g., CD4⁺ T cells, CD8⁺ T cells). In certain embodiments, the methods of the presently disclosed subject matter include measuring the expression level of TIM3, LAG3, and PD1 in T cells (e.g., CD4⁺ T cells, CD8⁺ T cells).

In certain embodiments, the methods of the presently disclosed subject matter include measuring the expression level of KLRG1, CTLA4, and LAG3 in T cells (e.g., CD4⁺ T cells, CD8⁺ T cells). In certain embodiments, the methods of the presently disclosed subject matter include measuring the expression level of KLRG1, CTLA4, and PD1 in T cells (e.g., CD4⁺ T cells, CD8⁺ T cells). In certain embodiments, the methods of the presently disclosed subject matter include measuring the expression level of KLRG1, LAG3, and PD1 in T cells (e.g., CD4⁺ T cells, CD8⁺ T cells). In certain embodiments, the methods of the presently disclosed subject matter include measuring the expression level of CTLA4, LAG3, and PD1 in T cells (e.g., CD4⁺ T cells, CD8⁺ T cells).

In certain embodiments, the methods of the presently disclosed subject matter include measuring the expression level of CD57, TIM3, KLRG1, and CTLA4 in T cells (e.g., CD4⁺ T cells, CD8⁺ T cells). In certain embodiments, the methods of the presently disclosed subject matter include measuring the expression level of CD57, TIM3, KLRG1, and LAG3 in T cells (e.g., CD4⁺ T cells, CD8⁺ T cells). In certain embodiments, the methods of the presently disclosed subject matter include measuring the expression level of CD57, TIM3, KLRG1, and PD1 in T cells (e.g., CD4⁺ T cells, CD8⁺ T cells). In certain embodiments, the methods of the presently disclosed subject matter include measuring the expression level of CD57, TIM3, CTLA4, and LAG3 in T cells (e.g., CD4⁺ T cells, CD8⁺ T cells). In certain embodiments, the methods of the presently disclosed subject matter include measuring the expression level of CD57, TIM3, CTLA4, and PD1 in T cells (e.g., CD4⁺ T cells, CD8⁺ T cells). In certain embodiments, the methods of the presently disclosed subject matter include measuring the expression level of CD57, TIM3, LAG3, and PD1 in T cells (e.g., CD4⁺ T cells, CD8⁺ T cells).

In certain embodiments, the methods of the presently disclosed subject matter include measuring the expression level of CD57, KLRG1, CTLA4, and LAG3 in T cells (e.g., CD4⁺ T cells, CD8⁺ T cells). In certain embodiments, the methods of the presently disclosed subject matter include measuring the expression level of CD57, KLRG1, CTLA4, and PD1 in T cells (e.g., CD4⁺ T cells, CD8⁺ T cells). In certain embodiments, the methods of the presently disclosed subject matter include measuring the expression level of CD57, KLRG1, LAG3, and PD1 in T cells (e.g., CD4⁺ T cells, CD8⁺ T cells). In certain embodiments, the methods of the presently disclosed subject matter include measuring the expression level of CD57, CTLA4, LAG3, and PD1 in T cells (e.g., CD4⁺ T cells, CD8⁺ T cells).

In certain embodiments, the methods of the presently disclosed subject matter include measuring the expression level of TIM3, KLRG1, CTLA4, and LAG3 in T cells (e.g., CD4⁺ T cells, CD8⁺ T cells). In certain embodiments, the methods of the presently disclosed subject matter include measuring the expression level of TIM3, KLRG1, CTLA4, and PD1 in T cells (e.g., CD4⁺ T cells, CD8⁺ T cells). In certain embodiments, the methods of the presently disclosed subject matter include measuring the expression level of TIM3, KLRG1, LAG3, and PD1 in T cells (e.g., CD4⁺ T cells, CD8⁺ T cells). In certain embodiments, the methods of the presently disclosed subject matter include measuring the expression level of TIM3, CTLA4, LAG3, and PD1 in T cells (e.g., CD4⁺ T cells, CD8⁺ T cells). In certain embodiments, the methods of the presently disclosed subject matter include measuring the expression level of KLRG1, CTLA4, LAG3, and PD1 in T cells (e.g., CD4⁺ T cells, CD8⁺ T cells).

In certain embodiments, the methods of the presently disclosed subject matter include measuring the expression level of CD57, TIM3, KLRG1, CTLA4, and LAG3 in T cells (e.g., CD4⁺ T cells, CD8⁺ T cells). In certain embodiments, the methods of the presently disclosed subject matter include measuring the expression level of CD57, TIM3, KLRG1, CTLA4, and PD1 in T cells (e.g., CD4⁺ T cells, CD8⁺ T cells). In certain embodiments, the methods of the presently disclosed subject matter include measuring the expression level of CD57, TIM3, KLRG1, LAG3, and PD1 in T cells (e.g., CD4⁺ T cells, CD8⁺ T cells). In certain embodiments, the methods of the presently disclosed subject matter include measuring the expression level of CD57, TIM3, CTLA4, LAG3, and PD1 in T cells (e.g., CD4⁺ T cells, CD8⁺ T cells). In certain embodiments, the methods of the presently disclosed subject matter include measuring the expression level of CD57, KLRG1, CTLA4, LAG3, and PD1 in T cells (e.g., CD4⁺ T cells, CD8⁺ T cells). In certain embodiments, the methods of the presently disclosed subject matter include measuring the expression level of TIM3, KLRG1, CTLA4, LAG3, and PD1 in T cells (e.g., CD4⁺ T cells, CD8⁺ T cells).

In certain embodiments, the methods of the presently disclosed subject matter include measuring the expression level of CD57, TIM3, KLRG1, CTLA4, LAG3, and PD1 in T cells (e.g., CD4⁺ T cells, CD8⁺ T cells).

In certain embodiments, the subject having cancer is responsive to an adoptive cell therapy (e.g., a cell disclosed in Section 2 or Section 2.4, or a composition disclosed in Section 4) if the expression level of CD57 in T cells (e.g., CD4⁺ T cells, CD8⁺ T cells) is increased compared to a reference sample. In certain embodiments, the subject having cancer is responsive to an adoptive cell therapy (e.g., a cell disclosed in Section 2 or Section 2.4, or a composition disclosed in Section 4) if the expression level of CD57 in CD4⁺ T cells is increased compared to a reference sample. In certain embodiments, the subject having cancer is responsive to an adoptive cell therapy (e.g., a cell disclosed in Section 2 or Section 2.4, or a composition disclosed in Section 4) if the expression level of CD57 in CD8⁺ T cells is increased compared to a reference sample. In certain embodiments, the subject having cancer is responsive to an adoptive cell therapy (e.g., a cell disclosed in Section 2 or Section 2.4, or a composition disclosed in Section 4) if the expression level of CD57 in CD4⁺ T cells and CD8⁺ T cells is increased compared to a reference sample.

In certain embodiments, the subject having cancer is responsive to an adoptive cell therapy (e.g., a cell disclosed in Section 2 or Section 2.4, or a composition disclosed in Section 4) if the expression level of TIM3 in T cells (e.g., CD4⁺ T cells, CD8⁺ T cells) is increased compared to a reference sample. In certain embodiments, the subject having cancer is responsive to an adoptive cell therapy (e.g., a cell disclosed in Section 2 or Section 2.4, or a composition disclosed in Section 4) if the expression level of TIM3 in CD4⁺ T cells is increased compared to a reference sample. In certain embodiments, the subject having cancer is responsive to an adoptive cell therapy (e.g., a cell disclosed in Section 2 or Section 2.4, or a composition disclosed in Section 4) if the expression level of TIM3 in CD8⁺ T cells is increased compared to a reference sample. In certain embodiments, the subject having cancer is responsive to an adoptive cell therapy (e.g., a cell disclosed in Section 2 or Section 2.4, or a composition disclosed in Section 4) if the expression level of TIM3 in CD4⁺ T cells and CD8⁺ T cells is increased compared to a reference sample.

In certain embodiments, the subject having cancer is responsive to an adoptive cell therapy (e.g., a cell disclosed in Section 2 or Section 2.4, or a composition disclosed in Section 4) if the expression level of KLRG1 in T cells (e.g., CD4⁺ T cells, CD8⁺ T cells) is reduced compared to a reference sample. In certain embodiments, the subject having cancer is responsive to an adoptive cell therapy (e.g., a cell disclosed in Section 2 or Section 2.4, or a composition disclosed in Section 4) if the expression level of KLRG1 in CD4⁺ T cells is reduced compared to a reference sample. In certain embodiments, the subject having cancer is responsive to an adoptive cell therapy (e.g., a cell disclosed in Section 2 or Section 2.4, or a composition disclosed in Section 4) if the expression level of KLRG1 in CD8⁺ T cells is reduced compared to a reference sample. In certain embodiments, the subject having cancer is responsive to an adoptive cell therapy (e.g., a cell disclosed in Section 2 or Section 2.4, or a composition disclosed in Section 4) if the expression level of KLRG1 in CD4⁺ T cells and CD8⁺ T cells is reduced compared to a reference sample.

In certain embodiments, the subject having cancer is responsive to an adoptive cell therapy (e.g., a cell disclosed in Section 2 or Section 2.4, or a composition disclosed in Section 4) if the expression level of CTLA4 in T cells (e.g., CD4⁺ T cells, CD8⁺ T cells) is reduced compared to a reference sample. In certain embodiments, the subject having cancer is responsive to an adoptive cell therapy (e.g., a cell disclosed in Section 2 or Section 2.4, or a composition disclosed in Section 4) if the expression level of CTLA4 in CD4⁺ T cells is reduced compared to a reference sample. In certain embodiments, the subject having cancer is responsive to an adoptive cell therapy (e.g., a cell disclosed in Section 2 or Section 2.4, or a composition disclosed in Section 4) if the expression level of CTLA4 in CD8⁺ T cells is reduced compared to a reference sample. In certain embodiments, the subject having cancer is responsive to an adoptive cell therapy (e.g., a cell disclosed in Section 2 or Section 2.4, or a composition disclosed in Section 4) if the expression level of CTLA4 in CD4⁺ T cells and CD8⁺ T cells is reduced compared to a reference sample.

In certain embodiments, the subject having cancer is responsive to an adoptive cell therapy (e.g., a cell disclosed in Section 2 or Section 2.4, or a composition disclosed in Section 4) if the expression level of LAG3 in T cells (e.g., CD4⁺ T cells, CD8⁺ T cells) is reduced compared to a reference sample. In certain embodiments, the subject having cancer is responsive to an adoptive cell therapy (e.g., a cell disclosed in Section 2 or Section 2.4, or a composition disclosed in Section 4) if the expression level of LAG3 in CD4⁺ T cells is reduced compared to a reference sample. In certain embodiments, the subject having cancer is responsive to an adoptive cell therapy (e.g., a cell disclosed in Section 2 or Section 2.4, or a composition disclosed in Section 4) if the expression level of LAG3 in CD8⁺ T cells is reduced compared to a reference sample. In certain embodiments, the subject having cancer is responsive to an adoptive cell therapy (e.g., a cell disclosed in Section 2 or Section 2.4, or a composition disclosed in Section 4) if the expression level of LAG3 in CD4⁺ T cells and CD8⁺ T cells is reduced compared to a reference sample.

In certain embodiments, the subject having cancer is responsive to an adoptive cell therapy (e.g., a cell disclosed in Section 2 or Section 2.4, or a composition disclosed in Section 4) if the expression level of PD1 in T cells (e.g., CD4⁺ T cells, CD8⁺ T cells) is increased compared to a reference sample. In certain embodiments, the subject having cancer is responsive to an adoptive cell therapy (e.g., a cell disclosed in Section 2 or Section 2.4, or a composition disclosed in Section 4) if the expression level of PD1 in CD4⁺ T cells is increased compared to a reference sample. In certain embodiments, the subject having cancer is responsive to an adoptive cell therapy (e.g., a cell disclosed in Section 2 or Section 2.4, or a composition disclosed in Section 4) if the expression level of PD1 in CD8⁺ T cells is increased compared to a reference sample. In certain embodiments, the subject having cancer is responsive to an adoptive cell therapy (e.g., a cell disclosed in Section 2 or Section 2.4, or a composition disclosed in Section 4) if the expression level of PD1 in CD4⁺ T cells and CD8⁺ T cells is increased compared to a reference sample.

In certain embodiments, the subject having cancer is responsive to an adoptive cell therapy (e.g., a cell disclosed in Section 2 or Section 2.4, or a composition disclosed in Section 4) if the expression level of at least one of CD57, TIM3, and PD1 in T cells (e.g., CD4⁺ T cells, CD8⁺ T cells) is increased compared to a reference sample. In certain embodiments, the subject having cancer is responsive to an adoptive cell therapy (e.g., a cell disclosed in Section 2 or Section 2.4, or a composition disclosed in Section 4) if the expression level of CD57, TIM3, and PD1 in CD4⁺ T cells is increased compared to a reference sample. In certain embodiments, the subject having cancer is responsive to an adoptive cell therapy (e.g., a cell disclosed in Section 2 or Section 2.4, or a composition disclosed in Section 4) if the expression level of CD57, TIM3, and PD1 in CD8⁺ T cells is increased compared to a reference sample. In certain embodiments, the subject having cancer is responsive to an adoptive cell therapy (e.g., a cell disclosed in Section 2 or Section 2.4, or a composition disclosed in Section 4) if the expression level of CD57, TIM3, PD1 in CD4⁺ T cells and CD8⁺ T cells is increased compared to a reference sample.

In certain embodiments, the subject having cancer is responsive to an adoptive cell therapy (e.g., a cell disclosed in Section 2 or Section 2.4, or a composition disclosed in Section 4) if the expression level of at least one of KLRG1, CTLA4, and LAG3 in T cells (e.g., CD4⁺ T cells, CD8⁺ T cells) is reduced compared to a reference sample. In certain embodiments, the subject having cancer is responsive to an adoptive cell therapy (e.g., a cell disclosed in Section 2 or Section 2.4, or a composition disclosed in Section 4) if the expression level of at least one of KLRG1, CTLA4, and LAG3 in CD4⁺ T cells is reduced compared to a reference sample. In certain embodiments, the subject having cancer is responsive to an adoptive cell therapy (e.g., a cell disclosed in Section 2 or Section 2.4, or a composition disclosed in Section 4) if the expression level of at least one of KLRG1, CTLA4, and LAG3 in CD8⁺ T cells is reduced compared to a reference sample. In certain embodiments, the subject having cancer is responsive to an adoptive cell therapy (e.g., a cell disclosed in Section 2 or Section 2.4, or a composition disclosed in Section 4) if the expression level of at least one of KLRG1, CTLA4, and LAG3 in CD4⁺ T cells and CD8⁺ T cells is reduced compared to a reference sample.

In certain embodiments, the subject having cancer is responsive to an adoptive cell therapy (e.g., a cell disclosed in Section 2 or Section 2.4, or a composition disclosed in Section 4) if
a) the expression level of at least one of CD57, TIM3, and PD1 in T cells (e.g., CD4⁺ T cells, CD8⁺ T cells) is increased compared to a reference sample; and/or
b) the expression level of at least one of KLRG1, CTLA4, and LAG3 in T cells (e.g., CD4⁺ T cells, CD8⁺ T cells) is reduced compared to the reference sample.

In certain embodiments, the subject having cancer is responsive to an adoptive cell therapy (e.g., a cell disclosed in Section 2 or Section 2.4, or a composition disclosed in Section 4) if
a) the expression level of at least one of CD57, TIM3, and PD1 in CD4⁺ T cells is increased compared to a reference sample; and/or
b) the expression level of at least one of KLRG1, CTLA4, and LAG3 in CD4⁺ T cells is reduced compared to the reference sample.

In certain embodiments, the subject having cancer is responsive to an adoptive cell therapy (e.g., a cell disclosed in Section 2 or Section 2.4, or a composition disclosed in Section 4) if
a) the expression level of at least one of CD57, TIM3, and PD1 in CD8⁺ T cells is increased compared to a reference sample; and/or
b) the expression level of at least one of KLRG1, CTLA4, and LAG3 in CD8⁺ T cells is reduced compared to the reference sample.

In certain embodiments, the subject having cancer is responsive to an adoptive cell therapy (e.g., a cell disclosed in Section 2 or Section 2.4, or a composition disclosed in Section 4) if
a) the expression level of at least one of CD57, TIM3, and PD1 in CD4⁺ T cells and CD8⁺ T cells is increased compared to a reference sample; and/or
b) the expression level of at least one of KLRG1, CTLA4, and LAG3 in CD4⁺ T cells and CD8⁺ T cells is reduced compared to the reference sample.

In certain embodiments, the subject having cancer has an increased likelihood to respond to an adoptive cell therapy (e.g., a cell disclosed in Section 2 or Section 2.4, or a composition disclosed in Section 4) if the expression level of CD57 in T cells (e.g., CD4⁺ T cells, CD8⁺ T cells) is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of CD57 in CD4⁺ T cells is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of CD57 in CD8⁺ T cells is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of CD57 in CD4⁺ T cells and CD8⁺ T cells is increased compared to a reference sample.

In certain embodiments, the subject having cancer has an increased likelihood to respond to an adoptive cell therapy (e.g., a cell disclosed in Section 2 or Section 2.4, or a composition disclosed in Section 4) if the expression level of TIM3 in T cells (e.g., CD4⁺ T cells, CD8⁺ T cells) is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of TIM3 in CD4⁺ T cells is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of TIM3 in CD8⁺ T cells is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of TIM3 in CD4⁺ T cells and CD8⁺ T cells is increased compared to a reference sample.

In certain embodiments, the subject having cancer has an increased likelihood to respond to an adoptive cell therapy (e.g., a cell disclosed in Section 2 or Section 2.4, or a composition disclosed in Section 4) if the expression level of KLRG1 in T cells (e.g., CD4⁺ T cells, CD8⁺ T cells) is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of KLRG1 in CD4⁺ T cells is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of KLRG1 in CD8⁺ T cells is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of KLRG1 in CD4⁺ T cells and CD8⁺ T cells is reduced compared to a reference sample.

In certain embodiments, the subject having cancer has an increased likelihood to respond to an adoptive cell therapy (e.g., a cell disclosed in Section 2 or Section 2.4, or a composition disclosed in Section 4) if the expression level of CTLA4 in T cells (e.g., CD4⁺ T cells, CD8⁺ T cells) is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of CTLA4 in CD4⁺ T cells is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of CTLA4 in CD8⁺ T cells is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of CTLA4 in CD4⁺ T cells and CD8⁺ T cells is reduced compared to a reference sample.

In certain embodiments, the subject having cancer has an increased likelihood to respond to an adoptive cell therapy (e.g., a cell disclosed in Section 2 or Section 2.4, or a composition disclosed in Section 4) if the expression level of LAG3 in T cells (e.g., CD4⁺ T cells, CD8⁺ T cells) is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of LAG3 in CD4⁺ T cells is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of LAG3 in CD8⁺ T cells is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of LAG3 in CD4⁺ T cells and CD8⁺ T cells is reduced compared to a reference sample.

In certain embodiments, the subject having cancer has an increased likelihood to respond to an adoptive cell therapy (e.g., a cell disclosed in Section 2 or Section 2.4, or a composition disclosed in Section 4) if the expression level of PD1 in T cells (e.g., CD4⁺ T cells, CD8⁺ T cells) is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of PD1 in CD4⁺ T cells is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of PD1 in CD8⁺ T cells is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of PD1 in CD4⁺ T cells and CD8⁺ T cells is increased compared to a reference sample.

In certain embodiments, the subject having cancer has an increased likelihood to respond to an adoptive cell therapy (e.g., a cell disclosed in Section 2 or Section 2.4, or a composition disclosed in Section 4) if the expression level of at least one of CD57, TIM3, and PD1 in T cells (e.g., CD4⁺ T cells, CD8⁺ T cells) is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of CD57, TIM3, and PD1 in CD4⁺ T cells is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of CD57, TIM3, and PD1 in CD8⁺ T cells is increased compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of CD57, TIM3, and PD1 in CD4⁺ T cells and CD8⁺ T cells is increased compared to a reference sample.

In certain embodiments, the subject having cancer has an increased likelihood to respond to an adoptive cell therapy (e.g., a cell disclosed in Section 2 or Section 2.4, or a composition disclosed in Section 4) if the expression level of at least one of KLRG1, CTLA4, and LAG3 in T cells (e.g., CD4⁺ T cells, CD8⁺ T cells) is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of at least one of KLRG1, CTLA4, and LAG3 in CD4⁺ T cells is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of at least one of KLRG1, CTLA4, and LAG3 in CD8⁺ T cells is reduced compared to a reference sample. In certain embodiments, the subject having cancer has an increased likelihood to respond to an adoptive cell therapy (e.g., a cell disclosed in Section 2 or Section 2.4, or a composition disclosed in Section 4) if the expression level of at least one of KLRG1, CTLA4, and LAG3 in CD4⁺ T cells and CD8⁺ T cells is reduced compared to a reference sample.

In certain embodiments, the subject having cancer has an increased likelihood to respond to an adoptive cell therapy (e.g., a cell disclosed in Section 2 or Section 2.4, or a composition disclosed in Section 4) if
a) the expression level of at least one of CD57, TIM3, and PD1 in T cells (e.g., CD4⁺ T cells, CD8⁺ T cells) is increased compared to a reference sample; and/or
b) the expression level of at least one of KLRG1, CTLA4, and LAG3 in T cells (e.g., CD4⁺ T cells, CD8⁺ T cells) is reduced compared to the reference sample.

In certain embodiments, the subject having cancer has an increased likelihood to respond to an adoptive cell therapy (e.g., a cell disclosed in Section 2 or Section 2.4, or a composition disclosed in Section 4) if
a) the expression level of at least one of CD57, TIM3, and PD1 in CD4⁺ T cells is increased compared to a reference sample; and/or
b) the expression level of at least one of KLRG1, CTLA4, and LAG3 in CD4⁺ T cells is reduced compared to the reference sample.

In certain embodiments, the subject having cancer has an increased likelihood to respond to an adoptive cell therapy (e.g., a cell disclosed in Section 2 or Section 2.4, or a composition disclosed in Section 4) if
a) the expression level of at least one of CD57, TIM3, and PD1 in CD8⁺ T cells is increased compared to a reference sample; and/or
b) the expression level of at least one of KLRG1, CTLA4, and LAG3 in CD8⁺ T cells is reduced compared to the reference sample.

In certain embodiments, the subject having cancer has an increased likelihood to respond to an adoptive cell therapy (e.g., a cell disclosed in Section 2 or Section 2.4, or a composition disclosed in Section 4) if
a) the expression level of at least one of CD57, TIM3, and PD1 in CD4⁺ T cells and CD8⁺ T cells is increased compared to a reference sample; and/or
b) the expression level of at least one of KLRG1, CTLA4, and LAG3 in CD4⁺ T cells and CD8⁺ T cells is reduced compared to the reference sample.

In certain embodiments, the subject having cancer is non-responsive to an adoptive cell therapy (e.g., a cell disclosed in Section 2 or Section 2.4) if the expression level of CD57 in T cells (e.g., CD4⁺ T cells, CD8⁺ T cells) is reduced compared to a reference sample. In certain embodiments, the subject having cancer is non-responsive to an adoptive cell therapy (e.g., a cell disclosed in Section 2 or Section 2.4) if the expression level of CD57 in CD4⁺ T cells is reduced compared to a reference sample. In certain embodiments, the subject having cancer is non-responsive to an adoptive cell therapy (e.g., a cell disclosed in Section 2 or Section 2.4) if the expression level of CD57 in CD8⁺ T cells is reduced compared to a reference sample. In certain embodiments, the subject having cancer is non-responsive to an adoptive cell therapy (e.g., a cell disclosed in Section 2 or Section 2.4) if the expression level of CD57 in CD4⁺ T cells and CD8⁺ T cells is reduced compared to a reference sample.

In certain embodiments, the subject having cancer is non-responsive to an adoptive cell therapy (e.g., a cell disclosed in Section 2 or Section 2.4) if the expression level of TIM3 in T cells (e.g., CD4⁺ T cells, CD8⁺ T cells) is reduced compared to a reference sample. In certain embodiments, the subject having cancer is non-responsive to an adoptive cell therapy (e.g., a cell disclosed in Section 2 or Section 2.4) if the expression level of TIM3 in CD4⁺ T cells is reduced compared to a reference sample. In certain embodiments, the subject having cancer is non-responsive to an adoptive cell therapy (e.g., a cell disclosed in Section 2 or Section 2.4) if the expression level of TIM3 in CD8⁺ T cells is reduced compared to a reference sample. In certain embodiments, the subject having cancer is non-responsive to an adoptive cell therapy (e.g., a cell disclosed in Section 2 or Section 2.4) if the expression level of TIM3 in CD4⁺ T cells and CD8⁺ T cells is reduced compared to a reference sample.

In certain embodiments, the subject having cancer is non-responsive to an adoptive cell therapy (e.g., a cell disclosed in Section 2 or Section 2.4) if the expression level of KLRG1 in T cells (e.g., CD4⁺ T cells, CD8⁺ T cells) is increased compared to a reference sample. In certain embodiments, the subject having cancer is non-responsive to an adoptive cell therapy (e.g., a cell disclosed in Section 2 or Section 2.4) if the expression level of KLRG1 in CD4⁺ T cells is increased compared to a reference sample. In certain embodiments, the subject having cancer is non-responsive to an adoptive cell therapy (e.g., a cell disclosed in Section 2 or Section 2.4) if the expression level of KLRG1 in CD8⁺ T cells is increased compared to a reference sample. In certain embodiments, the subject having cancer is non-responsive to an adoptive cell therapy (e.g., a cell disclosed in Section 2 or Section 2.4) if the expression level of KLRG1 in CD4⁺ T cells and CD8⁺ T cells is increased compared to a reference sample.

In certain embodiments, the subject having cancer is non-responsive to an adoptive cell therapy (e.g., a cell disclosed in Section 2 or Section 2.4) if the expression level of CTLA4 in T cells (e.g., CD4⁺ T cells, CD8⁺ T cells) is increased compared to a reference sample. In certain embodiments, the subject having cancer is non-responsive to an adoptive cell therapy (e.g., a cell disclosed in Section 2 or Section 2.4) if the expression level of CTLA4 in CD4⁺ T cells is increased compared to a reference sample. In certain embodiments, the subject having cancer is non-responsive to an adoptive cell therapy (e.g., a cell disclosed in Section 2 or Section 2.4) if the expression level of CTLA4 in CD8⁺ T cells is increased compared to a reference sample. In certain embodiments, the subject having cancer is non-responsive to an adoptive cell therapy (e.g., a cell disclosed in Section 2 or Section 2.4) if the expression level of CTLA4 in CD4⁺ T cells and CD8⁺ T cells is increased compared to a reference sample.

In certain embodiments, the subject having cancer is non-responsive to an adoptive cell therapy (e.g., a cell disclosed in Section 2 or Section 2.4) if the expression level of LAG3 in T cells (e.g., CD4⁺ T cells, CD8⁺ T cells) is increased compared to a reference sample. In certain embodiments, the subject having cancer is non-responsive to an adoptive cell therapy (e.g., a cell disclosed in Section 2 or Section 2.4) if the expression level of LAG3 in CD4⁺ T cells is increased compared to a reference sample. In certain embodiments, the subject having cancer is non-responsive to an adoptive cell therapy (e.g., a cell disclosed in Section 2 or Section 2.4) if the expression level of LAG3 in CD8⁺ T cells is increased compared to a reference sample. In certain embodiments, the subject having cancer is non-responsive to an adoptive cell therapy (e.g., a cell disclosed in Section 2 or Section 2.4) if the expression level of LAG3 in CD4⁺ T cells and CD8⁺ T cells is increased compared to a reference sample.

In certain embodiments, the subject having cancer is non-responsive to an adoptive cell therapy (e.g., a cell disclosed in Section 2 or Section 2.4) if the expression level of PD1 in T cells (e.g., CD4⁺ T cells, CD8⁺ T cells) is reduced compared to a reference sample. In certain embodiments, the subject having cancer is non-responsive to an adoptive cell therapy (e.g., a cell disclosed in Section 2 or Section 2.4) if the expression level of PD1 in CD4⁺ T cells is reduced compared to a reference sample. In certain embodiments, the subject having cancer is non-responsive to an adoptive cell therapy (e.g., a cell disclosed in Section 2 or Section 2.4) if the expression level of PD1 in CD8⁺ T cells is reduced compared to a reference sample. In certain embodiments, the subject having cancer is non-responsive to an adoptive cell therapy (e.g., a cell disclosed in Section 2 or Section 2.4) if the expression level of PD1 in CD4⁺ T cells and CD8⁺ T cells is reduced compared to a reference sample.

In certain embodiments, the subject having cancer is non-responsive to an adoptive cell therapy (e.g., a cell disclosed in Section 2 or Section 2.4) if the expression level of at least one of CD57, TIM3, and PD1 in T cells (e.g., CD4⁺ T cells, CD8⁺ T cells) is reduced compared to a reference sample. In certain embodiments, the subject having cancer is non-responsive to an adoptive cell therapy (e.g., a cell disclosed in Section 2 or Section 2.4) if the expression level of CD57, TIM3, and PD1 in CD4⁺ T cells is reduced compared to a reference sample. In certain embodiments, the subject having cancer is non-responsive to an adoptive cell therapy (e.g., a cell disclosed in Section 2 or Section 2.4) if the expression level of CD57, TIM3, and PD1 in CD8⁺ T cells is reduced compared to a reference sample. In certain embodiments, the subject having cancer is non-responsive to an adoptive cell therapy (e.g., a cell disclosed in Section 2 or Section 2.4) if the expression level of CD57, TIM3, and PD1 in CD4⁺ T cells and CD8⁺ T cells is reduced compared to a reference sample.

In certain embodiments, the subject having cancer is non-responsive to an adoptive cell therapy (e.g., a cell disclosed in Section 2 or Section 2.4) if the expression level of at least one of KLRG1, CTLA4, and LAG3 in T cells (e.g., CD4⁺ T cells, CD8⁺ T cells) is increased compared to a reference sample. In certain embodiments, the subject having cancer is non-responsive to an adoptive cell therapy (e.g., a cell disclosed in Section 2 or Section 2.4) if the expression level of at least one of KLRG1, CTLA4, and LAG3 in CD4⁺ T cells is increased compared to a reference sample. In certain embodiments, the subject having cancer is non-responsive to an adoptive cell therapy (e.g., a cell disclosed in Section 2 or Section 2.4) if the expression level of at least one of KLRG1, CTLA4, and LAG3 in CD8⁺ T cells is increased compared to a reference sample. In certain embodiments, the subject having cancer is non-responsive to an adoptive cell therapy (e.g., a cell disclosed in Section 2 or Section 2.4) if the expression level of at least one of KLRG1, CTLA4, and LAG3 in CD4⁺ T cells and CD8⁺ T cells is increased compared to a reference sample.

In certain embodiments, the subject having cancer is non-responsive to an adoptive cell therapy (e.g., a cell disclosed in Section 2 or Section 2.4) if
a) the expression level of at least one of CD57, TIM3, and PD1 in T cells (e.g., CD4⁺ T cells, CD8⁺ T cells) is reduced compared to a reference sample; and/or
b) the expression level of at least one of KLRG1, CTLA4, and LAG3 in T cells (e.g., CD4⁺ T cells, CD8⁺ T cells) is increased compared to the reference sample.

In certain embodiments, the subject having cancer is non-responsive to an adoptive cell therapy (e.g., a cell disclosed in Section 2 or Section 2.4) if
a) the expression level of at least one of CD57, TIM3, and PD1 in CD4⁺ T cells is reduced compared to a reference sample; and/or
b) the expression level of at least one of KLRG1, CTLA4, and LAG3 in CD4⁺ T cells is increased compared to the reference sample.

In certain embodiments, the subject having cancer is non-responsive to an adoptive cell therapy (e.g., a cell disclosed in Section 2 or Section 2.4) if
a) the expression level of at least one of CD57, TIM3, and PD1 in CD8⁺ T cells is reduced compared to a reference sample; and/or
b) the expression level of at least one of KLRG1, CTLA4, LAG3 in CD8⁺ T cells is increased compared to the reference sample.

In certain embodiments, the subject having cancer is non-responsive to an adoptive cell therapy (e.g., a cell disclosed in Section 2 or Section 2.4) if
a) the expression level of at least one of CD57, TIM3, and PD1 in CD4⁺ T cells and CD8⁺ T cells is reduced compared to a reference sample; and/or
b) the expression level of at least one of KLRG1, CTLA4, and LAG3 in CD4⁺ T cells and CD8⁺ T cells is increased compared to the reference sample.

In certain embodiments, the subject having cancer has a reduced likelihood to respond to an adoptive cell therapy (e.g., a cell disclosed in Section 2 or Section 2.4) if the expression level of CD57 in T cells (e.g., CD4⁺ T cells, CD8⁺ T cells) is reduced compared to a reference sample. In certain embodiments, the subject having cancer has a reduced likelihood to respond to an adoptive cell therapy (e.g., a cell disclosed in Section 2 or Section 2.4) if the expression level of CD57 in CD4⁺ T cells is reduced compared to a reference sample. In certain embodiments, the subject having cancer has a reduced likelihood to respond to an adoptive cell therapy (e.g., a cell disclosed in Section 2 or Section 2.4) if the expression level of CD57 in CD8⁺ T cells is reduced compared to a reference sample. In certain embodiments, the subject having cancer has a reduced likelihood to respond to an adoptive cell therapy (e.g., a cell disclosed in Section 2 or Section 2.4) if the expression level of CD57 in CD4⁺ T cells and CD8⁺ T cells is reduced compared to a reference sample.

In certain embodiments, the subject having cancer has a reduced likelihood to respond to an adoptive cell therapy (e.g., a cell disclosed in Section 2 or Section 2.4) if the expression level of TIM3 in T cells (e.g., CD4⁺ T cells, CD8⁺ T cells) is reduced compared to a reference sample. In certain embodiments, the subject having cancer has a reduced likelihood to respond to an adoptive cell therapy (e.g., a cell disclosed in Section 2 or Section 2.4) if the expression level of TIM3 in CD4⁺ T cells is reduced compared to a reference sample. In certain embodiments, the subject having cancer has a reduced likelihood to respond to an adoptive cell therapy (e.g., a cell disclosed in Section 2 or Section 2.4) if the expression level of TIM3 in CD8⁺ T cells is reduced compared to a reference sample. In certain embodiments, the subject having cancer has a reduced likelihood to respond to an adoptive cell therapy (e.g., a cell disclosed in Section 2 or Section 2.4) if the expression level of TIM3 in CD4⁺ T cells and CD8⁺ T cells is reduced compared to a reference sample.

In certain embodiments, the subject having cancer has a reduced likelihood to respond to an adoptive cell therapy (e.g., a cell disclosed in Section 2 or Section 2.4) if the expression level of KLRG1 in T cells (e.g., CD4⁺ T cells, CD8⁺ T cells) is increased compared to a reference sample. In certain embodiments, the subject having cancer has a reduced likelihood to respond to an adoptive cell therapy (e.g., a cell disclosed in Section 2 or Section 2.4) if the expression level of KLRG1 in CD4⁺ T cells is increased compared to a reference sample. In certain embodiments, the subject having cancer has a reduced likelihood to respond to an adoptive cell therapy (e.g., a cell disclosed in Section 2 or Section 2.4) if the expression level of KLRG1 in CD8⁺ T cells is increased compared to a reference sample. In certain embodiments, the subject having cancer has a reduced likelihood to respond to an adoptive cell therapy (e.g., a cell disclosed in Section 2 or Section 2.4) if the expression level of KLRG1 in CD4⁺ T cells and CD8⁺ T cells is increased compared to a reference sample.

In certain embodiments, the subject having cancer has a reduced likelihood to respond to an adoptive cell therapy (e.g., a cell disclosed in Section 2 or Section 2.4) if the expression level of CTLA4 in T cells (e.g., CD4⁺ T cells, CD8⁺ T cells) is increased compared to a reference sample. In certain embodiments, the subject having cancer has a reduced likelihood to respond to an adoptive cell therapy (e.g., a cell disclosed in Section 2 or Section 2.4) if the expression level of CTLA4 in CD4⁺ T cells is increased compared to a reference sample. In certain embodiments, the subject having cancer has a reduced likelihood to respond to an adoptive cell therapy (e.g., a cell disclosed in Section 2 or Section 2.4) if the expression level of CTLA4 in CD8⁺ T cells is increased compared to a reference sample. In certain embodiments, the subject having cancer has a reduced likelihood to respond to an adoptive cell therapy (e.g., a cell disclosed in Section 2 or Section 2.4) if the expression level of CTLA4 in CD4⁺ T cells and CD8⁺ T cells is increased compared to a reference sample.

In certain embodiments, the subject having cancer has a reduced likelihood to respond to an adoptive cell therapy (e.g., a cell disclosed in Section 2 or Section 2.4) if the expression level of LAG3 in T cells (e.g., CD4⁺ T cells, CD8⁺ T cells) is increased compared to a reference sample. In certain embodiments, the subject having cancer has a reduced likelihood to respond to an adoptive cell therapy (e.g., a cell disclosed in Section 2 or Section 2.4) if the expression level of LAG3 in CD4⁺ T cells is increased compared to a reference sample. In certain embodiments, the subject having cancer has a reduced likelihood to respond to an adoptive cell therapy (e.g., a cell disclosed in Section 2 or Section 2.4) if the expression level of LAG3 in CD8⁺ T cells is increased compared to a reference sample. In certain embodiments, the subject having cancer has a reduced likelihood to respond to an adoptive cell therapy (e.g., a cell disclosed in Section 2 or Section 2.4) if the expression level of LAG3 in CD4⁺ T cells and CD8⁺ T cells is increased compared to a reference sample.

In certain embodiments, the subject having cancer has a reduced likelihood to respond to an adoptive cell therapy (e.g., a cell disclosed in Section 2 or Section 2.4) if the expression level of PD1 in T cells (e.g., CD4⁺ T cells, CD8⁺ T cells) is reduced compared to a reference sample. In certain embodiments, the subject having cancer has a reduced likelihood to respond to an adoptive cell therapy (e.g., a cell disclosed in Section 2 or Section 2.4) if the expression level of PD1 in CD4⁺ T cells is reduced compared to a reference sample. In certain embodiments, the subject having cancer has a reduced likelihood to respond to an adoptive cell therapy (e.g., a cell disclosed in Section 2 or Section 2.4) if the expression level of PD1 in CD8⁺ T cells is reduced compared to a reference sample. In certain embodiments, the subject having cancer has a reduced likelihood to respond to an adoptive cell therapy (e.g., a cell disclosed in Section 2 or Section 2.4) if the expression level of PD1 in CD4⁺ T cells and CD8⁺ T cells is reduced compared to a reference sample.

In certain embodiments, the subject having cancer has a reduced likelihood to respond to an adoptive cell therapy (e.g., a cell disclosed in Section 2 or Section 2.4) if the expression level of at least one of CD57, TIM3, and PD1 in T cells (e.g., CD4⁺ T cells, CD8⁺ T cells) is reduced compared to a reference sample. In certain embodiments, the subject having cancer has a reduced likelihood to respond to an adoptive cell therapy (e.g., a cell disclosed in Section 2 or Section 2.4) if the expression level of CD57, TIM3, and PD1 in CD4⁺ T cells is reduced compared to a reference sample. In certain embodiments, the subject having cancer has a reduced likelihood to respond to an adoptive cell therapy (e.g., a cell disclosed in Section 2 or Section 2.4) if the expression level of CD57, TIM3, and PD1 in CD8⁺ T cells is reduced compared to a reference sample. In certain embodiments, the subject having cancer has a reduced likelihood to respond to an adoptive cell therapy (e.g., a cell disclosed in Section 2 or Section 2.4) if the expression level of CD57, TIM3, and PD1 in CD4⁺ T cells and CD8⁺ T cells is reduced compared to a reference sample.

In certain embodiments, the subject having cancer has a reduced likelihood to respond to an adoptive cell therapy (e.g., a cell disclosed in Section 2 or Section 2.4) if the expression level of at least one of KLRG1, CTLA4, and LAG3 in T cells (e.g., CD4⁺ T cells, CD8⁺ T cells) is increased compared to a reference sample. In certain embodiments, the subject having cancer has a reduced likelihood to respond to an adoptive cell therapy (e.g., a cell disclosed in Section 2 or Section 2.4) if the expression level of at least one of KLRG1, CTLA4, and LAG3 in CD4⁺ T cells is increased compared to a reference sample. In certain embodiments, the subject having cancer has a reduced likelihood to respond to an adoptive cell therapy (e.g., a cell disclosed in Section 2 or Section 2.4) if the expression level of at least one of KLRG1, CTLA4, and LAG3 in CD8⁺ T cells is increased compared to a reference sample. In certain embodiments, the subject having cancer has a reduced likelihood to respond to an adoptive cell therapy (e.g., a cell disclosed in Section 2 or Section 2.4) if the expression level of at least one of KLRG1, CTLA4, and LAG3 in CD4⁺ T cells and CD8⁺ T cells is increased compared to a reference sample.

In certain embodiments, the subject having cancer has a reduced likelihood to respond to an adoptive cell therapy (e.g., a cell disclosed in Section 2 or Section 2.4) if
a) the expression level of at least one of CD57, TIM3, and PD1 in T cells (e.g., CD4⁺ T cells, CD8⁺ T cells) is reduced compared to a reference sample; and/or
b) the expression level of at least one of KLRG1, CTLA4, and LAG3 in T cells (e.g., CD4⁺ T cells, CD8⁺ T cells) is increased compared to the reference sample.

In certain embodiments, the subject having cancer has a reduced likelihood to respond to an adoptive cell therapy (e.g., a cell disclosed in Section 2 or Section 2.4) if
a) the expression level of at least one of CD57, TIM3, and PD1 in CD4⁺ T cells is reduced compared to a reference sample; and/or
b) the expression level of at least one of KLRG1, CTLA4, and LAG3 in CD4⁺ T cells is increased compared to the reference sample.

In certain embodiments, the subject having cancer has a reduced likelihood to respond to an adoptive cell therapy (e.g., a cell disclosed in Section 2 or Section 2.4) if
a) the expression level of at least one of CD57, TIM3, and PD1 in CD8⁺ T cells is reduced compared to a reference sample; and/or
b) the expression level of at least one of KLRG1, CTLA4, and LAG3 in CD8⁺ T cells is increased compared to the reference sample.

In certain embodiments, the subject having cancer has a reduced likelihood to respond to an adoptive cell therapy (e.g., a cell disclosed in Section 2 or Section 2.4) if
a) the expression level of at least one of CD57, TIM3, and PD1 in CD4⁺ T cells and CD8⁺ T cells is reduced compared to a reference sample; and/or
b) the expression level of at least one of KLRG1, CTLA4, and LAG3 in CD4⁺ T cells and CD8⁺ T cells is increased compared to the reference sample.

In certain embodiments, the expression level of at least one biomarker (e.g., CD57, TIM3, KLRG1, CTLA4, LAG3, PD1) is increased compared to the reference sample when the expression level is at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold higher in comparison to the reference sample.

In certain embodiments, the expression level of at least one biomarker (e.g., CD57, TIM3, KLRG1, CTLA4, LAG3, PD1) is reduced compared to the reference sample when the expression level is at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold lower in comparison to the reference sample.

### 6. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a neoplasm in a subject. The presently disclosed cells and compositions comprising thereof can be used for prolonging the survival of a subject suffering from a neoplasm. In certain embodiments, each of the above-noted methods comprises administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the presently disclosed subject matter includes methods of inducing and/or increasing an immune response in a subject having cancer that is responsive to an adoptive cell therapy. In certain embodiments, the presently disclosed subject matter includes methods of reducing tumor burden in a subject having cancer that is responsive to an adoptive cell therapy. In certain embodiments, the presently disclosed subject matter includes methods of treating and/or preventing a neoplasm in a subject having cancer that is responsive to an adoptive cell therapy. In certain embodiments, the presently disclosed subject matter includes methods of prolonging the survival of a subject having cancer that is responsive to an adoptive cell therapy. In certain embodiments, the subject having cancer that is responsive to the adoptive cell therapy is identified by measuring the expression level of a biomarker (e.g., CD57, TIM3, KLRG1, CTLA4, LAG3, PD1, or a combination thereof) in immunoresponsive cells (e.g., CD4⁺ T cells, CD8⁺ T cells) as described in Section 5 above. In certain embodiments, each of the above-noted methods comprises administering the presently disclosed cells (e.g., a cell disclosed in Section 2 or Section 2.4) or a composition (e.g., a composition disclosed in Section 4) comprising thereof to the subject responsive to the adoptive cell therapy in order to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the presently disclosed subject matter includes methods of inducing and/or increasing an immune response in a subject having cancer that has an increased likelihood to respond to an adoptive cell therapy. In certain embodiments, the presently disclosed subject matter includes methods of reducing tumor burden in a subject having cancer that has an increased likelihood to respond to an adoptive cell therapy. In certain embodiments, the presently disclosed subject matter includes methods of treating and/or preventing a neoplasm in a subject having cancer that is responsive to an adoptive cell therapy. In certain embodiments, the presently disclosed subject matter includes methods of prolonging the survival in a subject having cancer that has an increased likelihood to respond to an adoptive cell therapy. In certain embodiments, the subject having cancer that has an increased likelihood to respond to the adoptive cell therapy is identified by measuring the expression level of a biomarker (e.g., CD57, TIM3, KLRG1, CTLA4, LAG3, PD1, or a combination thereof) in immunoresponsive cells (e.g., CD4⁺ T cells, CD8⁺ T cells) as described in Section 5 above. In certain embodiments, each of the above-noted methods comprises administering the presently disclosed cells (e.g., a cell disclosed in Section 2 or Section 2.4) or a composition (e.g., a composition disclosed in Section 4) comprising thereof to the subject having an increased likelihood to respond to the adoptive cell therapy in order to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the presently disclosed subject matter includes methods of inducing and/or increasing an immune response in a subject having cancer that is non-responsive to an adoptive cell therapy. In certain embodiments, the presently disclosed subject matter includes methods of reducing tumor burden in a subject having cancer that is non-responsive to an adoptive cell therapy. In certain embodiments, the presently disclosed subject matter includes methods of treating and/or preventing a neoplasm in a subject having cancer that is non-responsive to an adoptive cell therapy. In certain embodiments, the presently disclosed subject matter includes methods of prolonging the survival in a subject having cancer that is non-responsive to an adoptive cell therapy. In certain embodiments, the subject having cancer that is non-responsive to the adoptive cell therapy is identified by measuring the expression level of a biomarker (e.g., CD57, TIM3, KLRG1, CTLA4, LAG3, PD1, or a combination thereof) in immunoresponsive cells (e.g., CD4⁺ T cells, CD8⁺ T cells) as described in Section 5 above. In certain embodiments, each of the above-noted methods comprises administering the composition (e.g., a composition disclosed in Section 4) to the subject non-responsive to the adoptive cell therapy in order to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the presently disclosed subject matter includes methods of inducing and/or increasing an immune response in a subject having cancer that has a reduced likelihood to respond to an adoptive cell therapy. In certain embodiments, the presently disclosed subject matter includes methods of reducing tumor burden in a subject having cancer that has a reduced likelihood to respond to an adoptive cell therapy. In certain embodiments, the presently disclosed subject matter includes methods of treating and/or preventing a neoplasm in a subject having cancer that has a reduced likelihood to respond to an adoptive cell therapy. In certain embodiments, the presently disclosed subject matter includes methods of prolonging the survival in a subject having cancer that has a reduced likelihood to respond to an adoptive cell therapy. In certain embodiments, the subject having cancer that has a reduced likelihood to the adoptive cell therapy is identified by measuring the expression level of a biomarker (e.g., CD57, TIM3, KLRG1, CTLA4, LAG3, PD1, or a combination thereof) in immunoresponsive cells (e.g., CD4⁺ T cells, CD8⁺ T cells) as described in Section 5 above. In certain embodiments, each of the above-noted methods comprises administering the composition (e.g., a composition disclosed in Section 4) to the subject having a reduced likelihood to respond to the adoptive cell therapy in order to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the tumor and/or neoplasm is associated with CD19. In certain embodiments, the tumor and/or neoplasm expresses CD19. In certain embodiments, the tumor and/or neoplasm overexpresses CD19. In certain embodiments, the tumor and/or neoplasm that can be treated by the presently disclosed cells and compositions is a blood cancer. Non-limiting examples of blood cancer include multiple myeloma, leukemia, and lymphomas. Non-limiting examples of leukemia include acute myeloid leukemia (AML), chronic myeloid leukemia (CMIL), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. The lymphoma can be Hodgkin's lymphoma or non-Hodgkin's lymphoma. In certain embodiments, the lymphoma is B cell lymphoma (BCL).

In certain embodiments, the tumor and/or neoplasm is a B cell malignancy. Non-limiting examples of B cell malignancy include B cell lymphoma (BCL), B cell acute lymphocytic leukemia (ALL), B cell chronic lymphocytic leukemia (CLL), multiple myeloma (MM), CLL with Richter's transformation, and CNS lymphoma. B cell lymphoma includes B cell non-Hodgkin lymphoma (NHL) and B cell Hodgkin's lymphoma. In certain embodiments, the tumor and/or neoplasm is B cell lymphoma. In certain embodiments, the B cell lymphoma is relapsed or refractory (R/R) B cell lymphoma.

In certain embodiments, the subject is a human subject. The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

As a consequence of surface expression of a presently disclosed CD19-targeted chimeric receptor (e.g., a presently disclosed CD19-targeted CAR), adoptively transferred cells are endowed with augmented and selective cytolytic activity at the tumor site. Furthermore, subsequent to their localization to tumor and their proliferation, the cells turn the tumor site into a highly conductive environment for a wide range of cells involved in the physiological anti-tumor response.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the CD19-targeted chimeric receptor. The suicide gene can be included within the vector comprising nucleic acids encoding a presently disclosed CD19-targeted chimeric receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the CD19-targeted chimeric receptor. The incorporation of a suicide gene into a presently disclosed cell gives an added level of safety with the ability to eliminate the majority of receptor-expressing cells within a very short time period. A presently disclosed cell incorporated with a suicide gene can be pre-emptively eliminated at a given timepoint post the cell infusion, or eradicated at the earliest signs of toxicity.

### 7. Kits

In certain embodiments, the presently disclosed subject matter includes kits and materials for performing any of the methods disclosed herein. In certain embodiments, the kit comprises one or more specific reagents (e.g., antibody) for the detection of one of the biomarkers disclosed herein (e.g., disclosed in Section 5). In certain embodiments, the kit can include one or more containers, each with one or more of various materials or reagents utilized in the methods, including, for example, buffers, live/dead reagents, labels, isotype controls, test tubes, and the like.

Additionally or alternatively, the kit comprises an effective amount of a cell or a composition disclosed herein in unit dosage form. In certain embodiments, the kit comprises a sterile container that contains the therapeutic composition; such containers can be boxes, ampules, bottles, vials, tubes, bags, pouches, blister packs, or other suitable container forms known in the art. Such containers can be made of plastic, glass, laminated paper, metal foil, or other materials suitable for holding medicaments.

In certain embodiments, the kit comprises instructions for administering the cell or composition to a subject suffering from cancer. In certain embodiments, the instructions comprise at least one of the following: description of the therapeutic agent; dosage schedule and administration information; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container.

### 8. Exemplary Embodiments

In certain embodiments, the presently disclosed subject matter is directed to a method comprising: (a) measuring an expression level of at least one of CD57, TIM3, KLRG1, CTLA4, LAG3 and PD1 in an immunoresponsive cell of a subject; (b) comparing the expression level to a reference sample; and (c) identifying the subject as responsive or as having an increased likelihood to respond to an adoptive cell therapy.

In certain embodiments of the methods disclosed herein, the subject is responsive or has an increased likelihood to respond to the adoptive cell therapy if (a) the expression level of CD57 is increased compared to the reference sample; (b) the expression level of TIM3 is increased compared to the reference sample; (c) the expression level of KLRG1 is reduced compared to the reference sample; (d) the expression level of CTLA4 is reduced compared to the reference sample; (e) the expression level of LAG3 is reduced compared to the reference sample; and/or (f) the expression level of PD1 is increased compared to the reference sample.

In certain embodiments, the presently disclosed subject matter is directed to a method comprising: (a) measuring the expression level of at least one of CD57, TIM3, KLRG1, CTLA4, LAG3 and PD1 in an immunoresponsive cell of a subject; (b) comparing the expression level to a reference sample; and (c) identifying the subject as non-responsive or as having a reduced likelihood to respond to an adoptive cell therapy.

In certain embodiments of the methods disclosed herein, the subject is non-responsive or has a reduced likelihood to respond the adoptive cell therapy if (a) the expression level of CD57 is reduced compared to the reference sample; (b) the expression level of TIM3 is reduced compared to the reference sample; (c) the expression level of KLRG1 is increased compared to the reference sample; (d) the expression level of CTLA4 is increased compared to the reference sample; (e) the expression level of LAG3 is increased compared to the reference sample; and/or (f) the expression level of PD1 is reduced compared to the reference sample.

In certain embodiments of the methods disclosed herein, the adoptive cell therapy comprises a cell comprising a CD19-targeted chimeric receptor.

In certain embodiments of the methods disclosed herein, the method further comprises administering to the subject a cell comprising a CD19-targeted chimeric receptor or a composition comprising thereof, wherein the CD19-targeted chimeric receptor comprises an extracellular antigen-binding domain comprising a heavy chain variable region (V_{H}) comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12, and a light chain variable region (V_{L}) comprising a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15.

In certain embodiments of the methods disclosed herein, the extracellular antigen-binding domain comprises a scFv.

In certain embodiments of the methods disclosed herein, a) the heavy chain variable region comprises an amino acid sequence that is at least about 80% identical to the amino acid sequence set forth in SEQ ID NO: 16; and b) the heavy chain variable region comprises an amino acid sequence that is at least about 80% identical to the amino acid sequence set forth in SEQ ID NO: 17.

In certain embodiments of the methods disclosed herein, a) the heavy chain variable region comprises the amino acid sequence set forth in SEQ ID NO: 16; and b) the heavy chain variable region comprises the amino acid sequence set forth in SEQ ID NO: 17.

In certain embodiments of the methods disclosed herein, the chimeric receptor comprises a transmembrane domain comprising a CD28 polypeptide.

In certain embodiments of the methods disclosed herein, the chimeric receptor comprises an intracellular signaling domain comprising a CD3ζ polypeptide comprising a native ITAM1, an ITAM2 variant consisting of two loss-of-function mutations, and an ITAM3 consisting of two loss-of-function mutations.

In certain embodiments of the methods disclosed herein, the native ITAM1 consists of the amino acid sequence set forth in SEQ ID NO: 28, the ITAM2 variant consists of the amino acid sequence set forth in SEQ ID NO: 34, and the ITAM3 variant consists of the amino acid sequence set forth in SEQ ID NO: 38.

In certain embodiments of the methods disclosed herein, the chimeric receptor comprises an intracellular signaling domain comprising a CD3ζ polypeptide comprising the amino acid sequence set forth in SEQ ID NO: 40.

In certain embodiments of the methods disclosed herein, the intracellular signaling domain further comprises a CD28 polypeptide.

In certain embodiments of the methods disclosed herein, the chimeric receptor comprises the amino acid sequence set forth in SEQ ID NO: 45.

In certain embodiments of the methods disclosed herein, the chimeric receptor is expressed from a vector.

In certain embodiments of the methods disclosed herein, the vector is a retroviral vector.

In certain embodiments of the methods disclosed herein, the chimeric receptor is constitutively expressed on the surface of the cell.

In certain embodiments of the methods disclosed herein, the cell comprising the CD19-targeted chimeric receptor is an immunoresponsive cell.

In certain embodiments of the methods disclosed herein, the cell comprising the CD19-targeted chimeric receptor is a cell of the lymphoid lineage or a cell of the myeloid lineage.

In certain embodiments of the methods disclosed herein, the cell comprising the CD19-targeted chimeric receptor is selected from the group consisting of a T cell, a Natural Killer (NK) cell, a stem cell from which lymphoid cells may be differentiated, and a stem cell from which myeloid cells may be differentiated.

In certain embodiments of the methods disclosed herein, the cell comprising the CD19-targeted chimeric receptor is a T cell.

In certain embodiments of the methods disclosed herein, the T cell is selected from the group consisting of helper T cells, cytotoxic T cells, memory T cells, regulatory T cells, tumor-infiltrating lymphocyte (TIL), Natural Killer T cells, mucosal associated invariant T cells, and γδ T cells.

In certain embodiments of the methods disclosed herein, the cell comprising the CD19-targeted chimeric receptor is a NK cell.

In certain embodiments of the methods disclosed herein, the cell comprising the CD19-targeted chimeric receptor is derived from a stem cell.

In certain embodiments of the methods disclosed herein, the stem cell is a pluripotent stem cell.

In certain embodiments of the methods disclosed herein, the pluripotent stem cell is an embryoid stem cell or an induced pluripotent stem cell.

In certain embodiments of the methods disclosed herein, the cell comprising the CD19-targeted chimeric receptor is selected from a a CD57⁺ T cell, a TIM3⁺ T cell, a KLRG1⁻ T cell, a CTLA4⁻ T cell, a LAG3⁻ T cell, a PD1⁺ T cell, a CD57⁺ TIM3⁺ T cell, a CD57⁺ KLRG1⁻ T cell, a CD57⁺ CTLA4⁻ T cell, a CD57⁺ LAG3⁻ T cell, a CD57⁺ PD1⁺ T cell, a TIM3⁺ KLRG1⁻ T cell, a TIM3⁺ CTLA4⁻ T cell, a TIM3⁺ LAG3⁻ T cell, a TIM3⁺ PD1⁺ T cell, a KLRG1⁻ CTLA4⁻ T cell, a KLRG1⁻ LAG3⁻ T cell, a KLRG1⁻ PD1⁺ T cell, a CTLA4⁻ LAG3⁻ T cell, a CTLA4⁻ PD1⁺ T cell, a LAG3⁻ PD1⁺ T cell, a CD57⁺ TIM3⁺ KLRG1⁻ T cell, a CD57⁺ TIM3⁺ CTLA4⁻ T cell, a CD57⁺ TIM3⁺ LAG3⁻ T cell, a CD57⁺ TIM3⁺ PD1⁺ T cell, a CD57⁺ KLRG1⁻ CTLA4⁻ T cell, a CD57⁺ KLRG1⁻ LAG3⁻ T cell, a CD57⁺ KLRG1⁻ PD1⁺ T cell, a CD57⁺ CTLA4⁻ LAG3⁻ T cell, a CD57⁺ CTLA4⁻ PD1⁺ T cell, a CD57⁺ LAG3⁻ PD1⁺ T cell, a TIM3⁺ KLRG1⁻ CTLA4⁻ T cell, a TIM3⁺ KLRG1⁻ LAG3⁻ T cell, a TIM3⁺ CTLA4⁻ LAG3⁻ T cell, a TIM3⁺ KLRG1⁻ PD1⁺ T cell, a TIM3⁺ CTLA4⁻ LAG3⁻ T cell, a TIM3⁺ CTLA4⁻ PD1⁺ T cell, a TIM3⁺ LAG3⁻ PD1⁺ T cell, a KLRG1⁻ CTLA4⁻ LAG3⁻ T cell, a KLRG1⁻ CTLA4⁻ PD1⁺ T cell, a KLRG1⁻ LAG3⁻ PD1⁺ T cell, a CTLA4⁻ LAG3⁻ PD1⁺ T cell, a CD57⁺ TIM3⁺ KLRG1⁻ CTLA4⁻ T cell, a CD57⁺ TIM3⁺ KLRG1⁻ LAG3⁻ T cell, a CD57⁺ TIM3⁺ KLRG1⁻ PD1⁺ T cell, a CD57⁺ TIM3⁺ CTLA4⁻ LAG3⁻ T cell, a CD57⁺ TIM3⁺ CTLA4⁻ PD1⁺ T cell, a CD57⁺ TIM3⁺ LAG3⁻ PD1⁺ T cell, a CD57⁺ KLRG1⁻ CTLA4⁻ LAG3⁻ T cell, a CD57⁺ KLRG1⁻ CTLA4⁻ PD1⁺ T cell, a CD57⁺ KLRG1⁻ LAG3⁻ PD1⁺ T cell, a CD57⁺ CTLA4⁻ LAG3⁻ PD1⁺ T cell, a TIM3⁺ KLRG1⁻ CTLA4⁻ LAG3⁻ T cell, a TIM3⁺ KLRG1⁻ CTLA4⁻ LAG3⁻ T cell, a TIM3⁺ KLRG1⁻ CTLA4⁻ PD1⁺ T cell, a TIM3⁺ KLRG1⁻ LAG3⁻ PD1⁺ T cell, a TIM3⁺ CTLA4⁻ LAG3⁻ PD1⁺ T cell, a KLRG1⁻ CTLA4⁻ LAG3⁻ PD1⁺ T cell, a CD57⁺ TIM3⁺ KLRG1⁻ CTLA4⁻ LAG3⁻ T cell, a CD57⁺ TIM3⁺ KLRG1⁻ CTLA4⁻ PD1⁺ T cell, a CD57⁺ TIM3⁺ KLRG1⁻ LAG3⁻ PD1⁺ T cell, a CD57⁺ TIM3⁺ CTLA4⁻ LAG3⁻ PD1⁺ T cell, a CD57⁺ KLRG1⁻ CTLA4⁻ LAG3⁻ PD1⁺ T cell, a TIM3⁺ KLRG1⁻ CTLA4⁻ LAG3⁻ PD1⁺ T cell, a CD57⁺ TIM3⁺ KLRG1⁻ CTLA4⁻ LAG3⁻ PD1⁺ T cell, or a combination thereof.

In certain embodiments of the methods disclosed herein, the composition comprises at least about 5% of a CD57⁺ T cell comprising the CD19-targeted chimeric receptor, at least about 5% of a TIM3⁺ T cell comprising the CD19-targeted chimeric receptor, at least about 5% of a KLRG1⁻ T cell comprising the CD19-targeted chimeric receptor, at least about 5% of a CTLA4⁻ T cell comprising the CD19-targeted chimeric receptor, at least about 5% of a LAG3⁻ T cell comprising the CD19-targeted chimeric receptor, at least about 5% of a PD1⁺ T cell comprising the CD19-targeted chimeric receptor, at least about 5% of a CD57⁺ TIM3⁺ T cell comprising the CD19-targeted chimeric receptor, at least about 5% of a CD57⁺ KLRG1⁻ T cell comprising the CD19-targeted chimeric receptor, at least about 5% of a CD57⁺ CTLA4⁻ T cell comprising the CD19-targeted chimeric receptor, at least about 5% of a CD57⁺ LAG3⁻ T cell comprising the CD19-targeted chimeric receptor, at least about 5% of a CD57⁺ PD1⁺ T cell comprising the CD19-targeted chimeric receptor, at least about 5% of a TIM3⁺ KLRG1⁻ T cell comprising the CD19-targeted chimeric receptor, at least about 5% of a TIM3⁺ CTLA4⁻ T cell comprising the CD19-targeted chimeric receptor, at least about 5% of a TIM3⁺ LAG3⁻ T cell comprising the CD19-targeted chimeric receptor, at least about 5% of a TIM3⁺ PD1⁺ T cell comprising the CD19-targeted chimeric receptor, at least about 5% of a KLRG1⁻ CTLA4⁻ T cell comprising the CD19-targeted chimeric receptor, at least about 5% of a KLRG1⁻ LAG3⁻ T cell comprising the CD19-targeted chimeric receptor, at least about 5% of a KLRG1⁻ PD1⁺ T cell comprising the CD19-targeted chimeric receptor, at least about 5% of a CTLA4⁻ LAG3⁻ T cell comprising the CD19-targeted chimeric receptor, at least about 5% of a CTLA4⁻ PD1⁺ T cell comprising the CD19-targeted chimeric receptor, at least about 5% of a LAG3⁻ PD1⁺ T cell comprising the CD19-targeted chimeric receptor, at least about 5% of a CD57⁺ TIM3⁺ KLRG1⁻ T cell comprising the CD19-targeted chimeric receptor, at least about 5% of a CD57⁺ TIM3⁺ CTLA4⁻ T cell comprising the CD19-targeted chimeric receptor, at least about 5% of a CD57⁺ TIM3⁺ LAG3⁻ T cell comprising the CD19-targeted chimeric receptor, at least about 5% of a CD57⁺ TIM3⁺ PD1⁺ T cell comprising the CD19-targeted chimeric receptor, at least about 5% of a CD57⁺ KLRG1⁻ CTLA4⁻ T cell comprising the CD19-targeted chimeric receptor, at least about 5% of a CD57⁺ KLRG1⁻ LAG3⁻ T cell comprising the CD19-targeted chimeric receptor, at least about 5% of a CD57⁺ KLRG1⁻ PD1⁺ T cell comprising the CD19-targeted chimeric receptor, at least about 5% of a CD57⁺ CTLA4⁻ LAG3⁻ T cell comprising the CD19-targeted chimeric receptor, at least about 5% of a CD57⁺ CTLA4⁻ PD1⁺ T cell comprising the CD19-targeted chimeric receptor, at least about 5% of a CD57⁺ LAG3⁻ PD1⁺ T cell comprising the CD19-targeted chimeric receptor, at least about 5% of a TIM3⁺ KLRG1⁻ CTLA4⁻ T cell comprising the CD19-targeted chimeric receptor, at least about 5% of a TIM3⁺ KLRG1⁻ LAG3⁻ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ KLRG1⁻ PD1⁺ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ CTLA4⁻ PD1⁺ T cell comprising the CD19-targeted chimeric receptor, at least about 5% of a TIM3⁺ LAG3⁻ PD1⁺ T cell comprising the CD19-targeted chimeric receptor, at least about 5% of a KLRG1⁻ CTLA4⁻ LAG3⁻ T cell comprising the CD19-targeted chimeric receptor, at least about 5% of a KLRG1⁻ CTLA4⁻ PD1⁺ T cell comprising the CD19-targeted chimeric receptor, at least about 5% of a KLRG1⁻ LAG3⁻ PD1⁺ T cell comprising the CD19-targeted chimeric receptor, at least about 5% of a CTLA4⁻ LAG3⁻ PD1⁺ T cell comprising the CD19-targeted chimeric receptor, at least about 5% of a CD57⁺ TIM3⁺ KLRG1⁻ CTLA4⁻ T cell comprising the CD19-targeted chimeric receptor, at least about 5% of a CD57⁺ TIM3⁺ KLRG1⁻ LAG3⁻ T cell comprising the CD19-targeted chimeric receptor, at least about 5% of a CD57⁺ TIM3⁺ KLRG1⁻ PD1⁺ T cell comprising the CD19-targeted chimeric receptor, at least about 5% of a CD57⁺ TIM3⁺ CTLA4⁻ LAG3⁻ T cell comprising the CD19-targeted chimeric receptor, at least about 5% of a CD57⁺ TIM3⁺ CTLA4⁻ PD1⁺ T cell comprising the CD19-targeted chimeric receptor, at least about 5% of a CD57⁺ TIM3⁺ LAG3⁻ PD1⁺ T cell comprising the CD19-targeted chimeric receptor, at least about 5% of a CD57⁺ KLRG1⁻ CTLA4⁻ LAG3⁻ T cell comprising the CD19-targeted chimeric receptor, at least about 5% of a CD57⁺ KLRG1⁻ CTLA4⁻ PD1⁺ T cell comprising the CD19-targeted chimeric receptor, at least about 5% of a CD57⁺ KLRG1⁻ LAG3⁻ PD1⁺ T cell comprising the CD19-targeted chimeric receptor, at least about 5% of a CD57⁺ CTLA4⁻ LAG3⁻ PD1⁺ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ PD1⁺ T cell comprising the CD19-targeted chimeric receptor, at least about 5% of a TIM3⁺ KLRG1⁻ LAG3⁻ PD1⁺ T cell comprising the CD19-targeted chimeric receptor, at least about 5% of a TIM3⁺ CTLA4⁻ LAG3⁻ PD1⁺ T cell comprising the CD19-targeted chimeric receptor, at least about 5% of a KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ T cell comprising the CD19-targeted chimeric receptor, at least about 5% of a CD57⁺ TIM3⁺ KLRG1⁻ CTLA4⁻ PD1⁺ T cell comprising the CD19-targeted chimeric receptor, at least about 5% of a CD57⁺ TIM3⁺ KLRG1⁻ LAG3⁻ PD1⁺ T cell comprising the CD19-targeted chimeric receptor, at least about 5% of a CD57⁺ TIM3⁺ CTLA4⁻ LAG3⁻ PD1⁺ T cell comprising the CD19-targeted chimeric receptor, at least about 5% of a CD57⁺ KLRG1⁻ CTLA4⁻ LAG3⁻ PD1⁺ T cell comprising the CD19-targeted chimeric receptor, at least about 5% of a TIM3⁺ KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ PD1⁺ T cell comprising the CD19-targeted chimeric receptor, or a combination thereof.

In certain embodiments of the methods disclosed herein, the subject has a tumor and/or a neoplasm associated with CD19.

In certain embodiments of the methods disclosed herein, the tumor and/or neoplasm is a blood cancer.

In certain embodiments of the methods disclosed herein, the blood cancer is selected from the group consisting of multiple myeloma, leukemia, and lymphomas.

In certain embodiments of the methods disclosed herein, the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia.

In certain embodiments of the methods disclosed herein, the lymphoma is Hodgkin's lymphoma or non-Hodgkin's lymphoma.

In certain embodiments of the methods disclosed herein, the tumor and/or neoplasm is a B cell malignancy.

In certain embodiments of the methods disclosed herein, the B cell malignancy is selected from the group consisting of B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments of the methods disclosed herein, the tumor and/or neoplasm is B cell lymphoma.

In certain embodiments of the methods disclosed herein, the B cell lymphoma is relapsed or refractory (R/R) B cell lymphoma.

In certain embodiments of the methods disclosed herein, the expression level of at least one of CD57, TIM3, KLRG1, CTLA4, LAG3 and PD1 is measured on the surface of the immunoresponsive cell.

In certain embodiments of the methods disclosed herein, the expression level of at least one of CD57, TIM3, KLRG1, CTLA4, LAG3 and PD1 is measured by cytometry or by flow-cytometry.

In certain embodiments of the methods disclosed herein, the immunoresponsive cell is a T cell.

In certain embodiments of the methods disclosed herein, the T cell is selected from the group consisting of a helper T cell, a cytotoxic T cell, a memory T cell, a central memory T cell, a stem-like memory T cell, an effector memory T cell, a regulatory T cell, a tumor-infiltrating lymphocyte (TIL), a Natural killer T cells, a γδ T cell, a CD4+ T cells, and a CD8+ T cell.

In certain embodiments of the methods disclosed herein, the T cell is a CD4+ T cell or a CD8+ T cell.

In certain embodiments of the methods disclosed herein, the immunoresponsive cell is obtained from a sample of the subject.

In certain embodiments of the methods disclosed herein, the sample is selected from bone marrow, thymus, tumor biopsy, tumor fragments, enzymatically digested tumor tissue, dissociated/suspended cells, a lymph node sample, or a bodily fluid sample

In certain embodiments of the methods disclosed herein, the bodily fluid sample is a blood sample, an ascites sample, or a lymph sample.

In certain embodiments of the methods disclosed herein, the subject is a human subject.

In certain embodiments, the presently disclosed subject matter is directed to a composition comprising a cell comprising a CD19-targeted chimeric receptor, wherein the CD19-targeted chimeric receptor comprises an extracellular antigen-binding domain, a transmembrane domain, and an intracellular domain, wherein the extracellular antigen-binding domain comprises a heavy chain variable region (VH) comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12, and a light chain variable region (VL) comprising a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15.

In certain embodiments of the compositions disclosed herein, the extracellular antigen-binding domain comprises a scFv.

In certain embodiments of the compositions disclosed herein, a) the heavy chain variable region comprises an amino acid sequence that is at least about 80% identical to the amino acid sequence set forth in SEQ ID NO: 16; and b) the heavy chain variable region comprises an amino acid sequence that is at least about 80% identical to the amino acid sequence set forth in SEQ ID NO: 17.

In certain embodiments of the compositions disclosed herein, a) the heavy chain variable region comprises the amino acid sequence set forth in SEQ ID NO: 16; and b) the heavy chain variable region comprises the amino acid sequence set forth in SEQ ID NO: 17.

In certain embodiments of the compositions disclosed herein, the transmembrane domain comprises a CD28 polypeptide.

In certain embodiments of the compositions disclosed herein, the intracellular signaling domain comprises a CD3ζ polypeptide comprising a native ITAM1, an ITAM2 variant consisting of two loss-of-function mutations, and an ITAM3 consisting of two loss-of-function mutations.

In certain embodiments of the compositions disclosed herein, the native ITAM1 consists of the amino acid sequence set forth in SEQ ID NO: 28, the ITAM2 variant consists of the amino acid sequence set forth in SEQ ID NO: 34, and the ITAM3 variant consists of the amino acid sequence set forth in SEQ ID NO: 38.

In certain embodiments of the compositions disclosed herein, the intracellular signaling domain comprises a CD3ζ polypeptide comprising the amino acid sequence set forth in SEQ ID NO: 40.

In certain embodiments of the compositions disclosed herein, the intracellular signaling domain further comprises a CD28 polypeptide.

In certain embodiments of the compositions disclosed herein, the chimeric receptor comprises the amino acid sequence set forth in SEQ ID NO: 45.

In certain embodiments of the compositions disclosed herein, the chimeric receptor is expressed from a vector.

In certain embodiments of the compositions disclosed herein, the vector is a retroviral vector.

In certain embodiments of the compositions disclosed herein, the chimeric receptor is constitutively expressed on the surface of the cell.

In certain embodiments of the compositions disclosed herein, the cell is an immunoresponsive cell.

In certain embodiments of the compositions disclosed herein, the cell is a cell of the lymphoid lineage or a cell of the myeloid lineage.

In certain embodiments of the compositions disclosed herein, the cell is selected from the group consisting of a T cell, a Natural Killer (NK) cell, a stem cell from which lymphoid cells may be differentiated, and a stem cell from which myeloid cells may be differentiated.

In certain embodiments of the compositions disclosed herein, the cell is a T cell.

In certain embodiments of the compositions disclosed herein, the T cell is selected from the group consisting of helper T cells, cytotoxic T cells, memory T cells, regulatory T cells, tumor-infiltrating lymphocyte (TIL), Natural Killer T cells, mucosal associated invariant T cells, and TO T cells.

In certain embodiments of the compositions disclosed herein, the cell is an NK cell.

In certain embodiments of the compositions disclosed herein, the cell is derived from a stem cell.

In certain embodiments of the compositions disclosed herein, the stem cell is a pluripotent stem cell.

In certain embodiments of the compositions disclosed herein, the pluripotent stem cell is an embryoid stem cell or an induced pluripotent stem cell.

In certain embodiments of the compositions disclosed herein, the cell is selected from a CD57⁺ T cell, a TIM3⁺ T cell, a KLRG1⁻ T cell, a CTLA4⁻ T cell, a LAG3⁻ T cell, a PD1⁺ T cell, a CD57⁺ TIM3⁺ T cell, a CD57⁺ KLRG1⁻ T cell, a CD57⁺ CTLA4⁻ T cell, a CD57⁺ LAG3⁻ T cell, a CD57⁺ PD1⁺ T cell, a TIM3⁺ KLRG1⁻ T cell, a TIM3⁺ CTLA4⁻ T cell, a TIM3⁺ LAG3⁻ T cell, a TIM3⁺ PD1⁺ T cell, a KLRG1⁻ CTLA4⁻ T cell, a KLRG1⁻ LAG3⁻ T cell, a KLRG1⁻ PD1⁺ T cell, a CTLA4⁻ LAG3⁻ T cell, a CTLA4⁻ PD1⁺ T cell, a LAG3⁻ PD1⁺ T cell, a CD57⁺ TIM3⁺ KLRG1⁻ T cell, a CD57⁺ TIM3⁺ CTLA4⁻ T cell, a CD57⁺ TIM3⁺ LAG3⁻ T cell, a CD57⁺ TIM3⁺ PD1⁺ T cell, a CD57⁺ KLRG1⁻ CTLA4⁻ T cell, a CD57⁺ KLRG1⁻ LAG3⁻ T cell, a CD57⁺ KLRG1⁻ PD1⁺ T cell, a CD57⁺ CTLA4⁻ LAG3⁻ T cell, a CD57⁺ CTLA4⁻ PD1⁺ T cell, a CD57⁺ LAG3⁻ PD1⁺ T cell, a TIM3⁺ KLRG1⁻ CTLA4⁻ T cell, a TIM3⁺ KLRG1⁻ LAG3⁻ T cell, a TIM3⁺ CTLA4⁻ LAG3⁻ T cell, a TIM3⁺ KLRG1⁻ PD1⁺ T cell, a TIM3⁺ CTLA4⁻ LAG3⁻ T cell, a TIM3⁺ CTLA4⁻ PD1⁺ T cell, a TIM3⁺ LAG3⁻ PD1⁺ T cell, a KLRG1⁻ CTLA4⁻ LAG3⁻ T cell, a KLRG1⁻ CTLA4⁻ PD1⁺ T cell, a KLRG1⁻ LAG3⁻ PD1⁺ T cell, a CTLA4⁻ LAG3⁻ PD1⁺ T cell, a CD57⁺ TIM3⁺ KLRG1⁻ CTLA4⁻ T cell, a CD57⁺ TIM3⁺ KLRG1⁻ LAG3⁻ T cell, a CD57⁺ TIM3⁺ KLRG1⁻ PD1⁺ T cell, a CD57⁺ TIM3⁺ CTLA4⁻ LAG3⁻ T cell, a CD57⁺ TIM3⁺ CTLA4⁻ PD1⁺ T cell, a CD57⁺ TIM3⁺ LAG3⁻ PD1⁺ T cell, a CD57⁺ KLRG1⁻ CTLA4⁻ LAG3⁻ T cell, a CD57⁺ KLRG1⁻ CTLA4⁻ PD1⁺ T cell, a CD57⁺ KLRG1⁻ LAG3⁻ PD1⁺ T cell, a CD57⁺ CTLA4⁻ LAG3⁻ PD1⁺ T cell, a TIM3⁺ KLRG1⁻ CTLA4⁻ LAG3⁻ T cell, a TIM3⁺ KLRG1⁻ CTLA4⁻ LAG3⁻ T cell, a TIM3⁺ KLRG1⁻ CTLA4⁻ PD1⁺ T cell, a TIM3⁺ KLRG1⁻ LAG3⁻ PD1⁺ T cell, a TIM3⁺ CTLA4⁻ LAG3⁻ PD1⁺ T cell, a KLRG1⁻ CTLA4⁻ LAG3⁻ PD1⁺ T cell, a CD57⁺ TIM3⁺ KLRG1⁻ CTLA4⁻ LAG3⁻ T cell, a CD57⁺ TIM3⁺ KLRG1⁻ CTLA4⁻ PD1⁺ T cell, a CD57⁺ TIM3⁺ KLRG1⁻ LAG3⁻ PD1⁺ T cell, a CD57⁺ TIM3⁺ CTLA4⁻ LAG3⁻ PD1⁺ T cell, a CD57⁺ KLRG1⁻ CTLA4⁻ LAG3⁻ PD1⁺ T cell, a TIM3⁺ KLRG1⁻ CTLA4⁻ LAG3⁻ PD1⁺ T cell, a CD57⁺ TIM3⁺ KLRG1⁻ CTLA4⁻ LAG3⁻ PD1⁺ T cell, or a combination thereof.

In certain embodiments of the compositions disclosed herein, the composition comprises at least about 5% of a CD57⁺ T cell comprising the CD19-targeted chimeric receptor, at least about 5% of a TIM3⁺ T cell comprising the CD19-targeted chimeric receptor, at least about 5% of a KLRG1⁻ T cell comprising the CD19-targeted chimeric receptor, at least about 5% of a CTLA4⁻ T cell comprising the CD19-targeted chimeric receptor, at least about 5% of a LAG3⁻ T cell comprising the CD19-targeted chimeric receptor, at least about 5% of a PD1⁺ T cell comprising the CD19-targeted chimeric receptor, at least about 5% of a CD57⁺ TIM3⁺ T cell comprising the CD19-targeted chimeric receptor, at least about 5% of a CD57⁺ KLRG1⁻ T cell comprising the CD19-targeted chimeric receptor, at least about 5% of a CD57⁺ CTLA4⁻ T cell comprising the CD19-targeted chimeric receptor, at least about 5% of a CD57⁺ LAG3⁻ T cell comprising the CD19-targeted chimeric receptor, at least about 5% of a CD57⁺ PD1⁺ T cell comprising the CD19-targeted chimeric receptor, at least about 5% of a TIM3⁺ KLRG1⁻ T cell comprising the CD19-targeted chimeric receptor, at least about 5% of a TIM3⁺ CTLA4⁻ T cell comprising the CD19-targeted chimeric receptor, at least about 5% of a TIM3⁺ LAG3⁻ T cell comprising the CD19-targeted chimeric receptor, at least about 5% of a TIM3⁺ PD1⁺ T cell comprising the CD19-targeted chimeric receptor, at least about 5% of a KLRG1⁻ CTLA4⁻ T cell comprising the CD19-targeted chimeric receptor, at least about 5% of a KLRG1⁻ LAG3⁻ T cell comprising the CD19-targeted chimeric receptor, at least about 5% of a KLRG1⁻ PD1⁺ T cell comprising the CD19-targeted chimeric receptor, at least about 5% of a CTLA4⁻ LAG3⁻ T cell comprising the CD19-targeted chimeric receptor, at least about 5% of a CTLA4⁻ PD1⁺ T cell comprising the CD19-targeted chimeric receptor, at least about 5% of a LAG3⁻ PD1⁺ T cell comprising the CD19-targeted chimeric receptor, at least about 5% of a CD57⁺ TIM3⁺ KLRG1⁻ T cell comprising the CD19-targeted chimeric receptor, at least about 5% of a CD57⁺ TIM3⁺ CTLA4⁻ T cell comprising the CD19-targeted chimeric receptor, at least about 5% of a CD57⁺ TIM3⁺ LAG3⁻ T cell comprising the CD19-targeted chimeric receptor, at least about 5% of a CD57⁺ TIM3⁺ PD1⁺ T cell comprising the CD19-targeted chimeric receptor, at least about 5% of a CD57⁺ KLRG1⁻ CTLA4⁻ T cell comprising the CD19-targeted chimeric receptor, at least about 5% of a CD57⁺ KLRG1⁻ LAG3⁻ T cell comprising the CD19-targeted chimeric receptor, at least about 5% of a CD57⁺ KLRG1⁻ PD1⁺ T cell comprising the CD19-targeted chimeric receptor, at least about 5% of a CD57⁺ CTLA4⁻ LAG3⁻ T cell comprising the CD19-targeted chimeric receptor, at least about 5% of a CD57⁺ CTLA4⁻ PD1⁺ T cell comprising the CD19-targeted chimeric receptor, at least about 5% of a CD57⁺ LAG3⁻ PD1⁺ T cell comprising the CD19-targeted chimeric receptor, at least about 5% of a TIM3⁺ KLRG1⁻ CTLA4⁻ T cell comprising the CD19-targeted chimeric receptor, at least about 5% of a TIM3⁺ KLRG1⁻ LAG3⁻ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ KLRG1⁻ PD1⁺ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ CTLA4⁻ PD1⁺ T cell comprising the CD19-targeted chimeric receptor, at least about 5% of a TIM3⁺ LAG3⁻ PD1⁺ T cell comprising the CD19-targeted chimeric receptor, at least about 5% of a KLRG1⁻ CTLA4⁻ LAG3⁻ T cell comprising the CD19-targeted chimeric receptor, at least about 5% of a KLRG1⁻ CTLA4⁻ PD1⁺ T cell comprising the CD19-targeted chimeric receptor, at least about 5% of a KLRG1⁻ LAG3⁻ PD1⁺ T cell comprising the CD19-targeted chimeric receptor, at least about 5% of a CTLA4⁻ LAG3⁻ PD1⁺ T cell comprising the CD19-targeted chimeric receptor, at least about 5% of a CD57⁺ TIM3⁺ KLRG1⁻ CTLA4⁻ T cell comprising the CD19-targeted chimeric receptor, at least about 5% of a CD57⁺ TIM3⁺ KLRG1⁻ LAG3⁻ T cell comprising the CD19-targeted chimeric receptor, at least about 5% of a CD57⁺ TIM3⁺ KLRG1⁻ PD1⁺ T cell comprising the CD19-targeted chimeric receptor, at least about 5% of a CD57⁺ TIM3⁺ CTLA4⁻ LAG3⁻ T cell comprising the CD19-targeted chimeric receptor, at least about 5% of a CD57⁺ TIM3⁺ CTLA4⁻ PD1⁺ T cell comprising the CD19-targeted chimeric receptor, at least about 5% of a CD57⁺ TIM3⁺ LAG3⁻ PD1⁺ T cell comprising the CD19-targeted chimeric receptor, at least about 5% of a CD57⁺ KLRG1⁻ CTLA4⁻ LAG3⁻ T cell comprising the CD19-targeted chimeric receptor, at least about 5% of a CD57⁺ KLRG1⁻ CTLA4⁻ PD1⁺ T cell comprising the CD19-targeted chimeric receptor, at least about 5% of a CD57⁺ KLRG1⁻ LAG3⁻ PD1⁺ T cell comprising the CD19-targeted chimeric receptor, at least about 5% of a CD57⁺ CTLA4⁻ LAG3⁻ PD1⁺ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ PD1⁺ T cell comprising the CD19-targeted chimeric receptor, at least about 5% of a TIM3⁺ KLRG1⁻ LAG3⁻ PD1⁺ T cell comprising the CD19-targeted chimeric receptor, at least about 5% of a TIM3⁺ CTLA4⁻ LAG3⁻ PD1⁺ T cell comprising the CD19-targeted chimeric receptor, at least about 5% of a KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ T cell comprising the CD19-targeted chimeric receptor, at least about 5% of a CD57⁺ TIM3⁺ KLRG1⁻ CTLA4⁻ PD1⁺ T cell comprising the CD19-targeted chimeric receptor, at least about 5% of a CD57⁺ TIM3⁺ KLRG1⁻ LAG3⁻ PD1⁺ T cell comprising the CD19-targeted chimeric receptor, at least about 5% of a CD57⁺ TIM3⁺ CTLA4⁻ LAG3⁻ PD1⁺ T cell comprising the CD19-targeted chimeric receptor, at least about 5% of a CD57⁺ KLRG1⁻ CTLA4⁻ LAG3⁻ PD1⁺ T cell comprising the CD19-targeted chimeric receptor, at least about 5% of a TIM3⁺ KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ PD1⁺ T cell comprising the CD19-targeted chimeric receptor, or a combination thereof.

In certain embodiments of the compositions disclosed herein, the composition is a pharmaceutical composition further comprising a pharmaceutically acceptable carrier.

In certain embodiments of the compositions disclosed herein, the composition comprises between about 1×10⁶ and about 5×10⁸ cells.

In certain embodiments of the compositions disclosed herein, the composition comprises between about 1×10⁶ and about 1×10⁸ cells.

In certain embodiments of the compositions disclosed herein, the composition comprises between about 1×10⁶ and about 5×10⁷ cells.

In certain embodiments of the compositions disclosed herein, the composition comprises about 2.5×10⁷ cells.

In certain embodiments, the presently disclosed subject matter is directed to a method of reducing tumor burden in a subject, comprising administering to the subject a composition disclosed herein.

In certain embodiments of the methods disclosed herein, the method reduces the number of tumor cells, reduces tumor size, and/or eradicates the tumor in the subject.

In certain embodiments, the presently disclosed subject matter is directed to a method of increasing or lengthening survival of a subject having a neoplasm, comprising administering to the subject a composition disclosed herein.

In certain embodiments, the presently disclosed subject matter is directed to a method of treating and/or preventing a neoplasm in a subject, comprising administering to the subject a composition disclosed herein.

In certain embodiments of the methods disclosed herein, the tumor and/or neoplasm is associated with CD19.

In certain embodiments of the methods disclosed herein, the tumor and/or neoplasm is a blood cancer.

In certain embodiments of the methods disclosed herein, the blood cancer is selected from the group consisting of multiple myeloma, leukemia, and lymphomas.

In certain embodiments of the methods disclosed herein, the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia.

In certain embodiments of the methods disclosed herein, the lymphoma is Hodgkin's lymphoma or non-Hodgkin's lymphoma.

In certain embodiments of the methods disclosed herein, the tumor and/or neoplasm is a B cell malignancy.

In certain embodiments of the methods disclosed herein, the B cell malignancy is selected from the group consisting of B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments of the methods disclosed herein, the tumor and/or neoplasm is B cell lymphoma.

In certain embodiments of the methods disclosed herein, the B cell lymphoma is relapsed or refractory (R/R) B cell lymphoma.

In certain embodiments of the methods disclosed herein, the subject is a human subject.

In certain embodiments, the presently disclosed subject matter is directed to a method of reducing tumor burden in a subject that is non-responsive or has a reduced likelihood to respond to an adoptive cell therapy, comprising: (a) measuring the expression level of at least one of CD57, TIM3, KLRG1, CTLA4, LAG3 and PD1 in an immunoresponsive cell of a subject; (b) comparing the expression level to a reference sample; (c) identifying the subject as non-responsive or as having a reduced likelihood to respond to an adoptive cell therapy if (i) the expression level of CD57 is reduced compared to the reference sample, (ii) the expression level of TIM3 is reduced compared to the reference sample, (iii) the expression level of KLRG1 is increased compared to the reference sample, (iv) the expression level of CTLA4 is increased compared to the reference sample, (v) the expression level of LAG3 is increased compared to the reference sample, and/or (vi) the expression level of PD1 is reduced compared to the reference sample; and (d) administering to the subject a composition of any one of embodiments 48-76.

In certain embodiments of the methods disclosed herein, the method reduces the number of tumor cells, reduces tumor size, and/or eradicates the tumor in the subject.

In certain embodiments, the presently disclosed subject matter is directed to a method of increasing or lengthening survival of a subject that is non-responsive or has a reduced likelihood to respond to an adoptive cell therapy, comprising: (a) measuring the expression level of at least one of CD57, TIM3, KLRG1, CTLA4, LAG3 and PD1 in an immunoresponsive cell of a subject; (b) comparing the expression level to a reference sample; (c) identifying the subject as non-responsive or as having a reduced likelihood to respond to an adoptive cell therapy if (i) the expression level of CD57 is reduced compared to the reference sample, (ii) the expression level of TIM3 is reduced compared to the reference sample, (iii) the expression level of KLRG1 is increased compared to the reference sample, (iv) the expression level of CTLA4 is increased compared to the reference sample, (v) the expression level of LAG3 is increased compared to the reference sample, and/or (vi) the expression level of PD1 is reduced compared to the reference sample; and (d) administering to the subject a composition of any one of embodiments 48-76.

In certain embodiments, the presently disclosed subject matter is directed to a A method of treating and/or preventing a neoplasm in a subject that is non-responsive or has a reduced likelihood to respond to an adoptive cell therapy, comprising: (a) measuring the expression level of at least one of CD57, TIM3, KLRG1, CTLA4, LAG3 and PD1 in an immunoresponsive cell of a subject; (b) comparing the expression level to a reference sample; (c) identifying the subject as non-responsive or as having a reduced likelihood to respond to an adoptive cell therapy if (i) the expression level of CD57 is reduced compared to the reference sample, (ii) the expression level of TIM3 is reduced compared to the reference sample, (iii) the expression level of KLRG1 is increased compared to the reference sample, (iv) the expression level of CTLA4 is increased compared to the reference sample, (v) the expression level of LAG3 is increased compared to the reference sample and/or (vi) the expression level of PD1 is reduced compared to the reference sample; and (d) administering to the subject a composition of any one of embodiments 48-76.

In certain embodiments of the methods disclosed herein, the tumor and/or neoplasm is associated with CD19.

In certain embodiments of the methods disclosed herein, the tumor and/or neoplasm is a blood cancer.

In certain embodiments of the methods disclosed herein, the blood cancer is selected from the group consisting of multiple myeloma, leukemia, and lymphomas.

In certain embodiments of the methods disclosed herein, the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia.

In certain embodiments of the methods disclosed herein, the lymphoma is Hodgkin's lymphoma or non-Hodgkin's lymphoma.

In certain embodiments of the methods disclosed herein, the tumor and/or neoplasm is a B cell malignancy.

In certain embodiments of the methods disclosed herein, the B cell malignancy is selected from the group consisting of B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments of the methods disclosed herein, the tumor and/or neoplasm is B cell lymphoma.

In certain embodiments of the methods disclosed herein, the B cell lymphoma is relapsed or refractory (R/R) B cell lymphoma.

In certain embodiments of the methods disclosed herein, the subject is a human subject.

### In Certain Embodiments, the Presently Disclosed Subject Matter is Directed to a Kit for Performing any of the Methods Disclosed Herein. EXAMPLE

The presently disclosed subject matter will be better understood by reference to the following Example, which is provided as exemplary of the presently disclosed subject matter, and not by way of limitation.

### Example 1—Role of Cell Therapy Product Characteristics Inpatient Response to CD19(T2)28z1xx Chimeric Antigen Receptor (CAR) T Cells in Adult Patients with Relapsed or Refractory B-Cell Malignancies

CD19(T2)28z1xx is an autologous CD19 chimeric antigen receptor (CAR)-T cell therapy utilizing an optimized 1XX signaling domain for the CD3ζ immunoreceptor tyrosine-based activation motif (ITAM). This design is aimed at extending the functional persistence of CAR-T cells without compromising their potency (Feucht J et al. Nat Med 2019). Early encouraging clinical efficacy of CD19(T2)28z1xx CAR T cells has been demonstrated in patients with large B-cell lymphoma (Park J et al. ASH 2022). The present example is aimed at providing a better understanding of the clinical performance of CD19(T2)28z1xx CAR T cells by in depth characterization of the cellular products and clinical biomarkers.

A total of 28 adult patients with relapsed or refractory B-cell Lymphoma were enrolled in the phase I dose escalation/expansion study of CD19(T2)28z1XX CAR T cells. Responses were observed across all dose levels, 25×10⁶ to 200×10⁶ with 82.5% ORR and 71% CR rate. Immunophenotyping by flow cytometry was conducted on patient apheresis starting material (SM), un-transduced (UTD) cells, cell therapy product infusion material, and peripheral blood samples from patients on treatment.

In the final infused product samples, increased CAR⁺ and CAR⁻ CD8⁺ central memory and transitional memory T cells were present compared to naïve, stem cell memory, or terminal effector populations, but this did not correlate to patient response. This increase in central and transitional memory T cells was not observed in the starting material, indicating that this effect was likely driven by the production process and transduction with a 1XX construct, similar to trends that have been observed in preclinical studies (Feucht J et al. Nat Med 2019). CD57 and TIM3 expression on both CD4⁺ and CD8⁺ CAR⁺ T cells in the product show a positive association with clinical response, while the expression of the inhibitory receptors CTLA4 and LAG3 show a negative association with response. TIM3 has been shown to be expressed on activated T and NK cells (Wolf et al. Nat Rev Immunology, 2019). In a previous study in large B cell lymphoma patients treated with Axi-Cel, it was observed that CD4⁺ and CD8⁺ CAR T cells with high expression of CD57 and T-bet on day 7 associated with durable CR, the profile of which resembled that of terminally differentiated effector T cells with early senescence features rather than exhausted T cells (Good Z et al. Nat Med 2022). CD57 expression on T cells has also been shown to indicate enhanced cytotoxic potential (Kared H et al, Cancer Immunol Immunother, 2016). The product material had low numbers of dysregulated/exhausted T cells (and no significant difference between responders and non-responders), potentially indicative of a CD57⁺ senescent phenotype of cells in the product associating with patient response. This observation warrants further studies to establish the role of CD57⁺ cells in responses to CAR-T cell therapy in particular with a 1XX product.

In conclusion, the present example observed differential distribution of T cell phenotypes in the CAR-T infusion product likely owing to the transduction process. In addition, expression levels of CD57 and TIM3 on T cells in the product were associated with clinical responses, and CTLA4 and LAG3 were negatively associated with response. Interestingly, decreased LAG3 and KLRG1, as well as increased TIM3 expression on CD8 T cells in the starting material correlated with responses, indicating the potential utility of these markers measured on T cells at screening in predicting patient responses. Correlative analysis of T cell immunophenotypes observed in the product to clinical parameters, metabolic tumor volumes/tumor burden, induction of cytokines and chemokines is ongoing. These analyses can further contribute to the understanding of CD19 CAR-T with 1XX construct and predict patient response.

### Example 2—Immunophenotyping in Adult Patients with Relapsed or Refractory B-Cell Malignancies Receiving CD19(T2)28z1xx Chimeric Antigen Receptor (CAR) T Cells

The present example studied the immunophenotyping of cells from adult patients with relapsed or refractory B-cell malignancies that have received CD19(T2)28z1xx Chimeric Antigen Receptor (CAR) T Cells. In order to better characterize the cell population in these patients, multiple proteins were analyzed:

CD57 is a marker of terminally differentiated/senescent cells (can correlate with high KLRG1). It is associated with high cytotoxic potential and memory-like phenotype (Ramello et al, Front. Immunol., 2021, Zhang et al, EBioMedicine, 2021, Huang et al, J Immunother Cancer, 2020).

CTLA4 is a negative regulator of T cell activation (Walunas et al, Immunity, 1994).

PD1 is an important checkpoint that is also upregulated on activated cells (Dong et al, Oncotarget, 2017, Batorov et al, Annals of Oncology, 2019).

TIM3 is an immunomodulatory receptor. With PD1, TIM3 can denote highly exhausted cells. Further, TIM3 is upregulated early on activated cells (Batorov et al., Annals of Oncology, 2019, Das et al, Immunol Rev., 2017).

LAG3 negatively regulates T cell homeostasis by regulatory T cell-dependent and independent mechanisms (Workman et al, J Immunol, 2005).

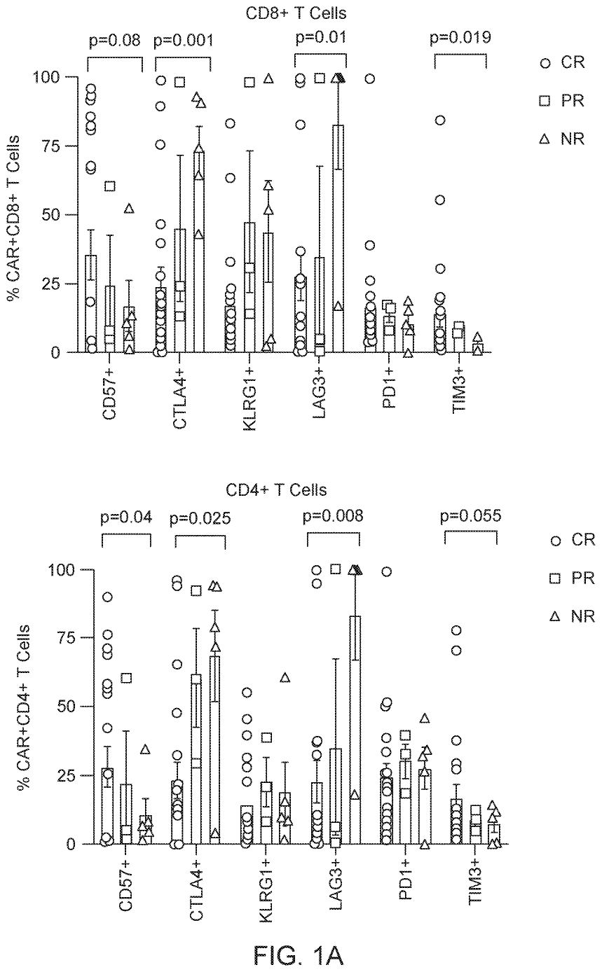

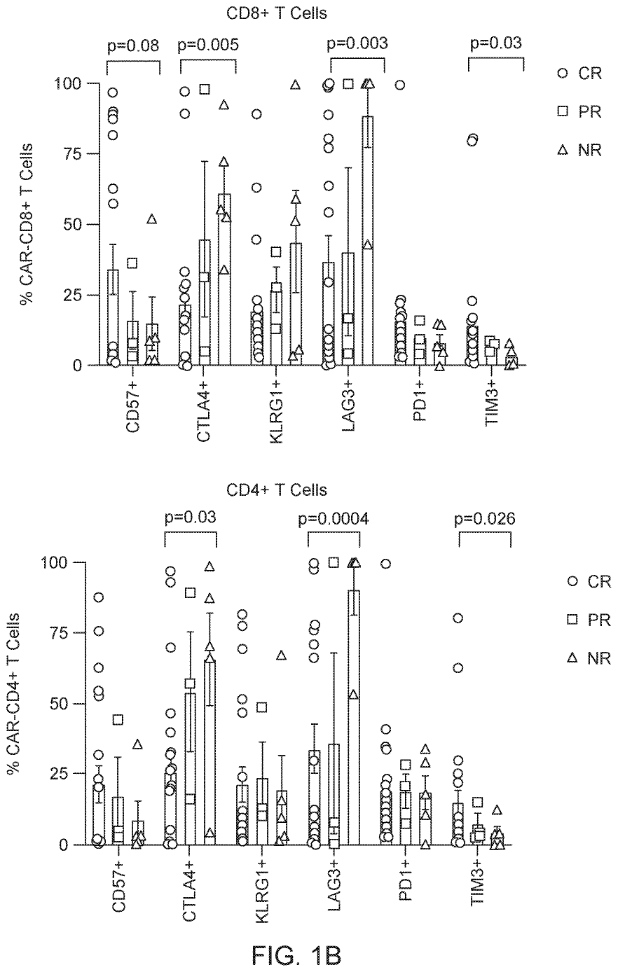

As seen in FIGS. 1A and 1B, a trend towards a positive association between response and CD57 and TIM3 expression on CAR⁺ CD4 and CD8 T cells was observed. Notably, this trend was significant for CD57 in CD4 and TIM3 on both CD4 and CD8 T cells. In addition, it was observed a trend towards a negative association between CTLA4 and LAG3 expression and response on CAR⁺ CD4 and CD8 T cells, which was significant for CTLA4 and LAG3 on both CD4 and CD8 T.

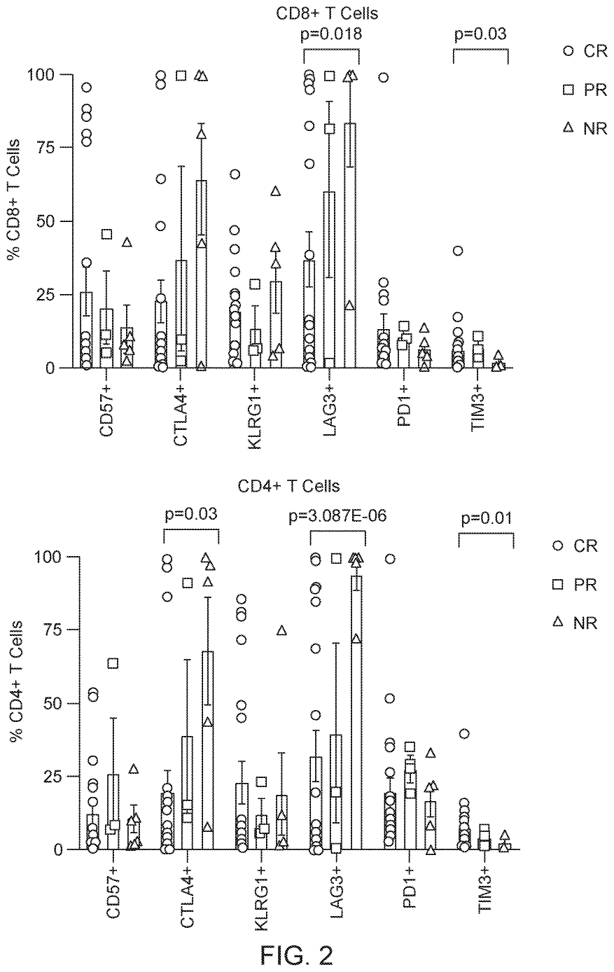

Next, the immunophenotyping was conducted on untransduced cells (UTD). As seen in FIG. 2, UTD cells had a similar trend in higher CD57% cells, PD1% cells, and TIM3% cells in complete response (CR) vs. non-response (NR) as seen in FIGS. 1A and 1B. UTD cells also showed a similar trend with a lower percentage of CTLA4⁺ and LAG3⁺ cells in CR vs. NR patients.

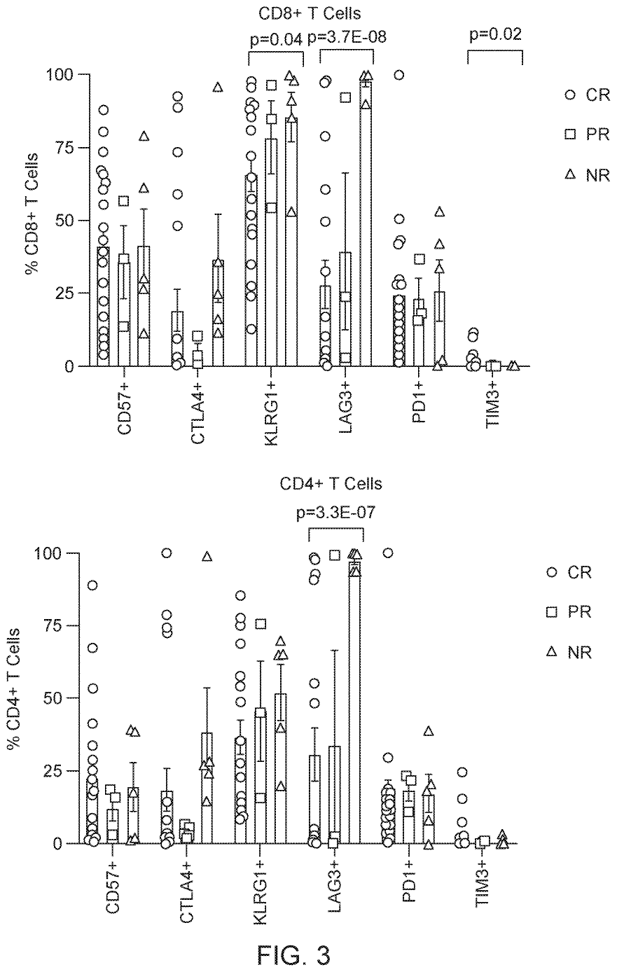

To verify whether these observations were due to the starting material, further analyses were conducted. The starting material did not show an association with response and CD57 or PD1 markers as observed with the product (FIG. 3). Importantly, there was a trend towards lower CTLA4 in CR vs. NR. Moreover, significant differences were observed between CR and NR in KLRG1, LAG3 and TIM3 expression in CD8 T cells and LAG3 in CD4 T cells.

Although the presently disclosed subject matter and certain of its advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

What is claimed is:

1. A method comprising:
(a) measuring an expression level of at least one of CD57, TIM3, KLRG1, CTLA4, LAG3 and PD1 in an immunoresponsive cell of a subject;
(b) comparing the expression level to a reference sample; and
(c) identifying the subject as (i) responsive or as having an increased likelihood to respond to an adoptive cell therapy, or (ii) non-responsive or as having a reduced likelihood to respond to an adoptive cell therapy.

2. The method of claim 1, wherein the subject is responsive or has an increased likelihood to respond to the adoptive cell therapy if
(a) the expression level of CD57 is increased compared to the reference sample;
(b) the expression level of TIM3 is increased compared to the reference sample;
(c) the expression level of KLRG1 is reduced compared to the reference sample;
(d) the expression level of CTLA4 is reduced compared to the reference sample;
(e) the expression level of LAG3 is reduced compared to the reference sample; and/or
(f) the expression level of PD1 is increased compared to the reference sample.

3. The method of claim 1, wherein the subject is non-responsive or has a reduced likelihood to respond the adoptive cell therapy if
(a) the expression level of CD57 is reduced compared to the reference sample;
(b) the expression level of TIM3 is reduced compared to the reference sample;
(c) the expression level of KLRG1 is increased compared to the reference sample;
(d) the expression level of CTLA4 is increased compared to the reference sample;
(e) the expression level of LAG3 is increased compared to the reference sample; and/or
(f) the expression level of PD1 is reduced compared to the reference sample.

4. The method of claim 1, wherein the adoptive cell therapy comprises a cell comprising a CD19-targeted chimeric receptor.

5. The method of claim 1, further comprising administering to the subject identified as responsive or as having an increased likelihood to respond to an adoptive cell therapy a cell comprising a CD19-targeted chimeric receptor or a composition comprising thereof, wherein the CD19-targeted chimeric receptor comprises an extracellular antigen-binding domain comprising a heavy chain variable region (V_{H}) comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12, and a light chain variable region (V_{L}) comprising a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15.

6. The method of claim 5, wherein a) the heavy chain variable region comprises the amino acid sequence set forth in SEQ ID NO: 16; and b) the heavy chain variable region comprises the amino acid sequence set forth in SEQ ID NO: 17.

7. The method of claim 5, wherein the cell comprising the CD19-targeted chimeric receptor is an immunoresponsive cell.

8. The method of claim 7, wherein the cell comprising the CD19-targeted chimeric receptor is selected from the group consisting of a T cell, a Natural Killer (NK) cell, a stem cell from which lymphoid cells may be differentiated, and a stem cell from which myeloid cells may be differentiated.

9. The method of claim 5, wherein the cell comprising the CD19-targeted chimeric receptor is selected from a CD57⁺ T cell, a TIM3⁺ T cell, a KLRG1⁻ T cell, a CTLA4⁻ T cell, a LAG3⁻ T cell, a PD1⁺ T cell, a CD57⁺ TIM3⁺ T cell, a CD57⁺ KLRG1⁻ T cell, a CD57⁺ CTLA4⁻ T cell, a CD57⁺ LAG3⁻ T cell, a CD57⁺ PD1⁺ T cell, a TIM3⁺ KLRG1⁻ T cell, a TIM3⁺ CTLA4⁻ T cell, a TIM3⁺ LAG3⁻ T cell, a TIM3⁺ PD1⁺ T cell, a KLRG1⁻ CTLA4⁻ T cell, a KLRG1⁻ LAG3⁻ T cell, a KLRG1⁻ PD1⁺ T cell, a CTLA4⁻ LAG3⁻ T cell, a CTLA4⁻ PD1⁺ T cell, a LAG3⁻ PD1⁺ T cell, a CD57⁺ TIM3⁺ KLRG1⁻ T cell, a CD57⁺ TIM3⁺ CTLA4⁻ T cell, a CD57⁺ TIM3⁺ LAG3⁻ T cell, a CD57⁺ TIM3⁺ PD1⁺ T cell, a CD57⁺ KLRG1⁻ CTLA4⁻ T cell, a CD57⁺ KLRG1⁻ LAG3⁻ T cell, a CD57⁺ KLRG1⁻ PD1⁺ T cell, a CD57⁺ CTLA4⁻ LAG3⁻ T cell, a CD57⁺ CTLA4⁻ PD1⁺ T cell, a CD57⁺ LAG3⁻ PD1⁺ T cell, a TIM3⁺ KLRG1⁻ CTLA4⁻ T cell, a TIM3⁺ KLRG1⁻ LAG3⁻ T cell, a TIM3⁺ CTLA4⁻ LAG3⁻ T cell, a TIM3⁺ KLRG1⁻ PD1⁺ T cell, a TIM3⁺ CTLA4⁻ LAG3⁻ T cell, a TIM3⁺ CTLA4⁻ PD1⁺ T cell, a TIM3⁺ LAG3⁻ PD1⁺ T cell, a KLRG1⁻ CTLA4⁻ LAG3⁻ T cell, a KLRG1⁻ CTLA4⁻ PD1⁺ T cell, a KLRG1⁻ LAG3⁻ PD1⁺ T cell, a CTLA4⁻ LAG3⁻ PD1⁺ T cell, a CD57⁺ TIM3⁺ KLRG1⁻ CTLA4⁻ T cell, a CD57⁺ TIM3⁺ KLRG1⁻ LAG3⁻ T cell, a CD57⁺ TIM3⁺ KLRG1⁻ PD1⁺ T cell, a CD57⁺ TIM3⁺ CTLA4⁻ LAG3⁻ T cell, a CD57⁺ TIM3⁺ CTLA4⁻ PD1⁺ T cell, a CD57⁺ TIM3⁺ LAG3⁻ PD1⁺ T cell, a CD57⁺ KLRG1⁻ CTLA4⁻ LAG3⁻ T cell, a CD57⁺ KLRG1⁻ CTLA4⁻ PD1⁺ T cell, a CD57⁺ KLRG1⁻ LAG3⁻ PD1⁺ T cell, a CD57⁺ CTLA4⁻ LAG3⁻ PD1⁺ T cell, a TIM3⁺ KLRG1⁻ CTLA4⁻ LAG3⁻ T cell, a TIM3⁺ KLRG1⁻ CTLA4⁻ LAG3⁻ T cell, a TIM3⁺ KLRG1⁻ CTLA4⁻ PD1⁺ T cell, a TIM3⁺ KLRG1⁻ LAG3⁻ PD1⁺ T cell, a TIM3⁺ CTLA4⁻ LAG3⁻ PD1⁺ T cell, a KLRG1⁻ CTLA4⁻ LAG3⁻ PD1⁺ T cell, a CD57⁺ TIM3⁺ KLRG1⁻ CTLA4⁻ LAG3⁻ T cell, a CD57⁺ TIM3⁺ KLRG1⁻ CTLA4⁻ PD1⁺ T cell, a CD57⁺ TIM3⁺ KLRG1⁻ LAG3⁻ PD1⁺ T cell, a CD57⁺ TIM3⁺ CTLA4⁻ LAG3⁻ PD1⁺ T cell, a CD57⁺ KLRG1⁻ CTLA4⁻ LAG3⁻ PD1⁺ T cell, a TIM3⁺ KLRG1⁻ CTLA4⁻ LAG3⁻ PD1⁺ T cell, a CD57⁺ TIM3⁺ KLRG1⁻ CTLA4⁻ LAG3⁻ PD1⁺ T cell, or a combination thereof.

10. The method of claim 5, wherein the composition comprises at least about 5% of a CD57⁺ T cell comprising the CD19-targeted chimeric receptor, at least about 5% of a TIM3⁺ T cell comprising the CD19-targeted chimeric receptor, at least about 5% of a KLRG1⁻ T cell comprising the CD19-targeted chimeric receptor, at least about 5% of a CTLA4⁻ T cell comprising the CD19-targeted chimeric receptor, at least about 5% of a LAG3⁻ T cell comprising the CD19-targeted chimeric receptor, at least about 5% of a PD1⁺ T cell comprising the CD19-targeted chimeric receptor, at least about 5% of a CD57⁺ TIM3⁺ T cell comprising the CD19-targeted chimeric receptor, at least about 5% of a CD57⁺ KLRG1⁻ T cell comprising the CD19-targeted chimeric receptor, at least about 5% of a CD57⁺ CTLA4⁻ T cell comprising the CD19-targeted chimeric receptor, at least about 5% of a CD57⁺ LAG3⁻ T cell comprising the CD19-targeted chimeric receptor, at least about 5% of a CD57⁺ PD1⁺ T cell comprising the CD19-targeted chimeric receptor, at least about 5% of a TIM3⁺ KLRG1⁻ T cell comprising the CD19-targeted chimeric receptor, at least about 5% of a TIM3⁺ CTLA4⁻ T cell comprising the CD19-targeted chimeric receptor, at least about 5% of a TIM3⁺ LAG3⁻ T cell comprising the CD19-targeted chimeric receptor, at least about 5% of a TIM3⁺ PD1⁺ T cell comprising the CD19-targeted chimeric receptor, at least about 5% of a KLRG1⁻ CTLA4⁻ T cell comprising the CD19-targeted chimeric receptor, at least about 5% of a KLRG1⁻ LAG3⁻ T cell comprising the CD19-targeted chimeric receptor, at least about 5% of a KLRG1⁻ PD1⁺ T cell comprising the CD19-targeted chimeric receptor, at least about 5% of a CTLA4⁻ LAG3⁻ T cell comprising the CD19-targeted chimeric receptor, at least about 5% of a CTLA4⁻ PD1⁺ T cell comprising the CD19-targeted chimeric receptor, at least about 5% of a LAG3⁻ PD1⁺ T cell comprising the CD19-targeted chimeric receptor, at least about 5% of a CD57⁺ TIM3⁺ KLRG1⁻ T cell comprising the CD19-targeted chimeric receptor, at least about 5% of a CD57⁺ TIM3⁺ CTLA4⁻ T cell comprising the CD19-targeted chimeric receptor, at least about 5% of a CD57⁺ TIM3⁺ LAG3⁻ T cell comprising the CD19-targeted chimeric receptor, at least about 5% of a CD57⁺ TIM3⁺ PD1⁺ T cell comprising the CD19-targeted chimeric receptor, at least about 5% of a CD57⁺ KLRG1⁻ CTLA4⁻ T cell comprising the CD19-targeted chimeric receptor, at least about 5% of a CD57⁺ KLRG1⁻ LAG3⁻ T cell comprising the CD19-targeted chimeric receptor, at least about 5% of a CD57⁺ KLRG1⁻ PD1⁺ T cell comprising the CD19-targeted chimeric receptor, at least about 5% of a CD57⁺ CTLA4⁻ LAG3⁻ T cell comprising the CD19-targeted chimeric receptor, at least about 5% of a CD57⁺ CTLA4⁻ PD1⁺ T cell comprising the CD19-targeted chimeric receptor, at least about 5% of a CD57⁺ LAG3⁻ PD1⁺ T cell comprising the CD19-targeted chimeric receptor, at least about 5% of a TIM3⁺ KLRG1⁻ CTLA4⁻ T cell comprising the CD19-targeted chimeric receptor, at least about 5% of a TIM3⁺ KLRG1⁻ LAG3⁻ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ KLRG1⁻ PD1⁺ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ CTLA4⁻ PD1⁺ T cell comprising the CD19-targeted chimeric receptor, at least about 5% of a TIM3⁺ LAG3⁻ PD1⁺ T cell comprising the CD19-targeted chimeric receptor, at least about 5% of a KLRG1⁻ CTLA4⁻ LAG3⁻ T cell comprising the CD19-targeted chimeric receptor, at least about 5% of a KLRG1⁻ CTLA4⁻ PD1⁺ T cell comprising the CD19-targeted chimeric receptor, at least about 5% of a KLRG1⁻ LAG3⁻ PD1⁺ T cell comprising the CD19-targeted chimeric receptor, at least about 5% of a CTLA4⁻ LAG3⁻ PD1⁺ T cell comprising the CD19-targeted chimeric receptor, at least about 5% of a CD57⁺ TIM3⁺ KLRG1⁻ CTLA4⁻ T cell comprising the CD19-targeted chimeric receptor, at least about 5% of a CD57⁺ TIM3⁺ KLRG1⁻ LAG3⁻ T cell comprising the CD19-targeted chimeric receptor, at least about 5% of a CD57⁺ TIM3⁺ KLRG1⁻ PD1⁺ T cell comprising the CD19-targeted chimeric receptor, at least about 5% of a CD57⁺ TIM3⁺ CTLA4⁻ LAG3⁻ T cell comprising the CD19-targeted chimeric receptor, at least about 5% of a CD57⁺ TIM3⁺ CTLA4⁻ PD1⁺ T cell comprising the CD19-targeted chimeric receptor, at least about 5% of a CD57⁺ TIM3⁺ LAG3⁻ PD1⁺ T cell comprising the CD19-targeted chimeric receptor, at least about 5% of a CD57⁺ KLRG1⁻ CTLA4⁻ LAG3⁻ T cell comprising the CD19-targeted chimeric receptor, at least about 5% of a CD57⁺ KLRG1⁻ CTLA4⁻ PD1⁺ T cell comprising the CD19-targeted chimeric receptor, at least about 5% of a CD57⁺ KLRG1⁻ LAG3⁻ PD1⁺ T cell comprising the CD19-targeted chimeric receptor, at least about 5% of a CD57⁺ CTLA4⁻ LAG3⁻ PD1⁺ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ PD1⁺ T cell comprising the CD19-targeted chimeric receptor, at least about 5% of a TIM3⁺ KLRG1⁻ LAG3⁻ PD1⁺ T cell comprising the CD19-targeted chimeric receptor, at least about 5% of a TIM3⁺ CTLA4⁻ LAG3⁻ PD1⁺ T cell comprising the CD19-targeted chimeric receptor, at least about 5% of a KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ T cell comprising the CD19-targeted chimeric receptor, at least about 5% of a CD57⁺ TIM3⁺ KLRG1⁻ CTLA4⁻ PD1⁺ T cell comprising the CD19-targeted chimeric receptor, at least about 5% of a CD57⁺ TIM3⁺ KLRG1⁻ LAG3⁻ PD1⁺ T cell comprising the CD19-targeted chimeric receptor, at least about 5% of a CD57⁺ TIM3⁺ CTLA4⁻ LAG3⁻ PD1⁺ T cell comprising the CD19-targeted chimeric receptor, at least about 5% of a CD57⁺ KLRG1⁻ CTLA4⁻ LAG3⁻ PD1⁺ T cell comprising the CD19-targeted chimeric receptor, at least about 5% of a TIM3⁺ KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ PD1⁺ T cell comprising the CD19-targeted chimeric receptor, or a combination thereof.

11. The method of claim 5, wherein the subject has a tumor and/or a neoplasm associated with CD19.

12. The method of claim 1, wherein the expression level of at least one of CD57, TIM3, KLRG1, CTLA4, LAG3 and PD1 is measured on the surface of the immunoresponsive cell or is measured by cytometry or by flow-cytometry.

13. The method of claim 7, wherein the immunoresponsive cell is a T cell.

14. The method of claim 13, wherein T cell is selected from the group consisting of a helper T cell, a cytotoxic T cell, a memory T cell, a central memory T cell, a stem-like memory T cell, an effector memory T cell, a regulatory T cell, a tumor-infiltrating lymphocyte (TIL), a Natural killer T cells, a γδ T cell, a CD4⁺ T cells, and a CD8⁺ T cell.

15. A composition comprising a cell comprising a CD19-targeted chimeric receptor, wherein the CD19-targeted chimeric receptor comprises an extracellular antigen-binding domain, a transmembrane domain, and an intracellular domain, wherein the extracellular antigen-binding domain comprises a heavy chain variable region (V_{H}) comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12, and a light chain variable region (V_{L}) comprising a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15.

16. The composition of claim 15, wherein the extracellular antigen-binding domain comprises a scFv.

17. The composition of claim 16, wherein a) the heavy chain variable region comprises the amino acid sequence set forth in SEQ ID NO: 16; and b) the heavy chain variable region comprises the amino acid sequence set forth in SEQ ID NO: 17.

18. The composition of claim 15, wherein the intracellular signaling domain comprises a CD3ζ polypeptide comprising a native ITAM1, an ITAM2 variant consisting of two loss-of-function mutations, and an ITAM3 consisting of two loss-of-function mutations.

19. The composition of claim 18, wherein the intracellular signaling domain comprises a CD3ζ polypeptide comprising the amino acid sequence set forth in SEQ ID NO: 40.

20. The composition of claim 15, wherein the chimeric receptor comprises the amino acid sequence set forth in SEQ ID NO: 45.

21. The composition of claim 15, wherein the cell is an immunoresponsive cell.

22. The composition of claim 21, wherein the cell is selected from the group consisting of a T cell, a Natural Killer (NK) cell, a stem cell from which lymphoid cells may be differentiated, and a stem cell from which myeloid cells may be differentiated.

23. The composition of claim 15, wherein the cell is selected from a CD57⁺ T cell, a TIM3⁺ T cell, a KLRG1⁻ T cell, a CTLA4⁻ T cell, a LAG3⁻ T cell, a PD1⁺ T cell, a CD57⁺ TIM3⁺ T cell, a CD57⁺ KLRG1 T cell, a CD57⁺ CTLA4⁻ T cell, a CD57⁺ LAG3⁻ T cell, a CD57⁺ PD1⁺ T cell, a TIM3⁺ KLRG1 T cell, a TIM3⁺ CTLA4⁻ T cell, a TIM3⁺ LAG3⁻ T cell, a TIM3⁺ PD1⁺ T cell, a KLRG1⁻ CTLA4⁻ T cell, a KLRG1⁻ LAG3⁻ T cell, a KLRG1⁻ PD1⁺ T cell, a CTLA4⁻ LAG3⁻ T cell, a CTLA4⁻ PD1⁺ T cell, a LAG3⁻ PD1⁺ T cell, a CD57⁺ TIM3⁺ KLRG1⁻ T cell, a CD57⁺ TIM3⁺ CTLA4⁻ T cell, a CD57⁺ TIM3⁺ LAG3⁻ T cell, a CD57⁺ TIM3⁺ PD1⁺ T cell, a CD57⁺ KLRG1⁻ CTLA4⁻ T cell, a CD57⁺ KLRG1⁻ LAG3⁻ T cell, a CD57⁺ KLRG1⁻ PD1⁺ T cell, a CD57⁺ CTLA4⁻ LAG3⁻ T cell, a CD57⁺ CTLA4⁻ PD1⁺ T cell, a CD57⁺ LAG3⁻ PD1⁺ T cell, a TIM3⁺ KLRG1⁻ CTLA4⁻ T cell, a TIM3⁺ KLRG1⁻ LAG3⁻ T cell, a TIM3⁺ CTLA4⁻ LAG3⁻ T cell, a TIM3⁺ KLRG1⁻ PD1⁺ T cell, a TIM3⁺ CTLA4⁻ LAG3⁻ T cell, a TIM3⁺ CTLA4⁻ PD1⁺ T cell, a TIM3⁺ LAG3⁻ PD1⁺ T cell, a KLRG1⁻ CTLA4⁻ LAG3⁻ T cell, a KLRG1⁻ CTLA4⁻ PD1⁺ T cell, a KLRG1⁻ LAG3⁻ PD1⁺ T cell, a CTLA4⁻ LAG3⁻ PD1⁺ T cell, a CD57⁺ TIM3⁺ KLRG1⁻ CTLA4⁻ T cell, a CD57⁺ TIM3⁺ KLRG1⁻ LAG3⁻ T cell, a CD57⁺ TIM3⁺ KLRG1⁻ PD1⁺ T cell, a CD57⁺ TIM3⁺ CTLA4⁻ LAG3⁻ T cell, a CD57⁺ TIM3⁺ CTLA4⁻ PD1⁺ T cell, a CD57⁺ TIM3⁺ LAG3⁻ PD1⁺ T cell, a CD57⁺ KLRG1⁻ CTLA4⁻ LAG3⁻ T cell, a CD57⁺ KLRG1⁻ CTLA4⁻ PD1⁺ T cell, a CD57⁺ KLRG1⁻ LAG3⁻ PD1⁺ T cell, a CD57⁺ CTLA4⁻ LAG3⁻ PD1⁺ T cell, a TIM3⁺ KLRG1⁻ CTLA4⁻ LAG3⁻ T cell, a TIM3⁺ KLRG1⁻ CTLA4⁻ LAG3⁻ T cell, a TIM3⁺ KLRG1⁻ CTLA4⁻ PD1⁺ T cell, a TIM3⁺ KLRG1⁻ LAG3⁻ PD1⁺ T cell, a TIM3⁺ CTLA4⁻ LAG3⁻ PD1⁺ T cell, a KLRG1⁻ CTLA4⁻ LAG3⁻ PD1⁺ T cell, a CD57⁺ TIM3⁺ KLRG1⁻ CTLA4⁻ LAG3⁻ T cell, a CD57⁺ TIM3⁺ KLRG1⁻ CTLA4⁻ PD1⁺ T cell, a CD57⁺ TIM3⁺ KLRG1⁻ LAG3⁻ PD1⁺ T cell, a CD57⁺ TIM3⁺ CTLA4⁻ LAG3⁻ PD1⁺ T cell, a CD57⁺ KLRG1⁻ CTLA4⁻ LAG3⁻ PD1⁺ T cell, a TIM3⁺ KLRG1⁻ CTLA4⁻ LAG3⁻ PD1⁺ T cell, a CD57⁺ TIM3⁺ KLRG1⁻ CTLA4⁻ LAG3⁻ PD1⁺ T cell, or a combination thereof.

24. The composition of claim 15, comprising at least about 5% of a CD57⁺ T cell comprising the CD19-targeted chimeric receptor, at least about 5% of a TIM3⁺ T cell comprising the CD19-targeted chimeric receptor, at least about 5% of a KLRG1⁻ T cell comprising the CD19-targeted chimeric receptor, at least about 5% of a CTLA4⁻ T cell comprising the CD19-targeted chimeric receptor, at least about 5% of a LAG3⁻ T cell comprising the CD19-targeted chimeric receptor, at least about 5% of a PD1⁺ T cell comprising the CD19-targeted chimeric receptor, at least about 5% of a CD57⁺ TIM3⁺ T cell comprising the CD19-targeted chimeric receptor, at least about 5% of a CD57⁺ KLRG1⁻ T cell comprising the CD19-targeted chimeric receptor, at least about 5% of a CD57⁺ CTLA4⁻ T cell comprising the CD19-targeted chimeric receptor, at least about 5% of a CD57⁺ LAG3⁻ T cell comprising the CD19-targeted chimeric receptor, at least about 5% of a CD57⁺ PD1⁺ T cell comprising the CD19-targeted chimeric receptor, at least about 5% of a TIM3⁺ KLRG1⁻ T cell comprising the CD19-targeted chimeric receptor, at least about 5% of a TIM3⁺ CTLA4⁻ T cell comprising the CD19-targeted chimeric receptor, at least about 5% of a TIM3⁺ LAG3⁻ T cell comprising the CD19-targeted chimeric receptor, at least about 5% of a TIM3⁺ PD1⁺ T cell comprising the CD19-targeted chimeric receptor, at least about 5% of a KLRG1⁻ CTLA4⁻ T cell comprising the CD19-targeted chimeric receptor, at least about 5% of a KLRG1⁻ LAG3⁻ T cell comprising the CD19-targeted chimeric receptor, at least about 5% of a KLRG1⁻ PD1⁺ T cell comprising the CD19-targeted chimeric receptor, at least about 5% of a CTLA4⁻ LAG3⁻ T cell comprising the CD19-targeted chimeric receptor, at least about 5% of a CTLA4⁻ PD1⁺ T cell comprising the CD19-targeted chimeric receptor, at least about 5% of a LAG3⁻ PD1⁺ T cell comprising the CD19-targeted chimeric receptor, at least about 5% of a CD57⁺ TIM3⁺ KLRG1⁻ T cell comprising the CD19-targeted chimeric receptor, at least about 5% of a CD57⁺ TIM3⁺ CTLA4⁻ T cell comprising the CD19-targeted chimeric receptor, at least about 5% of a CD57⁺ TIM3⁺ LAG3⁻ T cell comprising the CD19-targeted chimeric receptor, at least about 5% of a CD57⁺ TIM3⁺ PD1⁺ T cell comprising the CD19-targeted chimeric receptor, at least about 5% of a CD57⁺ KLRG1⁻ CTLA4⁻ T cell comprising the CD19-targeted chimeric receptor, at least about 5% of a CD57⁺ KLRG1⁻ LAG3⁻ T cell comprising the CD19-targeted chimeric receptor, at least about 5% of a CD57⁺ KLRG1⁻ PD1⁺ T cell comprising the CD19-targeted chimeric receptor, at least about 5% of a CD57⁺ CTLA4⁻ LAG3⁻ T cell comprising the CD19-targeted chimeric receptor, at least about 5% of a CD57⁺ CTLA4⁻ PD1⁺ T cell comprising the CD19-targeted chimeric receptor, at least about 5% of a CD57⁺ LAG3⁻ PD1⁺ T cell comprising the CD19-targeted chimeric receptor, at least about 5% of a TIM3⁺ KLRG1⁻ CTLA4⁻ T cell comprising the CD19-targeted chimeric receptor, at least about 5% of a TIM3⁺ KLRG1⁻ LAG3⁻ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ KLRG1⁻ PD1⁺ T cell comprising the CD19-targeted chimeric receptor, at least about 5% of a TIM3⁺ CTLA4⁻ LAG3⁻ T cell comprising the CD19-targeted chimeric receptor, at least about 5% of a TIM3⁺ CTLA4⁻ PD1⁺ T cell comprising the CD19-targeted chimeric receptor, at least about 5% of a TIM3⁺ LAG3⁻ PD1⁺ T cell comprising the CD19-targeted chimeric receptor, at least about 5% of a KLRG1⁻ CTLA4⁻ LAG3⁻ T cell comprising the CD19-targeted chimeric receptor, at least about 5% of a KLRG1⁻ CTLA4⁻ PD1⁺ T cell comprising the CD19-targeted chimeric receptor, at least about 5% of a KLRG1⁻ LAG3⁻ PD1⁺ T cell comprising the CD19-targeted chimeric receptor, at least about 5% of a CTLA4⁻ LAG3⁻ PD1⁺ T cell comprising the CD19-targeted chimeric receptor, at least about 5% of a CD57⁺ TIM3⁺ KLRG1⁻ CTLA4⁻ T cell comprising the CD19-targeted chimeric receptor, at least about 5% of a CD57⁺ TIM3⁺ KLRG1⁻ LAG3⁻ T cell comprising the CD19-targeted chimeric receptor, at least about 5% of a CD57⁺ TIM3⁺ KLRG1⁻ PD1⁺ T cell comprising the CD19-targeted chimeric receptor, at least about 5% of a CD57⁺ TIM3⁺ CTLA4⁻ LAG3⁻ T cell comprising the CD19-targeted chimeric receptor, at least about 5% of a CD57⁺ TIM3⁺ CTLA4⁻ PD1⁺ T cell comprising the CD19-targeted chimeric receptor, at least about 5% of a CD57⁺ TIM3⁺ LAG3⁻ PD1⁺ T cell comprising the CD19-targeted chimeric receptor, at least about 5% of a CD57⁺ KLRG1⁻ CTLA4⁻ LAG3⁻ T cell comprising the CD19-targeted chimeric receptor, at least about 5% of a CD57⁺ KLRG1⁻ CTLA4⁻ PD1⁺ T cell comprising the CD19-targeted chimeric receptor, at least about 5% of a CD57⁺ KLRG1⁻ LAG3⁻ PD1⁺ T cell comprising the CD19-targeted chimeric receptor, at least about 5% of a CD57⁺ CTLA4⁻ LAG3⁻ PD1⁺ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ LAG3⁻ T cell comprising the CD19-targeted chimeric receptor, at least about 5% of a TIM3⁺ KLRG1⁻ CTLA4⁻ PD1⁺ T cell comprising the CD19-targeted chimeric receptor, at least about 5% of a TIM3⁺ KLRG1⁻ LAG3⁻ PD1⁺ T cell comprising the CD19-targeted chimeric receptor, at least about 5% of a TIM3⁺ CTLA4⁻ LAG3⁻ PD1⁺ T cell comprising the CD19-targeted chimeric receptor, at least about 5% of a KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ T cell comprising the CD19-targeted chimeric receptor, at least about 5% of a CD57⁺ TIM3⁺ KLRG1⁻ CTLA4⁻ PD1⁺ T cell comprising the CD19-targeted chimeric receptor, at least about 5% of a CD57⁺ TIM3⁺ KLRG1⁻ LAG3⁻ PD1⁺ T cell comprising the CD19-targeted chimeric receptor, at least about 5% of a CD57⁺ TIM3⁺ CTLA4⁻ LAG3⁻ PD1⁺ T cell comprising the CD19-targeted chimeric receptor, at least about 5% of a CD57⁺ KLRG1⁻ CTLA4⁻ LAG3⁻ PD1⁺ T cell comprising the CD19-targeted chimeric receptor, at least about 5% of a TIM3⁺ KLRG1⁻ CTLA4⁻ LAG3⁻ PD1⁺ T cell comprising the CD19-targeted chimeric receptor, at least about 5% of a CD57⁺ TIM3⁺ KLRG1⁻ CTLA4⁻ LAG3⁻ PD1⁺ T cell comprising the CD19-targeted chimeric receptor, or a combination thereof.

25. The composition of claim 15, which is a pharmaceutical composition further comprising a pharmaceutically acceptable carrier.

26. The composition of claim 15, comprising between about 1×10⁶ and about 5×10⁸ cells, between about 1×10⁶ and about 1×10⁸ cells, between about 1×10⁶ and about 5×10⁷ cells, or about 2.5×10⁷ cells.

27. A method of reducing tumor burden in a subject, increasing or lengthening survival of a subject having a neoplasm, or treating and/or preventing a neoplasm in a subject comprising administering to the subject a composition of claim 15.

28. A method of reducing tumor burden, increasing or lengthening survival, and/or treating and/or preventing a neoplasm in a subject that is non-responsive or has a reduced likelihood to respond to an adoptive cell therapy, comprising:
(a) measuring the expression level of at least one of CD57, TIM3, KLRG1, CTLA4, LAG3 and PD1 in an immunoresponsive cell of a subject;
(b) comparing the expression level to a reference sample;
(c) identifying the subject as non-responsive or as having a reduced likelihood to respond to an adoptive cell therapy if
(i) the expression level of CD57 is reduced compared to the reference sample,
(ii) the expression level of TIM3 is reduced compared to the reference sample,
(iii) the expression level of KLRG1 is increased compared to the reference sample,
(iv) the expression level of CTLA4 is increased compared to the reference sample,
(v) the expression level of LAG3 is increased compared to the reference sample, and/or
(vi) the expression level of PD1 is reduced compared to the reference sample; and
(d) administering to the subject a composition of claim 15.

29. The method of claim 28, wherein the tumor and/or neoplasm is associated with CD19.

30. The method of claim 28, wherein the tumor and/or neoplasm is a blood cancer.

31. The method of claim 30, wherein the blood cancer is selected from the group consisting of multiple myeloma, leukemia, and lymphomas.

32. The method of claim 30, wherein the tumor and/or neoplasm is a B cell malignancy or a B cell lymphoma.

33. The method of claim 32, wherein
(a) the B cell malignancy is selected from the group consisting of B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma; or
(b) the B cell lymphoma is relapsed or refractory (R/R) B cell lymphoma.

34. The method of claim 30, wherein the subject is a human subject.

35. A kit for performing the method of claim 1.


SEQUENCE LISTING (48 sequences)

SEQ ID NO: 1 - protein, 19 aa, synthetic construct
GGGGSGGGGSGGGSGGGGS

SEQ ID NO: 2 - protein, 15 aa, synthetic construct
GGGGSGGGGSGGGGS

SEQ ID NO: 3 - protein, 24 aa, synthetic construct
GGGGSGGGGSGGGGSGGGSGGGGS

SEQ ID NO: 4 - protein, 29 aa, synthetic construct
GGGGSGGGGSGGGGSGGGGSGGGSGGGGS

SEQ ID NO: 5 - protein, 5 aa, synthetic construct
GGGGS

SEQ ID NO: 6 - protein, 10 aa, synthetic construct
GGGGSGGGGS

SEQ ID NO: 7 - protein, 18 aa, synthetic construct
ASTGGGGSGGGGSGGGGS

SEQ ID NO: 8 - protein, 557 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLAGSQSYEDMRGILYAAPQLRSIRGQPGPNHEEDADSYENMDNPDGPDPAWGGGGRMGTWSTR

SEQ ID NO: 9 - protein, 556 aa, Homo sapiens [sequence omitted]

SEQ ID NO: 10 - protein, 5 aa, synthetic construct
SYGMH

SEQ ID NO: 11 - protein, 17 aa, synthetic construct
LIWYDGSNKYYADSVKG

SEQ ID NO: 12 - protein, 11 aa, synthetic construct
PVEGLLRGFDY

SEQ ID NO: 13 - protein, 12 aa, synthetic construct
RASQSVSSSYLA

SEQ ID NO: 14 - protein, 7 aa, synthetic construct
GASSRAT

SEQ ID NO: 15 - protein, 9 aa, synthetic construct
QQAGAVPIT

SEQ ID NO: 16 - protein, 120 aa, synthetic construct
QVQLVESGGGVVQPGRSLRLSCAASGFTFSSYGMHWVRQAPGKGLEWVALIWYDGSNKYYADSVKGRFTISRDNSKNTLYLQMNSLRAEDTAVYYCAKPVEGLLRGFDYWGQGTLVTVSS

SEQ ID NO: 17 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAGAVPITFGGGTKVEIK

SEQ ID NO: 18 - protein, 243 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAGAVPITFGGGTKVEIKGGGGSGGGGSGGGGSQVQLVESGGGVVQPGRSLRLSCAASGFTFSSYGMHWVRQAPGKGLEWVALIWYDGSNKYYADSVKGRFTISRDNSKNTLYLQMNSLRAEDTAVYYCAKPVEGLLRGFDYWGQGTLVTVSS

SEQ ID NO: 19 - protein, 22 aa, synthetic construct
MDMRVPAQLLGLLLLWLPDTRC

SEQ ID NO: 20 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 21 - DNA, 213 bp, synthetic construct
  source 1..213: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 22 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 23 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 24 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 25 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 26 - protein, 117 aa, synthetic construct
ATTGAAGTTATGTATCCTCCTCCTTACCTAGACAATGAGAAGAGCAATGGAACCATTATCCATGTGAAAGGGAAACACCTTTGTCCAAGTCCCCTATTTCCCGGACCTTCTAAGCCC

SEQ ID NO: 27 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 22 aa, synthetic construct
QNQLYNELNLGRREEYDVLDKR

SEQ ID NO: 29 - DNA, 66 bp, synthetic construct
  source 1..66: mol_type = other DNA
cagaaccagctctataacgagctcaatctaggacgaagagaggagtacgatgttttggacaagaga

SEQ ID NO: 30 - protein, 22 aa, synthetic construct
QNQLFNELNLGRREEFDVLDKR

SEQ ID NO: 31 - DNA, 66 bp, synthetic construct
  source 1..66: mol_type = other DNA
cagaaccagctctttaacgagctcaatctaggacgaagagaggagttcgatgttttggacaagaga

SEQ ID NO: 32 - protein, 23 aa, synthetic construct
QEGLYNELQKDKMAEAYSEIGMK

SEQ ID NO: 33 - DNA, 69 bp, synthetic construct
  source 1..69: mol_type = other DNA
caggaaggcctgtacaatgaactgcagaaagataagatggcggaggcctacagtgagattgggatgaaa

SEQ ID NO: 34 - protein, 23 aa, synthetic construct
QEGLFNELQKDKMAEAFSEIGMK

SEQ ID NO: 35 - DNA, 69 bp, synthetic construct
  source 1..69: mol_type = other DNA
caggaaggcctgttcaatgaactgcagaaagataagatggcggaggccttcagtgagattgggatgaaa

SEQ ID NO: 36 - protein, 22 aa, synthetic construct
HDGLYQGLSTATKDTYDALHMQ

SEQ ID NO: 37 - DNA, 66 bp, synthetic construct
  source 1..66: mol_type = other DNA
cacgatggcctttaccagggtctcagtacagccaccaaggacacctacgacgcccttcacatgcag

SEQ ID NO: 38 - protein, 22 aa, synthetic construct
HDGLFQGLSTATKDTFDALHMQ

SEQ ID NO: 39 - DNA, 66 bp, synthetic construct
  source 1..66: mol_type = other DNA
cacgatggccttttccagggtctcagtacagccaccaaggacaccttcgacgcccttcacatgcag

SEQ ID NO: 40 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 41 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 42 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 43 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 44 - DNA, 126 bp, synthetic construct
  source 1..126: mol_type = other DNA
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 45 - protein, 488 aa, synthetic construct
MDMRVPAQLLGLLLLWLPDTRCEIVLTQSPGTLSLSPGERATLSCRASQSVSSSYLAWYQQKPGQAPRLLIYGASSRATGIPDRFSGSGSGTDFTLTISRLEPEDFAVYYCQQAGAVPITFGGGTKVEIKGGGGSGGGGSGGGGSQVQLVESGGGVVQPGRSLRLSCAASGFTFSSYGMHWVRQAPGKGLEWVALIWYDGSNKYYADSVKGRFTISRDNSKNTLYLQMNSLRAEDTAVYYCAKPVEGLLRGFDYWGQGTLVTVSSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 46 - DNA, 1464 bp, synthetic construct
  source 1..1464: mol_type = other DNA
atggatatgagagtaccagctcagctgctgggcctgctgcttttgtggttgccggacacacgctgtgagattgtcctgactcagtctcccgggactctgtccctcagccccggtgaacgcgctaccctttcatgcagagcctctcagtctgtgtccagcagctacctcgcatggtatcagcagaagcccggacaggctcccaggctgttgatctatggagctagtagtcgagcaacaggcatcccagatcgcttctcagggagcggttcaggtacagacttcacgctgacgatttcaaggctggaacccgaagattttgccgtctattattgtcaacaggcaggggctgtgccaatcactttcgggggcgggaccaaggtggaaatcaaaggaggcggaggaagtggaggaggagggagcggtggaggagggtcacaggtgcagctggtagaatctggcggaggggtcgttcaaccagggaggtcattgcggttgagctgcgcagcgagtggttttaccttcagcagttatggaatgcattgggtgagacaagcaccaggaaaaggtctggagtgggtggctttgatttggtacgacggcagtaataaatactacgccgattctgttaagggcagatttactatttctcgcgacaacagcaagaacacgctgtacctgcagatgaactctctgagagccgaagatacagcagtgtactattgtgctaagcccgtagaagggctcctgaggggattcgattattggggccagggtacgcttgtgacagtgtctagtcgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 47 - protein, 22 aa, synthetic construct
GSGATNFSLLKQAGDVEENPGP

SEQ ID NO: 48 - DNA, 66 bp, synthetic construct
  source 1..66: mol_type = other DNA
ggaagcggagctactaacttcagcctgctgaagcaggctggagacgtggaggagaaccctggaccc